(19) United States
(12) Patent Application Publication
(10) Pub. No.: US 2024/0142444 A1
(43) Pub. Date: May 2, 2024

(54) DIAGNOSTIC METHOD FOR INFECTIOUS DISEASES

(71) Applicant: GEORGE MASON UNIVERSITY, Fairfax, VA (US)

(72) Inventors: Lance LIOTTA, Fairfax, VA (US); Marissa A. HOWARD, Fairfax, VA (US); Alessandra Luchini KUNKEL, Fairfax, VA (US); Sameen YUSUF, Fairfax, VA (US); Sara SHARIF, Fairfax, VA (US); Ruben MAGNI, Fairfax, VA (US)

(21) Appl. No.: 17/622,403

(22) PCT Filed: Jun. 25, 2020
(86) PCT No.: PCT/US2020/039518

§ 371 Date: Dec. 23, 2021

Related U.S. Application Data
(60) Provisional application No. 62/866,287, filed on Jun. 25, 2019.

(51) Int. Cl. G01N 33/543; G01N 33/569

(52) U.S. Cl. CPC G01N 33/54346; G01N 33/56905; G01N 33/56911; G01N 33/5695; G01N 2333/20; G01N 2333/35

(57) ABSTRACT

The invention relates to a relates to a highly sensitive, non-invasive diagnostic method for detection infectious diseases. In one embodiment, the invention relates to novel rapid, self-working, visual field test for a panel of disease specific derived biomarkers. In one embodiment, the invention comprises a collecting device comprising a collapsible non hygroscopic net tethered with immobilized nanoparticles to capture and concentrate a target analyte present in a fluid; wherein the nanoparticle comprises a core and a shell, wherein the core comprises a molecular bait. The invention also relates to identification of biomarkers for identification of various pathogenic diseases.

### RELATED APPLICATION

The present application claims priority from U.S. provisional application No. 62/866,287 titled “DIAGNOSTIC PEPTIDES WITH ABSOLUTE SPECIFICITY FOR MYCOBACTERIUM TUBERCULOSIS AND TRYPANOSOMA CRUZI”, filed on Jun. 25, 2019.

### GOVERNMENT SUPPORT

This invention was made with government support under grant numbers 1 R21 AI138135-01A1, R21 HD097472-01, and 1 R01 A1136722-01A1 awarded by the National Institute of Health. The government has certain rights in the invention.

### SEQUENCE LISTING

The sequence listing associated with this application is provided in text format in lieu of a paper copy, and is hereby incorporated by reference into the specification. The name of the text file containing the sequence listing is GMUN-005-01US_Sequence_Listing.txt. The text file is 68.9 kb created on Dec. 25, 2023, and is being submitted electronically via EFS-Web.

### FIELD OF THE INVENTION

The invention relates to a highly sensitive, non-invasive diagnostic method for detection of infectious diseases. More specifically, the invention relates to a highly sensitive, multiplex urine test to attain specificity for Mycobacterium tuberculosis, Trypanosoma cruzi and Lyme disease.

The present invention relates to several novel peptide sequences and their variants that can be used as potential biomarker for detection and/or quantification of the disease in a subject.

The invention relates to novel rapid, self-working, visual field test for a panel of pathogen derived urinary biomarkers. In one embodiment, the invention relates to a method to identify peptide in mass spectrometry.

### BACKGROUND OF THE INVENTION

TB is one of the most important bacterial infections globally (9 million patients). WHO estimates that TB kills 1.8 million people yearly [1]. In 2015, 10.4 million people worldwide fell ill with tuberculosis. Of these, 4 million were never reported, diagnosed, or treated. Patients with undiagnosed active pulmonary TB can spread the disease to family and coworkers. The mortality rate for untreated TB is 68%, compared to 5% following treatment [1].

Childhood TB (0-15 yrs), a lethal disease if untreated, is underdiagnosed and undertreated, and is recognized as a modern public health emergency in countries with a high burden of TB. Exposure to an adult with active pulmonary TB increases the mortality by 70% in children under 5 yrs in high burden settings, and by eight-fold when the mother has TB. Since childhood TB reflects recent transmission, childhood infection burden is an accurate measure of TB disease control in a community, and infected children constitute the main reservoir for future cases.

Clinical evaluation of diseased patient such as TB at a reference hospital may include smear microscopy, mycobacterial culture, and nucleic acid amplification testing (GeneXpert), but the performance of these tests in children is very suboptimal due the paucibacillary nature of the infection and the inability of children to produce sputum. Gastric Aspirates (GA) of children are an invasive procedure with a sensitivity of only 30-50%, compared to ˜80% in adults, and can take 4-6 weeks.

Consequently, a reliable highly sensitive diagnostic screening test for active pulmonary tuberculosis (TB), that can be conducted in the field, in rural settings, in the home and in neighborhood clinics, in underdeveloped global regions is critically important for disease control [2, 3]. Since urine can be easily collected non-invasively, it is an ideal biofluid to detect TB antigens [4-6].

Unfortunately, TB urine testing has been hampered in the past because TB antigens exist in very low concentration, are masked by high abundant urinary resident proteins, and are subject to rapid degradation. Because past methods for protein discovery by mass spectrometry (MS) lack adequate sensitivity, the repertoire of TB antigens shed into the urine of patients with active disease is completely unknown. Commercial tests that screen for the presence of mannose-capped lipoarabinomannan (LAM), a lipoglycan thought to be essential for the virulence of TB [7, 8] lack adequate sensitivity for pulmonary TB, and cannot be used for HIV negative (85% of TB patients) population screening [4]. Urine PCR testing for TB has 50% sensitivity for detecting TB in HIV positive patients and can lacks adequate precision [9].

Chagas disease (ChD) is caused by Trypanosoma cruzi (T. cruzi) infection and is responsible for high mortality and morbidity among the world's poorest populations. 6 million to 7 million people worldwide are infected with the parasite. The disease is endemic in 21 countries of Latin America, where it causes more deaths than malaria, but can remain asymptomatic for many years. Chronic symptomatic disease, which can be fatal, develops in up to 30% of cases. The infection is transmissible via blood and organ donation and congenitally. In 2006, 1 in 23,000 blood donations tested positive for T. cruzi. Congenital transmission accounts for 25% of new infections with an estimate of 15,000 infected infants per year.

The prevalence of ChD in Latin American immigrants to the US can be as high as 5-10% depending on the geographical areas of origin. Prince Williams County has been the first county in the US where a congenital transmission of Chagas disease was reported. Mathematical models and studies in Europe and Switzerland support the economic benefit of a universal screening for ChD in immigrant populations. The earlier in life congenital infection is detected, the higher the efficacy and tolerability of treatment.

Detecting T. cruzi infection in the asymptomatic phase dramatically increases efficacy and tolerability of treatment, while therapeutic efficacy declines after disease progression to irreversible tissue damage. It is estimated that the number of T. cruzi infected patients in the United States surpasses 230,000. Nevertheless, lack of surveillance in the US population impedes a clear understanding of the reach of the problem and hampers effective management and treatment of affected patients. Under-diagnosis is motivated by 1) lack of health care provider awareness of the risk of ChD, 2) lack of awareness in the population, 3) T. cruzi high strain diversity, and 4) poor performance of current serological test for strains deriving from Central America and Mexico. High burden of disease is demonstrated in Hispanic non-US born populations and residents along the US-Mexico border.

It is predicted that the global tuberculosis testing market will reach $2.2 billion by 2020. In the US, 2.2 million tuberculosis tests are performed on foreign born individuals who seek green card or naturalization status. The number of people living with Chagas disease in the US is estimated to be 300,000. The global market for Chagas disease diagnosis is predicted to reach $400 million by 2025. The Tuberculosis and Chagas disease are expanding in the US and a non-invasive, accurate antigen test is not available. For selected categories of patients, including infants newborns, immunocompromised and elderly populations, the current detection systems lack sensitivity and specificity.

Similarly, with an estimate of 300,000 cases per year in the US, Lyme borreliosis is the most common vector-borne infection in North America. Despite the incidence of tick-borne infections and the enormous suffering they cause, progress in accurate diagnosis and durable treatment regimens has been greatly hindered by questions surrounding: a) the cause of persistent post-treatment Lyme symptoms, and, b) the prevalence and medical significance of coinfections by two or more tick-borne pathogens.

Post Treatment Lyme Disease Syndrome (PTLDS) defines a subset of patients who continue to experience a variety of symptoms such as joint pain, fever, neurologic impairment, neuropathy, fatigue, and depression^{4,13} following antibiotic therapy for Lyme disease. The cause of the persistent symptoms in PTLDS is unknown^{4,6,14}. Direct molecular evidence is lacking to verify that the symptoms are caused by persistence of an active tick-borne pathogen infection¹⁵, their persistence has been attributed either to immunologic and inflammatory phenomena that are triggered after a successfully treated infection, or to illnesses not associated with a tick-borne infection¹⁶. Recent molecular evidence suggested that post treatment persistence of Lyme arthritis symptoms maybe influenced by the persistence of Borrelia peptidoglycans in synovial fluid¹³⁶. Whether these biomolecules are derived from viable pathogens or persist in the body long after the infection has resolved remains to be determined.

Competing technologies for disease diagnosis are sputum culture, sputum smear microscopy, PCR based direct test on sputum, interferon releasing assay, skin test. Competing technologies for Chagas disease are blood smear microscopy, serology, and PCR based direct tests. Current screening programs have low sensitivity, and high cost, especially in infants, newborns, immunocompromised and elderly populations.

Current screening programs have very low sensitivity, high cost, requires trained personnel. A sensitive, specific and field-friendly screening test is urgently needed for effective disease screening in population. Thus there is a great need to make high specificity disease screening.

The present road blocks to urine screening for pulmonary TB, Chagas, Lyme or any similar diseases:

Low abundance (less than 1 ng/mL) of disease derived protein and glycan analytes shed into urine. No molecular discovery of novel antigens shed by pathogens in the urine of patients with active disease: past studies have been limited to a very few known pathogen antigens shed in vitro in microbiologic culture. Pathogen antigen shed in vivo from the human tissue microenvironment may be different than those shed in culture and may vary with disease stage or treatment status. Difficulty, cost, and hazard, of shipping and refrigerating urine collected in specimen containers. Geographically dispersed patients are reluctant or unable to travel to a clinic or hospital for screening.

Very low sensitivity of past disease antigen immunoassays, lateral flow assays, or PCR tests.

Failure to diagnose TB and ChD by testing the urine of HIV negative pulmonary TB and ChD positive patients.

Lack of trained individuals who can reliably conduct point of care community based diagnostic instruments, even if the technology used can be low cost and demonstrate adequate sensitivity.

Prior attempts to collect small volumes of urine on a card [24] can not physically contain enough analyte molecules to achieve sensitivity lower than 1 ug/mL. Urine TB and ChD analytes exist in an expected concentration less than one nanogram per mL [17].

### SUMMARY OF THE INVENTION

Nanotechnology harvests and concentrates analytes, making it possible for the first time to collect, discover, and measure low abundance TB derived proteins in urine for population screening.

In one embodiment, the present application relates to nanoparticles comprising a core and a shell, wherein the core comprises a molecular bait, and wherein the nanoparticle is configured to capture, concentrate and preserve a biomolecule, a nucleic acid, an exosome, and/or a virus.

In one embodiment, the nanoparticle is functionalized with the molecular bait, wherein the molecular bait captures target analytes in a solution displacing unwanted contaminating carrier protein.

In one embodiment, the capture of the biomolecule, the nucleic acid, an exosome, and/or the virus is from urine, blood, and/or saliva.

In one embodiment, the capture is achieved by sequestering target analytes from a whole volume of the urine, the blood, and/or the saliva and bringing the target analytes into a small volume within the nanoparticle.

In one embodiment, the core of the nanoparticle has surface area at least 1000 times greater than surface area of the shell of the nanoparticle.

In one embodiment, the nanoparticles are an open mesh, non-aggregating, colloidal and >95% open void.

In one embodiment, the nanoparticles are immobilized on a collapsible non hygroscopic net such that the target analyte is preserved in a dry state.

In one embodiment, a collection device comprising the collapsible non hygroscopic net is configured to collect a fluid sample such that the fluid sample is in concentrated and dried state.

In one embodiment, the fluid sample is urine, blood, and/or saliva.

In one embodiment, an enzyme is immobilized with the nanoparticles, wherein the enzyme is configured to produce an enzymatically amplified color reaction inside the nanoparticles containing the target analyte.

In one embodiment, the nanoparticle is configured for a visual lateral flow identification of the target analyte.

In one embodiment, the target analyte captured by the nanoparticle is configured to be displayed on a solid phase antibody for production of the enzymatically amplified color reaction inside the nanoparticles.

In one embodiment, the nanoparticles has sensitivity of about 95% and specificity about 80% of the target analytes from the whole volume of the urine, the blood, and/or the saliva. In an embodiment, the whole volume is defined as a total volume of the sample. Sensitivity (also called the true positive rate, the epidemiological/clinical sensitivity, the recall, or probability of detection in some fields) measures the proportion of actual positives that are correctly identified as such (e.g., the percentage of sick people who are correctly identified as having the condition). The higher the numerical value of sensitivity, the less likely diagnostic test returns false-positive results. For example, if sensitivity=95%, it means: when we conduct a diagnostic test on a patient with certain disease, there is 95% of chance, this patient will be identified as positive. Specificity (also called the true negative rate) measures the proportion of actual negatives that are correctly identified as such (e.g., the percentage of healthy people who are correctly identified as not having the condition).

In one embodiment, a method of testing for a bacterial and/or viral infectious disease comprising capturing, concentrating and/or preserving a biomolecule, a nucleic acid, an exosome, and/or a virus in a nanoparticle, wherein the nanoparticle comprises a core and a shell, wherein the core comprises a molecular bait, and wherein the nanoparticle is configured to capture, concentrate and preserve the biomolecule, the nucleic acid, the exosome, and/or the virus.

In one embodiment, the nanoparticle of the method is functionalized with the molecular bait, wherein the molecular bait captures target analytes in a solution displacing unwanted contaminating carrier protein.

In one embodiment, in the method the capturing of the biomolecule, the nucleic acid, an exosome, and/or the virus is from urine, blood, and/or saliva.

In one embodiment, in the method the capturing is achieved by sequestering target analytes from a whole volume of the urine, the blood, and/or the saliva and bringing the target analytes into a small volume within the nanoparticle.

In one embodiment, a method comprises: a) fabricating nanoparticles comprising a core and a shell, wherein the core comprises a molecular bait, and wherein the nanoparticle is configured to capture, concentrate and preserve the biomolecule, the nucleic acid, the exosome, and/or the virus; b) immobilizing the nanoparticle on a collapsible non hygroscopic net; c) collecting a fluid sample within a collecting device comprising the collapsible non hygroscopic net of step (b); d) sequestering target analytes from a whole volume of the fluid sample and bringing the target analytes into a small volume within the nanoparticles; e) analyzing target analytes present in the fluid sample.

In one embodiment, in the method the nanoparticles comprises an immobilized enzyme, wherein the immobilized enzyme is configured to produce an enzymatically amplified color reaction inside the nanoparticles containing the target analyte.

In one embodiment, in the method the nanoparticle is configured for a visual lateral flow identification of the target analyte.

In one embodiment, in the method the target analyte captured by the nanoparticle is configured to be displayed on a solid phase antibody for production of the enzymatically amplified color reaction inside the nanoparticles.

In one embodiment, in the method the nanoparticle is functionalized with the molecular bait, wherein the molecular bait captures target analytes in a solution displacing unwanted contaminating carrier proteins.

In one embodiment, in the method the fluid sample is urine, blood, and/or saliva of a subject.

In one embodiment, in the method the nanoparticles are an open mesh, non-aggregating, colloidal and >95% open void.

In one embodiment, in the method the nanoparticles has sensitivity of about 95% and specificity about 80% of the target analytes from the whole volume of the urine, the blood, and/or the saliva.

In one embodiment, a biomarker for identification of chagas disease comprises a peptide comprising an amino acid sequence selected from the group consisting of SEQ ID No. 1 to SEQ ID No. 229 and/or SEQ ID No. 269 to SEQ ID No. 286.

In one embodiment, a biomarker for identification of TB disease comprises a peptide comprising an amino acid sequence selected from the group consisting of SEQ ID No. 230 to SEQ ID No. 268 and/or SEQ ID No. 287 to SEQ ID No. 312.

In one embodiment, a biomarker for identification of lyme disease comprises a peptide comprising an amino acid sequence selected from the group consisting of SEQ ID No. 322 to SEQ ID No. 345.

In one embodiment, a collecting device comprises a collapsible non hygroscopic net immobilized with nanoparticles comprising a core and a shell, wherein the core comprises a molecular bait, and wherein the nanoparticle is configured to capture, concentrate and preserve the biomolecule, the nucleic acid, the exosome, and/or the virus; wherein the collecting device is configured to collect a fluid sample such that the fluid sample is in concentrated and dried state.

In one embodiment, the collection vessel collects the fluid sample is urine, blood, and/or saliva.

In one embodiment, in the collection vessel the capture is achieved by sequestering target analytes from a whole volume of the urine, the blood, and/or the saliva and bringing the target analytes into a small volume within the nanoparticle.

In one embodiment, in the collection vessel the nanoparticle comprises an immobilized enzyme configured to produce an enzymatically amplified color reaction inside the nanoparticles containing the target analytes.

In one embodiment, in the collection vessel the nanoparticle is configured for a visual lateral flow identification of the target analyte.

In one embodiment, in the collection vessel the target analyte captured by the nanoparticle is configured to be displayed on a solid phase antibody for production of the enzymatically amplified color reaction inside the nanoparticles.

In one embodiment, in the collection vessel the target analyte is configured to be analyzed using antibody free techniques and/or antibody-based techniques.

In one embodiment, in the collection vessel the target analyte is configured to be analyzed using mass spectrometry.

In one embodiment, in the method the target analyte is configured to be analyzed using antibody free techniques and/or antibody-based techniques.

In one embodiment, the present technology is a very low-cost technology, is that urine sampling can be done in the field, home, or primary clinic without the requirement to handle, transport, and refrigerate, liquid urine. This reduces delay time for diagnosis and wrongful treatment.

In one embodiment, in the present technology the urine collection and one step processing requires no electricity or expensive equipment, is disposable, and can be handled by untrained individuals.

In one embodiment, the present technology can be manufactured and implement the technology directly in-country, if required, to provide unrestrained access to the technology.

In one embodiment, the present innovation that the urine of patients with TB contains previously unknown peptides of proteins known to be associated with TB specific drug resistant mechanisms studied in TB cultures, or expected to be encoded by proteins associated with TB drug resistance associated genes.

Moreover, the present technology of nanoparticle-enhanced mass spectrometry method is for cardiomyopathy patients in endemic areas to identify biomarkers for cardiac symptoms.

In one embodiment, the peptides could also help with developing potential new vaccines.

In one embodiment, the goal of this study is to introduce a method for investigating candidate pathogen specific peptides in patients diagnosed with acute Borreliosis or suspected of tick-borne illnesses including Borreliosis, Babesiosis Anaplasmosis, Ehrlichiosis, Tick-borne encephalitis virus, Powassan Virus disease, Rickettsiosis, TBRF, Toxoplasma gondii and Tularemia, Mycobacterium tuberculosis, Toxoplasma gondii, and Trypanosoma cruzi, and Babesia microti. In one embodiment, the combined hydrogel particle pre-processing with a highly sensitive immunoassay to detect OspA, a relevant biomarker for Lyme borreliosis.

The analytical sensitivity of MS analysis is currently in the range of 10-100 ng/mL when analyzing complex matrices without pre-analytical processing, hence mass spectrometry analysis applied directly to body fluid samples lacks the sensitivity needed for low abundance pathogen derived protein detection.

In one embodiment, the pre-processing of the sample with affinity hydrogel particles concentrates the low abundance biomarkers to achieve mass spectrometry sensitivity in the low picogram/mL range. Additionally, the present method ensures linearity and precision of the assay in physiologically relevant protein concentration ranges.

In one embodiment, Affinity hydrogel particles consist of polymeric networks functionalized with high affinity chemical baits that capture, concentrate, and preserve solution phase analytes in one step, while excluding interfering high abundance proteins (Supplementary Methods) that would otherwise negatively affect the analytical sensitivity of mass spectrometry analysis.

### BRIEF DESCRIPTION OF THE DRAWINGS

FIG. 1 shows summary of the Invention. Under Aim 1.1 the technology is applied to measure LAM and ESAT6 in the urine of patients with TB and correlate the concentration levels with disease severity. Under Aim 1.2 discover and validate previously unknown urinary proteins shed by TB organisms, that provide fresh insights for diagnosis and treatment. Under Aim 2 create a novel collapsible urine collection cup for population screening and banking. Under Aim 3 optimize and validate a an enzymatically amplified one step visual lateral flow urine test that achieves a sensitivity 100 times greater than previous lateral flow technologies.

FIG. 2 shows road map for pathogenic disease such as TB and Chagas testing in urine. Upper left. Tuberculosis and ChD derived biomarkers are shed in the blood circulation, filtered through glomeruli filtration in the kidneys, and concentrated in the bladder. Upper right. Open cage nanocage is functionalized with high affinity bait that captures target analytes in solution displacing unwanted contaminating carrier proteins. Atomic force microscopy and scanning electron microscope images of Nanoparticles. Lower Left. Concentration for sensitivity enhancement is achieved by sequestering all of the target analytes from the whole volume of urine and concentrating them in a small volume for downstream analysis. Lower right. Mass spectrometry (MS) analysis of Mtb and T. cruzi peptides harvested from patient urine.

FIG. 3 shows Origami collection cup obviates the need for handling or shipping fluid urine for molecular diagnostic testing. Affinity capture nanoparticles are tethered to strands of glass wool fiber net that fills the volume of urine in the cup. When the urine is dumped out the net can be compressed flat for fluid-free mailing.

FIG. 4 shows Images of a working prototype of the Urigami device.

FIG. 5 shows User collection steps: 1) User urinates into the Urigami collection device containing the nanotechnology; an adhesive urine collection bag (denoted with *) will be used for infants. 2) User dumps out the urine after 1 minute and collapses the device flat. The analytes are captured and preserved in a dry state for simple mailing in a sealed flat confidential envelope;

FIG. 6 shows Novel rapid, self-working, visual field test for a panel of three tuberculosis derived urinary biomarkers. A affinity capture nanoparticle tethered glass fibers in a dry state. B submerged affinity net expands to occupy all fluid. C D, Urigami collapsible cup filled with glass wool. Plugging in the visual test compresses and activates the affinity net; biomarkers are eluted by release of extraction buffer contained in the tip. This prototype is configured to measure three biomarkers at once, radially.

FIG. 7 (A) shows Scanning EM of 0.6 micron nanoparticles coating glass fibers. (B) Volume reduction attained by the collapsible cup.

FIG. 8 shows manufacturing scheme for in country production of collection device and affinity net.

FIG. 9 shows Scheme for covalently attaching nanocages to aminosilanized glass wool fibers using 4,4′-Azobis(4-cyanovaleric acid) (blue chemical structure). Insert: glass wool before and after covalent functionalization with affinity hydrogel nanoparticles.

FIG. 10 shows (Top) Bioinformatics pipeline to authenticate protein annotation and peptide specificity. (Bottom) using taxonometric similarity of peptides for data visualization and authentication.

FIG. 11 shows number and cellular location of unique, pathogen derived proteins that were detected in the urine of infected patients, using affinity nanocapture, mass spectrometry analysis and bioinformatics authentication.

FIG. 12 shows Flow chart of the proprietary peptide authentication software.

FIG. 13 shows peptides derived from proteins associated with mechanism of drug resistance were detected in the urine of patients with culture proven drug resistance.

FIG. 14 shows Mtb proteins identified in the urine of active TB patients.

FIG. 15 shows TB antigen binding nanocages are seamlessly integrated into a novel lateral flow immunoassay for Tuberculosis rapid diagnostics. Nanocages capture urinary pathogen antigens and arrest on the antibody line. Horse radish peroxidase HRB mediated enzymatic amplification and large (1 mL) volume of sampled urine enhance the analytical sensitivity >100 fold if compared to conventional lateral flow immunoassays (target=1.25 pg/mL).

FIG. 16 shows scheme for covalently attaching nanocages to aminosilanized glass wool fibers. 4,4′-Azobis(4-cyanovaleric acid) (blue chemical structure) can be cleaved using heat 80° C.

FIG. 17 shows validation of 3D affinity net capturing 0.25 ng tuberculosis biomarker, ESAT-6, in PBS and Urine. PE: eluate in PBS. PS: supernatant in PBS. UE: eluate in human urine, US: supernatant in human urine.

FIG. 18 shows partially dissolvable nanocages captured antigen for antibody binding in a high sensitivity sandwich immunoassay. A) Significant change in hydrodynamic diameter after Nanocage oxidation. B) Oxidation of DHEA degradable cross-linker allows antibody entry (S=supernatant, E=eluate, lanes 2 and 3). Antibody is excluded before crosslink oxidation (lanes 4 and 5). C) Immuno macroarray demonstrating antigen bound to the chemical bait retains its capability to bind to the antibody. D) Anti-LAM antibody binds to LAM captured in the inner volume of nanocages (after pore dilation) 40 times higher than LAM bound to the cage surface (before pore dilation). E) and F) High affinity chemical baits improve sensitivity for antigen capture compared to conventional antibody. Eq 1 parameters: Top=1, Bottom=0.05, EC50=1e−12M, slope=0.7, G=1e−15M.

FIG. 19 shows HRP encapsulated in the inner volume of the cages retains full enzymatic activity, and when triggered with glucose oxidase, achieves a much higher sensitivity and dynamic range compared to the same number of HRP molecules in solution (A, B, C) Enzymatic amplification in the internal volume of the nanocages reaches a plateau at 30 seconds and enhances the signal 1000%. Urine containing ESAT6 yielded a dark detectable band on the test strip. The negative test result demonstrates that the nanoparticles produced no visually detectable line.

FIG. 20 shows Nanoparticle enriched urinary LAM test achieves 0.95 sensitivity and 0.80 specificity in TB positive, HIV negative patients and urinary LAM levels correlate with measures of disease severity. A. A novel copper containing dye, reactive blue 221 (RB221) displays very high binding affinity for LAM. B. RB221 functionalized nanoparticles capture all solution phase LAM in urine and attain a concentration yield >95%. C, D, E. CS-35 anti-LAM mAb does not cross-react with purified polysaccharides from N. meningitidis and S. pneumoniae. CS-35 was tested against purified polysaccharides deriving from four serougroups of N. meningitidis (spots 1 to 4 in C) and 13 serogroups of S. pneumoniae (spots 5-17 in C). F. Competition assay demonstrates that mAb clone CS-35 specific for LAM in urine. G-J Boxplot of urinary LAM in TB cases and controls, correlation of urine LAM with mycobacterial burden obtained from sputum microscopy, ROC plot of urine data, Correlation of urine LAM with weight loss. K. Bar plot of the intensities of LAM determined via immuno macroarray and ImageJ analysis for the pretreatment TB positive HIV negative patients.

FIG. 21 shows Examples of mass spectrometry spectra documenting the high quality and specificity of the data.

FIG. 22 shows 100% identity obtained with blast alignment of Mtb peptides found in the urine of TB patients with Mtb sequences retrieved from the Mtb NCBI database.

FIG. 23 shows Venn diagram shows partial overlap between adult and pediatric Mtb derived urinary markers.

FIG. 24 shows Tandem mass spectrometry analysis reveals ESAT6 from Mycobacterium tuberculosis in the urine of HIV negative, pediatric tuberculosis patients. Bottom. Nanocage enriched dot blot analysis reaches an analytical sensitivity of 0.05 pg/mL. CBBA denotes an abbreviation of a hospital name. Microbiologically confirmed tuberculosis patients are positive for urinary ESAT6.

FIG. 25 shows (B) Dose response using the affinity net technology of FIG. 2. Visual Sensitivity detection achieved is in the attogram per milliliter range after background subtraction. T=test, C=control. (C) Dose response sensitivity of 10 pg/mL for ESAT6 detection in urine using the affinity net technology of FIG. 2. (D) Mass spectrometry independent verification of ESAT6 in urine captured by the affinity net.

FIG. 26 shows Copper containing dyes have anti-bacterial properties.

FIG. 27 shows Proteins captured by the nanoparticles are protected from enzymatic degradation.

FIG. 28 shows Peptide identification and authentication algorithm.

FIG. 29 shows General Workflow: Concentration of T. cruzi derived analytes in urine and Mass Spectrometry sample preparation.

FIG. 30 shows Novel T. cruzi derived urinary peptides identified in the urine of congenitally infected CD newborn babies. Peptides were unambiguously attributed to proteins that are essential to T. cruzi viability and virulence.

FIG. 31 shows Summary of patient urine sample results. Out of the 16 positive samples, 100% had at least 1 unique peptide detected in the urine, 68.8% had 2 or more peptides, 50% had greater than 6, and 37.5% had 7 peptides or more.

FIG. 32 shows GPI-anchored trans-sialidase (TcTS) function in sialic acid transfer and host cell apoptosis.

FIG. 33 shows predicted retrotransposon hot spot protein (RHSP) structure using SWISS-MODEL protein structure homology-modelling server⁹ (GMQE=0.07, GMEAN=−3.37, Seq Identity=11.76%).

FIG. 34 shows A) A novel chemical bait has high affinity for glycans. B) Nanocage pre-processing increases the detection limit of western blot analysis for LPG 100 fold (5 mL initial urine volume). C) Nanocage test sensitivity and specificity for congenital Chagas.

FIG. 35 shows Experimental design for pathogen derived, urinary peptide identification and authentication. Experimental analysis and bioinformatics pipeline was applied to a first set of n=110 individuals, n=10 acute Lyme borreliosis patients according to CDC criteria and n=100 negative controls, comprising healthy and diseased non-Lyme participants. Parameters for peptide identification and authentications were established in this first phase. The method was verified in an independent set of n=298 participants, including n=148 non acute patients suspected of tick-borne illness and n=150 healthy and diseased controls. Urinary peptides were validated by means of orthogonal methods including western blot analysis, parallel reaction monitoring, and a Babesia animal model. PTLDS=post treatment Lyme disease syndrome.

FIG. 36 shows Borrelia and Babesia peptides. (A) Borrelia peptides (160) identified in patients affected by acute Lyme borreliosis, post treatment Lyme disease syndrome or suspected of tick-borne illnesses. Proteins marked with a * were unambiguously (100% sequence identity with the pathogen with less than 90% homology with human and other organisms) attributed to the following species: Borrelia hermsii, Borrelia turicatae, Borrelia duttonii, Borrelia miyamotoi, Borrelia recurrentis. Unmarked proteins were attributed to the following species: Borrelia burgdorferi, Borrelia mayonii, Borrelia garinii, Borrelia afzelii, Borrelia bavariensis, Borrelia spielmani. (B) Babesia peptides (n=62) identified in patients diagnosed with post treatment Lyme disease syndrome. 37/148 non acute patients had at least one Babesia urinary peptide, and 17/148 non acute patients had two or more peptides.

FIG. 37 shows Pathogen and peptide distribution. (A) Pathogen peptides were found in the urine of 40% of non-acute tick borne disease patients (cut off >2 peptides per organism). 32% of patients had urinary peptides deriving from 1 pathogen. 7% of patients presented peptides from two pathogens and less than 1% from three pathogens. (B) Borrelia peptides were found in 48/148 patients, Babesia peptides in 17/148, Ehrlichia peptides in in 2/148, Anaplasma peptides in 1/148, and Bartonella peptides 4/148. (C) OspC was the most represented protein in the peptide repertoire derived from acute Lyme borreliosis and non-acute tick-borne disease patients. While 109 peptides were identified once, multiple peptides were identified for Outer surface protein C, Variable large protein, Transcription elongation factor GreA, UTP-glucose-1-phosphate uridyly transferase (15, 4, 4, 3, and 3 peptide hits, respectively).

FIG. 38 shows MS/MS findings verification. (A) Parallel reaction monitoring (PRM) verification of a peptide deriving from Outer Surface Protein A (OspA). OspA peptide AVEIKTLDELK (SEQ ID NO. 345) was found in the discovery phase and confirmed by PRM in 2 non acute patient samples. (B) The presence of OspC (samples 107214, 108369, 108350), OspA (samples 108319, 108028) and Flagellin (sample 109126) was confirmed in by means of western blot analysis (MW=molecular weight; PC=positive control; NC=negative control). Positive bands were found at 31 kDa, 27 kDa, and 18 kDa for OspA, OspC and Flagellin respectively. Since the positive control used in the western blot for flagellin is a flagellin—maltose-binding protein fusion protein, multiple bands above 40 kDa were detected (right image, PC lane). Patient 109126 yielded a band reactive to the anti-flagellin antibody at a lower molecular weight than the expected band at 34 kDa for the full length protein. This might be due to a truncated form of the protein. OspA blot: blots were cropped from different parts of the gel. (C) Babesia peptides were identified in the blood and urine of a hamster animal model at early33 and late stages of disease with high sensitivity. (D) Verification of MS analysis results of two surface/secreted B. microti antigens, BmSA1 and BMR1_03g00947, by Western blotting of hamster RBC lysates.

### DETAILED DESCRIPTION

The practice of the present invention will employ, unless otherwise indicated, conventional techniques of nanotechnology, nano-engineering, molecular biology (including recombinant techniques), microbiology, cell biology, biochemistry, immunology, and pharmacology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); and Remington, The Science and Practice of Pharmacy, 22^{th} ed., (Pharmaceutical Press and Philadelphia College of Pharmacy at University of the Sciences 2012).

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

To facilitate understanding of the invention, a number of terms and abbreviations as used herein are defined below as follows:

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

As defined herein, “approximately”, “about” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Nanoparticle: The term “nanoparticle” as used herein refers to nanostructure, particles, vesicles, or fragments thereof having at least one dimension (e.g., height, length, width, or diameter) of between about 1 nm and about 10 μm. For systemic use, an average diameter of about 50 nm to about 500 nm, or 100 nm to 250 nm may be preferred. The term “nanostructure”, “nanocages’, “nanocage” includes, but is not necessarily limited to, particles and engineered features. The particles and engineered features can have, for example, a regular or irregular shape. Such particles are also referred to as nanoparticles. The layer of nanoparticles can be implemented with nanoparticles in a monolayer or with a layer having agglomerations of nanoparticles. In some embodiments, the nanoparticle comprising or consisting an inner core covered by an outer surface.

As used herein, a subject in need refers to an animal, a non-human mammal or a human. A subject in need refers to a patient.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Throughout this disclosure, the term ‘nanocage’ or ‘nanocage’ or ‘nanoaffinity’ or ‘nanoparticle affinity’ or ‘nanocapture’ or “affinity hydrogel particles” are used interchangeably. The term “nanocage” and “cage” are used interchangeably throughout the specification.

Throughout this disclosure, the term Mycobacterium tuberculosis, TB, tb, Mtb have been used interchangeably,

Throughout this disclosure, chemical bait, affinity bait, molecular bait has been used interchangeably. Throughout this disclosure, collapsible cup, collection cup, origami are used interchangeably.

Other objects, advantages and features of the present invention will become apparent from the following specification taken in conjunction with the accompanying drawings.

Exosomes are membrane bound extracellular vesicles (EVs) that are produced in the endosomal compartment of most eukaryotic cells. The multivesicular body (MVB) is an endosome defined by intraluminal vesicles (ILVs) that bud inward into the endosomal lumen.

Biomolecules are the molecules present in organisms that are essential to one or more typically biological processes. Capture is defined as formation of a bond with the molecular bait with the target analyte. In one embodiment, the capture can be defined as formation of an affinity bond of the target analyte with the molecular bait. In one embodiment, the capture is defined as formation of bond with the nanoparticle and the target analyte.

Biomarker Harvesting Nanoparticle Technology (“Nanocage”) that can Capture, Concentrate, and Preserve, Biomolecules, Nucleic Acids, Exosomes, and Whole Viruses

In one embodiment, the goal of this study is to introduce a method for investigating candidate pathogen specific peptides in patients diagnosed with pathogenic illness such as but not limited to Borreliosis, Babesiosis Anaplasmosis, Ehrlichiosis, Tick-borne encephalitis virus, Powassan Virus disease, Rickettsiosis, TBRF, Toxoplasma gondii and Tularemia, Mycobacterium tuberculosis, Toxoplasma gondii, and Trypanosoma cruzi, and Babesia microti. In one embodiment, the combined hydrogel particle pre-processing with a highly sensitive immunoassay to detect OspA, a relevant biomarker for Lyme borreliosis.

The analytical sensitivity of MS analysis is currently in the range of 10-100 ng/mL when analyzing complex matrices without pre-analytical processing, hence mass spectrometry analysis applied directly to body fluid samples lacks the sensitivity needed for low abundance pathogen derived protein detection.

The molecular bait is defined as a molecule to capture the target analyte present in a sample. In an embodiment, the molecular bait forms an affinity bond with the target analyte in a sample. In an embodiment, the molecular bait is a chemical molecule as chemical bait. In an embodiment, the molecular bait is non-chemical bait. The molecular bait is also defined as affinity bait, chemical bait.

In one embodiment, the pre-processing of the sample with affinity hydrogel particles concentrates the low abundance biomarkers to achieve mass spectrometry sensitivity in the low picogram/mL range. Additionally, the present method ensures linearity and precision of the assay in physiologically relevant protein concentration ranges.

In one embodiment, affinity hydrogel particles consist of polymeric networks functionalized with high affinity chemical baits that capture, concentrate, and preserve solution phase analytes in one step, while excluding interfering contaminants such as high abundance proteins that would otherwise negatively affect the analytical sensitivity of mass spectrometry analysis.

In an embodiment, the term “contaminant” refers to any foreign or unwanted molecule present in a solution. The contaminant may be a biological macromolecule that is present in the sample of the protein to be purified, such as DNA, RNA, or a protein other than the protein to be purified. Contaminants include, for example, unwanted protein variants, such as aggregated proteins, misfolded proteins, poorly disulfide-bonded proteins, high molecular weight species; other from host cells that secrete the protein being purified. Proteins, host cell DNA, components from cell culture media, molecules that are part of an absorbent set for affinity chromatography that leaches into the sample during the previous purification step, such as protein A; endotoxin; nucleic acid; virus; Or a fragment of any of the above.

Functionalization refers to the surface modification of nanoparticle, which includes conjugation of chemicals or bio molecules on to the surface like folic acid, biotin molecules, oligo nucleotides, peptides, antibodies, etc., to enhance the properties and hit the target with high precision. Functionalization of nanoparticle allows to inculcate properties that are specifically interested to be incorporated in nanoparticles.

In an embodiment, the nanocage technology is a hydrogel nanocage affinity hydrogel particles with molecular bait harvesting technology.

The nanocages are hollow, porous nanoparticles ranging in size from between about 1 nm and about 10 μm.

In an embodiment, the hydrogel nanocage affinity bait harvesting technology, and used the technology to successfully detect very low abundance (picogram/mL) pathogen shed antigens in urine or other body fluids with high sensitivity and specificity.

In one aspect, the present invention provides hydrogel nanocage affinity bait comprises a) an inner core, and b) an outer Shell.

In an embodiment, the nanocage in the present composition can have any suitable size. For example, the nanocage can have a diameter from about 10 nm to about 10 μm. In certain embodiments, the diameter of the nanocage is about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, and 10 μm.

In an embodiment, the nanocage or nanoparticle in the present composition can have any suitable shape, including but not limited to, sphere, square, rectangle, triangle, circular disc, cube-like shape, cube, rectangular parallelepiped (cuboid), cone, cylinder, prism, pyramid, right-angled circular cylinder and other regular or irregular shape.

In an embodiment, the nanoparticles are open mesh, non-aggregating, colloidal and >95% open void. The open mesh is a space or an opening formed due to network of the hydrogel polymer of the nanoparticles. An open mesh can be such as a scrim, reticulated or honeycomb mesh. The open voids are formed in open mesh. The nanoparticle comprises an open mesh such as a scrim, reticulated or honeycomb mesh, wherein the open voids pass directly through the nanoparticle occupy a significant portion of the surface. For example, the percentage of the surface of the nanoparticle occupied by the open void may be at least 70%, at least about 80% or at least about 90% or more than 95%. As used herein, the non-aggregating is the state of “dispersed” bioparticulates.

The internal high affinity bait core of the nanoparticle has a surface area >1000 greater that the surface of the nanoparticle. The internal captured analytes are shielded by the surrounding shell.

In an embodiments, the baits are immobilized into the core of the nanoparticle where they affinity capture analytes of interest and efficiently dissociate the analytes from unwanted interfering protein. Within the internal space of the hydrogel nanoparticles there is a vast excess of chemical bait with respect to analyte molecules. This excess of chemical bait: 1) stoichiometrically favors analyte-bait association on-rate, and 2) keeps analyte non-covalently attached to the particle in time, because a analyte molecule that dissociates from a bait will find nearby bait molecules that will trap it. The internal captured analytes are shielded by the surrounding shell have created hydrogel nanocage affinity bait biomarker harvesting technology, and have used the sensitivity-amplification (1000 fold) attribute of the technology to successfully detect very low abundance, previously invisible (picogram/mL), pathogen shed antigens in urine with high sensitivity and specificity.

In an embodiment, the hydrogel nanocage affinity bait biomarker harvesting technology has sensitivity amplification of 500 fold, 700 fold, 900 fold, 1000 fold, 1200 fold, 1500 fold, 2000 fold, 2500 fold, 3000 fold.

In an embodiment, molecular bait known in prior arts such as bind with the pathogen derived proteins, lipophosphoglycans, and glycans with vert high affinity. In an embodiment, the affinity is around (10⁻¹³).

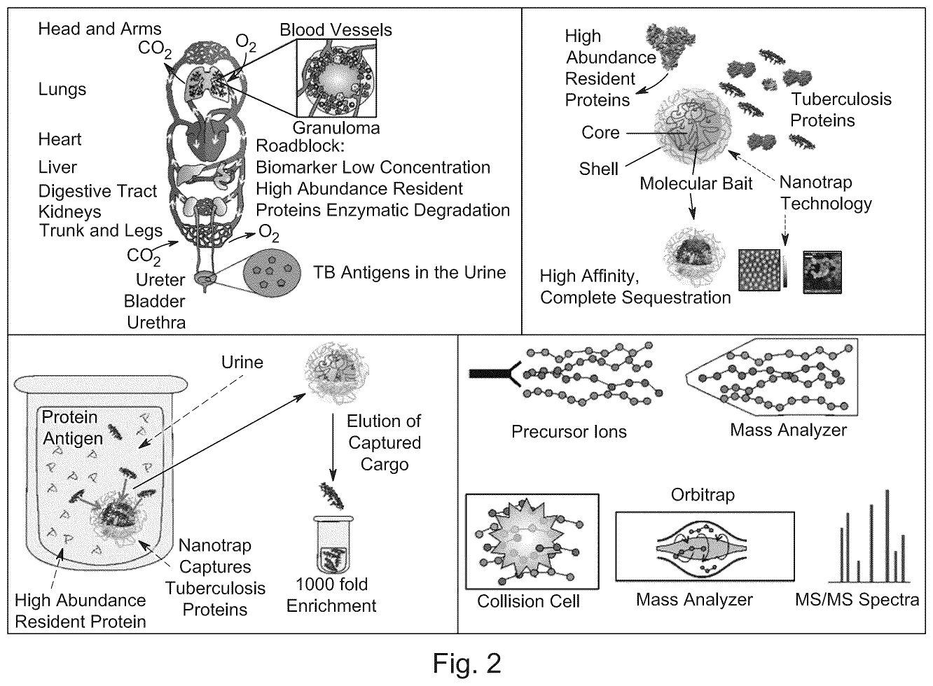

In an embodiment, the affinity is around (10⁻¹², 10⁻¹³, 10⁻¹⁴). The baits are immobilized into the core of the nanoparticle where the affinity capture analytes away from unwanted interfering proteins (FIG. 2).

In an embodiment, the chemical baits are selected from but not limited to reactive blue 221 (RB221), trypan blue (TB), Bismarck brown (BB), and Alcian Blue.

In an embodiment, the internal surface area of the hydrogel polymer mesh nanocages is thousands of times greater than the surface of an equivalent solid particle. In an embodiment, the internal surface area of the hydrogel polymer mesh nanocages can be 500 times, 750 times, 1000 times, 1200 times, 1500 times, 2000 times greater than the surface of an equivalent solid particle.

In an embodiment, the effective pore size of the particles is a function of hydrogel polymer crosslinks. Rendering the crosslinks degradable provides a means to induce the nanocages to open up and display the captured sequestered analyte cargo.

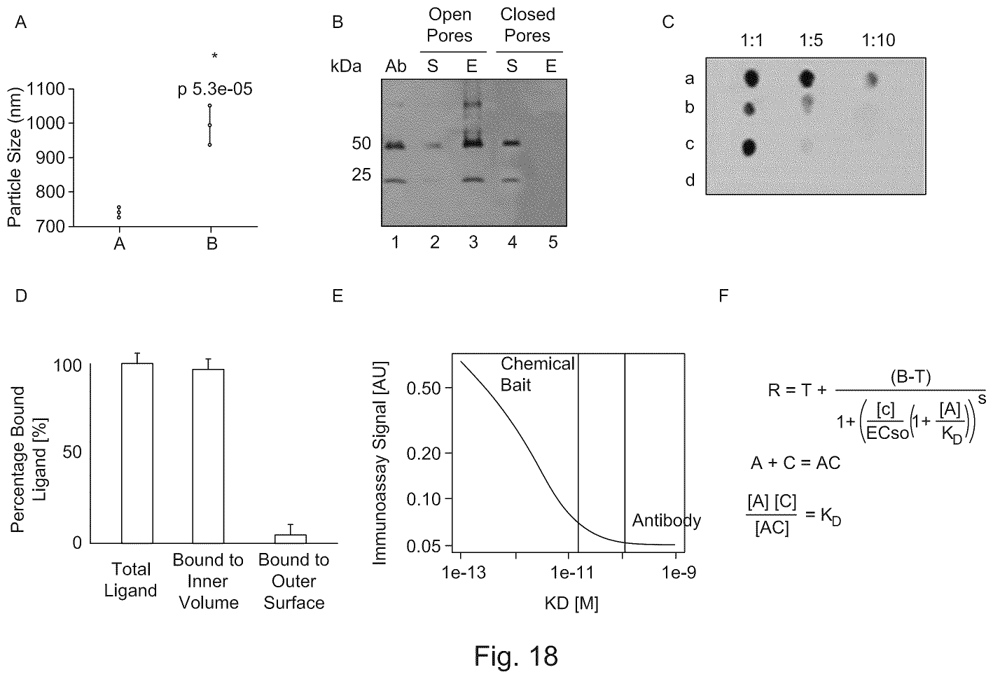

In an embodiment, FIG. 18 shows partially dissolvable nanocages captured antigen for antibody binding in a high sensitivity sandwich immunoassay. A) Significant change in hydrodynamic diameter after Nanocage oxidation. B) Oxidation of DHEA degradable cross-linker allows antibody entry (S=supernatant, E=eluate, lanes 2 and 3). Antibody is excluded before crosslink oxidation (lanes 4 and 5). C) Immuno macroarray demonstrating antigen bound to the chemical bait retains its capability to bind to the antibody. D) Anti-LAM antibody binds to LAM captured in the inner volume of nanocages (after pore dilation) 40 times higher than LAM bound to the cage surface (before pore dilation). E) and F) High affinity chemical baits improve sensitivity for antigen capture compared to conventional antibody. Eq 1 parameters: Top=1, Bottom=0.05, EC50=1e−12M, slope=0.7, G=1e−15M.

The invention comprises specific amino acid sequences liberated by enzymatic digestion of bacterial and protozoan proteins that are absolutely specific for the pathogenic strains such as Mycobacterium tuberculosis and Trypanosoma cruzi.

In an embodiment, novel previously unknown, urinary peptides have been identified in TB infected patients derived from pathogenic Mycobacterium tuberculosis (Mtb) in adults and in pediatric HIV negative patients with active pulmonary TB.

In an embodiment, novel previously unknown, urinary peptides have been identified in Chagas, lyme patients.

### Collection Vessel “Origami Cup”

In an embodiment, the nanotechnology harvest is integrated into a novel “Origami” collection cup. In an embodiment, the collection cup collects body fluid. In an embodiment, Urigami is a combination of the words “urine” and “origami” because the device collapse like an origami and can be used in urine to capture molecular information. In an embodiment, the Urigami collects urine and compresses the body fluid flat for fluid-free mailing.

In an embodiment, the collection device in the present composition can have any suitable shape and size, including but not limited to, cup, strip, sphere, square, rectangle, triangle, circular disc, cube-like shape, cube, rectangular parallelepiped (cuboid), cone, cylinder, prism, pyramid, right-angled circular cylinder and other regular or irregular shape.

In an embodiment, the collection device can have any size to collect a fluid and can collect the fluid in any range such as but not limited to 10 ml, 20 ml, 30 ml, 50 ml, 75 ml, 100 ml, >100 ml, >200 ml, >300 ml, >400 ml, >500 ml, >600 ml, 700 ml, >800, >900 ml, >1000 ml.

In an embodiment, the collection device is in form of a cup collects urine >1000 ml of urinary fluid analytes into a flat envelope for mailing that permits field or home collection of >100 mL content of urine analytes mailed in an envelope, obviating the need for shipment and refrigeration of urine fluid.

In one embodiment, the nanoparticles are immobilized on a collapsible non-hygroscopic net such that the target analyte is preserved in a dry state. The non-hygroscopic is a general term used to describe materials that does not retain moisture. In one embodiment, the non-non-hygroscopic can be defined as a material that may retain less than about 15% about 10% or about 7% or about 5% or about 3% moisture by mass, between 40 and 90% RH at room temperature. Collapsible herein refers to easily foldable.

In one embodiment, the collection vessel comprising the collapsible non hygroscopic net is configured to collect a fluid sample such that the fluid sample is in concentrated and dried state.

In an embodiment, the invention comprises a collecting vessel comprising an affinity net tethered with nanocages configured to capture and concentrate a target analyte present in a fluid. In an embodiment, the affinity net is a glass wool or glass fiber net. In an embodiment, the affinity net is non hygroscopic net.

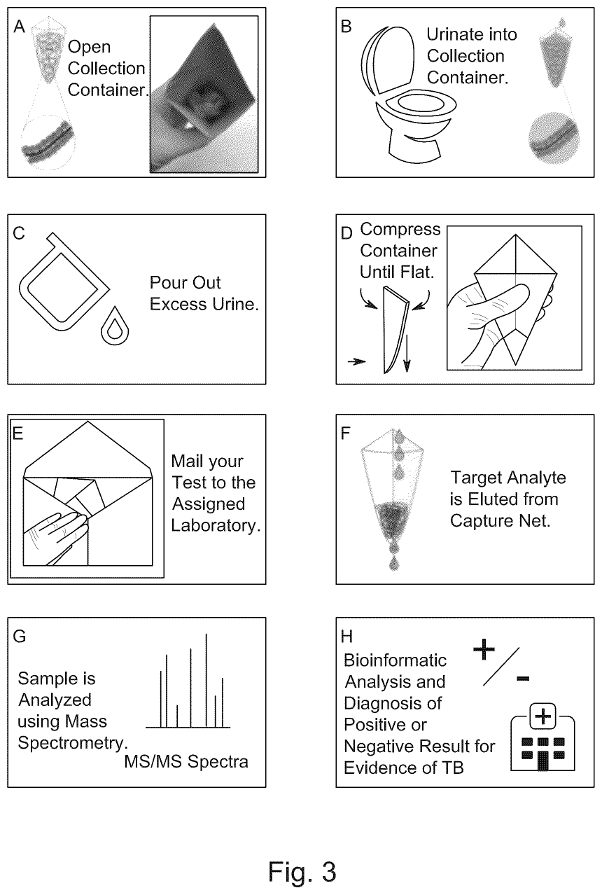

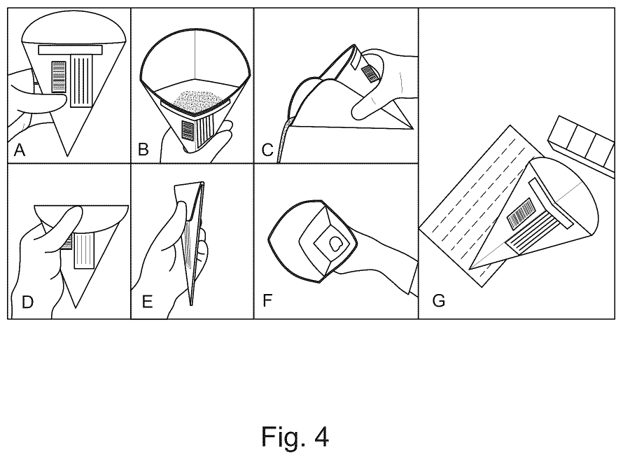

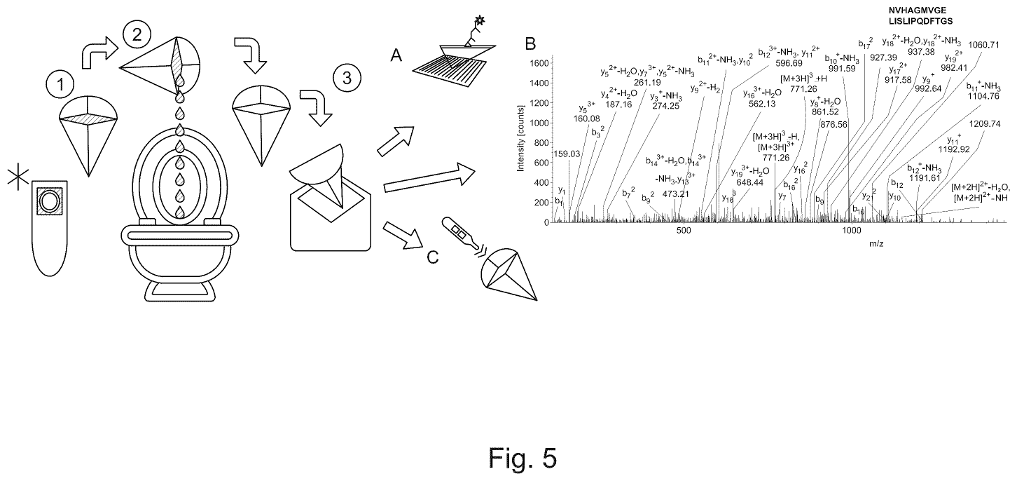

In one embodiment, the collection vessel tethers the biomarker harvesting nanoparticles of FIG. 2 onto a 3-D glass fiber net (as shown in FIG. 3) that is within the collapsible collection vessel. The patient opens the cup and deposits a urine sample into it. The net inside the container expands to fill the entire urine fluid volume in the cup. (FIG. 4-6). The tethered nanoparticles immediately sequester (via the high affinity bait) the fluid phase pathogenic analytes such as TB analytes throughout the urine fluid volume, depleting the urine of the TB target analytes. Thus, the analytes in the fluid are transferred to the solid phase net which stays inside the cup. The patient dumps out the urine fluid from the cup, leaving the net with the sequestered TB analytes inside the cup. The net does not imbibe the urine fluid. The patient folds the container (FIG. 3D) (Empty of Urine Fluid) flat and seals it in a mailing envelop which is sent to the Clinic or Diagnostic Lab.

In an embodiment, the harvested analyte is eluted and concentrated into a small volume. The small volume may be volume ≤100, ≤50 μl, ≤20 μl, ≤15 μl, ≤10 μl, ≤5 μl for the diagnostic purpose. For example: The harvested TB analytes are eluted off the net into a small volume thereby concentrating all of the TB analytes in the original 60 mL into a small volume <10 microliters for analysis by Mass Spectrometry or Immunoassay.

The low-cost collection vessel is simply mailed or given to the subject, who collects the sample into the dry pre-addressed envelope (FIG. 3E). The private identifying information remains on the collapsible cup. In this manner, urine screening samples collected by individuals with no formal training can confidentially mail in a test kit sample. A dry state is defined as fluid free state. In an embodiment, a dry state may be a state when the volume of the sample has reduced more than about 50 times, about 60 times, about 70 times, about 80 times or about 90 times, about 95 times from the initial volume of sample. In an embodiment, in the dry state, the amount of weight difference between initial weight of sample and after drying of sample is 35 times, 40 times, 50 times, 60 times, 80 times.

In an embodiment, fluid such as urine is not shipped or mailed, and no bacterial contamination is likely.

In an embodiment, “origami” collection envelope folds around 100 mL of urine fluid biomarkers into a dry confidential envelop for secure mail service transport, completely obviating the need for liquid or frozen urine handling shipment or storage, achieving virtually 100% yield of known TB antigens.

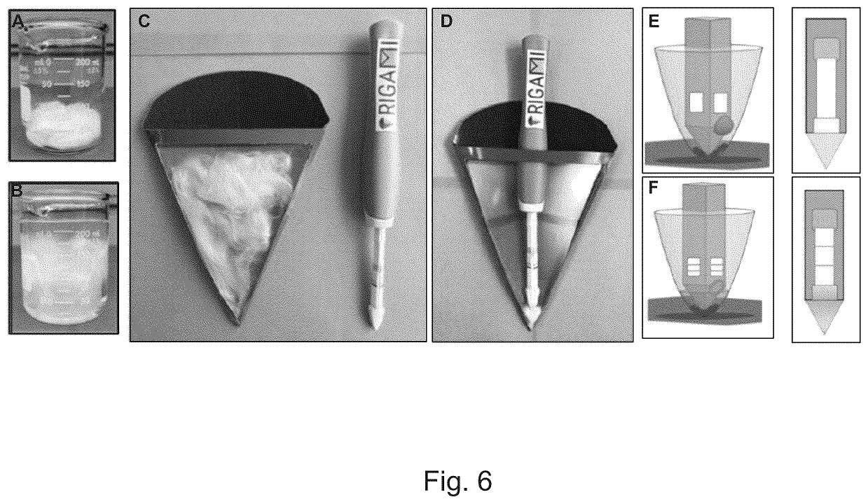

In an embodiment, disposable low-cost urine sampling envelop rapidly harvests and separates in one step all relevant biomarkers of urine, and then collapses into dry sealed envelope for mailing, banking and surveillance. For example: FIG. 6 shows a prototype of origami cup configured to measure three biomarkers at once, radially. As shown in FIG. 6. the disposable self-working immunoassay is inserted into the collapsible collection envelop. The act of insertion compresses the nanoparticle affinity net that has retained the biomarkers such as TB markers and transfers the urine markers to the tip of the test. The test is visually read in a manner identical to that used now for a pregnancy test.

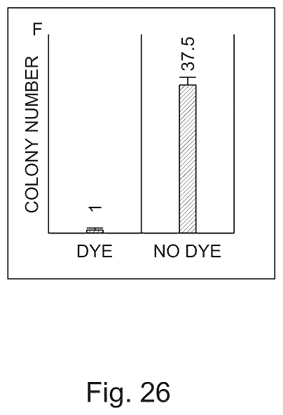

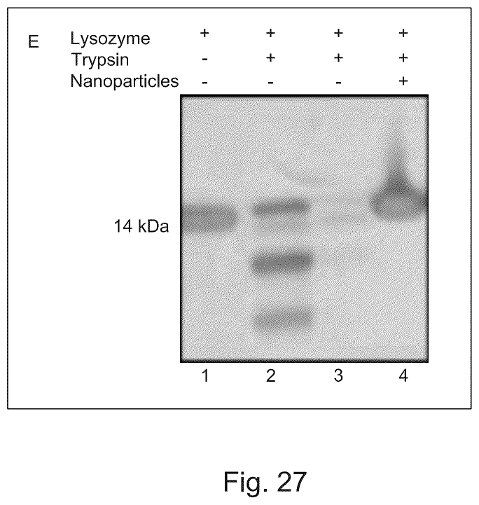

In an embodiment, the nanoparticle harvested analytes are preserved against degradation. Pathogenic organisms such as TB do not survive desiccated environment eliminating the extremely low likelihood that the urine analytes captured in the device pose an infectious hazard. Although liquid urine is not considered infectious for viable TB, we have an extra measure of safety in the design of urine collection envelope. Various bacterial suppressors for microbial growth can be used for inhibition of undesired growth of any microbe. For example: Since the dye chemistry uses copper intercalated dyes (copper surfaces are antibacterial as shown in FIG. 26), an added benefit is the suppression of bacterial growth within the dry sealed flat envelope.

In an embodiment, a multifunctional nanoparticle buoyant cage contains a new class of solid phase affinity copper dye which binds carbohydrate antigens with an affinity hundreds of times higher than existing antibodies or lectins.

In an embodiment, this technology in one step, in solution, to affinity capture, concentrate, and displace from interfering biomolecules, antigens of pathogenic microbes such as Mtb antigens present in patient's undiluted urine. The biomolecule can be defined as molecules present in organisms that are essential to one or more biological processes.

FIG. 3: Origami collection cup obviates the need for handling or shipping fluid urine for molecular diagnostic testing. Affinity capture nanoparticles are tethered to strands of glass wool fiber net that fills the volume of urine in the cup. When the urine is dumped out the net can be compressed flat for fluid-free mailing.

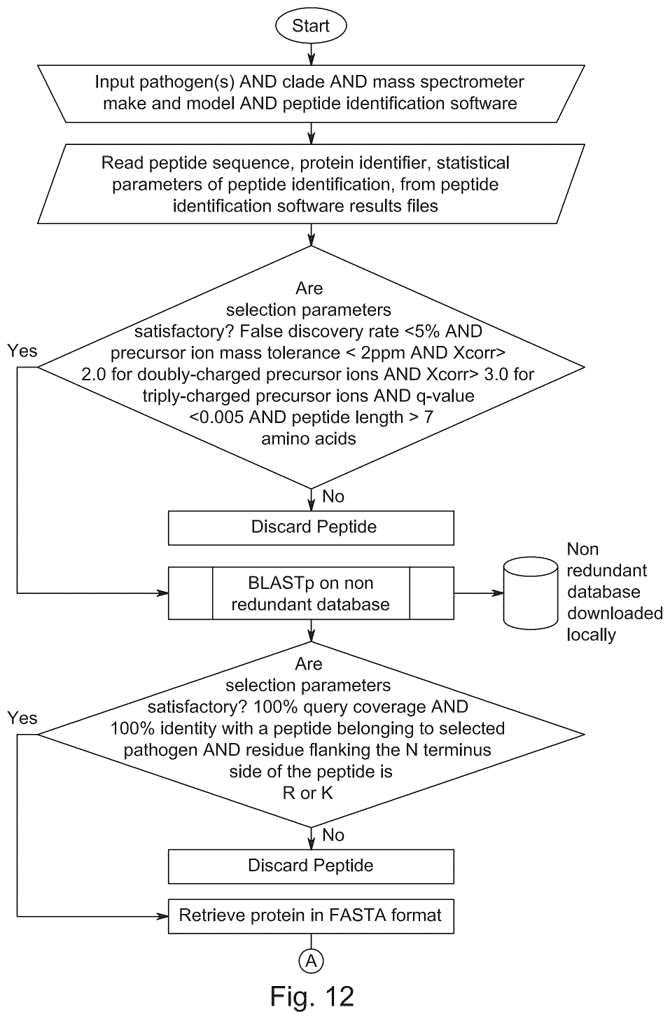

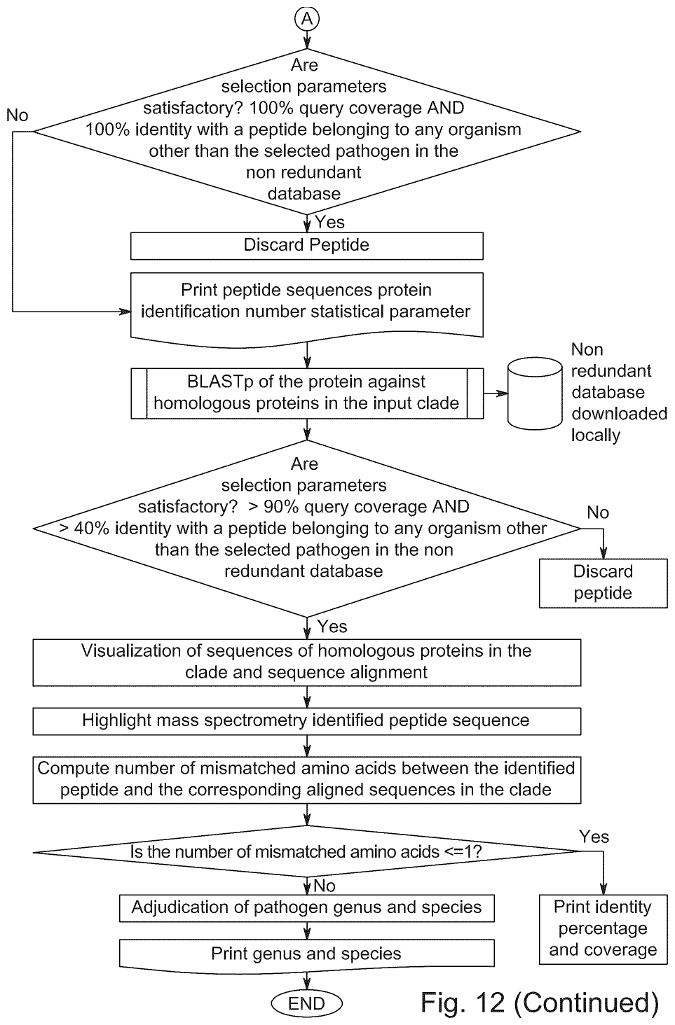

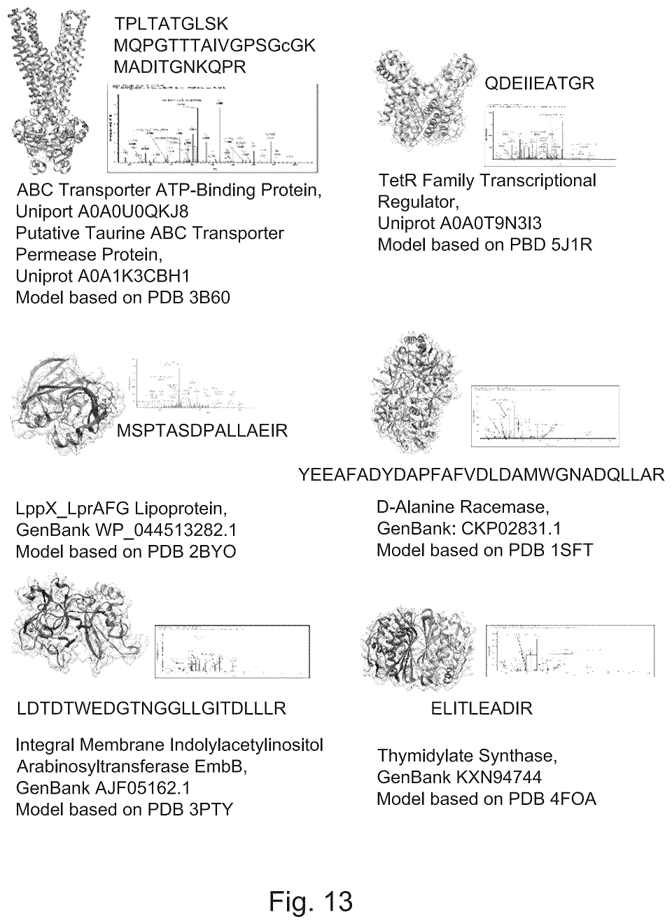

The affinity net is glass wool tethered with Nanocage particles housed in a waterproof collapsible collection cup. Glass wool (10 mg, Ohio Valley Specialty™ Untreated Glass Wool, Fisher Scientific) will be acid treated (33% HCl for 2 hours at room temperature) to enable nanoparticle attachment. After the glass wool has dried from acid treatment, 3 mL of nanoparticles (10 mg/mL) will be incubated with the glass wool for 1 hour at room temperature. The dyed glass wool will then be dried in an oven at 85° C. for 15 minutes. Washes will then be performed with MilliQ water in order to remove excess nanoparticles. Alternatively, glass wool will be treated with 3-aminopropyltriethoxysilane and 4,4′-Azobis(4-cyanovaleric acid) will be used to covalently attach amine containing nanocages. Cages will be covalently bound to the glass fibers by reversible cross linkers, that are detachable using heat (80° C., FIG. 12). A sheet of waterproof material will be folded into the desired container shape (FIG. 3). The nanoparticle-containing glass wool will be placed inside the waterproof container (FIG. 3). Urine will be added to the container, let incubate with the affinity nanoparticles tethered to the glass wool for 10 minutes and then discarded. Water washes and elution will be performed as described above; separation of the glass wool from solution will be achieved by simple compression (FIG. 2).

In an embodiment, the collapsible container is described in FIG. 3.

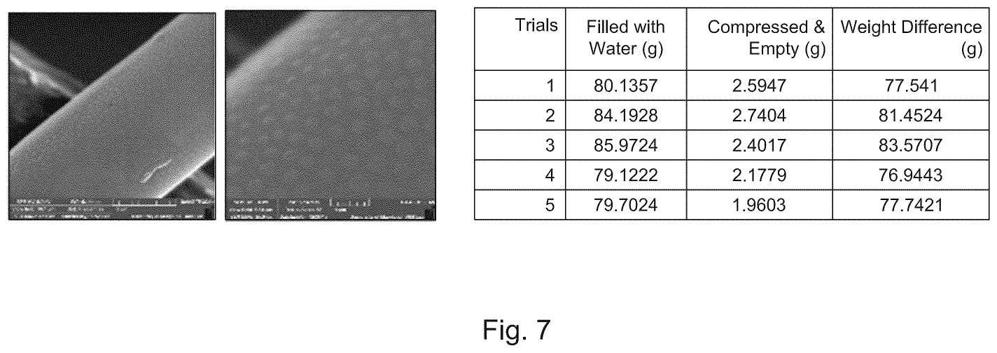

The Scanning electron microscopy (SEM) documents association of the hydrogel nanoparticles with the glass wool fibers as shown in (FIG. 7A).

In an embodiment, the collapsible container concentrates the volume of urine. The volume concentration factor reproducibility determines the sensitivity enhancement of the technology. The 7B shows the volume reduction attained by the collapsible cup. In an embodiment, the volume reduction leads to net reduction in weight of the collapsible cup. In an embodiment, the weight reduction varies from 30 to 100 times, 35 to 100 times, 40 to 100 times, 50 to 100 times.

In an embodiment, the volume concentration factor reproducibility highly optimal amplification of sensitivity. The volume concentration increases the amplification factor by a 100-fold, 150-fold, 200-fold, 250-fold.

In an embodiment, the volume concentration factor reproducibility highly optimal has a 100-fold amplification of sensitivity and a precision of less than 10% CV with a sensitivity of 15 picograms/mL 2SD above.

In an embodiment, the origami cup has a shelf life over a period of 6 months, 12 months, 18 months, over 24 months.

In an embodiment, the affinity chemistry-based nanotechnology overcomes roadblocks to achieve a highly sensitive and specific pathogen test. In an embodiment, the affinity chemistry-based nanotechnology overcomes roadblocks to achieve a highly sensitive and specific pathogen test for known TB antigens LAM and ESAT-6.

### Detection

In an embodiment, the target analyte captured and sequestered inside nanoparticles is processed to get a result with 100-fold, 150-fold, 200-fold, 250-fold higher sensitivity compared to existing technology. In an embodiment, the target analyte is processed by for mass spectrometry analysis. In an embodiment, the target analyte is analyzed with a visual lateral flow assay. In general terms, lateral flow assay use immunoassay technology using nitrocellulose membrane, coloured nanoparticles (or labels), and typically antibodies, to produce results which may be visualized.

In an embodiment, the origami device is processed to get a result with 100-fold, 150-fold, 200-fold, 250-fold higher sensitivity compared to existing technology. In an embodiment, the origami device is processed by sending the folded device at room temperature to a centralized laboratory for immune-blot analysis. In an embodiment, the origami device is processed by for mass spectrometry analysis. In an embodiment, the sample in the origami device is analyzed with a visual lateral flow assay.

After high affinity, capture of the analyte, high specificity is achieved by the antibody detection method or mass spectrometry applied to the analyte captured by the nanoparticles.

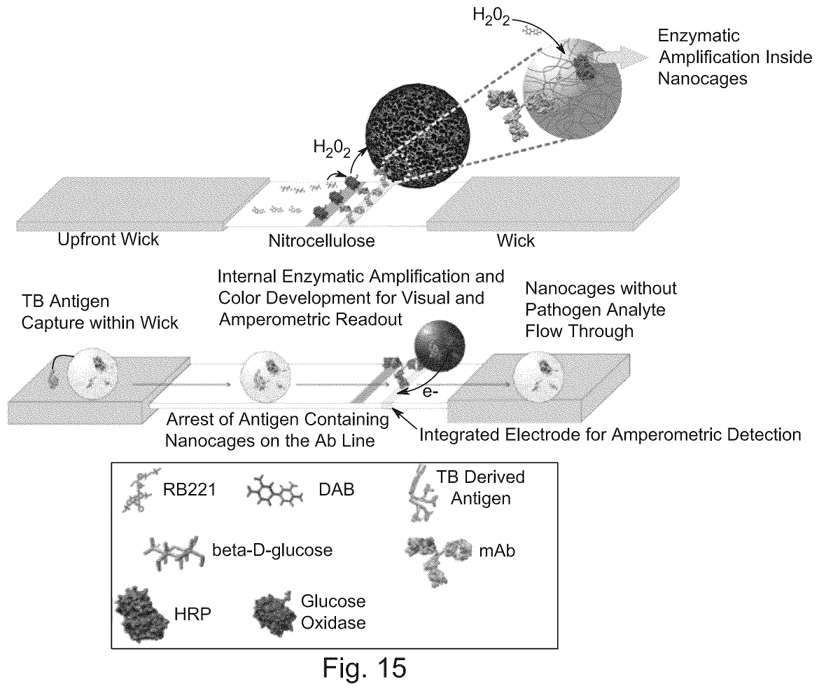

Conventional Lateral flow immunoassays are a robust well established platform. Nevertheless, they lack adequate sensitivity to detect low abundance (<1.0 μg/mL) of pathogenic antigens such as TB antigen in urine. The novel one-step self-working, enzymatically amplified visual, lateral flow immunoassay of this invention (FIG. 15) achieves a sensitivity at least one hundred times higher than conventional assays. In an embodiment, the invention provides an approximate 100 fold amplification of the sensitivity of visual lateral flow immunoassays for urinary TB screening.

In an embodiment, the novel Visual One Step Self-Working Lateral Flow Disposable Immunoassay is integrated with the collection device of FIG. 2 or 3.

In an embodiment, the solution phase enzyme amplification occurs inside the nanocage nanoparticle volume.

In an embodiment, the invention provides a novel Visual One Step Self-Working Lateral Flow Disposable Immunoassay (as shown in FIG. 15).

In one embodiment, enzyme immunoassay is employed for visual detection of the captured analyte molecule within nanoparticles. In an embodiment, enzymes are covalently immobilized in the particles while retaining full enzymatic activity. The same nanocage particle will capture the pathogen derived analyte with high affinity. The enzymatically amplified color reaction occurs inside the nanoparticles containing the captured analyte only when the nanoparticles are bound to the antibody detection line in a lateral flow format.

The working principle is illustrated in FIG. 15. The HRP (Horse Radish Peroxidase) is covalently immobilized in the particles while retaining full enzymatic activity. The same nanocage particle will capture the pathogen derived analyte with high affinity. The enzymatically amplified color reaction occurs inside the nanoparticles containing the captured analyte only when the nanoparticles are bound to the antibody detection line in a lateral flow format.

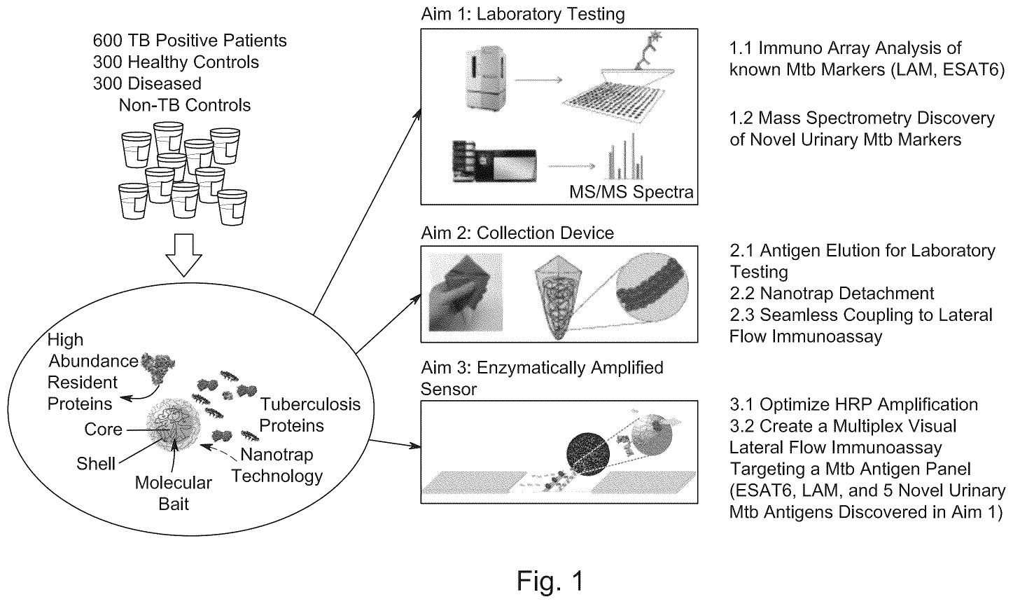

In an embodiment, the invention constitutes a new class of sandwich immunoassay that employs a novel high affinity chemical dye bait to capture the pathogen carbohydrate analyte in solution, and then, to display the captured analyte to a solid phase antibody (FIG. 1,3, 15). A solid state antibody can be defined as adsorption of an antibody on the surfaces of a matrix. The matrix can then be used as a solid phase anti-antibody in any immunological antibody-antigen assay employing a primary antibody. Alternatively, the solid phase anti-antibody could be allowed to react with a solution containing a primary antibody and the resulting solid phase antibody used in a direct or indirect immunoassay for antigen.

Nanocage nanoparticles will contain HRP for enzymatic amplification inside the nanoparticle which is triggered only for Nanocages that have arrested on the solid phase antibody. The advantage of the lateral flow point of care format is that it is a one step, self-performing test which does not require addition of reagents and human training. The basic parameters that influence the sensitivity of any lateral flow test are: 1) affinity of the capture antibody, 2) solubilization and transit time of analyte in the conjugate pad, 3) total volume (total number of analyte molecules introduced in the test), and 4) visual detection sensitivity.

The limits the sensitivity of currently used visually read lateral flow immunoassays to 10-50 ng/mL. The capture antibody will be replaced by a special affinity dye bait that captures the analyte with 100 fold greater affinity. The binding of antigen to bait will take place in solution in the wick, with no diffusion boundaries. The volume of the sample moving through the detection line will be increased from 0.05 mL to 1.0 mL (20 fold). The visual detection sensitivity of the HRP amplified color is 1000 times greater than conventional lateral flow solid gold particles because the color saturates through the full volume of the hydrogel particle. Nanocage particles are absorbed in the up-front wick of the LFI device (FIG. 15). Upon contact with the introduced sample, the nanoparticles immediately affinity-sequester desired analytes away from unwanted large proteins and massively concentrate the sequestered biomarkers. The test time is 15 minutes to allow all the urine to wick through the test.

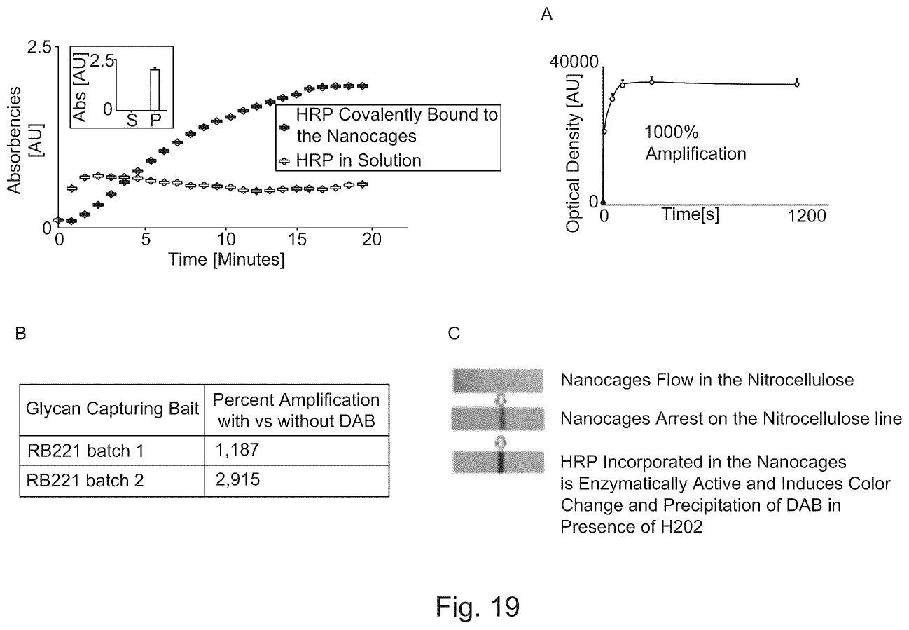

As shown in FIG. 19, HRP encapsulated in the inner volume of the cages retains full enzymatic activity, and when triggered with glucose oxidase, achieves a much higher sensitivity and dynamic range compared to the same number of HRP molecules in solution. The enzymatic amplification in the internal volume of the nanocages reaches a plateau at 30 seconds and enhances the signal 1000%. Urine containing ESAT6 yielded a dark detectable band on the test strip. The negative test result demonstrates that the nanoparticles produced no visually detectable line.

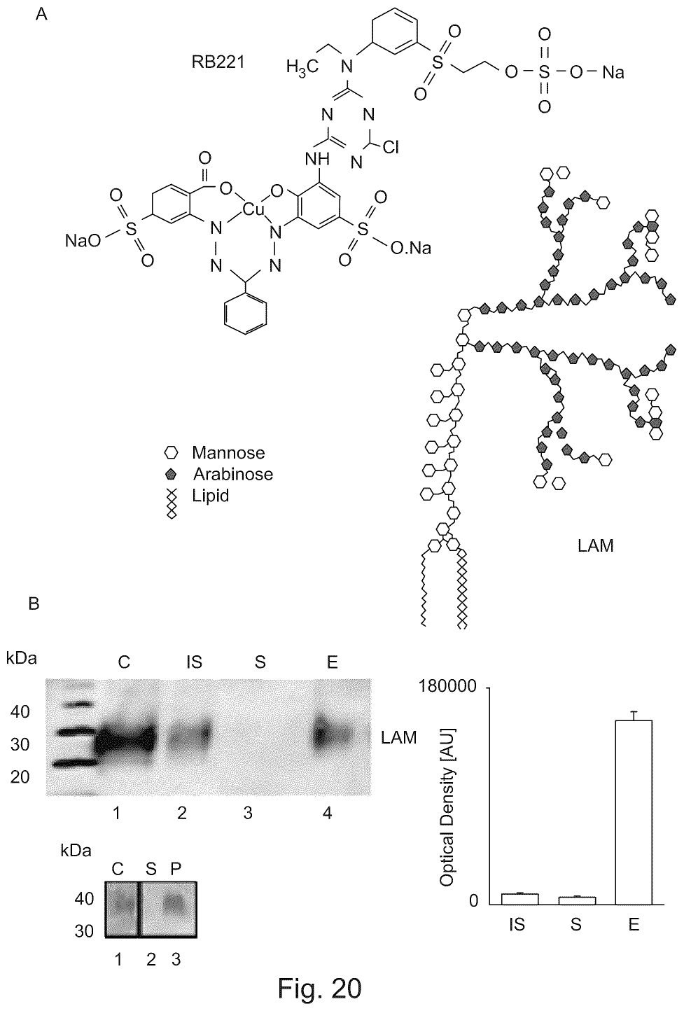

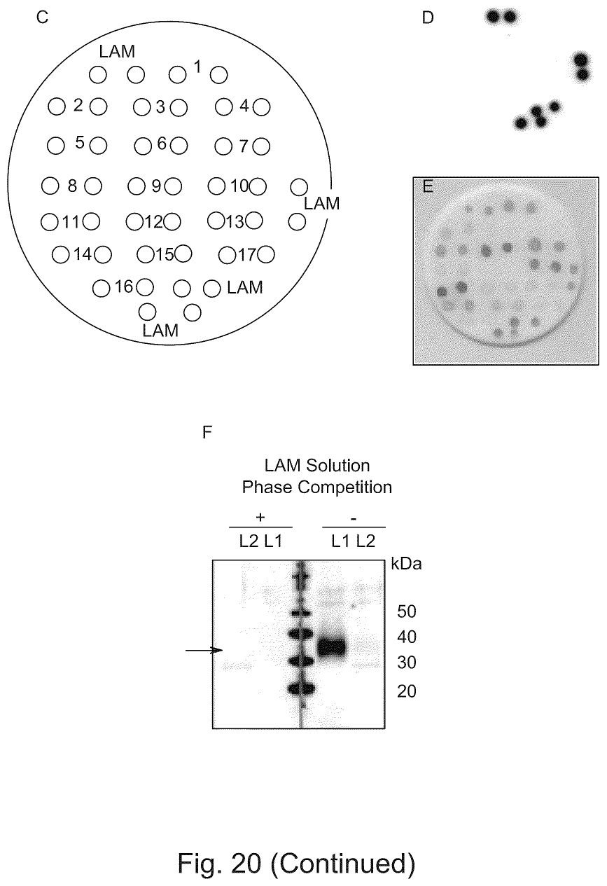

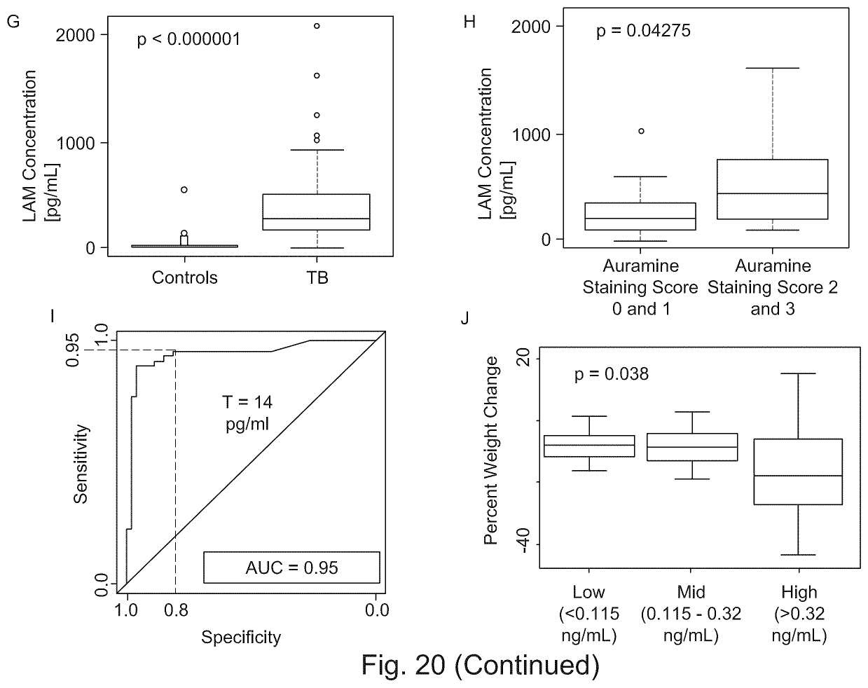

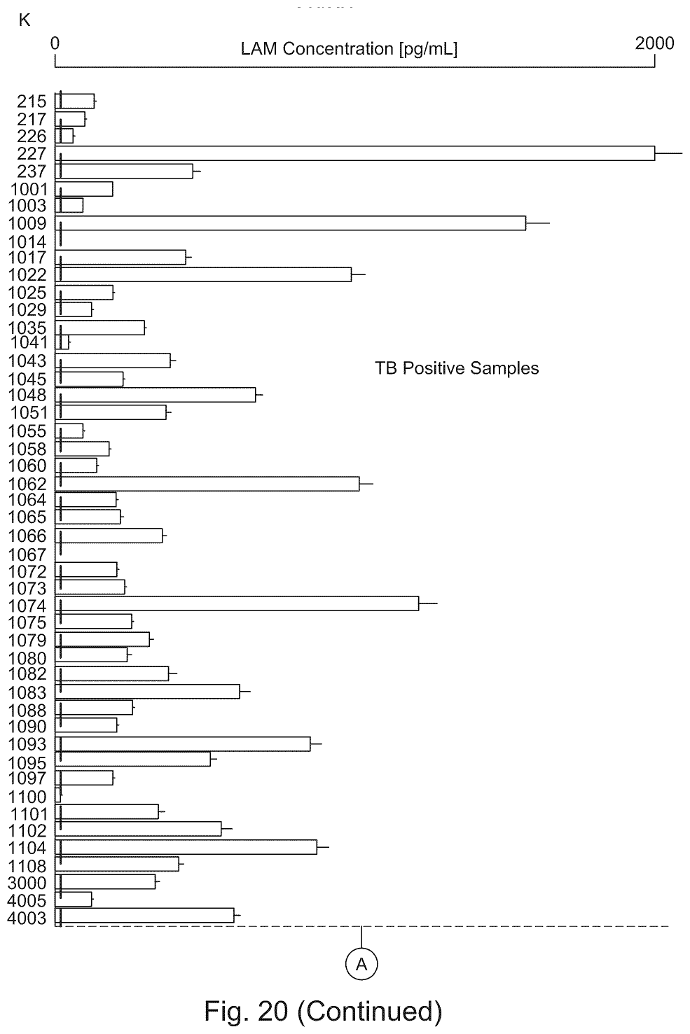

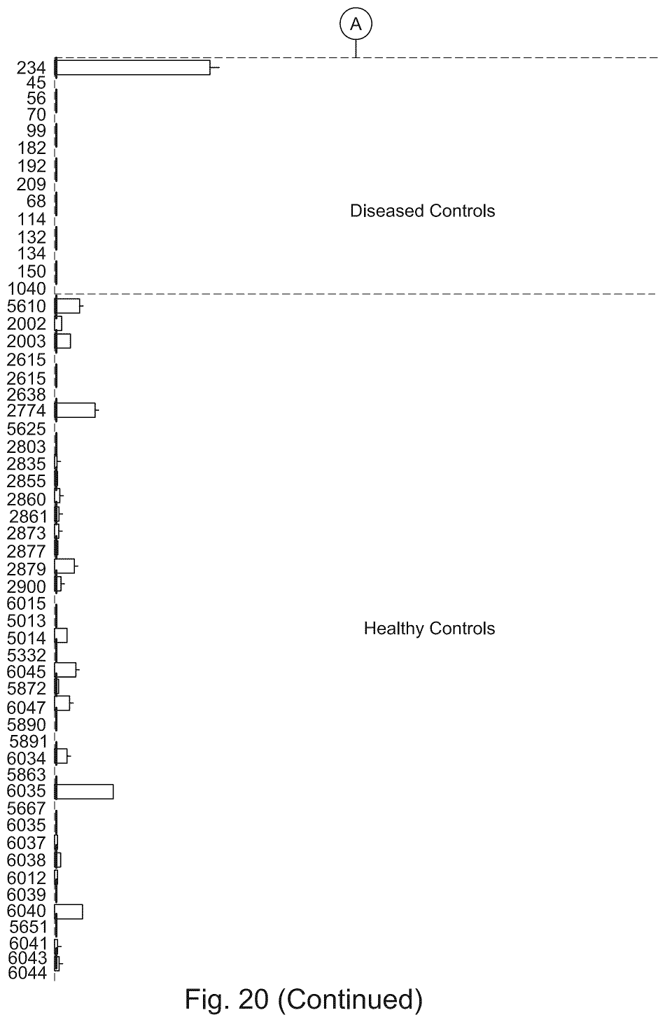

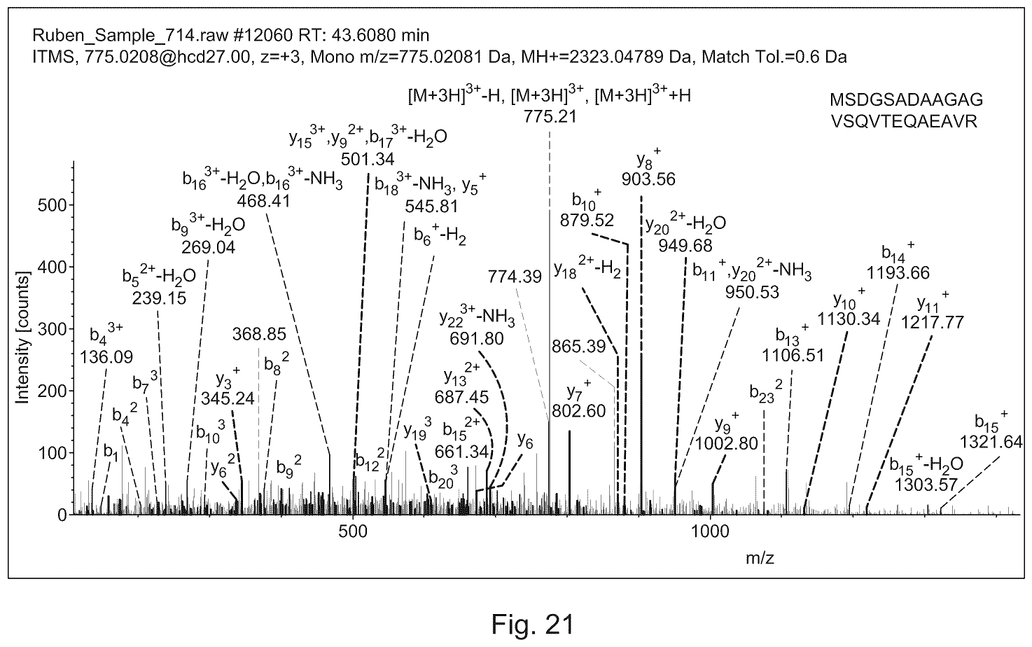

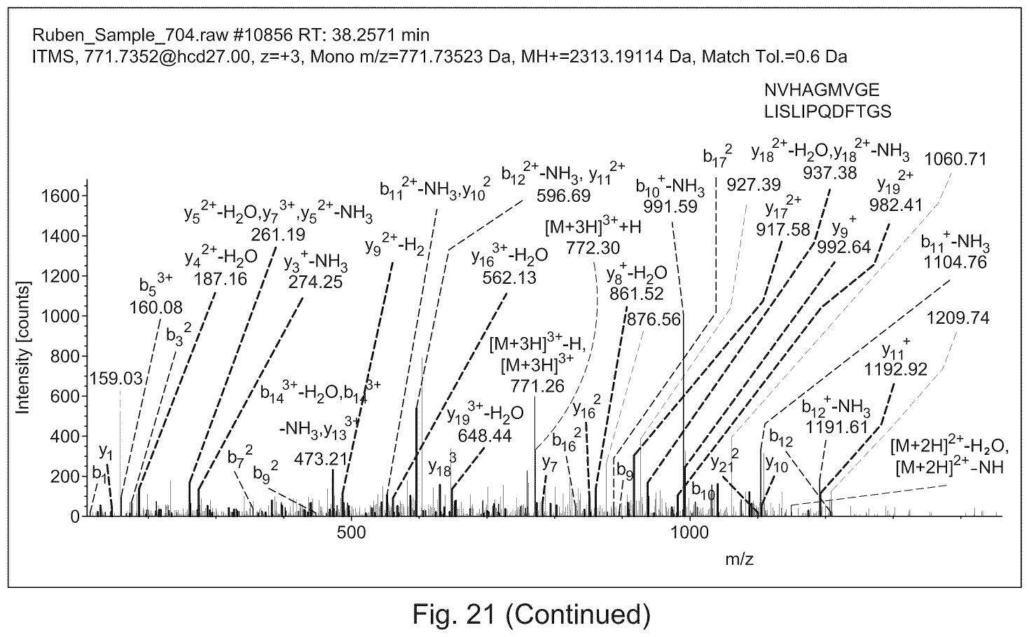

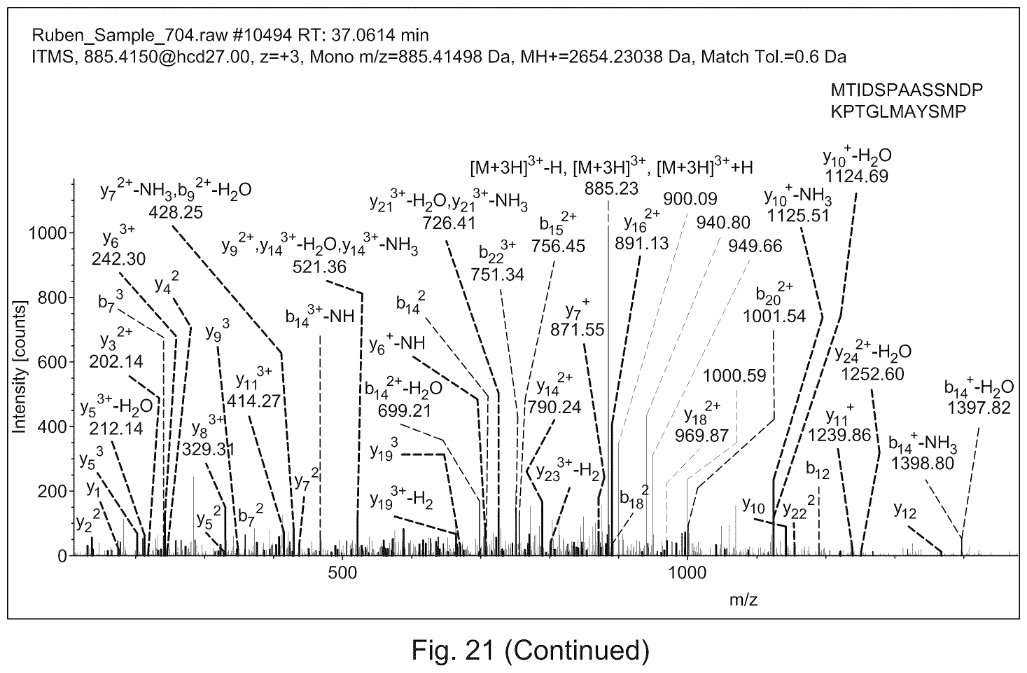

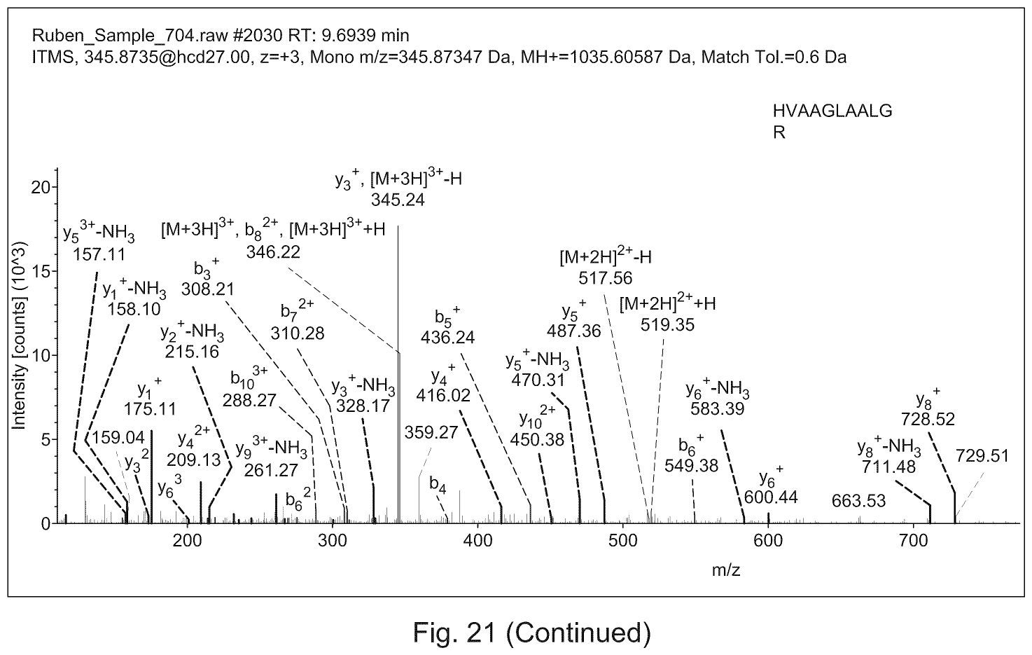

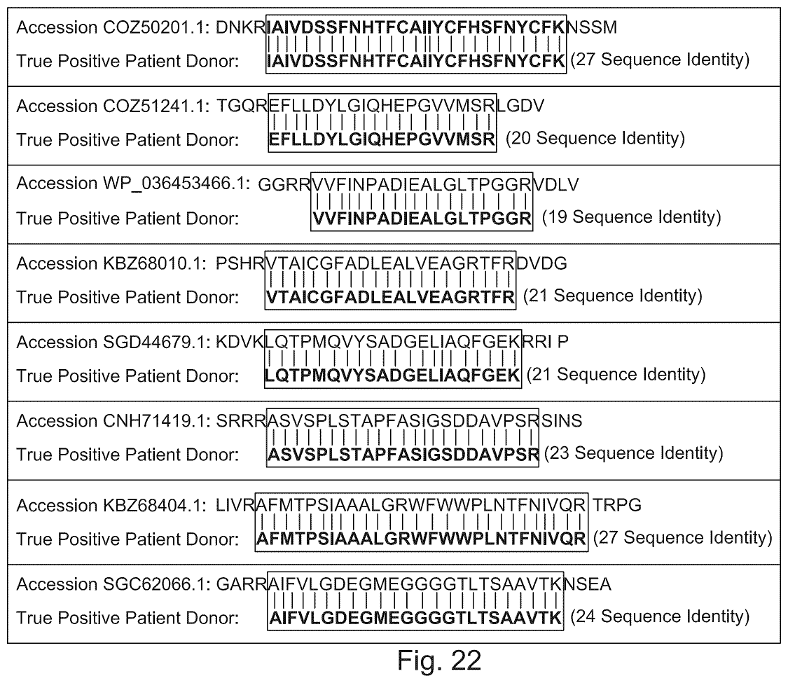

In an embodiment, a nanoparticle buoyant cage has a high sensitivity (95%) and specificity (80%), compared to diseased and healthy controls, revealing, for the first time, a significant correlation of the urinary concentration of antigen with disease severity as shown in FIG. 20.

In an embodiment, nanocages capture and immunoassay detection of pathogen antigens able to withstand 3+ high hemolysis, elevated nitrates and elevated total proteins and still maintain high specificity.

In an embodiment, the performance of the visually amplified lateral flow test for known antigens is compared to the laboratory microarray immunoassay, or mass spectrometry MS data on the same patients.

In an embodiment, the nanocage technology uses MS to attain high specificity for detection of the infectious disease. MS has the potential for virtual absolute specificity, does not require antibodies, is label free, low cost per sample and can measure dozens to hundreds of analytes in one sample. Mass spectrometry enhanced by nanotechnology can achieve previously unattainable sensitivity for characterizing urinary pathogen-derived peptides. In an embodiment, mass spectrometry enhanced by affinity hydrogel particles (analytical sensitivity=approx. 2.5 pg/mL) study pathogen-specific proteins shed in the urine of patients.

In an embodiment, the nanocage technology to create a highly sensitive, multiplex urine test that uses mass spectrometry (MS) to attain high specificity for detection of disease.

In an embodiment, the sensitivity greater is than 80 fold, 90 fold, 100 fold, 200 fold, 300 fold, 500 fold. The invention reduces the background, with a yield close to 100 percent to achieve a new class of urinary pathogenic antigen testing.

The hydrogel nanocage affinity bait biomarker harvesting technology have the sensitivity-amplification (1000 fold) attribute of the technology to successfully detect very low abundance, previously invisible (picogram/mL), pathogen shed antigens in urine with high sensitivity and specificity.

Biomarkers

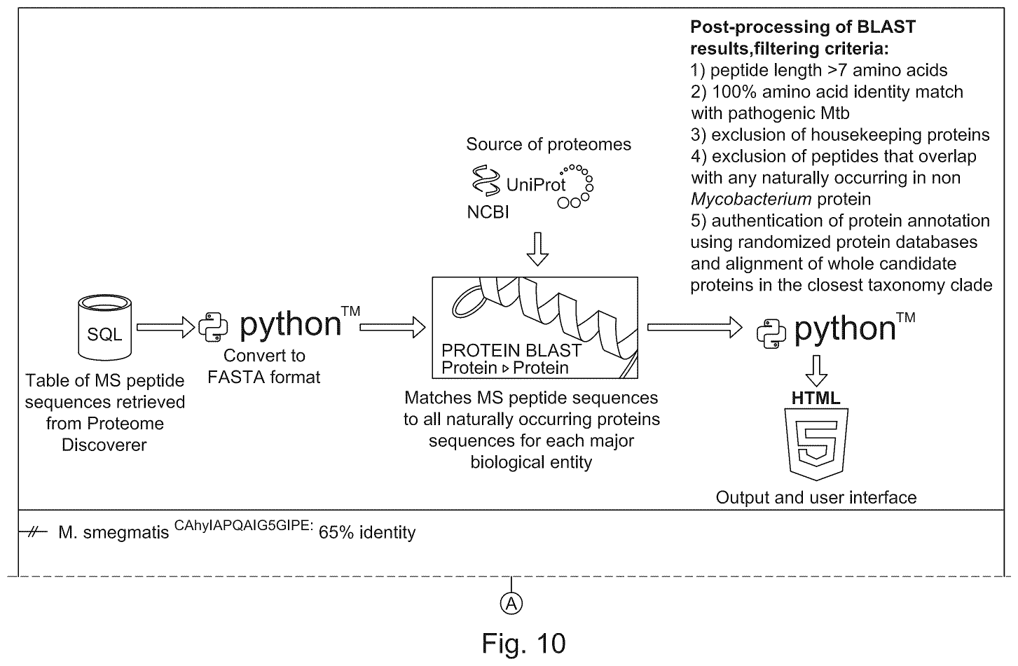

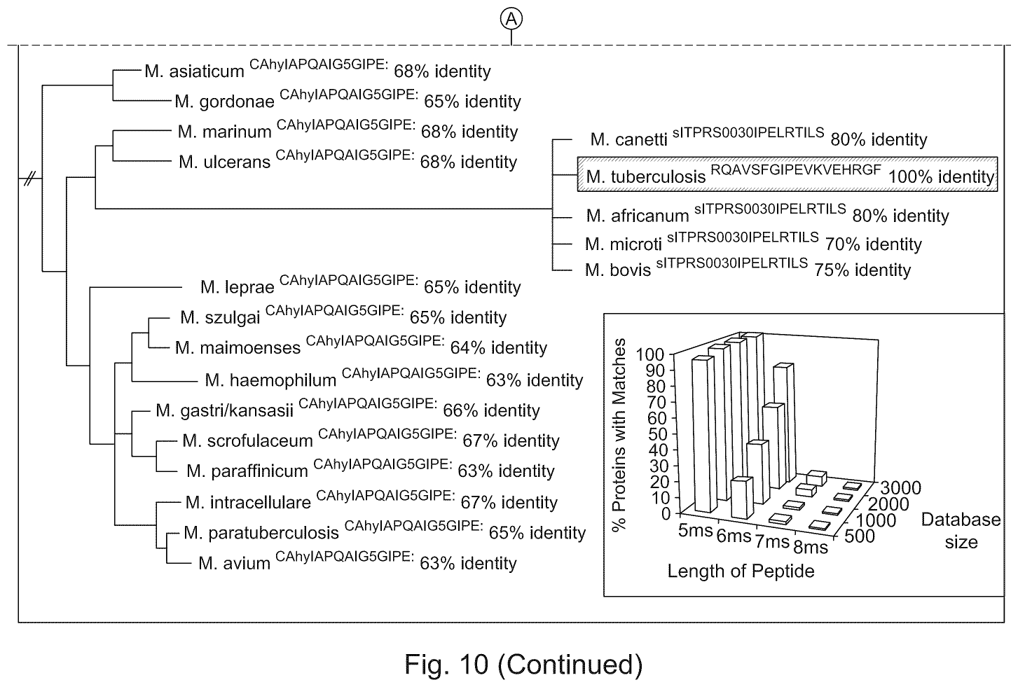

We used a special bioinformatics method for adjudicating the specificity of urinary peptides that includes a taxonomic validation among pathogenic disease organism databases to maximize specificity with a goal of almost zero false positives (as shown in FIG. 10).

In an embodiment, a large number of highly specific urinary peptides derived from TB, ChD or lyme organisms in the urine of subjects such as adults and children with documented active disease, including cases before and after therapy were found.

In an embodiment, the bioinformatic method for adjudicating the specificity of urinary peptides includes a taxonomic validation among any infectious organism databases to maximize specificity with a goal of zero false positives.

In an embodiment, peptides are authenticated on the basis of strict statistical and physicochemical parameters. Specificity of amino acid sequence and database annotation of the protein are authenticated via blast analysis. Phylogenic analysis is performed to attribute the peptide to a specific genus, species and strain with one amino acid mismatch tolerance.

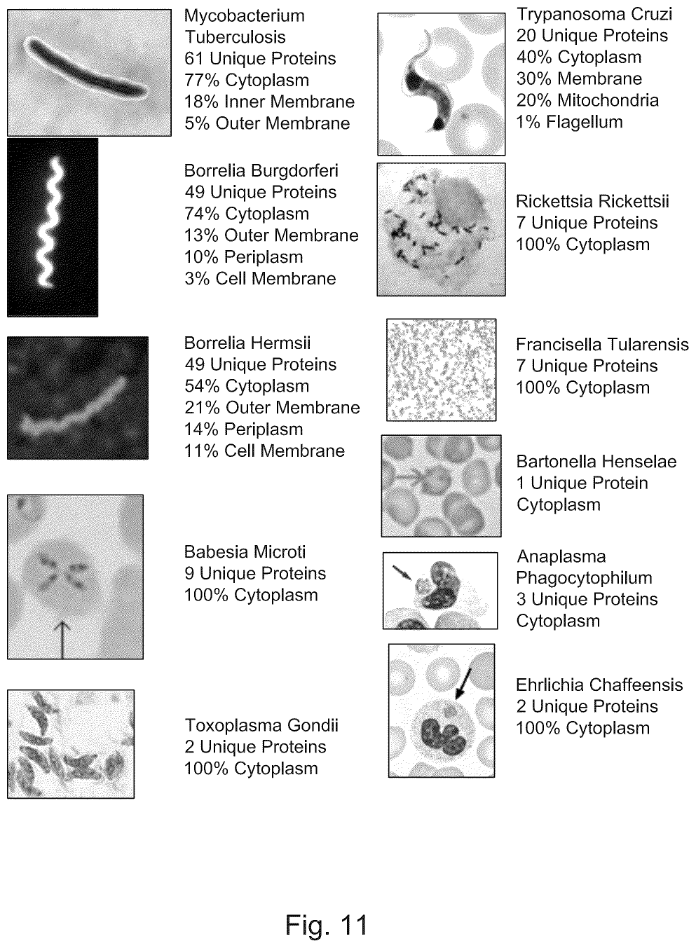

In an embodiment, the combination of nanoparticle harvesting technology with Mass spectrometer has revealed an abundance of urine peptides derived specifically from a wide variety of pathogens. For Example: in FIG. 11 shows number and cellular location of unique, pathogen derived proteins that were detected in the urine of infected patients, using affinity nanocapture, mass spectrometry analysis and bioinformatics authentication.

In an embodiment, the peptides derived from pathogen derived proteins detected in the urine of infected patients is a potential biomarker for the presence of the pathogen in a subject.

In an embodiment, the peptides derived from pathogen derived proteins detected in the urine of infected patients is a potential candidate for vaccine against the particular pathogen.

In an embodiment, peptide comprising an amino acid sequence selected from the group consisting of SEQ ID NO: 1 to SEQ ID NO: 229 or a variant sequence thereof which is at least 77%, preferably at least 88%, homologous (preferably at least 77% or at least 88% identical) to SEQ ID NO: 1 to SEQ ID NO: 229, wherein said is a biomarker for identification and diagnosis for Chagas disease, wherein said peptide is not the underlying full-length polypeptide.

TABLE 1
List of Chagas peptides as potential biomarkers

| SEQ. | | |
|---|---|---|
| ID. | | |
| NO | Peptide sequence | Accession |
| 1 | AAFAAEAAR | A0A3S5IS68 |
| 2 | ACDVSEAKVEDSWSRR | EKF32052 |
| 3 | ADVAYTTIMENTSSYFTMNYFVVLM | A0A422P4I7 |
| 4 | AEEAAAMLR | A0A2V2WFL9 |
| 5 | AEQITALLR | Q4CMZ3 |
| 6 | AESSVSVSR | K2LXB4 |
| 7 | AGAFVGSEK | A0A2V2WWR6 |
| 8 | AGDDEIQR | A0A2V2WZ77 |
| 9 | ALMDTNNFPAHVGAGER | A0A422MX69 |
| 10 | AMMRPDLQYTEVLLR | Q4CQE0 |
| 11 | APEQTGPFAADVLCIILLYIVVDMR | A0A2V2W3H5 |
| 12 | AQDISDLDLGR | R7WS61 |
| 13 | AQPLETELR | A0A422Q558 |
| 14 | AVEMVGADGTDTMFVD | R7WSB9 |
| 15 | AVSTVTTSPAG | A0A2V2VC31 |
| 16 | AVVDGLLR | A0A422Q7A8 |
| 17 | AVYVNSAR | A0A2V2WJ77 |
| 18 | CFHDADVVIGPHGSNLANAM | K2NS51 |
| 19 | CGGCLCCSGR | V5CIK9 |
| 20 | CLFNSTVLLFAVVMMCC | A0A2V2W7TO |
| 21 | CLTLNSSNPTAIMAAASVMQN | A0A422NQ50 |
| 22 | CPVEEGGANFSVGQR | Q4CTD9 |
| 23 | CSMIIYSADNGSTWSLSK | A0A2V2WCI3 |
| 24 | CYYLALLR | K2NW65 |
| 25 | DAGTIAGLNVIR | A0A2V2X690 |
| 26 | DATSSANSLR | A0A3R7KQD9 |
| 27 | DAVDTFGNFAEDVER | A0A422PFM9 |
| 28 | DGGTTTPDDDR | A0A3R7NKR6 |
| 29 | DHATWVADPAAPPSSR | R7WQV6 |
| 30 | DIAPYVAGGGAEARAEVAR | R7WQL0 |
| 31 | DMIMYPGPSAAAHR | EKF38730 |
| 32 | DNLWAPVVLAQVCANYGV | Q4D3S5 |
| 33 | DSPDAPTPAPSEPAPPPAPGPVHPPAPGDDDLD | R7WHJ0 |
| | DL | |
| 34 | DYAVAFHSTVSLADGSMLLAK | A0A2V2WST1 |
| 35 | EAEGMGYYHVSGGLPQSCGTEDHRPALHLR | V5B158 |
| 36 | EAFSLFDKDGDGTITTK | A0A0M4R526 |
| 37 | EDAANNYAR | Q27352 |
| 38 | EDKESEGEVLVLTR | Q4DIY6 |
| 39 | EEPNSDTEGEEAGVQEE | A0A3S5IRA7 |
| 40 | EEVEELVGFAPGGVCPFGVNENVRIYLDIS | K2N0F5 |
| 41 | EGILLEGSTLK | A0A2V2WF25 |
| 42 | EGVVLGADTR | V5BCU3 |
| 43 | EHLFSMDVGDSLLFR | A0A3R7PLX7 |
| 44 | EINGTTQYVQR | K2NG58 |
| 45 | ELDNQVK | A0A2V2UI95 |
| 46 | EMCTLQLQLQQTPEKETFEK | A0A422NKG4 |
| 47 | EMLYGVALLSAALQQAEK | K2NLI1 |
| 48 | EQWALHGMMLNLSVPSR | A0A3S5IS55 |
| 49 | ESGVDMLK | A0A2V2X1J4 |
| 50 | EVDEQMLNVQNK | P08562 |
| 51 | FFAMAFGLFVLFVWMCCHCCR | A0A2V2V1P0 |
| 52 | FFHVNNTNLFVLMDGVSMFTEG | A0A2V2XE54 |
| 53 | FGAVMLLATLQAYTIR | A0A3R7M247 |
| 54 | FGGMDCPDSVLAR | A0A2V2UW02 |
| 55 | FIMFFCGCAVLLGLNCR | V5BB77 |
| 56 | FITATVGDGDDK | A0A2V2W6N9 |
| 57 | FLDFVAR | A0A422NUW1 |
| 58 | FLFCLQETVGSSSMDYCYEKQQ | A0A2V2WMA5 |
| 59 | FMSTHEEQLEHSQ | A0A2V2WZD1 |
| 60 | FSDVVAGYIDSK | A0A2V2UV04 |
| 61 | FTGDGETLLEAPCVAETTLR | Q4CW04 |
| 62 | FVDPSGLTR | A0A3R7NAT9 |
| 63 | FVGGAEAAVSSGVVMR | K2MZT4 |
| 64 | GASAIPCPADMQAGK | A0A2V2XBV5 |
| 65 | GDDEDDDDDDDEEEA | A0A2V2VGA8 |
| 66 | GDGTWYDCNEGETAAPSGSR | A0A3R7M574 |
| 67 | GEAVMDLLLAGCVPSGANPDSGVITFMK | Q4D1D9 |
| 68 | GEDGSNNAVLEK | Q4D036 |
| 69 | GEIIESNIR | A0A2V2UH63 |
| 70 | GIGITLDEK | A0A3R7LC43 |
| 71 | GILLFGPPGTGK | A0A2V2UT50 |
| 72 | GLHLMAGR | K2NEJ7 |
| 73 | GLLLFGPPGTGK | A0A2V2V1P1 |
| 74 | GSGAVMGDGTIVFPMQAKNK | A0A2V2UTV8 |
| 75 | GSMGSVGFAATLLHSMELFER | K2MS94 |
| 76 | GSSGTATWGTTIVAAAEEFRAK | R7WKU9 |
| 77 | GVGTGVLMSR | R7WL85 |
| 78 | GYAYRDTEMENVVMDICYNMGMGAQFGGKY | Q4CQW5 |
| 79 | HAFGVPSYQYSIYNIADPLCSLF | A0A2V2VZB1 |
| 80 | HEVHSAVYDLTR | A0A3R7K6Z2 |
| 81 | HHSDVVAFYDAFIHPEEELEVETR | A0A422NTS1 |
| 82 | HLVLSTLSASELQHAHDGDPMEGFFCSE | Q4CVI3 |
| 83 | HSHSAIIVEGQHDFAVWDSSY | K2LZA4 |
| 84 | HTYDGDGDDDENFLQVTMATTTMGNYEVMK | A0A2V2X313 |
| 85 | HVMFFIAAVFFIWILVVDLIAVR | Q4DPH1 |
| 86 | IEDNTMSSVNGVILFGDTTVR | A0A2V2VZW2 |
| 87 | IEQWLECQR | K2NL75 |
| 88 | IFEDSEGITNAEETEVR | A0A422P0J7 |
| 89 | IFETILEMVYGLVLDS | A0A2V2XMI5 |
| 90 | IGEVIGPK | R7WP63 |
| 91 | IGGIGTVPVGR | Q0IK13 |
| 92 | IGVSELQR | A0A2V2XMU1 |
| 93 | IIDAPGHR | Q0IK13 |
| 94 | IISNASCTTNCLAPLAK | A0A3R7NEP8 |
| 95 | ILSFIEAILVQGQTDPP | A0A422NMF0 |
| 96 | INIADFIR | A0A2V2VN52 |
| 97 | IVVLGSGGVGK | A0A3R7MZE9 |
| 98 | KMLLEHFLLLAE | A0A422P455 |
| 99 | KPGSMVFTPPPTAVPLDDYR | R7WRZ0 |
| 100 | KPQTNWMEVPFEHR | K2N8N4 |
| 101 | LALSEVELWR | A0A422NHA3 |
| 102 | LCCCPSVFATNVEK | K2MS25 |
| 103 | LCDFGLAR | A0A2V2X529 |
| 104 | LDGAAQQLGGDAGAAAAAVGCSHELL | A0A3R7MXH1 |
| 105 | LDLAELLR | A0A2V2VB13 |
| 106 | LEEEDFLK | Q4E5C2 |
| 107 | LFGVTTLDLVR | A0A2V2XBW8 |
| 108 | LGEIVTTIPTIGFNVETVEYK | A0A2V2W496 |
| 109 | LGGLSGLVPGR | R7WMN9 |
| 110 | LGGTCVNVGCVPK | Q95NT5 |
| 111 | LGTEAPAVAIGEDSVQWK | R7WLW0 |
| 112 | LGTVPLSR | A0A422MPU6 |
| 113 | LIAASQAR | Q4DS89 |
| 114 | LLEAGLAGK | A0A422Q6S8 |
| 115 | LLILAEGR | A0A2V2VIP2 |
| 116 | LLSFYSIIASR | Q4E414 |
| 117 | LMEEEYEGDTRPPDMVSM | A0A2V2XE34 |
| 118 | LNGCAESAEAR | Q4E3F8 |
| 119 | LPAIQGPR | Q4DPM2 |
| 120 | LQGLTEELR | A0A3R7LJP3 |
| 121 | LQIWDTAGQER | A0A2V2XQ03 |
| 122 | LSVLIAGATGSIGR | A0A422PQ39 |
| 123 | LSVNYLQDEDAILLDFEVVT | V5B7F7 |
| 124 | LVTAEGHEPTCDETPMLR | A0A3R7LRS1 |
| 125 | MCQCGAVWVSCHVMVADSVPVCMR | A0A3R7KI25 |
| 126 | MDWYTGLGITPENLR | R7WKE5 |
| 127 | MEGNHANESNNNNNNGEAG | A0A3R7LHM4 |
| 128 | MEVVTSDGMMPSLIGQM | K2NMB3 |
| 129 | MGTITQGMFAGSFIGVVYTA | A0A3R7NR55 |
| 130 | MLEILLYLPSSSGSAY | A0A422NXA4 |
| 131 | MLTSYSDDEALLDAIMAGASGFVLK | R7WR50 |
| 132 | MLVTEATPPLNLDQLR | A0A3R7MBB1 |
| 133 | MQEAKPVTSGSGSGGGGANEAEAK | A0A3R7KUN1 |
| 134 | MSHDSALTQTLDETGVER | A0A3R7L3V1 |
| 135 | NDQWARPPR | V5B354 |
| 136 | NGNTSGGSDMIAFPDSTC | V5BVU1 |
| 137 | NGTEGALADIANR | A0A3R7RQU7 |
| 138 | NLDIERPTYTNLNR | Q27352 |
| 139 | NMAELGRMVAMVH | K2N1W1 |
| 140 | NQSSGAPTFSFYLCK | A0A3R7LMF9 |
| 141 | NVSSSNHGAVMEAR | A0A2V2W053 |
| 142 | NVTFSGGGMILTVLIGAMTGDVANATF | Q4D516 |
| 143 | NVVQEIEGR | A0A422N6T2 |
| 144 | PIIAQVLEMEYANNGDLR | A0A3R7KXD9 |
| 145 | PTINGTGENLDGDEEEGDVSQDESQEDR | Q4DGU9 |
| 146 | QALCVTEAESFSLIINMMFQDR | A0A3R7S1U0 |
| 147 | QAPLHVSGADV | A0A422P347 |
| 148 | QDDLENVPGPPAAPDLR | A0A2V2UNK0 |
| 149 | QEETTEQEPEK | K2MVQ9 |
| 150 | QEFVDLTK | A0A422MP99 |
| 151 | QEQMECIPTR | Q4DP60 |
| 152 | QGDITMPSSLLEK | A0A2V2VM12 |
| 153 | QGETTGSEPPTLINAFYPGD | A0A2V2XBI5 |
| 154 | QGILQTYVK | Q4E5S1 |
| 155 | QHYQQQPPR | A0A2V2X6F3 |
| 156 | QITQVYGFYDECLR | Q7Z1M4 |
| 157 | QITVNDLPVGR | Q4CVR5 |
| 158 | QLAITEFCDVMYGQR | V5BNX3 |
| 159 | QLEAGGHR | A0A3R7MK53 |
| 160 | QLLEQDVR | A0A3R7KLQ6 |
| 161 | QMHLLAR | A0A422NDF9 |
| 162 | QNVLLFEDTLWTEMVHK | K2NB36 |
| 163 | QQAGAQSPSCPR | K2NTB9 |
| 164 | QSEEAETTTNAEQAVR | K2NWF8 |
| 165 | QSNELLSIIEDAK | Q4CVE1 |
| 166 | QTELDLAR | A0A422NCY8 |
| 167 | QVNYGMAATTAAGLPVNNANGSTGLEYK | A0A3R7K553 |
| 168 | QWMDDAVEGSELHFVLDDADMLEDDAVHVNH | A0A3R7MQC3 |
| 169 | SALGALGVPK | A0A422MYC8 |
| 170 | SAPLLILR | A0A2V2VR51 |
| 171 | SAVVAVLR | A0A422PRQ9 |
| 172 | SIATPGCDSPLR | A0A3R7N5G3 |
| 173 | SMDDIVIQGMNLLSAACR | A0A422P9Z9 |
| 174 | SQELQQDCIDCAAHALHELGLR | A0A422PVR2 |
| 175 | SSVLMEDGTLVFPLMATNAAK | A0PGC0 |
| 176 | STSSVSASLSMETVTAPADGEH | V5APH8 |
| 177 | SYSILDPHVSNEPESAESSFSNGSL | A0A2V2XAQ5 |
| 178 | TDALEFLVDLAAVIGYR | A0A2V2VCF2 |
| 179 | TGATIGFACK | Q4CWY4 |
| 180 | THAVLLLIGSR | A0A422PRYO |
| 181 | THHCYEGVTFSGVGAVLTFFLN | A0A2V2UU67 |
| 182 | THQLILQMNTNEWYVYFDGMQVYDGYYMGSLFD | K2NAQ3 |
| | E | |
| 183 | TLEEALR | A0A3R7M910 |
| 184 | TTHQLAIVLQNGNQGSAYVDGR | Q4E506 |
| 185 | TTPSYVAFTDTER | A0A2V2URZ0 |
| 186 | TVEAEAAHGTVTR | A0A2V2VIQ4 |
| 187 | TVYSVVVWYAEDHHWQELR | A0A2V2XJY7 |
| 188 | VAESVPLPK | K2MYN1 |
| 189 | VAGGVSAPTAK | Q4CV63 |
| 190 | VATGTILSHR | A0A3R7NRI4 |
| 191 | VDAANLIVLVVAVA | R7WKV5 |
| 192 | VDAETVEQLR | A0A3R7M124 |
| 193 | VDGQSIVASSGDR | Q4CY52 |
| 194 | VEECVPGGFSPRPSHS | A0A3R7NES5 |
| 195 | VEIAEILR | A0A2V2UWC4 |
| 196 | VEIIANDQGNR | A0A2V2URZ0 |
| 197 | VEQLVSAVEGQK | A0A2V2VKF8 |
| 198 | VGSSAMVTMVR | K2M2G1 |
| 199 | VHNLSLNTPK | A0A2V2WMX3 |
| 200 | VIVEGGGIIVK | K2MRM0 |
| 201 | VLLLGDSGVGK | A0A422MTA4 |
| 202 | VLYVDSGPYEEVDTAR | A0A422NHE5 |
| 203 | VMIFCGTGGGAQAAVEEPSSDPK | A0A2V2X7C4 |
| 204 | VMYLMEAETGLLYLF | A0A422Q9H5 |
| 205 | VNDLFER | A0A2V2W875 |
| 206 | VQWACAPHVMRLAILADVWH | A0A2V2W977 |
| 207 | VSGMAEDVIR | Q4DUT5 |
| 208 | VTDFLLSR | K2N9N3 |
| 209 | VTVLSSQAEDPQVR | A0A3R7RLF4 |
| 210 | VVDAADADLVGSL | A0A2V2VNJ1 |
| 211 | VVHGSVIYMLVGMYNR | V5AJC5 |
| 212 | VVIAYEPVWAIGTGK | A0A2V2VCE4 |
| 213 | VVLLGDSGVGK | A0A2V2W0E5 |
| 214 | VVPAPDFAEAALAAVTPR | R7WVP1 |
| 215 | WGVAQQTL | A0A422Q8Q9 |
| 216 | WMQEVTVYATAMQAVR | K2MKR1 |
| 217 | WSSSLEGNLWK | A0A3R7M3K7 |
| 218 | WTGENLVPPAPCANLSGK | A0A2V2VH22 |
| 219 | YDANYVLTTEDF | A0A2V2X972 |
| 220 | YEPAGDQPAAIDDLER | R7WWJ8 |
| 221 | YEYTSGDLGCCITQIHMFLEK | A0A3R7L734 |
| 222 | YHFYNRSYLTGEK | A0A422Q8W4 |
| 223 | YIGGAAGANSGGDANK | V5BIH4 |
| 224 | YLAYLFQ | A0A422QAL6 |
| 225 | YLFGDFVAR | A0A3R7N7Q6 |
| 226 | YLGLLENIR | V5DAW6 |
| 227 | YLQLVFR | A0A2V2V695 |
| 228 | YLYEALR | A0A422NG75 |
| 229 | YQGVTEEIATIKNTMSGVAAR | A0A422NI10 |

In an embodiment, Mtb proteins were identified in the urine of active TB patients. Mtb derived markers include membrane bound channels and receptors, secreted proteins related to Mtb virulence, essential transcriptional regulators, signal transduction proteins, metabolic enzymes, essential stress response factors, enzyme necessary for DNA precursor biosynthesis, ribosomal proteins, drug targets. Peptides derived from the Mtb protein are potential biomarkers for TB disease.

The present invention relates to a peptide comprising an amino acid sequence selected from the group consisting of SEQ ID NO: 230 to SEQ ID NO: 268 or a variant sequence thereof which is at least 77%, preferably at least 88%, homologous (preferably at least 77% or at least 88% identical) to SEQ ID NO: 230 to SEQ ID NO: 268 of Table 2, wherein said is a biomarker for identification and diagnosis for TB disease, wherein said peptide is not the underlying full-length polypeptide.

TABLE 2
List of TB peptides as potential biomarkers

| SEQ. | | |
|---|---|---|
| ID. | | Accession |
| NO. | Peptide sequence | number |
| 230 | TPLTATGLSK | A0A049DCB4 |
| 231 | AGQRWISIVEPLR | G1BSG1 |
| 232 | VTRETLALLIDLAR | A0A1A9E4L1 |
| 233 | SLWLAVVAGMIVAMLVYVLA | H6WSE2 |
| 234 | LQTPMQVYSADGELIAQFGEK | A0A1K3J6H0 |
| 235 | MLECYLQLSR | A0A0T9V8Q5 |
| 236 | AAEQYWQAEQATNGYMLK | G8IR01 |
| 237 | EIRPNLDELETGALSPYPIMR | B2HKU7 |
| 238 | AIFVLGDEGMEGGGGTLTSAAVTK | A0A1K3CM05 |
| 239 | WFAELTTK | A0A081HW89 |
| 240 | DKAALSSLR | A0A051TRQ9 |
| 241 | QDEIIEATGR | A0AOT9N313 |
| 242 | MQAAVAQGR | A0A0Q3Z2T0 |
| 243 | NDLGVFDFR | A0A1K3H559 |
| 244 | AIAENPDDIAEYCTSGLTLTGFLGLSDTPR | A0A081I3K4 |
| 245 | VAVIELDADR | A0A051UHN0 |
| 246 | VLAAASSPEK | A0A049DVS9 |
| 247 | EFLLDYLGIQHEPGVVMSR | A0A0T9CW32 |
| 248 | VVFINPADIEALGLTPGGR | A0A081I8B0 |
| 249 | FSVAELVRPWLDTWR | A0A049DCE4 |
| 250 | IAIVDSSFNHTFCAIIYCFHSFNYCFK | A0A0T9LEC8 |
| 251 | VTAICGFADLEALVEAGRTFR | A0A051UG30 |
| 252 | GQHFCIGYHLARLEISVLLQEWFR | B2HLW7 |
| 253 | LTGADFTR | G7CF58 |
| 254 | QALLTGR | B2HHV4 |
| 255 | MADITGNKQPR | A0A1K3CBH1 |
| 256 | ASVSPLSTAPFASIGSDDAVPSR | A0A0T9PM68 |
| 257 | MQPGTTTAIVGPSGCGK | AOA0U0QKJ8 |
| 258 | AFMTPSIAAALGRWFWWPLNTFNIVQR | A0A051UGR6 |
| 259 | LLQMVIMPLVFASILSAVAR | A0A1K3EYR7 |
| 260 | MDPDALVAQLVDEAQNVLR | G8I3L7 |
| 261 | VSVVSLPR | A0A0T9M2X3 |
| 262 | DFHAGFMSSGDVGYLDENGR | A0A089QP93 |
| 263 | LATVIAAMCAGADCIDDVDVLR | G7CE61 |
| 264 | DHPAPLIVR | A0A0U0QND5 |
| 265 | YLIGCDGGR | A0A051TZ12 |
| 266 | TGLSPSDMDVACMYDCFTYTVMATMEGFGFCE | A0A051TXH8 |
| | K | |
| 267 | SVTAAEGDALAR | B2I428 |
| 268 | GARVPALDLEQMQVIYQMR | A0A0T9JZE4 |

In an embodiment, peptide comprising an amino acid sequence selected from the group consisting of SEQ ID NO: 269 to SEQ ID NO: 286 or a variant sequence thereof which is at least 77%, preferably at least 88%, homologous (preferably at least 77% or at least 88% identical) to SEQ ID NO: 269 to SEQ ID NO: 286, wherein said is a biomarker for identification and diagnosis for Chagas disease, wherein said peptide is not the underlying full-length polypeptide.

TABLE 3
List of Chagas peptides as potential biomarkers

| SEQ. ID. No | Protein | Peptide Sequence |
|---|---|---|
| 269 | transcript = TcCLB.507011.40: mRNA \| | YVSMGTNPHPSQSMSPVSM |
| | gene = TcCLB.507011.40 \| | R |
| | organism = Trypanosoma_cruzi_CL_Brener_ | |
| | Esmeraldo-like \| | |
| | gene_product = hypothetical protein, | |
| | conserved \| | |
| | transcript_product = hypothetical protein, | |
| | conserved \| location = TcChr39-S: 1342675- | |
| | 1344600(+) \| protein_length = 641 \| | |
| | sequence_SO = chromosome \| | |
| | SO = protein_coding \| is_pseudo = false | |
| 270 | transcript = BCY84_17297-t36_1 \| | RFVHRDIKPSNILISR |
| | gene = BCY84_17297 \| | |
| | organism = Trypanosoma_cruzi_cruzi_strain_ | |
| | Dm28c \| gene_product = protein | |
| | kinase, cdc2-related kinase \| | |
| | transcript_product = protein kinase,cdc2- | |
| | related kinase \| | |
| | location = MBSY01000635: 171329- | |
| | 172321(−) \| protein_length = 330 \| | |
| | sequence_SO = contig \| SO = protein_coding \| | |
| | is_pseudo = false | |
| 271 | transcript = BCY84_18641-t36_1 \| | YAQFMESFSNFDFSIMWYCN |
| | gene = BCY84_18641 \| | AGCLSTDVPR |
| | organism = Trypanosoma_cruzi_cruzi_strain_ | |
| | Dm28c \| gene_product = hypothetical | |
| | protein \| transcript_product = hypothetical | |
| | protein \| | |
| | location = MBSY01000689: 399673- | |
| | 404679(+) \| protein_length = 1668 \| | |
| | sequence_SO = contig \| SO = protein_coding \| | |
| | is_pseudo = false | |
| 272 | transcript = BCY84_22768-t36_1 \| | HFTGSTAPR |
| | gene = BCY84_22768 \| | |
| | organism = Trypanosoma_cruzi_cruzi_strain_ | |
| | Dm28c \| gene_product = hypothetical | |
| | protein \| transcript_product = hypothetical | |
| | protein \| | |
| | location = MBSY01000794: 48618- | |
| | 63323(+) \| protein_length = 4901 \| | |
| | sequence_SO = contig \| SO = protein_coding \| | |
| | is_pseudo = false | |
| 273 | transcript = TcCLB.506367.40: mRNA \| | LHLTGRLIAK |
| | gene = TcCLB.506367.40 \| | |
| | organism = Trypanosoma_cruzi_CL_Brener_ | |
| | Esmeraldo-like \| | |
| | gene_product = hypothetical protein, | |
| | conserved \| | |
| | transcript_product = hypothetical protein, | |
| | conserved \| location = TcChr39-S: 398908- | |
| | 400392(−) \| protein_length = 494 \| | |
| | sequence_SO = chromosome \| | |
| | SO = protein_coding \| is_pseudo = false | |
| 274 | transcript = BCY84_15012-t36_1 \| | TAGDAAAAGGPR |
| | gene = BCY84_15012 \| | |
| | organism = Trypanosoma_cruzi_cruzi_strain_ | |
| | Dm28c \| gene_product = hypothetical | |
| | protein \| transcript_product = hypothetical | |
| | protein \| | |
| | location = MBSY01000599: 157191- | |
| | 157955(−) \| protein_length = 254 \| | |
| | sequence_SO = contig \| SO = protein_coding \| | |
| | is_pseudo = false | |
| 275 | transcript = BCY84_01456-t36_1 \| | FLACVNDGGHCSLFTQHEFP |
| | gene = BCY84_01456 \| | SEDEFSTDFFNEHGK |
| | organism = Trypanosoma_cruzi_cruzi_strain_ | |
| | Dm28c \| gene_product = hypothetical | |
| | protein \| transcript_product = hypothetical | |
| | protein \| location = MBSY01000059: 5809- | |
| | 7914(−) \| protein_length = 701 \| | |
| | sequence_SO = contig \| SO = protein_coding \| | |
| | is_pseudo = false | |
| 276 | hypothetical protein [Trypanosoma cruzi | CTVDISTAGSTETNIGDISCR |
| | strain CL Brener] | |
| 277 | hypothetical protein [Trypanosoma cruzi | AVQAMCRDER |
| | strain CL Brener] | |
| 278 | hypothetical protein [Trypanosoma cruzi | LIDFVENNAMK |
| | strain CL Brener] | |
| 279 | mucin-associated surface protein (MASP) | GAGALEKVK |
| | [Trypanosoma cruzi strain CL Brener] | |
| 280 | transcript = BCY84_08138-t36_1 \| | NNDGATGATR |
| | gene = BCY84_08138 \| | |
| | organism = Trypanosoma_cruzi_cruzi_strain_ | |
| | Dm28c \| gene_product = trans-sialidase \| | |
| | transcript_product = trans-sialidase \| | |
| | location = MBSY01000386: 3903-6602(−) \| | |
| | protein_length = 899 \| sequence_SO = contig \| | |
| | SO = protein_coding \| is_pseudo = false | |
| 281 | hypothetical protein [Trypanosoma cruzi | KEAEAENALNR |
| | strain CL Brener] | |
| 282 | transcript = TcCLB.507083.90: mRNA \| | RPRLVYSEDSEVR |
| | gene = TcCLB.507083.90 \| | |
| | organism = Trypanosoma_cruzi_CL_Brener_ | |
| | Esmeraldo-like \| | |
| | gene_product = hypothetical protein, | |
| | conserved \| | |
| | transcript_product = hypothetical protein, | |
| | conserved \| location = TcChr25-S: 339208- | |
| | 340893(+) \| protein_length = 561 \| | |
| | sequence_SO = chromosome \| | |
| | SO = protein_coding \| is_pseudo = false | |
| 283 | transcript = BCY84_13262-t36_1 \| | IDLLQAELHK |
| | gene = BCY84_13262 \| | |
| | organism = Trypanosoma_cruzi_cruzi_strain_ | |
| | Dm28c \| gene_product = hypothetical | |
| | protein \| transcript_product = hypothetical | |
| | protein \| | |
| | location = MBSY01000938: 10794-11717(−) \| | |
| | protein_length = 307 \| | |
| | sequence_SO = contig \| SO = protein_coding \| | |
| | is_pseudo = false | |
| 284 | transcript = BCY84_01068-t36_1 \| | DSESHAESDRLKR |
| | gene = BCY84_01068 \| | |
| | organism = Trypanosoma_cruzi_cruzi_strain_ | |
| | Dm28c \| gene_product = heat shock 70 | |
| | kDa protein, mitochondrial precursor \| | |
| | transcript_product = heat shock 70 kDa | |
| | protein, mitochondrial precursor \| | |
| | location = MBSY01000053: 615988- | |
| | 617955(−) \| protein_length = 655 \| | |
| | sequence_SO = contig \| SO = protein_coding \| | |
| | is_pseudo = false | |
| 285 | mucin-associated surface protein (MASP) | MNPEKEKNGR |
| | [Trypanosoma cruzi strain CL Brener] | |
| 286 | hypothetical protein [Trypanosoma cruzi | QPHERMYCGTIK |
| | strain CL Brener] | |

In an embodiment, peptide comprising an amino acid sequence selected from the group consisting of SEQ ID NO: 287 to SEQ ID NO: 311 or a variant sequence thereof which is at least 77%, preferably at least 88%, homologous (preferably at least 77% or at least 88% identical) to SEQ ID NO: 287 to SEQ ID NO: 311, wherein said is a biomarker for identification and diagnosis for TB disease, wherein said peptide is not the underlying full-length polypeptide.

TABLE 4
List of TB peptides as potential biomarkers

| SEQ. ID. NO. | Validated Proteins Description | Peptide Seq |
|---|---|---|
| 287 | response regulator with CheY-like | EELMLSVWGTGQGVSR |
| | receiver domain and winged-helix | |
| | DNA-binding domain | |
| 288 | GlcNAc-binding protein A | TSNWEYFIGGTK |
| | precursor [Mycobacterium | |
| | tuberculosis] | |
| 289 | thymidylate synthase % 2C ThyA | ELITLEADIR |
| | [Mycobacterium tuberculosis] | |
| 290 | PAS/PAC sensor hybrid histidine | TVQDGRDKAVDDLATHVMAR |
| | kinase [Mycobacterium | |
| | tuberculosis] | |
| 291 | 4-alpha-glucanotransferase | WLQWQLDEQLCR |
| | [Mycobacterium tuberculosis | |
| | CAS/NITR204] | |
| 292 | Uncharacterised protein | MLPVNDVNNAGLMAQIPAVLAGP |
| | [Mycobacterium tuberculosis] | NR |
| 293 | peptidyl-prolyl cis-trans isomerase | MFGTIQADGLTTLDK |
| | B [Mycobacterium tuberculosis] | |
| 294 | Uncharacterised protein | VEEFLDLPR |
| | [Mycobacterium tuberculosis] | |
| 295 | Uncharacterised protein | MSTGIAAMVVQFGLAMMPLGR |
| | [Mycobacterium tuberculosis] | |
| 296 | Uncharacterised protein | IATVVVGVR |
| | [Mycobacterium tuberculosis] | |
| 297 | RHS repeat-associated core domain | NQTLLLQGK |
| | [Mycobacterium tuberculosis] | |
| 298 | Conserved exported protein of | YIDGLTIDR |
| | uncharacterised function | |
| | [Mycobacterium | |
| 299 | RNA-directed DNA polymerase | AWDGGDGALVR |
| | [Mycobacterium tuberculosis] | |
| 235 | DitF protein [Mycobacterium | MLECYLQLSR |
| | tuberculosis] | |
| 300 | Uncharacterised protein | MNPPMTFSVPNTTATNR |
| | [Mycobacterium tuberculosis] | |
| 301 | transposase IS4 family protein | VDPQVVVGLLVDR |
| | [Mycobacterium tuberculosis] | |
| 302 | Uncharacterised protein | NSWSMLLLMTSISIEK |
| | [Mycobacterium tuberculosis] | |
| 303 | hypothetical protein K875_05463 | SWVIESMPSVAIDGIPK |
| | [Mycobacterium [tuberculosis] | |
| 304 | hypothetical protein CAB90_02843 | LETDRAHHNQLVGDR |
| | [Mycobacterium tuberculosis] | |
| 305 | chromosome segregation protein | DELANAQAALR |
| | SMC [Mycobacterium tuberculosis | |
| 306 | hypothetical protein K875_03544 | HPGLKVIVGHMGELLPYCFSR |
| | [Mycobacterium [tuberculosis] | |
| 307 | Uncharacterised protein | TLTEGGAGHYVSLLTNR |
| | [Mycobacterium tuberculosis] | |
| 308 | transcriptional regulator % 2C luxR | AEATAADALAR |
| | family [Mycobacterium | |
| | tuberculosis] | |
| 309 | COX07571.1 Uncharacterised | LDSPDTSNAPR |
| | protein [Mycobacterium | |
| | tuberculosis] | |
| 310 | polynucleotide phosphorylase | IGEVIGPK |
| | [Mycobacterium tuberculosis] | |
| 311 | hypothetical protein K875_04662 | LGDDDVTPK |
| | [Mycobacterium [tuberculosis] | |

In an embodiment, peptide comprising an amino acid sequence selected from the group consisting of SEQ ID NO: 230 to SEQ ID NO: 268 and 312 or a variant sequence thereof which is at least 77%, preferably at least 88%, homologous (preferably at least 77% or at least 88% identical) to SEQ ID NO: 230 to SEQ ID NO: 268 and 312, wherein said is a biomarker for identification and diagnosis for TB disease, wherein said peptide is not the underlying full-length polypeptide.

TABLE 5
List of TB peptides as potential biomarkers

| | Validated Protein | |
|---|---|---|
| SEQ. ID. No | Description | Peptide Sequence |
| 235 | DitF protein | MLECYLQLSR |
| | OS = Mycobacterium | |
| | tuberculosis | |
| | GN = ERS075342_10708 | |
| | PE = 4 SV = 1 | |
| 236 | Gp11 OS = Mycobacterium | AAEQYWQAEQATNGYMLK |
| | phage fionn GN = 11 PE = 4 | |
| | SV = 1 | |
| 237 | Acyl-CoA dehydrogenase | EIRPNLDELETGALSPYPIMR |
| | FadE21 | |
| | OS = Mycobacterium | |
| | marinum (strain ATCC | |
| | BAA-535 / M) GN = fadE21 | |
| | PE = 3 SV = 1 | |
| 238 | Uncharacterized protein | AIFVLGDEGMEGGGGTLTSAAVTK |
| | OS = Mycobacterium | |
| | tuberculosis | |
| | GN = SAMEA2682835_00833 | |
| | PE = 4 SV = 1 | |
| 239 | Uncharacterized protein | WFAELTTK |
| | OS = Mycobacterium sp. | |
| | TKK-01-0059 | |
| | GN = K883_04441 PE = 4 | |
| | SV = 1 | |
| 240 | Uncharacterized protein | DKAALSSLR |
| | OS = Mycobacterium | |
| | tuberculosis TKK-01-0051 | |
| | GN = K875_04984 PE = 4 | |
| | SV = 1 | |
| 241 | Tetr-family transcriptional | QDEIIEATGR |
| | regulator | |
| | OS = Mycobacterium | |
| | tuberculosis | |
| | GN = ERS075342_07798 | |
| | PE = 4 SV = 1 | |
| 242 | Uncharacterized protein | MQAAVAQGR |
| | OS = Mycobacterium | |
| | tuberculosis | |
| | GN = ERS007661_02799 | |
| | PE = 4 SV = 1 | |
| 243 | Organic solvent tolerance | NDLGVFDFR |
| | protein OS = Mycobacterium | |
| | tuberculosis GN = 1ptD | |
| | PE = 3 SV = 1 | |
| 244 | Uncharacterized protein | AIAENPDDIAEYCTSGLTLTGFLGLSDTPR |
| | OS = Mycobacterium sp. | |
| | TKK-01-0059 | |
| | GN = K883_01262 PE = 4 | |
| | SV = 1 | |
| 245 | Diacylglycerol O- | VAVIELDADR |
| | acyltransferase | |
| | OS = Mycobacterium | |
| | tuberculosis TKK-01-0051 | |
| | GN = K875_00641 PE = 3 | |
| | SV = 1 | |
| 246 | Uncharacterized protein | VLAAASSPEK |
| | OS = Mycobacterium avium | |
| | XTB13-223 | |
| | GN = X425_01922 PE = 4 | |
| | SV = 1 | |
| 247 | Uncharacterized protein | EFLLDYLGIQHEPGVVMSR |
| | OS = Mycobacterium | |
| | tuberculosis | |
| | GN = ERS027644_02250 | |
| | PE = 4 SV = 1 | |
| 248 | Formate dehydrogenase H | VVFINPADIEALGLTPGGR |
| | FdhF OS = Mycobacterium | |
| | sp. TKK-01-0059 | |
| | GN = K883_00274 PE = 3 | |
| | SV = 1 | |
| 249 | Uncharacterized protein | FSVAELVRPWLDTWR |
| | OS = Mycobacterium avium | |
| | XTB13-223 | |
| | GN = X425_04258 PE = 4 | |
| | SV = 1 | |
| 250 | Uncharacterized protein | IAIVDSSFNHTFCAIIYCFHSFNYCFK |
| | OS = Mycobacterium | |
| | tuberculosis | |
| | GN = ERS007672_06446 | |
| | PE = 4 SV = 1 | |
| 251 | Oxidoreductase | VTAICGFADLEALVEAGRTFR |
| | OS = Mycobacterium | |
| | tuberculosis TKK-01-0051 | |
| | GN = K875_00555 PE = 4 | |
| | SV = 1 | |
| 252 | Cytochrome P450 191A3 | GQHFCIGYHLARLEISVLLQEWFR |
| | Cyp191A3 | |
| | OS = Mycobacterium | |
| | marinum (strain ATCC | |
| | BAA-535 / M) | |
| | GN = cyp191A3 PE = 3 SV = 1 | |
| 253 | Uncharacterized protein | LTGADFTR |
| | OS = Mycobacterium | |
| | thermoresistibile (strain | |
| | ATCC 19527 / DSM 44167 / | |
| | CIP 105390 / JCM 6362 / | |
| | NCTC 10409 / 316) | |
| | GN = KEK_08147 PE = 4 | |
| | SV = 1 | |
| 254 | Arylsulfatase AtsD | QALLTGR |
| | OS = Mycobacterium | |
| | marinum (strain ATCC | |
| | BAA-535 / M) GN = atsD | |
| | PE = 4 SV = 1 | |
| 255 | Putative taurine ABC | MADITGNKQPR |
| | transporter permease | |
| | protein OS = Mycobacterium | |
| | tuberculosis GN = ssuC | |
| | PE = 3 SV = 1 | |
| 256 | Uncharacterized protein | ASVSPLSTAPFASIGSDDAVPSR |
| | OS = Mycobacterium | |
| | tuberculosis | |
| | GN = ERS075342_08795 | |
| | PE = 4 SV = 1 | |
| 257 | ABC transporter ATP- | MQPGTTTAIVGPSGCGK |
| | binding protein | |
| | OS = Mycobacterium | |
| | tuberculosis GN = irtB PE = 4 | |
| | SV = 1 | |
| 312 | Uncharacterized protein | ATTTDMLR |
| | OS = Mycobacterium sp. | |
| | TKK-01-0059 | |
| | GN = K883_01691 PE = 4 | |
| | SV = 1 | |
| 258 | Uncharacterized protein | AFMTPSIAAALGRWFWWPLNTFNIVQR |
| | OS = Mycobacterium | |
| | tuberculosis TKK-01-0051 | |
| | GN = K875_00952 PE = 4 | |
| | SV = 1 | |
| 259 | Sodium: dicarboxylate | LLQMVIMPLVFASILSAVAR |
| | symporter | |
| | OS = Mycobacterium | |
| | tuberculosis GN = tcyP | |
| | PE = 4 SV = 1 | |
| 260 | Gp79 OS = Mycobacterium | MDPDALVAQLVDEAQNVLR |
| | phage Hedgerow GN = 79 | |
| | PE = 4 SV = 1 | |
| 261 | Cell envelope-related | VSVVSLPR |
| | transcriptional attenuator | |
| | OS = Mycobacterium | |
| | tuberculosis GN = ywtF_3 | |
| | PE = 4 SV = 1 | |
| 262 | Acyl-CoA synthetase | DFHAGFMSSGDVGYLDENGR |
| | OS = Mycobacterium | |
| | tuberculosis (strain ATCC | |
| | 25618 / H37Rv) | |
| | GN = LH57_07805 PE = 4 | |
| | SV = 1 | |
| 264 | Uncharacterized protein | DHPAPLIVR |
| | OS = Mycobacterium | |
| | tuberculosis | |
| | GN = ERS007672_01114 | |
| | PE = 4 SV = 1 | |
| 265 | Uncharacterized protein | YLIGCDGGR |
| | OS = Mycobacterium | |
| | tuberculosis TKK-01-0051 | |
| | GN = K875_03148 PE = 4 | |
| | SV = 1 | |
| 266 | Uncharacterized protein | TGLSPSDMDVACMYDCFTYTVMATMEGF |
| | OS = Mycobacterium | GFCEK |
| | tuberculosis TKK-01-0051 | |
| | GN = K875_04012 PE = 4 | |
| | SV = 1 | |
| 267 | Uncharacterized protein | SVTAAEGDALAR |
| | OS = Mycobacterium | |
| | marinum (strain ATCC | |
| | BAA-535 / M) | |
| | GN = MMAR_p20 PE = 4 | |
| | SV = 1 | |
| 268 | GntR family transcriptional | GARVPALDLEQMQVIYQMR |
| | regulator | |
| | OS = Mycobacterium | |
| | tuberculosis GN = mcbR_1 | |
| | PE = 4 SV = 1 | |
| 230 | ABC transporter ATP- | TPLTATGLSK |
| | binding protein | |
| | OS = Mycobacterium avium | |
| | XTB 13-223 | |
| | GN = X425_04233 PE = 4 | |
| | SV = 1 | |
| 231 | Gp54 OS = Mycobacterium | AGQRWISIVEPLR |
| | phage Yoshi GN = 54 PE = 4 | |
| | SV = 1 | |
| 232 | Glucose-6-phosphate | VTRETLALLIDLAR |
| | isomerase | |
| | OS = Mycobacterium bovis | |
| | GN = pgi PE = 3 SV = 1 | |
| 233 | Gp40 OS = Mycobacterium | SLWLAVVAGMIVAMLVYVLA |
| | phage Spartacus GN = 40 | |
| | PE = 4 SV = 1 | |

In an embodiment, peptides such as Seq. ID No. such as 288, 244, 245, 246, 248, 249, 251, 252 and 243 are biomarkers for mycobacterium genus including pathogenic and non-pathogenic species.

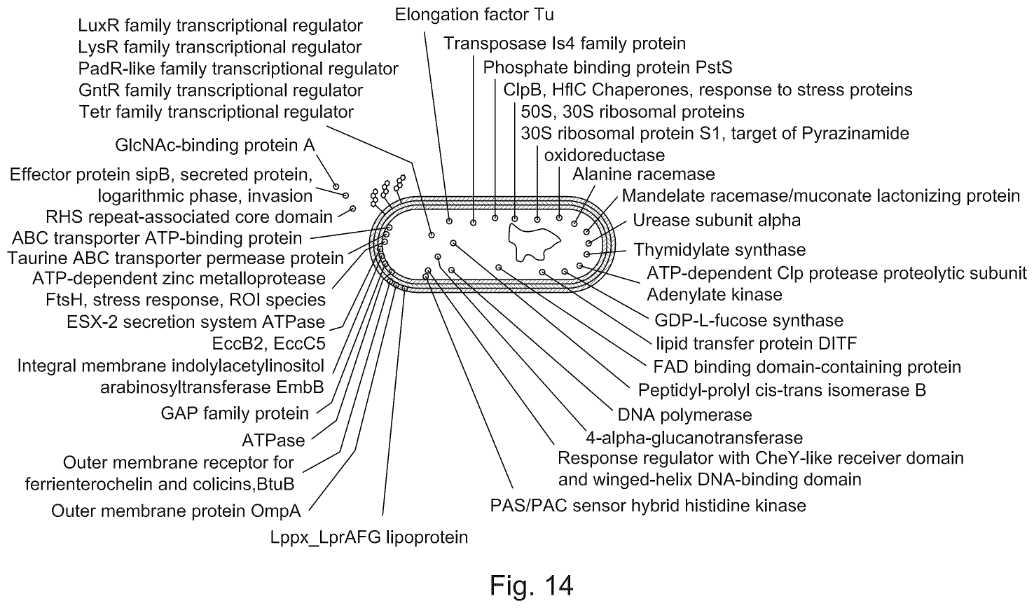

In one embodiment, peptides derived from proteins associated with mechanism of drug resistance in TB were detected in the urine of patients as shown in FIG. 14. In FIG. 14, proteins marked in dark grey are mechanistically associated with drug resistance, and include efflux pumps, enzymes required for cell wall integrity, mutated drug targets. Mtb derived markers include membrane bound channels and receptors, secreted proteins related to Mtb virulence, essential transcriptional regulators, signal transduction proteins, metabolic enzymes, essential stress response factors, enzyme necessary for DNA precursor biosynthesis, ribosomal proteins, drug targets.

In one embodiment, the identified peptide was twin-arginine translocation (Tat) protein (IFTEPAGDAAQGTEQRK (SEQ. ID No. 313), Accession No. CNE85390.1) whose function is to facilitate the export of tertiary proteins across the cytoplasmic membrane for Mtb. This protein is especially important for pathogenic strains of mycobacterium since virulence factors must be exported out of the bacterial cell.

In one embodiment, the identified peptide was ABC transporter ATP-binding protein (MQPGTTTAIVGPSGCGK (SEQ. ID No. 257), Accession No. CFE39497.1). The ABC Transporter protein uses ATP hydrolysis to drive transport of substrates across the cellular membrane, and are the overarching class for drug transporting proteins related to Mtb resistance to major antibiotics.

### Working Example

The invention is illustrated by the following working examples. The following examples as described are not intended to be construed as limiting the scope of the present invention.

General Material and Methods

Nanocage fabrication: Poly(NIPAm-co-BAC-co-AA)N-Isopropylacrylamide (NIPAm, 4.5 g, 39 mmol), bis(acryloyl)cystamine (BAC 236 mg, 0.9 mmol) and allylamine (AA, 338 μl, 4.5 mmol) will be dissolved in 150 mL of water, and filtered using a 0.45 μm nitrocellulose membrane disk filter. The system will be purged with nitrogen for 30 minutes at room temperature and then heated to 50° C. N,N,N′,N′-tetramethylethylenediamine (TEMED, Thermo Fisher, 19.4 mg, 0.17 mmol) will be added to the solution and after 10 minutes potassium persulfate (50 mg, 0.18 mmol) will be added to initiate the polymerization. The system will be held at 50° C. under nitrogen for 4 hours prior to washing and size characterization by light scattering and AFM.

Covalent incorporation of binding baits: the following four dye baits will be coupled to the nanoparticles and tested for affinity and yield of extraction as described previously [17, 31, 32]: reactive blue 221 (RB221), trypan blue (TB), Bismarck brown (BB), and Alcian Blue. For TB glycans, RB221 will be coupled to the cages as follows: 0.3 g of RB221 powder will be mixed to a solution obtained by adding 0.66 g of Na2CO3 to 50 ml of DI water and stirring at medium rate until completely dissolved. The solution will be filtered (0.45 μm pore size). 50 ml of cage suspension will be added and allowed to incubate overnight at room temperature. RB221 coupled cages will be washed five times (54,400 rcf, 50 min, 25° C.) and re-suspended in 50 mL of DI water.

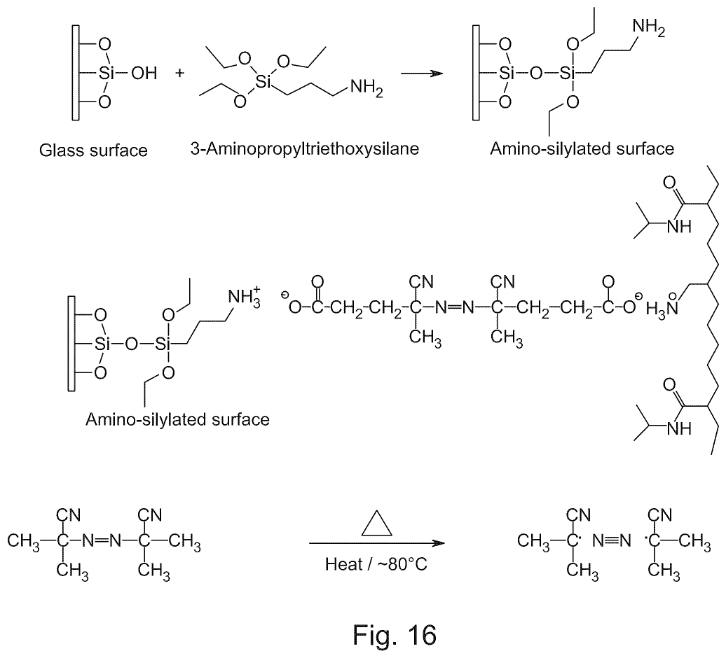

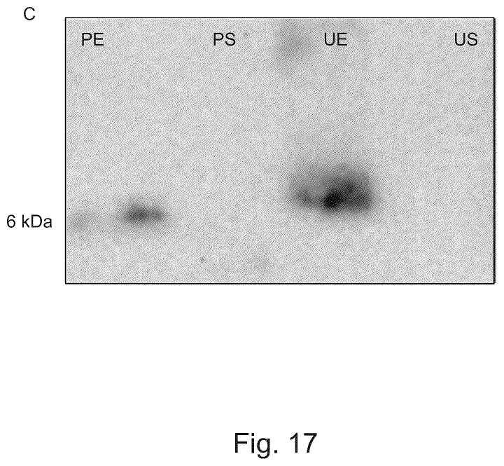

“Urigami” Collapsible affinity net urine collection vessel. The affinity net is glass wool tethered with Nanocage particles housed in a waterproof collapsible collection cup. Glass wool (10 mg, Ohio Valley Specialty™ Untreated Glass Wool, Fisher Scientific) will be acid treated (33% HCl for 2 hours at room temperature) to enable nanoparticle attachment. After the glass wool has dried from acid treatment, 3 mL of nanoparticles (10 mg/mL) will be incubated with the glass wool for 1 hour at room temperature. The dyed glass wool will then be dried in an oven at 85° C. for 15 minutes. Washes will then be performed with MilliQ water in order to remove excess nanoparticles. Alternatively, glass wool will be treated with 3-aminopropyltriethoxysilane and 4,4′-Azobis(4-cyanovaleric acid) will be used to covalently attach amine containing nanocages. Cages will be covalently bound to the glass fibers by reversible cross linkers, that are detachable using heat (80° C., FIG. 16). A sheet of waterproof material will be folded into the desired container shape (FIG. 3). The nanoparticle-containing glass wool will be placed inside the waterproof container (FIG. 3). Urine will be added to the container, let incubate with the affinity nanoparticles tethered to the glass wool for 10 minutes and then discarded. Water washes and elution will be performed as described above; separation of the glass wool from solution will be achieved by simple compression (FIG. 2).

Fabrication of visual lateral flow one step urine test employing Nanocages: An enzymatically amplified color reaction occurs inside the nanoparticles containing the captured pathogen analyte for the nanoparticles bound to the antibody detection line (FIG. 15): Integrating the Nanocage technology into a new type of lateral flow immunoassay.

Pore dilation of cages in order to allow antibody access to captured analytes (FIG. 18). Nanocages will be reduced by a 10% sodium dithionite solution in water for 2 minutes. Cages will be washed five times by centrifugation (19,000 rpm, 50 min, 25° C.).

Covalent bonding of HRP into the nanocage. HRP will be linked to the cages using the Lightning kit (Aviva Biosciences). 100 μL of 5 mg/mL HRP solution in PBS will be mixed with Nanocages (100 μL, 5 mg/mL dry weight concentration) and allowed to incubate at room temperature for 15 minutes prior to washing, impregnation in the wick and freeze drying.

Nitrocellulose, Wick and Cassette: Microporous nitrocellulose membranes (pore sizes in the range 5-30 microns) will be screened in order to identify the optimal performance: rapid flow, good wettability, and good visual resolution. Test reagents and effects of reagents location in the test will be investigated (GE healthcare Life Sciences, Millipore, etc.). Cellulose and polymer (Polypropylene, PTFE, PVDF, Porex, Rayon, polyester, viscose) based wicking materials will be investigated (GE Healthcare Life Sciences, Interstate Specialty Products). Thickness will range from 500 μm to 3000 μm. Custom made cassettes capable to incorporate larger than standard wick volumes will be designed with Google SketchUp and 3D printed with equipment at Mason or through 3D printing service (Shapeways, Inc.).

HRP substrate. 3,3′-diaminodbenzidine (DAB) will be adsorbed in the input wick. DAB is a sensitive colorimetric substrate that is oxidized by H2O2 in the presence of HRP and produces an intense brown, insoluble polymeric product. Sensitivity can be enhanced further by addition of metals. Other colorimetric substrates will be investigated: 4-Chloro-1-naphthol (4CN, insoluble purple product); 3,3′,5,5′-Tetramethylbenzidine (TMB, soluble blue product); 2,2′-Azino-di(3-ethylbenzthiazoline-6sulfonate) (ABTS, soluble green color); o-Phenylenediamine dihydrochloride (orange-brown soluble product).

Analytical sensitivity of the novel lateral flow immunoassay. Glycan binding nanocages preloaded with HRP will be adsorbed in the upfront wick. Model solutions containing different concentrations of pathogen antigens in healthy volunteer urines will be used to assess the analytical sensitivity of the prototype device. Interfering substances will be mixed to the model solutions in order to assess and minimize nonspecific background signal.

Immuno macroarray analysis of urinary antigen: Cages will be separated from urine by centrifugation, washed with DI water and mixed with 10 μL of Novex 2× Tris-Glycine SDS Sample Buffer (Thermo Fisher Scientific) containing 10% (v/v) 2-mercaptoethanol and incubated at 100° C. for 2 minutes. The cage suspension will be centrifuged (16,100 rcf, 25° C., 10 minutes) and the supernatant was saved and subjected to detergent removal (HiPPR Detergent Removal Resin Column Kit, Thermo Scientific) according to the vendor's instruction and using 100 μl of bead suspension. Aliquots of 4 p L of the resulting purified elution will be robotically deposited on PVDF membranes previously activated with methanol and rinsed with DI water [33]. Membranes will be allowed to dry at room temperature and then stained using antibody, HRP labelled anti mouse antibody, and enhanced chemiluminescence system (Supersignal West Dura, Thermo Fisher Scientific).

Mass Spectrometry: Antibody independent TB specific antigen urine test for clinical validation and discovery. Antigens captured in the nanocages will be eluted with 1% Rapigest (Waters, Millford, MA) in 50 mM ammonium bicarbonate with 10 mM TCEP for 10 minutes at 100° C. Eluates will be alkylated with 50 mM iodoacetamide for 15 minutes in the dark at room temperature, then diluted 10-fold in 50 mM ammonium bicarbonate to lower the Rapigest concentration to 0.1%. Samples will be then digested with trypsin overnight at 370 C. Digestion will be halted by adding trifluoroacetic acid to a final concentration of 0.1%. Samples will be desalted with C-18 spin columns (Thermo Fisher), dried by vacuum centrifugation, and then reconstituted in 0.1% formic acid in water. Trypsin digested eluate samples will be analyzed on an Orbitrap Fusion mass spectrometer (ThermoFisher Scientific, Waltham, MA, USA) equipped with a nanospray EASY-nLC 1200 HPLC system (Thermo Fisher Scientific, Waltham, MA, USA). Peptides will be separated using a reversed-phase PepMap RSLC 75 μm i.d.×15 cm long with 2 μm, C18 resin LC column (ThermoFisher Scientific, Waltham, MA, USA). The mobile phase will consist of 0.1% aqueous formic acid (mobile phase A) and 0.1% formic acid in 80% acetonitrile (mobile phase B). After sample injection, the peptides will be eluted by using a linear gradient from 5% to 50% B over 15 min and ramping to 100% B for an additional 2 min. The flow rate will be set at 300 nL/min. The Orbitrap Fusion will be operated in a data dependent mode in which one full MS scan (60,000 resolving power) from 300 Da to 1500 Da using quadrupole isolation, will be followed by MS/MS scans in which the most abundant molecular ions will be dynamically selected by Top Speed, and fragmented by collision-induced dissociation (CID) using a normalized collision energy of 35%. “Peptide Monoisotopic Precursor Selection” and “Dynamic Exclusion” (8 sec duration), will be enabled, as will be the charge state dependency so that only peptide precursors with charge states from +2 to +4 will be selected and fragmented by CID.

MS Bioinformatics pipeline. High-confidence peptide identifications will be obtained by applying the following filter criteria to the search results: Xcorr versus charge 1.9, 2.2, 3.5 for 1+, 2+, 3+ ions; ΔCn >0.1; probability of randomized identification e0.01. Acceptable false discovery rate (FDR) based on forward-reverse decoy will be <1% [35]. The following pre-analytical filtering criteria will be applied: 1) absence of carryover as determined by analyzing a blank sample with a 90-minute gradient, and 2) manual validation of each peptide spectra.

Statistical Plan Under CAP/CLIA laboratory compliance including blinded proficiency testing, statistical analysis will be conducted using the guidelines in the FDA CDRH guidance document for diagnostic test evaluation. Regression analysis will be performed with STATA 13. The relationship between covariates and outcome will be explored using linear, logistic, and ordinal regression. Forward and backward stepwise regression will be used to optimize covariate selection. The covariates of interest are: indicators of socioeconomic status, clinical symptoms, such as cough, fever, and weight loss; as well as indicators of appetite.

### Example 1: Urine Tb Screening in Adults

In one embodiment, the invention relates to harvesting and detection of known TB antigens lipoarabinomannan (LAM) and ESAT-6 in HIV negative culture positive pulmonary TB.

The clinical validation of our technology (N=1200 banked specimens with full clinical characterization), and to create additional novel technology, to address urgent roadblocks for urine TB screening in the laboratory and in the field (FIG. 1).

Tuberculosis patient characterization. Urine samples (N=600) were collected from hospitalized patients in Peru. Diagnosis of active pulmonary tuberculosis for these hospitalized patients was performed by analyzing sputum samples by means of Auramine stain for acid-fast bacilli, and using the microscopic observation broth-drug susceptibility assay (MODS). The relative intensity of Auramine staining for acid fast organisms was scored from 0 to 3 with 3 being the highest [28]. Specimens were collected under informed consent; the study received IRB approval at the Universidad Peruana Cayetano Heredia (Lima, Peru) and Johns Hopkins Blooomberg School of Public Health (Baltimore, MD). Clinical and demographic data were collected from patients following recruitment to the study. This included age, sex, previous TB diagnosis, weight, appetite, self-reported symptoms including cough, hemoptysis, fever, fatigue, and average number of cough in prior 24 hours. Appetite was assessed using the Simplified Nutritional Appetite Questionnaire (SNAQ), which has been used and validated to assess appetite and weight loss in ambulatory patients in a range of settings. Urine samples were immediately centrifuged at 3,000 rcf for 10 min, and the supernatants were stored in liquid nitrogen or −80° C. until use. Urine specimens from 300 hospitalized diseased controls and 300 healthy volunteer (Table 6) negative controls were collected from the same geographical area. Patient urine samples were qualified before the analysis by urinary dipstick testing (Multistix GP, Siemens) for hematuria, proteinuria, cystitis, and specific gravity analysis for each case. Diseased non-TB patients were ill with a variety of severe systemic, pulmonary, and urinary tract diseases including pneumonia, lung cancer, pyelonephritis, genitourinary infection, sepsis, cryptosporidiosis, cerebral toxoplasmosis, giardiasis, colon cancer with gastroenteritis, and liver failure.

TABLE 6
Information of TB patients and controls.

| | Median age - | |
|---|---|---|
| | years (IQR) | Sex: M/F |
| Tuberculosis patients | 29 (22-37) | 410/190 |
| (microbiologically | | |
| proven), N = 600 | | |
| Healthy volunteers N = 300 | 26 (22-37) | 170/130 |
| Diseased, tuberculosis negative | 32 (28-51) | 185/105 |
| controls, N = 300 | | |

•‘N’ signify number

TABLE 7
Microbiological and clinical data of N = 600

| Microbiological and | |
|---|---|
| Clinical Data, N = 600 | N |
| Auramine Sputum Smear | |
| Microscopy Results | |
| 0 (%) | 120 |
| 1 (%) | 190 |
| 2 (%) | 110 |
| 3 (%) | 90 |
| Paucibacillary (%) | 90 |
| MODS | |
| Positive | 600 |
| Negative | 0 |
| Mycobacterium tuberculosis | |
| isolate sensitive to: | |
| Isoniazid | 580 |
| Rifampicin | 580 |
| Weight in kilograms | 52.9 |
| SNAQ Composite Score | 13.2 |
| Self- reported Symptoms | |
| Cough | |
| Yes | 430 |
| No | 170 |
| Hemoptysis | |
| Yes | 160 |
| No | 440 |
| Fever | |
| Yes | 410 |
| No | 190 |
| Fatigue | |
| Yes | 310 |
| No | 290 |

Example 1 was executed using general materials and methods discussed prior in the application. The antibodies for LAM and ESAT-6 were tested and calibrated prior usage in the experiment. Anti-LAM and anti-ESAT6 monoclonal antibodies were obtained from BEI Resources (clone CS-35) and Abcam (clone 11G4), respectively and yielded a highly specific single band for LAM and ESAT6 with no detectable background in urine matrix. Glucose Oxidase, anti LAM mouse antibody (BEI resources) or anti LPG mouse antibody will be diluted 1:10 in PBS for a final concentration of 0.1 mg/mL. 0.01 mL of the diluted mAb solution will be striped on nitrocellulose (1×4 cm, Millipore) using an automated striper and dried at 37° C. in a forced air oven (Fisher scientific Isotemp). The nitrocellulose membrane will be blocked with 50 mg/mL PEG 8000 for 30 minutes, rinsed and dried. H2O2 delivery rate will be balanced by optimizing the glucose: GO ratio.

Immuno macroarray analysis of urinary LAM and ESAT6. Purified Lipoarabinomannan (LAM) from Mycobacterium tuberculosis strain H37Rv and Anti LAM antibody will be obtained from BEI Resources (Cat No. NR-14848, and NR-13811 LAM mAb clone CS-35). ESAT6 recombinant protein and anti-ESAT6 monoclonal antibody clone 11G4 will be obtained from Abcam (catalog No. ab124574 and ab26246). Membranes will be allowed to dry at room temperature and then stained using anti-LAM CS-35 or anti-ESAT6 mAb, HRP labelled anti mouse antibody, and enhanced chemiluminescence system (Supersignal West Dura, Thermo Fisher Scientific).

Mass Spectrometry: Tandem mass spectra will be searched against the NCBI Mycobacterium tuberculosis databases using Proteome Discover v 2.1 with SEQUEST using tryptic cleavage constraints. Mass tolerance for precursor ions will be 5 ppm, and mass tolerance for fragment ions will be 0.05 Da. Data will be analyzed with oxidation (+15.9949 Da) on methionine as a variable post translation modification, and carbamidomethyl cysteine (+57.0215) as a fixed modification. A 1% false discovery rate (FDR) will be used as a cut-off value for reporting peptide spectrum matches (PSM) from the database. Tandem mass spectra will be searched against the UNIPROT, NCBI and Gene Expression Omnibus (GEO, accession number GSE62152[34]) Mycobacterium tuberculosis databases with Proteome Discoverer software using tryptic cleavage constraints.

Statistical Plan: Primary outcome is detection of LAM and/or ESAT6 in patient urine. LAM and ESAT6 will be considered as linear, binary (urinary LAM cutpoint=115 pg/mL ESAT cutpoint=100 pg/mL), and ordinal outcome (urinary LAM<115 pg/mL, 115 pg/mL<urinary LAM <320 pg/mL, urinary LAM >320 pg/mL).

### Results of Example 1:

Test Target Criteria. Disposable lateral flow urine RDT. Time to result=15 minutes. Readout=visual band compared to control band on same strip. Power requirements=none. Operating temperature=+5° C. to +70° C. with 70% humidity. Stability=2 years at 0° C. to 40° C. in foil package. Reagent integration=self-contained. Diagnostic sensitivity for pulmonary TB compared positive smear and positive sputum culture=90% (>68% for smear-negative culture-positive patients). Specificity=90% compared to microbiological reference standard [2].

In one embodiment, the technology had sensitivity >95% and a specificity of >80% for active pulmonary TB in HIV negative patients, compared to non-TB diseased controls.

Importantly, we show that the concentration of urinary LAM correlates significantly with the clinical severity of the disease.

FIG. 15 shows TB antigen binding nanocages are seamlessly integrated into a novel lateral flow immunoassay for Tuberculosis rapid diagnostics. Nanocages capture urinary pathogen antigens and arrest on the antibody line. Horse radish peroxidase HRB mediated enzymatic amplification and large (1 mL) volume of sampled urine enhance the analytical sensitivity >100 fold compared to conventional lateral flow immunoassays (target=1.25 pg/mL).

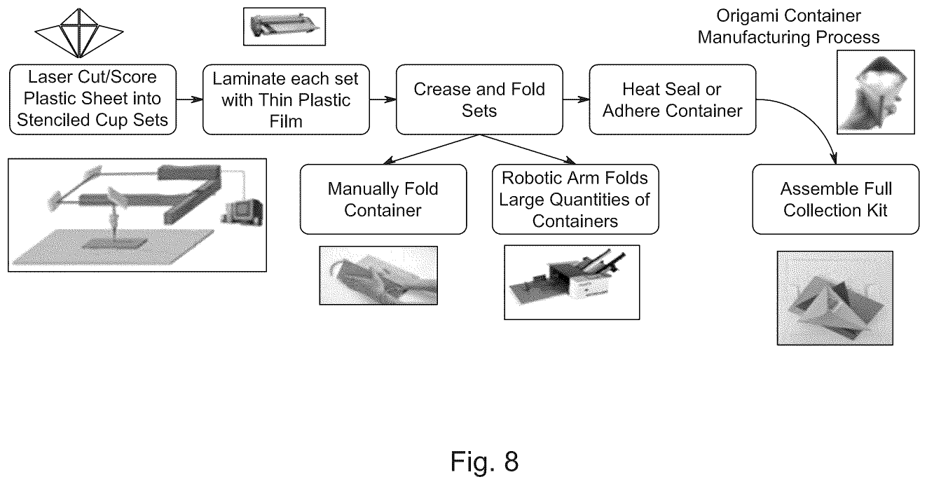

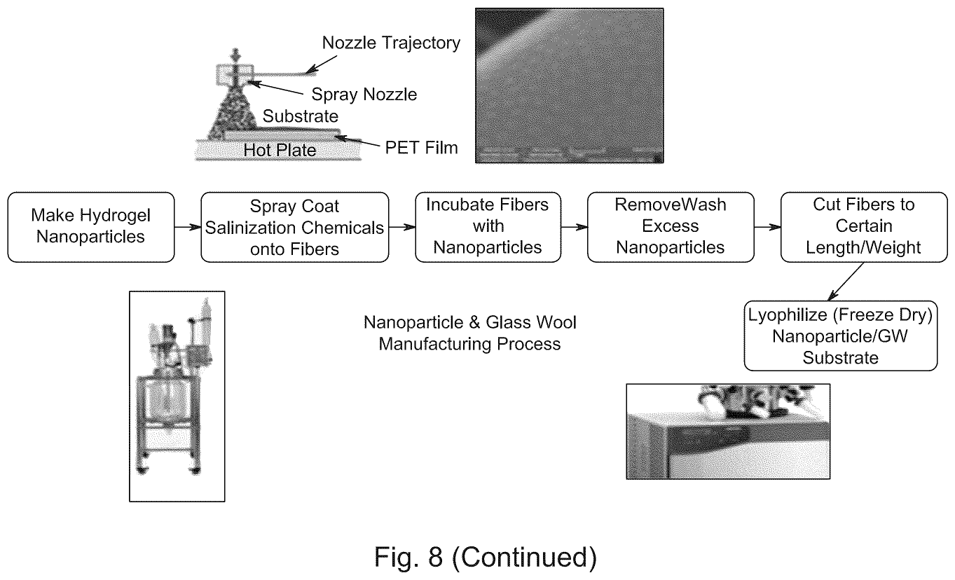

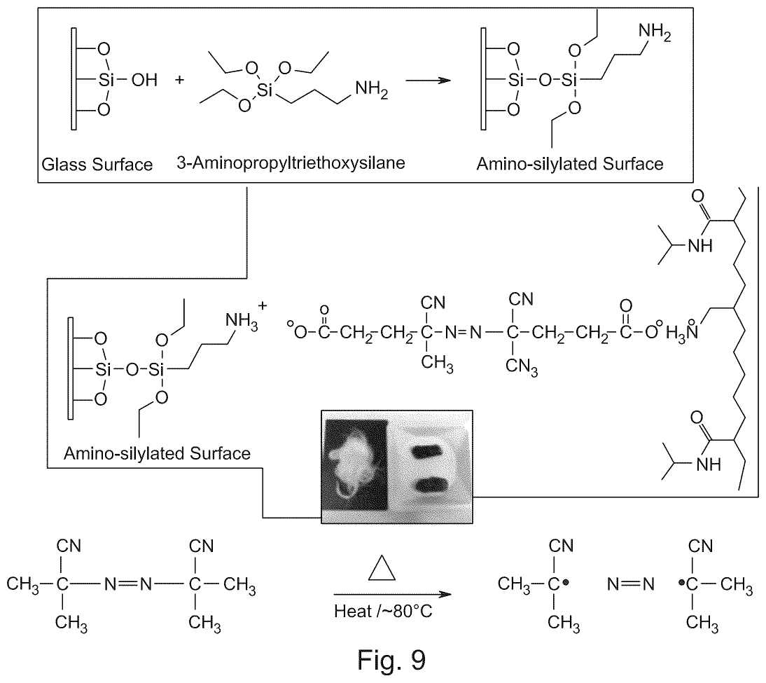

The FIG. 18 and FIG. 19 shows the result of immune assay. The internal surface area of the hydrogel polymer mesh Nanocages is thousands of times greater than the surface of an equivalent solid particle. The effective pore size of the particles is a function of hydrogel polymer crosslinks. Rendering the crosslinks degradable provides a means to induce nanocages to open up and display the captured sequestered analyte (TB antigen) cargo. Partially degradable Nanocage cages were created incorporating a cleavable cross linker (1,2-Dihydroxyethylene bis-acrylamide, DHEA) under oxidizing conditions (FIG. 18A, B, C) or (N, N′-bis(acryloyl)cystamine, BAC) under reducing conditions (FIG. 18D). In this novel workflow, Nanocage cages were mixed with urine containing the antigen of interest and the solution phase antigen was captured within the particles. Cages were then exposed to degradative conditions. The degradable crosslinks were cleaved causing an effective increase in pore size, exposing the captured antigens in the internal volume. Antibodies were used to probe the exposed captured antigen directly within the cages for LAM (FIG. 18D) and ESAT6 (FIG. 18C). This novel class of cages permit non-elution immune-based detection of captured analytes directly sequestered and highly concentrated inside the cages (FIG. 8E, F). The workflow is greatly simplified because the need for elution is obviated. The FIG. 19 documents the feasibility and sensitivity of incorporating the enzymatic amplification reaction within the volume of the nanoparticles (working principle in FIG. 15).

In FIG. 20 LAM was quantitatively measured in the urine with a sensitivity >95% and a specificity >80% (p<0.00001, N=101) in a concentration range of 14 to 2000 picograms per mL, as compared to non-TB, healthy and diseased, age matched controls (ROC AUC=0.95, 95% CI: 0.9005-0.9957). Urinary LAM was elevated in patients with a higher mycobacterial burden (p<0.043, N=42), a higher proportion of weight loss (p<0.03, N=37), or cough (p=0.042, N=50). ROC curve is a graphic presentation of the relationship between both sensitivity and specificity and it helps to decide the optimal model through determining the best threshold for the diagnostic test.

We demonstrated that the mAb is specific for Mtb LAM in urine in comparison to polysaccharides purified from multiple serotypes of N. meningitidis and S. pneumoniae. We screened a high number of anti-LAM antibodies. In particular, the human mAb clone A194-01 demonstrated significantly improved affinity for Mtb LAM with respect to clone CS-35. An optimized anti-LAM sandwich immunoassay comprising capture (CS-35 or FIND 28) and detection (A194-01) antibodies. The novel sandwich immunoassay attains high sensitivity in bacterial and urine LAM and it can readily translated to a rapid test, lateral flow immunoassay as shown in FIG. 20.

Identification of novel Mtb proteins in the urine of tuberculosis patients: characterized TB patients were analyzed with the Nanocage enhanced mass spectrometry workflow. Several new peptides were identified according to the criteria of Bioinformatics pipeline. In addition, to the preanalytical filtering criteria for MS bioinformatics pipeline for identification of peptides, the following post-analytical filtering criteria is applied: 1) peptide length >7 amino acids. 2) 100% amino acid identity match with pathogenic Mtb, 3) exclusion of housekeeping proteins (tubulin, actin, ubiquitin), 4) exclusion of peptides that overlap with any naturally occurring in non-Mycobacterium protein (100% identity match), 5) authentication of protein annotation using randomized protein databases and alignment of whole candidate proteins in the closest taxonomy clade (JalView), and 6) a patient will be considered positive for Mtb infection if >=2 peptides uniquely belonging to Mtb according to the present criteria will be identified.

Several proteins of potential high biological importance were found. These include GAP family protein an integral membrane protein required for glycolipid transport to the cell surface, PadR-like family transcriptional regulator, which has been associated to a defective form of the mycobacterial cell wall in response to antimicrobial factors, and a number of proteins associated to glucose, nitrogen and lipid metabolism. Table 8 gives a list of Mtb proteins identified in the urine of active pulmonary TB patients.

TABLE 8
Mtb proteins identified in the urine of active pulmonary TB patients.
True Positive Patients

| Definition [Mtb] | Accession Number | Q Value |
|---|---|---|
| Twin-arginine translocation | CNE65390.1 | 0.01668 |
| pathway signal (virulence | | |
| factor) | | |
| ABC transporter ATP- | CFE39497.1 | 0.04403 |
| binding protein (multidrug | | |
| resistance) | | |
| cell envelope-related | CNF54817.1 | 0.003363 |
| transcriptional attenuator | | |
| arylsulfatase AtsD | AMC71783.1 | 0.008922 |
| Antirestriction protein | SGC98382.1 | 0.01380 |
| (immune evasion) | | |
| Tetr-family transcriptional | CNG74091.1 | 0.004629 |
| regulator (multidrug | | |
| resistance) | | |
| Organic solvent tolerance | SGD19216.1 | 0.004629 |
| protein (nutrient deprivation) | | |
| acyl-CoA dehydrogenase | WP_094357863.1 | 0.02466 |
| (metabolic activity) | | |
| acyl-CoA synthetase | WP_052629386.1 | 0.1144 |
| (dormancy / latent | | |
| infection) | | |

### Example 2: Detection Test for Active Tb in Children and Adults

The method employed to execute Example 2 was similar to sections of General Material and Methods and Example 1. The urine samples (N=300) were collected from hospitalized patients in Peru. Diagnosis of active pulmonary tuberculosis was done according to Example 1.

A total of 60 Mycobacterium tuberculosis specific proteins were identified in urine samples from 150 culture and PCR positive TB patients (FIG. 14), following the workflow of FIG. 1 and authentication by the Bioinformatics pipeline. As shown in FIG. 14, Proteins marked in purple are mechanistically associated with drug resistance, and include efflux pumps, enzymes required for cell wall integrity, mutated drug targets. Mtb derived markers include membrane bound channels and receptors, secreted proteins related to Mtb virulence, essential transcriptional regulators, signal transduction proteins, metabolic enzymes, essential stress response factors, enzyme necessary for DNA precursor biosynthesis, ribosomal proteins, drug targets. Markers include GAP family protein, WP_096872076.1, an integral membrane protein required for glycolipid transport to the cell surface, PadR-like family transcriptional regulator, CNF95600.1, which has been associated to a defective form of the mycobacterial cell wall in response to antimicrobial factors. A number of secreted proteins and proteins associated to glucose, nitrogen and lipid metabolism were identified including LysR family transcriptional regulator, CND61526.1; 4-alpha-glucanotransferase, AGL27231.1; Alpha/beta hydrolase (DUF1023), CMM76917.1; Cytochrome P450 [mycobacterium sp.], Tetr Family Transcriptional Regulator, WP_102419657.1; ESAT-6 like Protein ESXK, AFF13548.1; WXG 100 Family Type VII Secretion Target, WP_068114113.1. twin-arginine translocation (Tat) protein (IFTEPAGDAAQGTEQRK (SEQ ID No. 313), Accession No. CNE85390.1) whose function is to facilitate the export of tertiary proteins across the cytoplasmic membrane for Mtb. This protein transport virulence factors out of the bacterial cell. Outer membrane receptor for ferrienterochelin and colicins, BtuB is a transporter required for import of essential micronutrient through the waxy Mtb cell wall, a drug target. GlcNAc-binding protein A (GbpA) is a secreted attachment factor that binds to N-acetyl-D-glucosamine, a sugar residue that is present on the surface of epithelial cells, is required for cell adhesion and invasion and can be a virulence factor. “PAS/PAC sensor hybrid histidine kinase” and “response regulator with CheY-like receiver domain and winged-helix DNA-binding domain” are two interacting proteins in the Mtb signal transduction system. Phosphorylation of the CheY receiver domain by the PAS/PAC kinase induces a conformational shift in the DNA binding domain that promotes association with cognate DNA. Peptidyl-prolyl cis-trans isomerase B is a cyclophilin that induces isomerization of peptide bonds preceding prolyl residues and regulates protein folding. This enzyme demonstrated also chaperon-like activity and it is considered a stress responder that contributes to Mtb virulence. This is an immunogenic protein that can modulate host immune response and possibly improve persistence of the bacterium. Among the analyzed samples, seven urine samples derived from pediatric HIV negative, culture and PCR positive TB patients. A total of 60 Mycobacterium tuberculosis specific proteins were identified (FIG. 14).

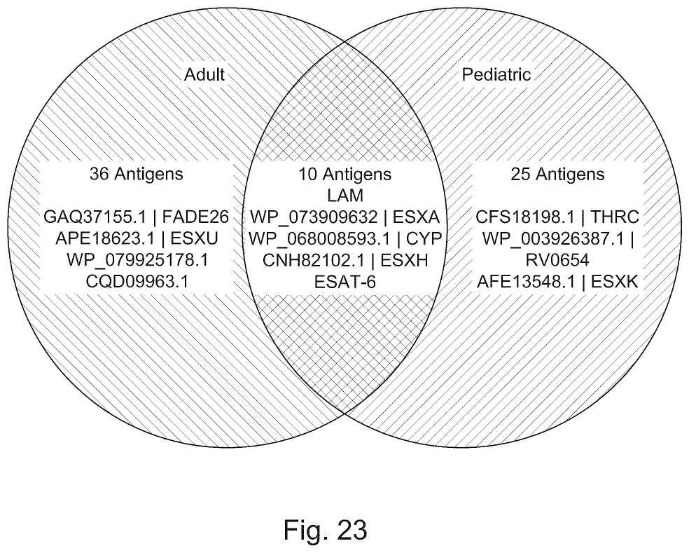

Ten antigens were in common between pediatric and adult patients as shown in FIG. 23.

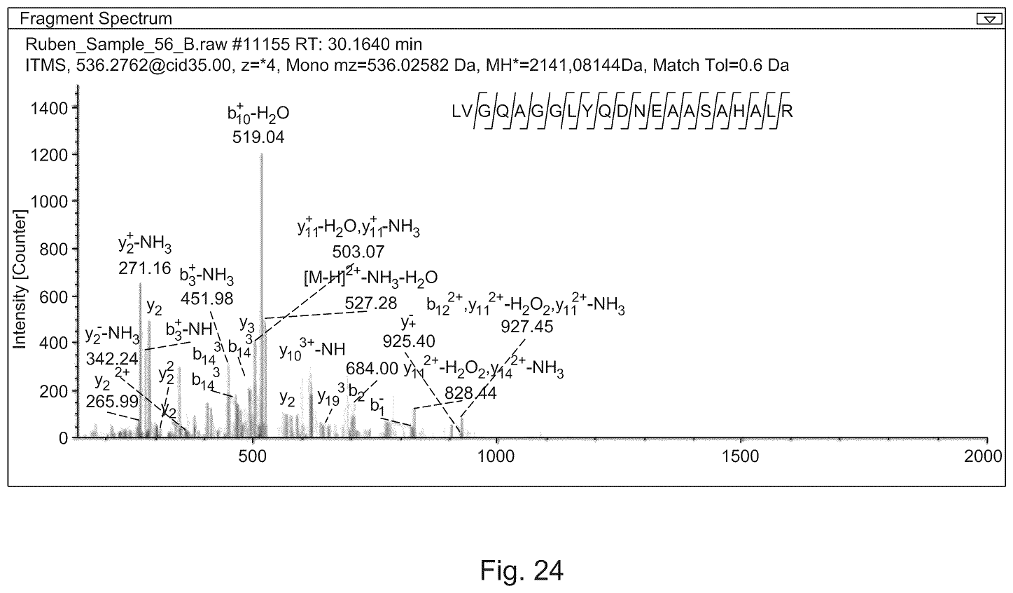

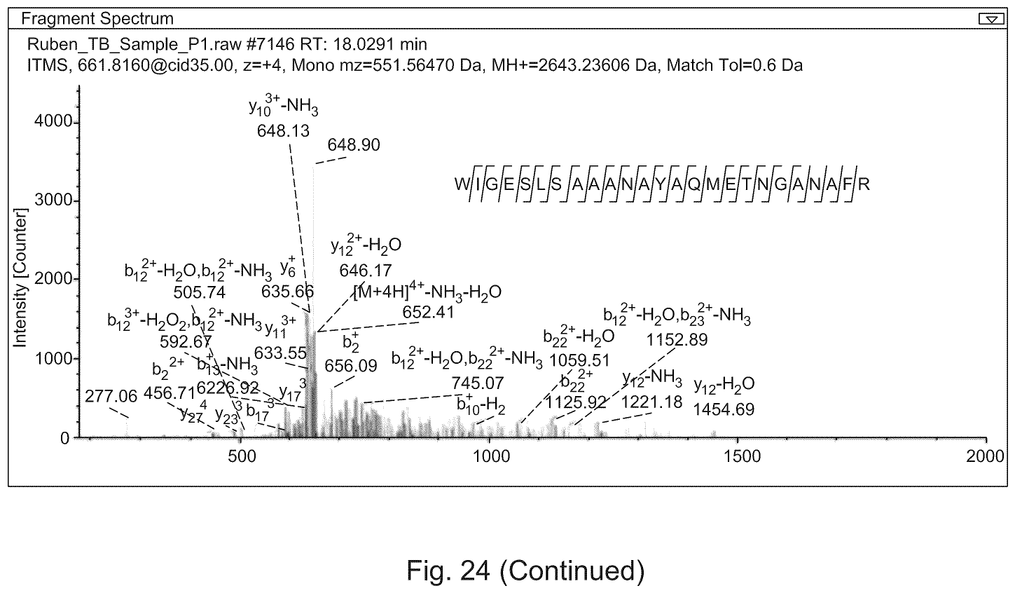

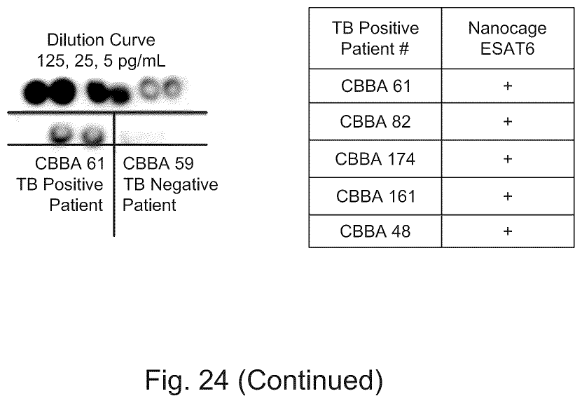

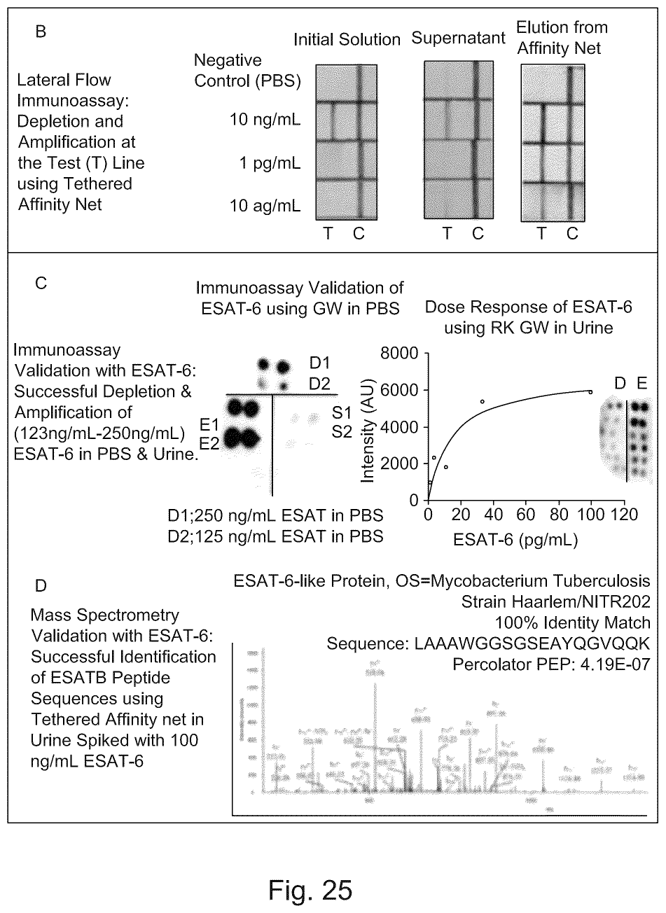

MS and immunoassay identification of TB antigen ESAT6 in the urine of HIV negative microbiologically confirmed, pediatric and adult TB patients. Seven archived, sputum culture positive children, all contained ESAT6 in the urine. Immuno array analysis using anti-ESAT6 antibody clone 11G4 (Abcam, cat number ab26246) reached an analytical sensitivity of 0.05 pg/mL (1 mL volume, 100 fold concentration factor). Five microbiologically confirmed, adult TB patients were positive for the Nanocage ESAT6 urinary test. FIG. 24 shows Tandem mass spectrometry analysis reveals ESAT6 from Mycobacterium tuberculosis in the urine of HIV negative, pediatric tuberculosis patients. FIG. 24 below shows Nanocage enriched dot blot analysis reaches an analytical sensitivity of 0.05 pg/mL. Microbiologically confirmed tuberculosis patients are positive for urinary ESAT6.

Scanning EM documents association of the hydrogel nanoparticles with the glass wool fibers (FIG. 7). The volume concentration factor reproducibility, which determines the sensitivity enhancement, is highly optimal (FIG. 7), achieving completed depletion of solution phase ESAT-6 (FIG. 7) with a 100 fold amplification of sensitivity and a precision of less than 10% CV with a sensitivity of 15 picograms/mL 2SD above background (FIG. 7).

CBBA is an abbreviation of a hospital from where samples were collected.

Table 9 shows the identification of TB peptides in the samples. The parameters are Internal controls of of commercial software that is called Proteome Discoverer, genes per peptide. Accession number is an identification number that you can use in the database to identify the sequence and the description is what we know of course about the protein. We have a Q value that is a modify P value that corrects for multiple tests. It is also defined as the minimal false discovery rate at which the identification is considered correct. PEP score is a measure of how well the spectrum matches with the peptide sequence and the higher, the better. The number of peptides it means that the number of unique peptides that we identified with that sequence. The PSM is a pesticide, spectrum matches. Those are how many times that sequence, that ion has hit the detector. pI is an isoelectric point. AA is amino acid. kDa is kilodalton, MW is molecular weight.

TABLE 9
Identification of TB peptides in the samples.

| | | | | | | | | | No. of | No. | | |
|---|---|---|---|---|---|---|---|---|---|---|---|---|
| S. | | | Exp. q- | Sum PEP | | No. of | No. of | No. of Unique | Protein | of | | |
| No. | Accession | Description | value | Score | Coverage | Peptides | PSMs | Peptides | Groups | AAs | MW kDa | cal. pI |
| 1 | A0A0T9V8Q5 | DitF protein | 0.01666 | 1.4541 | 23.255 | 1 | 1 | 1 | 1 | 43 | 4.696 | 7.3 |
| | | OS = | | | | | | | | | | |
| | | Mycobacterium | | | | | | | | | | |
| | | tuberculosis | | | | | | | | | | |
| | | GN = ERS075342_ | | | | | | | | | | |
| | | 10708 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | mLEc YL | M1 | 1 | 1 | A0A0T9V8Q5 | A0A0T9V8Q5 | 0 | 2 | 0.3032 | 0 |
| | (A2) | | QLSRª | (Oxidation); | | | | | | | | |
| | | | (SEQ ID | C4 | | | | | | | | |
| | | | No. 235) | (Carbamido- | | | | | | | | |
| | | | | methyl) | | | | | | | | |
| 2 | G8IR01 | Gp11 | 0.02466 | 1.3393 | 1.958 | 1 | 1 | 1 | 1 | 919 | 100.801 | 5.88 |
| | | OS = | | | | | | | | | | |
| | | Mycobacterium phage | | | | | | | | | | |
| | | fionn GN = 11 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | AAEQY | M16 | 1 | 1 | G8IR01 | G8IR01 | 0 | 2 | 0.1039 | 0 |
| | (A2) | | WQAEQ | (Oxidation) | | | | | | | | |
| | | | ATNGYm | | | | | | | | | |
| | | | LK^{b} | | | | | | | | | |
| | | | (SEQ ID | | | | | | | | | |
| | | | No. 236) | | | | | | | | | |
| 3 | B2HKU7 | Acyl-CoA | 0.02466 | 1.2737 | 5.097 | 1 | 1 | 1 | 1 | 412 | 44.795 | 5.97 |
| | | dehydrogenase | | | | | | | | | | |
| | | FadE21 | | | | | | | | | | |
| | | OS = | | | | | | | | | | |
| | | Mycobacterium | | | | | | | | | | |
| | | marinum | | | | | | | | | | |
| | | (strain ATCC | | | | | | | | | | |
| | | BAA-535 / M) | | | | | | | | | | |
| | | GN = fadE21 | | | | | | | | | | |
| | | PE = 3 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | EIRPNLD | M20 | 1 | 1 | B2HKU7 | B2HKU7 | 0 | 3 | 0.1946 | 0 |
| | (A2) | | ELETGA | (Oxidation) | | | | | | | | |
| | | | LSPYPIm | | | | | | | | | |
| | | | R^{b} | | | | | | | | | |
| | | | (SEQ ID | | | | | | | | | |
| | | | No. 237) | | | | | | | | | |
| 4 | G7CAU4 | Adenosylmethionine- | 0.02728 | 1.1864 | 1.5625 | 1 | 1 | 1 | 1 | 448 | 48.166 | 5.63 |
| | | 8- | | | | | | | | | | |
| | | amino-7- | | | | | | | | | | |
| | | oxononanoate | | | | | | | | | | |
| | | aminotransferase | | | | | | | | | | |
| | | OS = | | | | | | | | | | |
| | | Mycobacterium | | | | | | | | | | |
| | | thermoresistibile | | | | | | | | | | |
| | | (strain | | | | | | | | | | |
| | | ATCC 19527 / | | | | | | | | | | |
| | | DSM 44167 / | | | | | | | | | | |
| | | CIP 105390 / | | | | | | | | | | |
| | | JCM 6362 / | | | | | | | | | | |
| | | NCTC 10409 / | | | | | | | | | | |
| | | 316) GN = bioA | | | | | | | | | | |
| | | PE = 3 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | VTALET | | 1 | 1 | G7CAU4 | G7CAU4 | 0 | 2 | 0.2108 | 0 |
| | (A2) | | R^{c} | | | | | | | | | |
| | | | (SEQ ID | | | | | | | | | |
| | | | No. 314) | | | | | | | | | |
| 5 | A0A1K3CM05 | Uncharacterized | 0.03901 | 1.1304 | 7.7419 | 1 | 1 | 1 | 1 | 310 | 33.209 | 5.11 |
| | | protein | | | | | | | | | | |
| | | OS = | | | | | | | | | | |
| | | Mycobacterium | | | | | | | | | | |
| | | tuberculosis | | | | | | | | | | |
| | | GN = SAMEA2682835_ | | | | | | | | | | |
| | | 00833 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | AIFVLG | M10 | 1 | 1 | A0A1K3CM05 | A0A1K3CM05 | 0 | 3 | 0.2956 | 0 |
| | (A2) | | DEGmEG | (Oxidation) | | | | | | | | |
| | | | GGGTLT | | | | | | | | | |
| | | | SAAVTK^{a} | | | | | | | | | |
| | | | (SEQ ID | | | | | | | | | |
| | | | No. 238) | | | | | | | | | |
| 6 | A0A081HW89 | Uncharacterized | 0.01380 | 1.0188 | 2.216 | 1 | 1 | 1 | 1 | 361 | 40.723 | 9.64 |
| | | protein | | | | | | | | | | |
| | | OS = | | | | | | | | | | |
| | | Mycobacterium sp. | | | | | | | | | | |
| | | TKK-01-0059 | | | | | | | | | | |
| | | GN = K883_04441 | | | | | | | | | | |
| | | PE = 4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Selected | WFAELT | | 1 | 2 | A0A081HW89 | A0A081HW89; | 0 | 2 | | 0.0123 |
| | (A2) | | TK^{b} | | | | | A0A081I0S7 | | | | |
| | | | (SEQ ID | | | | | | | | | |
| | | | No. 239) | | | | | | | | | |
| | | | | | | | | | No. of | No. | | |
| | | | Exp. q- | Sum PEP | | No. of | No. of | No. of Unique | Protein | of | MW | |
| | Accession | Description | value | Score | Coverage | Peptides | PSMs | Peptides | Groups | AAs | [kDa] | calc. pI |
| 7 | A0A051TRQ9 | Uncharacterized | 0.00358 | 1.6784 | 1.927 | 1 | 2 | 1 | 1 | 467 | 50.317 | 5.34 |
| | | protein | | | | | | | | | | |
| | | OS = | | | | | | | | | | |
| | | Mycobacterium | | | | | | | | | | |
| | | tuberculosis | | | | | | | | | | |
| | | TKK-01-0051 | | | | | | | | | | |
| | | GN = K875_04984 | | | | | | | | | | |
| | | PE-4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | DKAALS | | 1 | 1 | A0A051TRQ9 | A0A051TRQ9 | 1 | 2 | 0.2976 | 0 |
| | (A2) | | SLRª | | | | | | | | | |
| | | | (SEQ ID | | | | | | | | | |
| | | | No. 240) | | | | | | | | | |
| | Sequest HT | Unambiguous | DKAALS | | 1 | 1 | A0A051TRQ9 | A0A051TRQ9 | 1 | 2 | 0.4576 | 0 |
| | (A2) | | SLRª | | | | | | | | | |
| | | | (SEQ ID | | | | | | | | | |
| | | | No. 240) | | | | | | | | | |
| 8 | A0A0T9N3I3 | Tetr-family | 0.00462 | 1.47534 | 3.937 | 1 | 1 | 1 | 1 | 254 | 27.568 | 5.27 |
| | | transcriptional | | | | | | | | | | |
| | | regulator | | | | | | | | | | |
| | | OS = | | | | | | | | | | |
| | | Mycobacterium | | | | | | | | | | |
| | | tuberculosis | | | | | | | | | | |
| | | GN = ERS075342_ | | | | | | | | | | |
| | | 07798 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | QDEIIEA | | 1 | 1 | A0A0T9N313 | A0A0T9N313 | 0 | 2 | 0.1829 | 0 |
| | (A2) | | TGRª | | | | | | | | | |
| | | | (SEQ ID | | | | | | | | | |
| | | | No. 241) | | | | | | | | | |
| 9 | A0A0Q3Z2T0 | Uncharacterized | 0.00462 | 1.45717 | 20.454 | 1 | 1 | 1 | 1 | 44 | 4.765 | 11.41 |
| | | protein | | | | | | | | | | |
| | | OS = | | | | | | | | | | |
| | | Mycobacterium | | | | | | | | | | |
| | | tuberculosis | | | | | | | | | | |
| | | GN = ERS007661_ | | | | | | | | | | |
| | | 02799 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | MQAAV | | 1 | 1 | A0A0Q3Z2T0 | A0A0Q3Z2T0 | 0 | 2 | 0.2521 | 0 |
| | (A2) | | AQGRª | | | | | | | | | |
| | | | (SEQ ID | | | | | | | | | |
| | | | No. 242) | | | | | | | | | |
| 10 | A0A1K3H559 | Organic | 0.00462 | 1.43997 | 1.140 | 1 | 1 | 1 | 1 | 789 | 89.898 | 5.38 |
| | | solvent | | | | | | | | | | |
| | | tolerance | | | | | | | | | | |
| | | protein | | | | | | | | | | |
| | | OS = | | | | | | | | | | |
| | | Mycobacterium | | | | | | | | | | |
| | | tuberculosis | | | | | | | | | | |
| | | GN = 1ptD | | | | | | | | | | |
| | | PE = 3 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | NDLGVF | | 1 | 1 | A0A1K3H559 | A0A1K3H559 | 0 | 2 | 0.2133 | 0 |
| | (A2) | | DFRª | | | | | | | | | |
| | | | (SEQ ID | | | | | | | | | |
| | | | No. 243) | | | | | | | | | |
| 11 | A0A1K3J6H0 | Transglycosylase | 0.01700 | 1.30891 | 2.570 | - | 1 | 1 | 1 | 817 | 89.79 | 6.61 |
| | | OS = | | | | 1 | | | | | | |
| | | Mycobacterium | | | | | | | | | | |
| | | tuberculosis | | | | | | | | | | |
| | | GN = mrcA | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | LQTPMQ | | 1 | 1 | A0A1K3J6H0 | A0A1K3J6H0 | 0 | 3 | 0.2448 | 0 |
| | (A2) | | VYSADG | | | | | | | | | |
| | | | ELIAQFG | | | | | | | | | |
| | | | EKª | | | | | | | | | |
| | | | (SEQ ID | | | | | | | | | |
| | | | No. 234) | | | | | | | | | |
| 12 | A0A081I3K4 | Uncharacterized | 0.01868 | 1.3003 | 2.029 | 1 | 1 | 1 | 1 | 1478 | 152.374 | 6.87 |
| | | protein | | | | | | | | | | |
| | | OS = | | | | | | | | | | |
| | | Mycobacterium sp. | | | | | | | | | | |
| | | TKK-01-0059 | | | | | | | | | | |
| | | GN = K883_01262 | | | | | | | | | | |
| | | PE-4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | AIAENP | C13 | 1 | 1 | A0A08113K4 | A0A08113K4 | 0 | 4 | 0.1707 | 0 |
| | (A2) | | DDIAEYc | (Carbamido- | | | | | | | | |
| | | | TSGLTL | methyl) | | | | | | | | |
| | | | TGFLGL | | | | | | | | | |
| | | | SDTPR^{b} | | | | | | | | | |
| | | | (SEQ ID | | | | | | | | | |
| | | | No. 244) | | | | | | | | | |
| | | | | | | | | | No. of | No. | | |
| | | | Exp. q- | Sum PEP | | No. of | No. of | No. of Unique | Protein | of | MW | |
| | Accession | Description | value | Score | Coverage | Peptides | PSMs | Peptides | Groups | AAs | [kDa] | calc. pI |
| 13 | A0A051UHN0 | Diacylglycerol | 0.00125 | 2.0223 | 2.132 | 1 | 2 | 1 | 1 | 469 | 51.923 | 6.96 |
| | | O- | | | | | | | | | | |
| | | acyltransferase | | | | | | | | | | |
| | | OS = | | | | | | | | | | |
| | | Mycobacterium | | | | | | | | | | |
| | | tuberculosis | | | | | | | | | | |
| | | TKK-01-0051 | | | | | | | | | | |
| | | GN = K875_00641 | | | | | | | | | | |
| | | PE = 3 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | VAVIEL | | 1 | 1 | A0A051UHN0 | A0A051UHN0 | 0 | 2 | 0.3205 | 0 |
| | (A2) | | DADR^{b} | | | | | | | | | |
| | | | (SEQ ID | | | | | | | | | |
| | | | No. 245) | | | | | | | | | |
| | Sequest HT | Unambiguous | VAVIEL | | 1 | 1 | A0A051UHN0 | A0A051UHN0 | 0 | 2 | 0.3186 | 0 |
| | (A2) | | DADR | | | | | | | | | |
| | | | (SEQ ID | | | | | | | | | |
| | | | No. 245) | | | | | | | | | |
| 14 | A0A1K3LFE0 | Heme/hemopexin | 0.00125 | 1.8136 | 2.0408 | 1 | 1 | 1 | 1 | 980 | 111.633 | 6.68 |
| | | utilization | | | | | | | | | | |
| | | protein C | | | | | | | | | | |
| | | OS = | | | | | | | | | | |
| | | Mycobacterium | | | | | | | | | | |
| | | tuberculosis | | | | | | | | | | |
| | | GN = hxuC | | | | | | | | | | |
| | | PE = 3 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | QASAWT | | 1 | 1 | A0A1K3LFE0 | A0A1K3LFE0 | 0 | 2 | 0.2788 | 0 |
| | (A2) | | PMLSAS | | | | | | | | | |
| | | | VHLTDD | | | | | | | | | |
| | | | LR^{c} | | | | | | | | | |
| | | | (SEQ ID | | | | | | | | | |
| | | | No. 315) | | | | | | | | | |
| 15 | A0A049DVS9 | Uncharacterized | 0.00125 | 1.7239 | 3.105 | 1 | 1 | 1 | 1 | 322 | 33.956 | 5.83 |
| | | protein | | | | | | | | | | |
| | | OS = | | | | | | | | | | |
| | | Mycobacterium avium | | | | | | | | | | |
| | | XTB13-223 | | | | | | | | | | |
| | | GN = X425_01922 | | | | | | | | | | |
| | | PE = 4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | VLAAAS | | 1 | 4 | A0A049DVS9 | B2HLI5; | 0 | 2 | 0.2227 | 0 |
| | (A2) | | SPEK^{b} | | | | | A0A081I611; | | | | |
| | | | (SEQ ID | | | | | A0A051TJY1; | | | | |
| | | | No. 246) | | | | | A0A049DVS9 | | | | |
| 16 | A0A0T9CW32 | Uncharacterized | 0.00125 | 1.6848 | 10.919 | 1 | 1 | 1 | 1 | 174 | 19.284 | 10.87 |
| | | protein | | | | | | | | | | |
| | | OS = | | | | | | | | | | |
| | | Mycobacterium | | | | | | | | | | |
| | | tuberculosis | | | | | | | | | | |
| | | GN = ERS027644_ | | | | | | | | | | |
| | | 02250 | | | | | | | | | | |
| | | PE-4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | EFLLDY | | 1 | 3 | A0A0T9CW32 | A0A0U0R9S4; | 0 | 3 | 0.2259 | 0 |
| | (A2) | | LGIQHEP | | | | | A0A0T9CW32; | | | | |
| | | | GVVMSR^{a} | | | | | A0A0U0UFW7 | | | | |
| | | | (SEQ ID | | | | | | | | | |
| | | | No. 247) | | | | | | | | | |
| 17 | A0A081I8B0 | Formate | 0.00125 | 1.53685 | 2.461 | 1 | 1 | 1 | 1 | 772 | 83.571 | 6.93 |
| | | dehydrogenase | | | | | | | | | | |
| | | H FdhF | | | | | | | | | | |
| | | OS = | | | | | | | | | | |
| | | Mycobacterium sp. | | | | | | | | | | |
| | | TKK-01-0059 | | | | | | | | | | |
| | | GN = K883_00274 | | | | | | | | | | |
| | | PE = 3 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | VVFINP | | 1 | 1 | A0A08118B0 | A0A081I8B0 | 0 | 3 | 0.0958 | 0 |
| | (A2) | | ADIEAL | | | | | | | | | |
| | | | GLTPGG | | | | | | | | | |
| | | | R^{b} | | | | | | | | | |
| | | | (SEQ ID | | | | | | | | | |
| | | | No. 248) | | | | | | | | | |
| 18 | A0A049DCE4 | Uncharacterized | 0.00125 | 1.52665 | 4.559 | 1 | 1 | 1 | 1 | 329 | 33.359 | 7.31 |
| | | protein | | | | | | | | | | |
| | | OS = | | | | | | | | | | |
| | | Mycobacterium avium | | | | | | | | | | |
| | | XTB13-223 | | | | | | | | | | |
| | | GN = X425_04258 | | | | | | | | | | |
| | | PE = 4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | FSVAEL | | 1 | 1 | A0A049DCE4 | A0A049DCE4 | 0 | 3 | 0.061 | 0 |
| | (A2) | | VRPWLD | | | | | | | | | |
| | | | TWR^{b} | | | | | | | | | |
| | | | (SEQ ID | | | | | | | | | |
| | | | No. 249) | | | | | | | | | |
| 19 | A0A051TUK1 | Uncharacterized | 0.00997 | 1.3483 | 1.736 | 1 | 1 | 1 | 1 | 403 | 43.563 | 5.26 |
| | | protein | | | | | | | | | | |
| | | OS = | | | | | | | | | | |
| | | Mycobacterium | | | | | | | | | | |
| | | tuberculosis | | | | | | | | | | |
| | | TKK-01-0051 | | | | | | | | | | |
| | | GN = K875_03594 | | | | | | | | | | |
| | | PE = 4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Selected | SVALNL | | 1 | 1 | A0A051TUK1 | A0A051TUK1 | 0 | 2 | 0 | 0 |
| | (A2) | | R^{c} | | | | | | | | | |
| | | | (SEQ ID | | | | | | | | | |
| | | | No. 316) | | | | | | | | | |
| 20 | A0AQU0UGJ6 | Uncharacterized | 0.01523 | 1.2294 | 56.25 | 1 | 1 | 1 | 1 | 48 | 5.605 | 8.7 |
| | | protein | | | | | | | | | | |
| | | OS = | | | | | | | | | | |
| | | Mycobacterium | | | | | | | | | | |
| | | tuberculosis | | | | | | | | | | |
| | | GN = ERS007672_ | | | | | | | | | | |
| | | 06446 | | | | | | | | | | |
| | | PE-4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | IAIVDSS | C13 | 1 | 1 | A0A0U0UGJ6 | A0A0U0UGJ6 | 0 | 3 | 0.527 | 0 |
| | (A2) | | FNHTFc | (Carbamido- | | | | | | | | |
| | | | AIIYcFH | methyl); | | | | | | | | |
| | | | SFNYcFK^{a} | C18 | | | | | | | | |
| | | | (SEQ ID | (Carbamido- | | | | | | | | |
| | | | No. 250) | methyl); | | | | | | | | |
| | | | | C25 | | | | | | | | |
| | | | | (Carbamido- | | | | | | | | |
| | | | | methyl) | | | | | | | | |
| 21 | A0A0T9LEC8 | Twin-arginine | 0.01667 | 1.1813 | 9.189 | 1 | 1 | 1 | 1 | 185 | 19.386 | 9.48 |
| | | translocation | | | | | | | | | | |
| | | pathway signal | | | | | | | | | | |
| | | OS = | | | | | | | | | | |
| | | Mycobacterium | | | | | | | | | | |
| | | tuberculosis | | | | | | | | | | |
| | | GN = ERS075342_ | | | | | | | | | | |
| | | 03632 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | IFTEPAG | | 1 | 1 | A0A0T9LEC8 | A0A0T9LEC8 | 1 | 2 | 0.139 | 0 |
| | (A2) | | DAAQGT | | | | | | | | | |
| | | | EQRK^{c} | | | | | | | | | |
| | | | (SEQ ID | | | | | | | | | |
| | | | No. 313) | | | | | | | | | |
| 22 | A0A051UG30 | Oxidoreductase | 0 | 2.2788 | 4.545 | 1 | 1 | 1 | 1 | 462 | 48.66 | 5.5 |
| | | OS = | | | | | | | | | | |
| | | Mycobacterium | | | | | | | | | | |
| | | tuberculosis | | | | | | | | | | |
| | | TKK-01-0051 | | | | | | | | | | |
| | | GN = K875_00555 | | | | | | | | | | |
| | | PE = 4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | VTAIcGF | C5 | 1 | 1 | A0A051UG30 | A0A051UG30 | 1 | 3 | 0.2081 | 0 |
| | (A2) | | ADLEAL | (Carbamido- | | | | | | | | |
| | | | VEAGRT | methyl) | | | | | | | | |
| | | | FR^{b} | | | | | | | | | |
| | | | (SEQ ID | | | | | | | | | |
| | | | No. 251) | | | | | | | | | |
| 23 | B2HLW7 | Cytochrome | 0 | 1.4999 | 5.970 | 1 | 1 | 1 | 1 | 402 | 45.163 | 5.62 |
| | | P450 191A3 | | | | | | | | | | |
| | | Cyp191A3 | | | | | | | | | | |
| | | OS = | | | | | | | | | | |
| | | Mycobacterium | | | | | | | | | | |
| | | marinum | | | | | | | | | | |
| | | (strain ATCC | | | | | | | | | | |
| | | BAA-535 / M) | | | | | | | | | | |
| | | GN = cyp191A3 | | | | | | | | | | |
| | | PE = 3 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | GQHFcIG | C5 | 1 | 1 | B2HLW7 | B2HLW7 | 1 | 4 | 0.1179 | 0 |
| | (A2) | | YHLARL | (Carbamido- | | | | | | | | |
| | | | EISVLLQ | methyl) | | | | | | | | |
| | | | EWFR^{b} | | | | | | | | | |
| | | | (SEQ ID | | | | | | | | | |
| | | | No. 252) | | | | | | | | | |
| 24 | A0A1K3ETE3 | Conserved | 0.00279 | 1.2315 | 6.060 | 1 | 1 | 1 | 1 | 297 | 31.832 | 6.11 |
| | | protein of | | | | | | | | | | |
| | | uncharacterised | | | | | | | | | | |
| | | function | | | | | | | | | | |
| | | BmrU | | | | | | | | | | |
| | | OS = | | | | | | | | | | |
| | | Mycobacterium | | | | | | | | | | |
| | | tuberculosis | | | | | | | | | | |
| | | GN = yegS | | | | | | | | | | |
| | | PE = 3 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | SLWNSL | | 1 | 1 | A0A1K3ETE3 | A0A1K3ETE3 | 0 | 2 | 0.2865 | 0 |
| | (A2) | | FNEGEA | | | | | | | | | |
| | | | EGVIMK^{c} | | | | | | | | | |
| | | | (SEQ ID | | | | | | | | | |
| | | | No. 317) | | | | | | | | | |
| 25 | G7CF58 | Uncharacterized | 0.00632 | 1.5040 | 2.352 | 1 | 1 | 1 | 1 | 340 | 34.455 | 8.94 |
| | | protein | | | | | | | | | | |
| | | OS = | | | | | | | | | | |
| | | Mycobacterium | | | | | | | | | | |
| | | thermoresistibile | | | | | | | | | | |
| | | (strain | | | | | | | | | | |
| | | ATCC 19527 / | | | | | | | | | | |
| | | DSM 44167 / | | | | | | | | | | |
| | | CIP 105390 / | | | | | | | | | | |
| | | JCM 6362 / | | | | | | | | | | |
| | | NCTC 10409 / | | | | | | | | | | |
| | | 316) | | | | | | | | | | |
| | | GN = KEK_08147 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | LTGADF | | 1 | 1 | G7CF58 | G7CF58 | 0 | 2 | 0.3177 | 0 |
| | (A2) | | TR^{b} | | | | | | | | | |
| | | | (SEQ ID | | | | | | | | | |
| | | | No. 253) | | | | | | | | | |
| 26 | A0A1K3J6H0 | Transglycosylase | 0.00892 | 1.3894 | 2.570 | 1 | 1 | 1 | 1 | 817 | 89.79 | 6.61 |
| | | OS = | | | | | | | | | | |
| | | Mycobacterium | | | | | | | | | | |
| | | tuberculosis | | | | | | | | | | |
| | | GN = mrcA | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | LQTPMQ | | 1 | 1 | A0A1K3J6H0 | A0A1K3J6H0 | 0 | 3 | 0.2733 | 0 |
| | (A2) | | VYSADG | | | | | | | | | |
| | | | ELIAQFG | | | | | | | | | |
| | | | EKª | | | | | | | | | |
| | | | (SEQ ID | | | | | | | | | |
| | | | No. 234) | | | | | | | | | |
| 27 | B2HHV4 | Arylsulfatase | 0.00892 | 1.3784 | 0.871 | 1 | 1 | 1 | 1 | 803 | 87.66 | 5.91 |
| | | AtsD | | | | | | | | | | |
| | | OS = | | | | | | | | | | |
| | | Mycobacterium | | | | | | | | | | |
| | | marinum | | | | | | | | | | |
| | | (strain ATCC | | | | | | | | | | |
| | | BAA-535 / M) | | | | | | | | | | |
| | | GN = atsD | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | QALLTG | | 1 | 13 | B2HHV4 | A0A0T9D371; | 0 | 2 | 0.2189 | 0 |
| | (A2) | | R^{a} | | | | | A0A1A9T8A0; | | | | |
| | | | (SEQ ID | | | | | A0A0T9ASE6; | | | | |
| | | | No. 254) | | | | | A0A0U0RKC9; | | | | |
| | | | | | | | | R4LZQ1; | | | | |
| | | | | | | | | A0A109SV27; | | | | |
| | | | | | | | | A0A120IVP6; | | | | |
| | | | | | | | | A0A0H3MAW3; | | | | |
| | | | | | | | | A0A0E7T2U1; | | | | |
| | | | | | | | | A0A045IQX6; | | | | |
| | | | | | | | | GOTQE3; | | | | |
| | | | | | | | | A0A0T9X8C5; | | | | |
| | | | | | | | | B2HHV4 | | | | |
| 28 | A0A1K3CBH1 | Putative | 0.00892 | 1.3783 | 4.150 | 1 | 1 | 1 | 1 | 265 | 28.642 | 10.15 |
| | | taurine ABC | | | | | | | | | | |
| | | transporter | | | | | | | | | | |
| | | permease | | | | | | | | | | |
| | | protein | | | | | | | | | | |
| | | OS = | | | | | | | | | | |
| | | Mycobacterium | | | | | | | | | | |
| | | tuberculosis | | | | | | | | | | |
| | | GN = ssuC | | | | | | | | | | |
| | | PE = 3 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | MADITG | | 1 | 1 | A0A1K3CBH1 | A0A1K3CBH1 | 1 | 2 | 0.1832 | 0 |
| | (A2) | | NKQPR^{a} | | | | | | | | | |
| | | | (SEQ ID | | | | | | | | | |
| | | | No. 255) | | | | | | | | | |
| 29 | A0A0T9PM68 | Uncharacterized | 0.03561 | 1.2855 | 16.197 | 1 | 1 | 1 | 1 | 142 | 14.926 | 9.64 |
| | | protein | | | | | | | | | | |
| | | OS = | | | | | | | | | | |
| | | Mycobacterium | | | | | | | | | | |
| | | tuberculosis | | | | | | | | | | |
| | | GN = ERS075342_ | | | | | | | | | | |
| | | 08795 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | ASVSPLS | | 1 | 1 | A0A0T9PM68 | A0A0T9PM68 | 0 | 2 | 0.1853 | 0 |
| | (A2) | | TAPFASI | | | | | | | | | |
| | | | GSDDAV | | | | | | | | | |
| | | | PSRª | | | | | | | | | |
| | | | (SEQ ID | | | | | | | | | |
| | | | No. 256) | | | | | | | | | |
| 30 | A0A0U0QKJ8 | ABC | 0.04402 | 1.2565 | 7.589 | 1 | 1 | 1 | 1 | 224 | 23.734 | 5.6 |
| | | transporter | | | | | | | | | | |
| | | ATP-binding | | | | | | | | | | |
| | | protein | | | | | | | | | | |
| | | OS = | | | | | | | | | | |
| | | Mycobacterium | | | | | | | | | | |
| | | tuberculosis | | | | | | | | | | |
| | | GN = irtB PE = 4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | MQPGTT | C15 | 1 | 1 | A0A0U0QKJ8 | A0A0U0QKJ8 | 0 | 2 | 0.5023 | 0 |
| | (A2) | | TAIVGPS | (Carbamido- | | | | | | | | |
| | | | GcGK^{a} | methyl) | | | | | | | | |
| | | | (SEQ. ID | | | | | | | | | |
| | | | No. 257) | | | | | | | | | |
| 31 | A0A081I4T0 | Uncharacterized | 0.04457 | 1.24161 | 5.333 | 1 | 1 | 1 | 1 | 150 | 16.58 | 5.9 |
| | | protein | | | | | | | | | | |
| | | OS = | | | | | | | | | | |
| | | Mycobacterium sp. | | | | | | | | | | |
| | | TKK-01-0059 | | | | | | | | | | |
| | | GN = K883_01691 | | | | | | | | | | |
| | | PE = 4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | ATTTDM | | 1 | 1 | A0A081I4T0 | A0A081I4T0 | 0 | 2 | 0.1943 | 0 |
| | (A2) | | LR | | | | | | | | | |
| 32 | A0A0T9M5C9 | FadD27 | 0 | 1.4458 | 3.344 | 1 | 1 | 1 | 1 | 299 | 32.704 | 10.67 |
| | | OS = | | | | | | | | | | |
| | | Mycobacterium | | | | | | | | | | |
| | | tuberculosis | | | | | | | | | | |
| | | GN = ERS075342_ | | | | | | | | | | |
| | | 05377 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | EVADAV | | 1 | 1 | A0A0T9M5C9 | A0A0T9M5C9 | 0 | 2 | 0.3194 | 0 |
| | (A2) | | LWPR^{c} | | | | | | | | | |
| | | | (SEQ. ID | | | | | | | | | |
| | | | No. 318) | | | | | | | | | |
| 33 | A0A0T9KY47 | DNA | 0.05104 | 1.1290 | 1.005 | 1 | 2 | 0 | 0 | 696 | 76.847 | 7.59 |
| | | topoisomerase | | | | | | | | | | |
| | | (ATP- | | | | | | | | | | |
| | | hydrolyzing) | | | | | | | | | | |
| | | OS = | | | | | | | | | | |
| | | Mycobacterium | | | | | | | | | | |
| | | tuberculosis | | | | | | | | | | |
| | | GN = gyrB_1 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Rejected | VLNEQL | | 0 | 1 | | A0A0T9KY47 | 0 | 2 | | 0.0444 |
| | (A2) | | R^{c} | | | | | | | | | |
| | | | (SEQ. ID | | | | | | | | | |
| | | | No. 319) | | | | | | | | | |
| | Sequest HT | Rejected | VLNEQL | | 0 | 1 | | A0A0T9KY47 | 0 | 2 | 0.0152 | 0 |
| | (A2) | | R^{c} | | | | | | | | | |
| | | | (SEQ. ID | | | | | | | | | |
| | | | No. 319) | | | | | | | | | |
| 34 | A0A0U0QK90 | Uncharacterized | 0.05104 | 1.1258 | 4.929 | 1 | 1 | 1 | 1 | 142 | 15.236 | 11.96 |
| | | protein | | | | | | | | | | |
| | | OS = | | | | | | | | | | |
| | | Mycobacterium | | | | | | | | | | |
| | | tuberculosis | | | | | | | | | | |
| | | GN = ERS007672_ | | | | | | | | | | |
| | | 00869 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | LSVAVI | | 1 | 3 | | A0A0U0QK90; | 0 | 2 | 0.2792 | 0 |
| | (A2) | | R | | | | | A0A0U0UPZ5; | | | | |
| | | | (SEQ. ID | | | | | A0A0U0QT26 | | | | |
| | | | No. 320) | | | | | | | | | |
| 35 | A0AQU0UPZ5 | Uncharacterized | 0.05104 | 1.1258 | 12.727 | 1 | 1 | 1 | 1 | 55 | 6.045 | 12 |
| | | protein | | | | | | | | | | |
| | | OS = | | | | | | | | | | |
| | | Mycobacterium | | | | | | | | | | |
| | | tuberculosis | | | | | | | | | | |
| | | GN = ERS007726_ | | | | | | | | | | |
| | | 04149 | | | | | | | | | | |
| | | PE-4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | LSVAVI | | 1 | 3 | | A0A0U0QK90; | 0 | 2 | 0.2792 | 0 |
| | (A2) | | R^{c} | | | | | A0A0U0UPZ5; | | | | |
| | | | (SEQ. ID | | | | | A0A0U0QT26 | | | | |
| | | | No. 320) | | | | | | | | | |
| 36 | A0A0U0QT26 | Uncharacterized | 0.051044084 | 1.1258 | 7.865 | 1 | 1 | 1 | 1 | 89 | 9.772 | 12.21 |
| | | protein | | | | | | | | | | |
| | | OS = | | | | | | | | | | |
| | | Mycobacterium | | | | | | | | | | |
| | | tuberculosis | | | | | | | | | | |
| | | GN = ERS007657_ | | | | | | | | | | |
| | | 03479 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | LSVAVI | | 1 | 3 | | A0A0U00QK90; | 0 | 2 | 0.2792 | 0 |
| | (A2) | | R^{c} | | | | | A0A0U0UPZ5; | | | | |
| | | | (SEQ. ID | | | | | A0A0U0QT26 | | | | |
| | | | No. 320) | | | | | | | | | |
| 37 | A0A051UGR6 | Uncharacterized | 0 | 1.2431 | 2.821 | 1 | 1 | 1 | 1 | 957 | 104.145 | 6.62 |
| | | protein | | | | | | | | | | |
| | | OS = | | | | | | | | | | |
| | | Mycobacterium | | | | | | | | | | |
| | | tuberculosis | | | | | | | | | | |
| | | TKK-01-0051 | | | | | | | | | | |
| | | GN = K875_00952 | | | | | | | | | | |
| | | PE = 4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | AFmTPSI | M3 | 1 | 1 | A0A051UGR6 | A0A051UGR6 | 1 | 4 | 0.1184 | 0 |
| | (A2) | | AAALGR | (Oxidation) | | | | | | | | |
| | | | WFWWP | | | | | | | | | |
| | | | LNTFNIV | | | | | | | | | |
| | | | QRª | | | | | | | | | |
| | | | (SEQ. ID | | | | | | | | | |
| | | | No. 258) | | | | | | | | | |
| 38 | A0A1K3EYR7 | Sodium: | 0.11441 | 0.92009 | 4.3103 | 1 | 1 | 1 | 1 | 464 | 48.701 | 9.04 |
| | | dicarboxylate | | | | | | | | | | |
| | | symporter | | | | | | | | | | |
| | | OS = | | | | | | | | | | |
| | | Mycobacterium | | | | | | | | | | |
| | | tuberculosis | | | | | | | | | | |
| | | GN = tcyP | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | LLQmVI | M4 | 1 | 1 | A0A1K3EYR7 | A0A1K3EYR7 | 0 | 4 | 0.2295 | 0 |
| | (A2) | | mPLVFA | (Oxidation); | | | | | | | | |
| | | | SILSAVA | M7 | | | | | | | | |
| | | | R^{a} | (Oxidation) | | | | | | | | |
| | | | (SEQ. ID | | | | | | | | | |
| | | | No. 259) | | | | | | | | | |
| 39 | A0A0T9K2L6 | Glycoside | 0 | 2.7880 | 1.080 | 1 | 1 | 1 | 1 | 648 | 69.221 | 7.2 |
| | | hydrolase, | | | | | | | | | | |
| | | family 3-like | | | | | | | | | | |
| | | protein | | | | | | | | | | |
| | | OS = | | | | | | | | | | |
| | | Mycobacterium | | | | | | | | | | |
| | | tuberculosis | | | | | | | | | | |
| | | GN = bg1X_1 | | | | | | | | | | |
| | | PE = 3 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Selected | DPGLVE | | 1 | 1 | A0A0T9K2L6 | A0A0T9K2L6 | 0 | 2 | 0.0149 | 0 |
| | (A2) | | R^{c} | | | | | | | | | |
| | | | (SEQ. ID | | | | | | | | | |
| | | | No. 321) | | | | | | | | | |
| 40 | G8I3L7 | Gp79 | 0.00238 | 1.7937 | 13.286 | 1 | 2 | 1 | 1 | 143 | 15.855 | 4.98 |
| | | OS = | | | | | | | | | | |
| | | Mycobacterium phage | | | | | | | | | | |
| | | Hedgerow | | | | | | | | | | |
| | | GN = 79 PE-4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | mDPDAL | M1 | 1 | 2 | G8I3L7 | G3M4D5; | 0 | 3 | 0.0822 | 0 |
| | (A2) | | VAQLVD | (Oxidation) | | | | G8I3L7 | | | | |
| | | | EAQNVL | | | | | | | | | |
| | | | R^{b} | | | | | | | | | |
| | | | (SEQ. ID | | | | | | | | | |
| | | | No. 260) | | | | | | | | | |
| | Sequest HT | Unambiguous | mDPDAL | M1 | 1 | 2 | G8I3L7 | G3M4D5; | 0 | 3 | 0.1359 | 0 |
| | (A2) | | VAQLVD | (Oxidation) | | | | G8I3L7 | | | | |
| | | | EAQNVL | | | | | | | | | |
| | | | R^{b} | | | | | | | | | |
| | | | (SEQ. ID | | | | | | | | | |
| | | | No. 260) | | | | | | | | | |
| 41 | A0A0T9M2X3 | Cell envelope- | 0.00336 | 1.5430 | 1.4285 | 1 | 1 | 1 | 1 | 560 | 59.4 | 9.91 |
| | | related | | | | | | | | | | |
| | | transcriptional | | | | | | | | | | |
| | | attenuator | | | | | | | | | | |
| | | OS = | | | | | | | | | | |
| | | Mycobacterium | | | | | | | | | | |
| | | tuberculosis | | | | | | | | | | |
| | | GN = ywtF_3 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | VSVVSL | | 1 | 1 | A0A0T9M2X3 | A0A0T9M2X3 | 0 | 2 | 0.0741 | 0 |
| | (A2) | | PRª | | | | | | | | | |
| | | | (SEQ. ID | | | | | | | | | |
| | | | No. 261) | | | | | | | | | |
| 42 | A0A089QP93 | Acyl-CoA | 0.01513 | 1.3715 | 3.7383 | 1 | 1 | 1 | 1 | 535 | 57.98 | 6.61 |
| | | synthetase | | | | | | | | | | |
| | | OS = | | | | | | | | | | |
| | | Mycobacterium | | | | | | | | | | |
| | | tuberculosis | | | | | | | | | | |
| | | (strain ATCC | | | | | | | | | | |
| | | 25618 / | | | | | | | | | | |
| | | H37Rv) | | | | | | | | | | |
| | | GN = LH57_07805 | | | | | | | | | | |
| | | PE = 4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | DFHAGF | | 1 | 9 | A0A089QP93 | A0A0T9XJJ8; | 0 | 3 | 0.5504 | 0 |
| | (A2) | | MSSGDV | | | | | R4MIN4; | | | | |
| | | | GYLDEN | | | | | G0TIN1; | | | | |
| | | | GR^{a} | | | | | A0A0T9D8R9; | | | | |
| | | | (SEQ. ID | | | | | A0A0T9YKC3; | | | | |
| | | | No. 262) | | | | | A0A089QP93; | | | | |
| | | | | | | | | O06831; | | | | |
| | | | | | | | | A0A0G4E256; | | | | |
| | | | | | | | | A0A0U0QZH2 | | | | |
| 43 | A0A0T9N3I3 | Tetr-family | 0.02466 | 1.2457 | 3.9370 | 1 | 1 | 1 | 1 | 254 | 27.568 | 5.27 |
| | | transcriptional | | | | | | | | | | |
| | | regulator | | | | | | | | | | |
| | | OS = | | | | | | | | | | |
| | | Mycobacterium | | | | | | | | | | |
| | | tuberculosis | | | | | | | | | | |
| | | GN = ERS075342_ | | | | | | | | | | |
| | | 07798 | | | | | | | | | | |
| | | PE-4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | QDEIIEA | | 1 | 1 | A0A0T9N313 | A0A0T9N313 | 0 | 2 | 0.309 | 0 |
| | (A2) | | TGRª | | | | | | | | | |
| | | | (SEQ ID | | | | | | | | | |
| | | | No. 241) | | | | | | | | | |
| 44 | A0AQU0QND5 | Uncharacterized | 0.00227 | 1.4190 | 7.6923 | 1 | 1 | 1 | 1 | 117 | 12.861 | 6.68 |
| | | protein | | | | | | | | | | |
| | | OS = | | | | | | | | | | |
| | | Mycobacterium | | | | | | | | | | |
| | | tuberculosis | | | | | | | | | | |
| | | GN = ERS007672_ | | | | | | | | | | |
| | | 01114 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | DHPAPLI | | 1 | 2 | A0A0U0QND5 | A0AQU0QND5; | 0 | 2 | 0.2393 | 0 |
| | (A2) | | VR | | | | | A0A0U0TPK3 | | | | |
| | | | (SEQ ID | | | | | | | | | |
| | | | No. 264) | | | | | | | | | |
| 45 | A0A051TZ12 | Uncharacterized | 0.00227 | 1.2916 | 1.8145 | 1 | 1 | 1 | 1 | 496 | 54.103 | 6.42 |
| | | protein | | | | | | | | | | |
| | | OS = | | | | | | | | | | |
| | | Mycobacterium | | | | | | | | | | |
| | | tuberculosis | | | | | | | | | | |
| | | TKK-01-0051 | | | | | | | | | | |
| | | GN = K875_03148 | | | | | | | | | | |
| | | PE = 4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | YLIGcDG | C5 | 1 | 1 | A0A051TZ12 | A0A051TZ12 | 0 | 2 | 0.2587 | 0 |
| | (A2) | | GR | (Carbamido- | | | | | | | | |
| | | | (SEQ ID | methyl) | | | | | | | | |
| | | | No. 265) | | | | | | | | | |
| 46 | A0A051TXH8 | Uncharacterized | 0.00227 | 1.2503 | 8.7301 | 1 | 1 | 1 | 1 | 378 | 40.861 | 5.41 |
| | | protein | | | | | | | | | | |
| | | OS = | | | | | | | | | | |
| | | Mycobacterium | | | | | | | | | | |
| | | tuberculosis | | | | | | | | | | |
| | | TKK-01-0051 | | | | | | | | | | |
| | | GN = K875_04012 | | | | | | | | | | |
| | | PE = 4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | TGLSPS | C12 | 1 | 1 | A0A051TXH8 | A0A051TXH8 | 0 | 3 | 0.1154 | 0 |
| | (A2) | | DMDVAc | (Carbamido- | | | | | | | | |
| | | | MYDcFT | methyl); | | | | | | | | |
| | | | YTVmAT | C16 | | | | | | | | |
| | | | MEGFGF | (Carbamido- | | | | | | | | |
| | | | cEK | methyl); | | | | | | | | |
| | | | (SEQ ID | M22 | | | | | | | | |
| | | | No. 266) | (Oxidation); | | | | | | | | |
| | | | | C31 | | | | | | | | |
| | | | | (Carbamido- | | | | | | | | |
| | | | | methyl) | | | | | | | | |
| 47 | B2I428 | Uncharacterized | 0.03794 | 1.0608 | 8.2191 | 1 | 1 | 1 | 1 | 146 | 15.631 | 11.66 |
| | | protein | | | | | | | | | | |
| | | OS = | | | | | | | | | | |
| | | Mycobacterium | | | | | | | | | | |
| | | marinum | | | | | | | | | | |
| | | (strain ATCC | | | | | | | | | | |
| | | BAA-535 / M) | | | | | | | | | | |
| | | GN = MMAR | | | | | | | | | | |
| | | p20 PE-4 | | | | | | | | | | |
| | | SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | SVTAAE | | 1 | 1 | B2I428 | B2I428 | 0 | 2 | 0.1241 | 0 |
| | (A2) | | GDALAR | | | | | | | | | |
| | | | (SEQ ID | | | | | | | | | |
| | | | No. 267) | | | | | | | | | |
| 48 | A0A0T9JZE4 | GntR family | 0.03805 | 1.0086 | 8.2608 | 1 | 1 | 1 | 1 | 230 | 25.888 | 6.44 |
| | | transcriptional | | | | | | | | | | |
| | | regulator | | | | | | | | | | |
| | | OS = | | | | | | | | | | |
| | | Mycobacterium | | | | | | | | | | |
| | | tuberculosis | | | | | | | | | | |
| | | GN = mcbR_1 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | GARVPA | M18 | 1 | 1 | A0A0T9JZE4 | A0A0T9JZE4 | 1 | 4 | 0.1311 | 0 |
| | (A2) | | LDLEQM | (Oxidation) | | | | | | | | |
| | | | QVIYQm | | | | | | | | | |
| | | | R | | | | | | | | | |
| | | | (SEQ ID | | | | | | | | | |
| | | | No. 268) | | | | | | | | | |
| 49 | G7CE61 | Transposase, | 0.00619 | 1.1665 | 4.8351 | 1 | 1 | 1 | 1 | 455 | 48.849 | 9.55 |
| | | IS4 family | | | | | | | | | | |
| | | protein | | | | | | | | | | |
| | | OS = | | | | | | | | | | |
| | | Mycobacterium | | | | | | | | | | |
| | | thermoresistibile | | | | | | | | | | |
| | | (strain | | | | | | | | | | |
| | | ATCC 19527 / | | | | | | | | | | |
| | | DSM 44167 / | | | | | | | | | | |
| | | CIP 105390 / | | | | | | | | | | |
| | | JCM 6362 / | | | | | | | | | | |
| | | NCTC 10409 / | | | | | | | | | | |
| | | 316) | | | | | | | | | | |
| | | GN = KEK_06363 | | | | | | | | | | |
| | | PE = 4 SV = 1 | | | | | | | | | | |
| | | | | | No. of | | Master | | No. of | | | |
| | Identifying | PSM | Annotated | | Protein | No. of | Protein | Protein | Missed | | | |
| | Node | Ambiguity | Sequence | Mod. | Groups | Proteins | Accessions | Accessions | Cleavages | Charge | DeltaScore | DeltaCn |
| | Sequest HT | Unambiguous | LATVIA | C9 | 1 | 1 | G7CE61 | G7CE61 | 0 | 4 | 0.2028 | 0 |
| | (A2) | | AMcAGA | (Carbamido- | | | | | | | | |
| | | | DcIDDV | methyl); | | | | | | | | |
| | | | DVLR | C14 | | | | | | | | |
| | | | (SEQ ID | (Carbamido- | | | | | | | | |
| | | | No. 263) | methyl) | | | | | | | | |

* superscript ^{a} = Validated through BLAST for Mycobacterium tuberculosis, ^{b} = Validated through BLAST for Mycobacterium genus including pathogenic and non pathogenic species, ^{c} = Rejected; Mod. is Modification

### Example 3: Screening Test for Chagas Disease

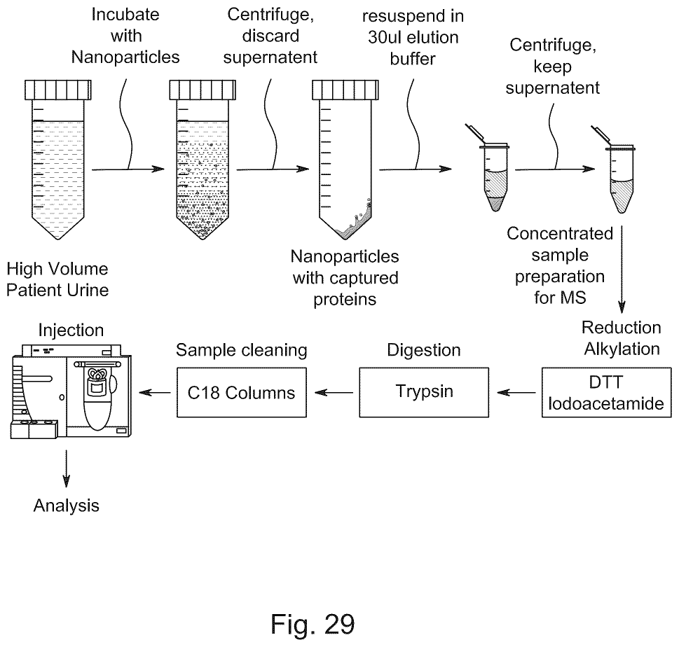

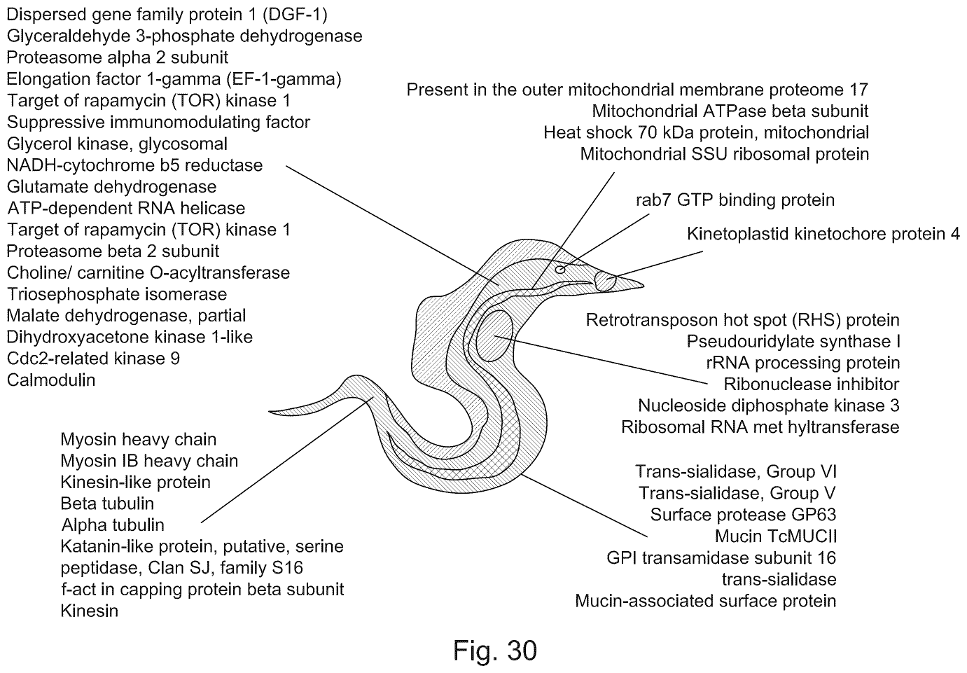

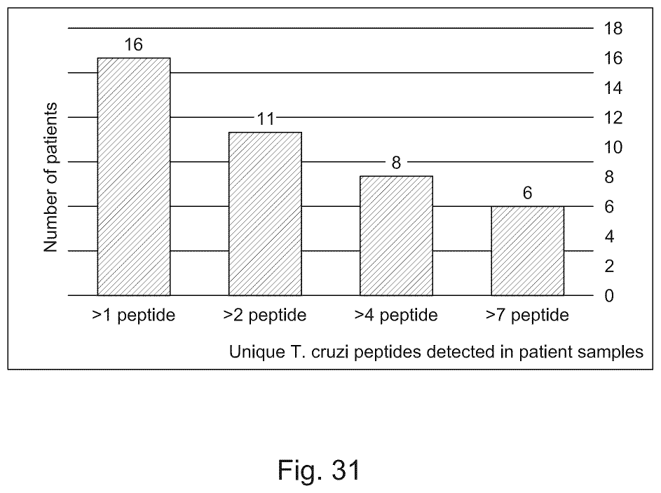

Urine samples were collected from infants (less than one month of age) with and without CD congenital infection at the Percy Bolen Maternidad hospital in Santa Cruz, Bolivia. Positive samples were characterized by being positive for two of the following independent methods: serology, PCR, and micromethod. The study included 16 positive and 11 negative infant samples. The concentration of T. cruzi derived analytes in urine and Mass Spectrometry sample preparation was done according to FIG. 29. High volume urine samples were incubated with Remazol Brilliant Blue (RBB) nanoparticles, then eluted with 30 uL elution buffer. The protein-concentrated samples were treated with DTT (reduction), Iodoacetamide (alkylation), then trypsin (digestion). TFA was added to stop the digestion, then the samples were injected into the Orbitrap Fusion mass spectrometer.

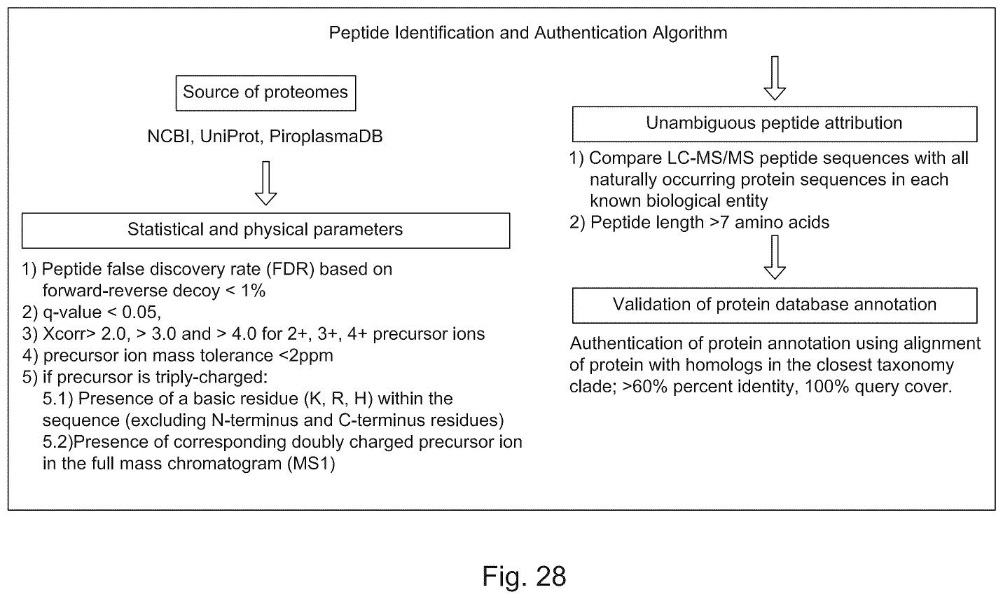

Novel T. cruzi specific urinary markers are identified with high sensitivity and specificity in patients congenitally infected with Chagas disease. Following the workflow described in FIG. 29 and Bioinformatics pipeline reported in FIG. 28, a total of 307 unique T. cruzi derived peptides were identified in 16 Chagas patients living in endemic areas.

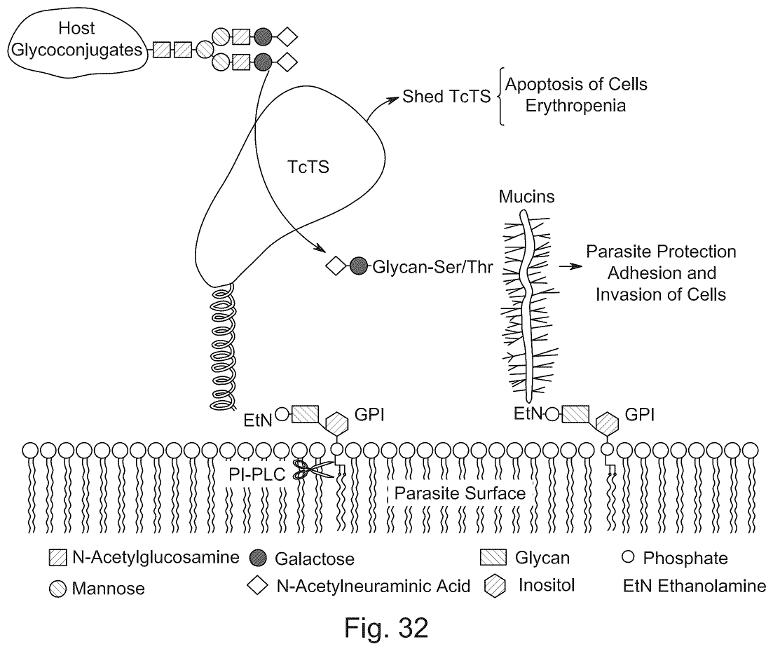

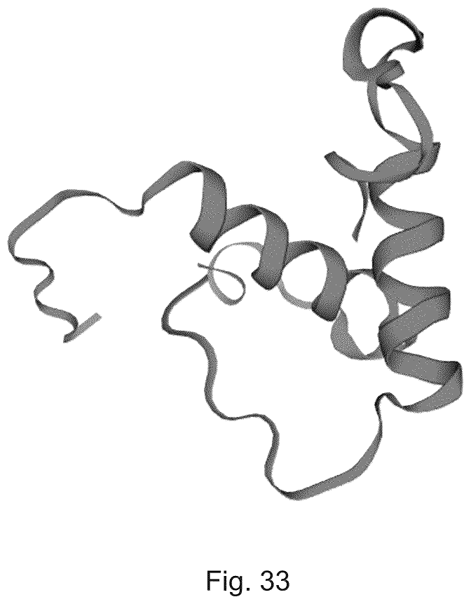

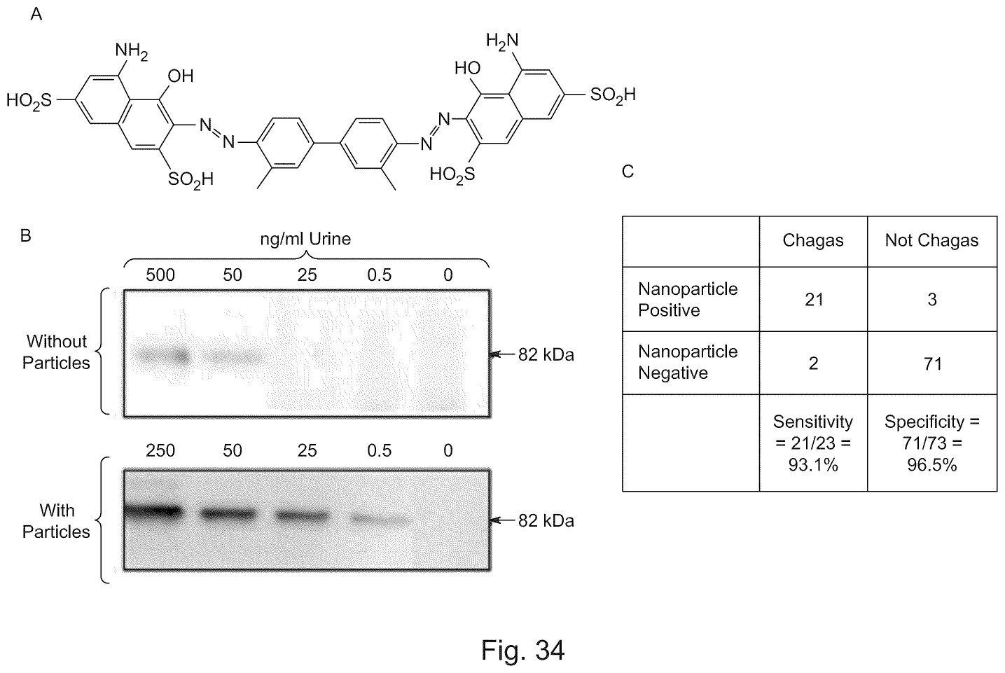

The telomeric and subtelomeric regions of T. cruzi's chromosomes are enriched in retrotransposon hot spot (RHS) protein and trans-sialidase-like protein pseudogenes which suggests a function in generation of new variants of surface proteins (involved in invasion of host cell). New protein variants are hypothesized to be mobilized by retrotransposon elements. Other studies also revealed the unique RHS proteins' role in transcription elongation and mRNA export of trypanosomes, which suggests the parasite's divergent from other eukaryotes in the universal transcription process as shown in FIGS. 32 and 33. The FIG. 34 shows A) A novel chemical bait has high affinity for glycans. B) Nanocage pre-processing increases the detection limit of western blot analysis for LPG 100 fold (5 mL initial urine volume). C) Nanocage test sensitivity and specificity for congenital Chagas.

The Table 10 below shows identification of antigen in Chagas sample by MS. Accession number, Q value, PEP score, PSM, pI, AA, kDa, MW has same meaning has defined in Example 2, Table 9.

TABLE 10
Identification of Chagas peptides in the samples.

| | | | Exp. | Sum | | | | No. of | No. of | No | | |
|---|---|---|---|---|---|---|---|---|---|---|---|---|
| S. | | | q- | PEP | Cover- | No. of | No. of | Unique | Protein | of | MW | calc. |
| No. | Accession | Description | value | Score | age | Peptide | PSMs | Peptide | Groups | AAs | kDa | pI |
| 1 | TcCLB.5 | transcript = | | 0.047 | 3.120 | 1 | 1 | 1 | 1 | 641 | 72.1 | 5.91 |
| | 07011.40: | TcCLB. | | | | | | | | | | |
| | mRNA- | 507011.40 | | | | | | | | | | |
| | p1 | \| mRNA \| | | | | | | | | | | |
| | | gene = | | | | | | | | | | |
| | | TcCLB. | | | | | | | | | | |
| | | 507011.40 | | | | | | | | | | |
| | | \| organism = | | | | | | | | | | |
| | | Trypanosoma_ | | | | | | | | | | |
| | | cruzi_CL_ | | | | | | | | | | |
| | | Brener_ | | | | | | | | | | |
| | | Esmeraldo- | | | | | | | | | | |
| | | like \| | | | | | | | | | | |
| | | gene_ | | | | | | | | | | |
| | | product = | | | | | | | | | | |
| | | hypothetical | | | | | | | | | | |
| | | protein, | | | | | | | | | | |
| | | conserved \| | | | | | | | | | | |
| | | transcript_ | | | | | | | | | | |
| | | product = | | | | | | | | | | |
| | | hypothetical | | | | | | | | | | |
| | | protein, | | | | | | | | | | |
| | | conserved \| | | | | | | | | | | |
| | | location = | | | | | | | | | | |
| | | TcChr39- | | | | | | | | | | |
| | | S:1342675- | | | | | | | | | | |
| | | 1344600(+) \| | | | | | | | | | | |
| | | protein_ | | | | | | | | | | |
| | | length = 641 | | | | | | | | | | |
| | | \| sequence_ | | | | | | | | | | |
| | | SO = | | | | | | | | | | |
| | | chromosome \| | | | | | | | | | | |
| | | SO = | | | | | | | | | | |
| | | protein_ | | | | | | | | | | |
| | | coding \| | | | | | | | | | | |
| | | is_pseudo = | | | | | | | | | | |
| | | false | | | | | | | | | | |
| | | | Anno- | | No. of | No. | Master | | No. of | | | |
| | Identi- | | tated | | Pro- | of | Protein | Protein | Missed | | | |
| | fying | PSM | Se- | | tein | Pro- | Acces- | Acces- | Cleav- | | Delta | Delta |
| | Node | Ambiguity | quence | Mod. | Groups | teins | sions | sion | ages | Charge | Score | Cn |
| | Sequest | Selected | YVSM | | 1 | 1 | TcCLB | TcCLB. | 0 | 3 | 0.0263 | 0 |
| | HT (A2) | | GTNPH | | | | .50701 | 507011. | | | | |
| | | | PSQSM | | | | 1.40:m | 40:mRN | | | | |
| | | | SPVSM | | | | RNA- | A-p1 | | | | |
| | | | R | | | | p1 | | | | | |
| | | | (SEQ | | | | | | | | | |
| | | | ID NO. | | | | | | | | | |
| | | | 269) | | | | | | | | | |
| 2 | BCY84_1 | transcript = | | 0.214 | 4.8484 | 1 | 1 | 1 | 1 | 330 | 37.4 | 7.06 |
| | 7297- | BCY84_17297- | | | | | | | | | | |
| | t36_1-p1 | t36_1 \| | | | | | | | | | | |
| | | gene = | | | | | | | | | | |
| | | BCY84_ | | | | | | | | | | |
| | | 17297 \| | | | | | | | | | | |
| | | organism = | | | | | | | | | | |
| | | Trypanosoma_ | | | | | | | | | | |
| | | cruzi_cruzi_ | | | | | | | | | | |
| | | strain_Dm28c | | | | | | | | | | |
| | | \| gene_ | | | | | | | | | | |
| | | product = | | | | | | | | | | |
| | | protein | | | | | | | | | | |
| | | kinase, | | | | | | | | | | |
| | | cdc2-related | | | | | | | | | | |
| | | kinase \| | | | | | | | | | | |
| | | transcript_ | | | | | | | | | | |
| | | product = | | | | | | | | | | |
| | | protein | | | | | | | | | | |
| | | kinase, | | | | | | | | | | |
| | | cdc2- | | | | | | | | | | |
| | | related | | | | | | | | | | |
| | | kinase \| | | | | | | | | | | |
| | | location = | | | | | | | | | | |
| | | MBSY | | | | | | | | | | |
| | | 01000635: | | | | | | | | | | |
| | | 171329- | | | | | | | | | | |
| | | 172321(−) \| | | | | | | | | | | |
| | | protein_ | | | | | | | | | | |
| | | length = | | | | | | | | | | |
| | | 330 \| | | | | | | | | | | |
| | | sequence_ | | | | | | | | | | |
| | | SO = | | | | | | | | | | |
| | | contig \| | | | | | | | | | | |
| | | SO = | | | | | | | | | | |
| | | protein_ | | | | | | | | | | |
| | | coding \| | | | | | | | | | | |
| | | is_pseudo = | | | | | | | | | | |
| | | false | | | | | | | | | | |
| | Sequest | Unambiguous | RFVHR | | 1 | 3 | BCY84 | BCY84 | 2 | 2 | 0.1111 | 0 |
| | HT (A2) | | DIKPS | | | | 17297- | _17297- | | | | |
| | | | NILISR | | | | t36_ | t36_1- | | | | |
| | | | (SEQ | | | | 1-p1 | p1; | | | | |
| | | | ID NO. | | | | | XP_814 | | | | |
| | | | 270) | | | | | 575.1; | | | | |
| | | | | | | | | XP_806 | | | | |
| | | | | | | | | 767.1 | | | | |
| 3 | BCY84_1 | transcript = | | 0.155 | 1.7985 | 1 | 1 | 1 | 1 | 1668 | 183.8 | 6.24 |
| | 8641- | BCY84_18641- | | | | | | | | | | |
| | t36_1-p1 | t36_1 \| | | | | | | | | | | |
| | | gene = | | | | | | | | | | |
| | | BCY84_ | | | | | | | | | | |
| | | 18641 \| | | | | | | | | | | |
| | | organism = | | | | | | | | | | |
| | | Trypanosoma_ | | | | | | | | | | |
| | | cruzi_cruzi_ | | | | | | | | | | |
| | | strain_Dm28c | | | | | | | | | | |
| | | \| gene_ | | | | | | | | | | |
| | | product = | | | | | | | | | | |
| | | hypothetical | | | | | | | | | | |
| | | protein \| | | | | | | | | | | |
| | | transcript_ | | | | | | | | | | |
| | | product = | | | | | | | | | | |
| | | hypothetical | | | | | | | | | | |
| | | protein \| | | | | | | | | | | |
| | | location = | | | | | | | | | | |
| | | MBSY | | | | | | | | | | |
| | | 01000689: | | | | | | | | | | |
| | | 399673- | | | | | | | | | | |
| | | 404679(+) \| | | | | | | | | | | |
| | | protein_ | | | | | | | | | | |
| | | length = | | | | | | | | | | |
| | | 1668 \| | | | | | | | | | | |
| | | sequence_ | | | | | | | | | | |
| | | SO = | | | | | | | | | | |
| | | contig \| | | | | | | | | | | |
| | | SO = | | | | | | | | | | |
| | | protein_ | | | | | | | | | | |
| | | coding \| | | | | | | | | | | |
| | | is_pseudo = | | | | | | | | | | |
| | | false | | | | | | | | | | |
| | Sequest | Unambiguous | YAQF | C19(Car | 1 | 1 | BCY84 | BCY84 | 0 | 3 | 0.1034 | 0 |
| | HT (A2) | | MESFS | bamidom | | | _18641- | 18641- | | | | |
| | | | NFDFS | ethyl); | | | t36_1- | t36_1- | | | | |
| | | | IMWYc | C23(Car | | | p1 | p1 | | | | |
| | | | NAGcL | bamidom | | | | | | | | |
| | | | STDVP | ethyl) | | | | | | | | |
| | | | R | | | | | | | | | |
| | | | (SEQ | | | | | | | | | |
| | | | ID NO. | | | | | | | | | |
| | | | 271) | | | | | | | | | |
| 4 | BCY84_2 | transcript = | | 0 | 0.1836 | 1 | 1 | 1 | 1 | 4901 | 535.8 | 6.55 |
| | 2768- | BCY84_22768- | | | | | | | | | | |
| | t36_1-p1 | t36_1 \| | | | | | | | | | | |
| | | gene = | | | | | | | | | | |
| | | BCY84_ | | | | | | | | | | |
| | | 22768 \| | | | | | | | | | | |
| | | organism = | | | | | | | | | | |
| | | Trypanosoma_ | | | | | | | | | | |
| | | cruzi_cruzi_ | | | | | | | | | | |
| | | strain_Dm28c | | | | | | | | | | |
| | | \| gene_ | | | | | | | | | | |
| | | product = | | | | | | | | | | |
| | | hypothetical | | | | | | | | | | |
| | | protein \| | | | | | | | | | | |
| | | transcript_ | | | | | | | | | | |
| | | product = | | | | | | | | | | |
| | | hypothetical | | | | | | | | | | |
| | | protein \| | | | | | | | | | | |
| | | location = | | | | | | | | | | |
| | | MBSY | | | | | | | | | | |
| | | 01000794: | | | | | | | | | | |
| | | 48618- | | | | | | | | | | |
| | | 63323(+) \| | | | | | | | | | | |
| | | protein_ | | | | | | | | | | |
| | | length = | | | | | | | | | | |
| | | 4901 \| | | | | | | | | | | |
| | | sequence_ | | | | | | | | | | |
| | | SO = | | | | | | | | | | |
| | | contig \| | | | | | | | | | | |
| | | SO = | | | | | | | | | | |
| | | protein_ | | | | | | | | | | |
| | | coding \| | | | | | | | | | | |
| | | is_pseudo = | | | | | | | | | | |
| | | false | | | | | | | | | | |
| | Sequest | Unambiguous | HFTGS | | 1 | 2 | BCY84 | TcCLB. | 0 | 3 | 0.1579 | 0 |
| | HT (A2) | | TAPR | | | | _22768- | 507083. | | | | |
| | | | (SEQ | | | | t36_1- | 109:mR | | | | |
| | | | ID NO. | | | | p1 | NA-p1; | | | | |
| | | | 272) | | | | | BCY84 | | | | |
| | | | | | | | | _22768- | | | | |
| | | | | | | | | t36_1- | | | | |
| | | | | | | | | p1 | | | | |
| 5 | TcCLB.5 | transcript = | | 0.091 | 2.0242 | 1 | 1 | 1 | 1 | 494 | 54.3 | 6.19 |
| | 06367.40: | TcCLB. | | | | | | | | | | |
| | mRNA- | 506367.40: | | | | | | | | | | |
| | p1 | mRNA \| | | | | | | | | | | |
| | | gene = | | | | | | | | | | |
| | | TcCLB. | | | | | | | | | | |
| | | 506367.40 | | | | | | | | | | |
| | | \| organism = | | | | | | | | | | |
| | | Trypanosoma_ | | | | | | | | | | |
| | | cruzi_CL_ | | | | | | | | | | |
| | | Brener_ | | | | | | | | | | |
| | | Esmeraldo- | | | | | | | | | | |
| | | like | | | | | | | | | | |
| | | \| gene_ | | | | | | | | | | |
| | | product = | | | | | | | | | | |
| | | hypothetical | | | | | | | | | | |
| | | protein, | | | | | | | | | | |
| | | conserved \| | | | | | | | | | | |
| | | transcript_ | | | | | | | | | | |
| | | product = | | | | | | | | | | |
| | | hypothetical | | | | | | | | | | |
| | | protein, | | | | | | | | | | |
| | | conserved \| | | | | | | | | | | |
| | | location = | | | | | | | | | | |
| | | TcChr39-S: | | | | | | | | | | |
| | | 398908- | | | | | | | | | | |
| | | 400392(-) \| | | | | | | | | | | |
| | | protein_ | | | | | | | | | | |
| | | length = 494 | | | | | | | | | | |
| | | \| sequence_ | | | | | | | | | | |
| | | SO = | | | | | | | | | | |
| | | chromosome | | | | | | | | | | |
| | | \| SO = | | | | | | | | | | |
| | | protein_ | | | | | | | | | | |
| | | coding \| | | | | | | | | | | |
| | | is_pseudo = | | | | | | | | | | |
| | | false | | | | | | | | | | |
| | Sequest | Unambiguous | LHLTG | | 1 | 1 | TcCLB | TcCLB. | 1 | 2 | 0.3333 | 0 |
| | HT (A2) | | RLIAK | | | | .50636 | 506367. | | | | |
| | | | (SEQ | | | | 7.40:m | 40:mRN | | | | |
| | | | ID NO. | | | | RNA- | A-p1 | | | | |
| | | | 273) | | | | p1 | | | | | |
| 6 | BCY84_1 | transcript = | | 0.064 | 4.7244 | 1 | 1 | 1 | 1 | 254 | 28.4 | 9.6 |
| | 5012- | BCY84_15012- | | | | | | | | | | |
| | t36_1-p1 | t36_1 \| | | | | | | | | | | |
| | | gene = | | | | | | | | | | |
| | | BCY84_15012 | | | | | | | | | | |
| | | \| organism = | | | | | | | | | | |
| | | Trypanosoma_ | | | | | | | | | | |
| | | cruzi_cruzi_ | | | | | | | | | | |
| | | strain_Dm28c | | | | | | | | | | |
| | | \| gene_ | | | | | | | | | | |
| | | product = | | | | | | | | | | |
| | | hypothetical | | | | | | | | | | |
| | | protein \| | | | | | | | | | | |
| | | transcript_ | | | | | | | | | | |
| | | product = | | | | | | | | | | |
| | | hypothetical | | | | | | | | | | |
| | | protein \| | | | | | | | | | | |
| | | location = | | | | | | | | | | |
| | | MBSY | | | | | | | | | | |
| | | 01000599: | | | | | | | | | | |
| | | 157191- | | | | | | | | | | |
| | | 157955(-) \| | | | | | | | | | | |
| | | protein_ | | | | | | | | | | |
| | | length = | | | | | | | | | | |
| | | 254 \| | | | | | | | | | | |
| | | sequence_ | | | | | | | | | | |
| | | SO = | | | | | | | | | | |
| | | contig \| | | | | | | | | | | |
| | | SO = | | | | | | | | | | |
| | | protein_ | | | | | | | | | | |
| | | coding \| | | | | | | | | | | |
| | | is_pseudo = | | | | | | | | | | |
| | | false | | | | | | | | | | |
| | Sequest | Unambiguous | TAGDA | | 1 | 3 | BCY84 | TcCLB. | 0 | 2 | 0.1571 | 0 |
| | HT (A2) | | AAAG | | | | _15012- | 510149. | | | | |
| | | | GPR | | | | t36_1- | 100:mR | | | | |
| | | | (SEQ | | | | p1 | NA-p1; | | | | |
| | | | ID NO. | | | | | XP_818 | | | | |
| | | | 274) | | | | | 640.1; | | | | |
| | | | | | | | | BCY84 | | | | |
| | | | | | | | | _15012- | | | | |
| | | | | | | | | t36_1- | | | | |
| | | | | | | | | p1 | | | | |
| 7 | BCY84_0 | transcript = | | 0.053 | 4.9928 | 1 | 1 | 1 | 1 | 701 | 79.5 | 6.6 |
| | 1456- | BCY84_01456- | | | | | | | | | | |
| | t36_1-p1 | t36_1 \| | | | | | | | | | | |
| | | gene = | | | | | | | | | | |
| | | BCY84_ | | | | | | | | | | |
| | | 01456 \| | | | | | | | | | | |
| | | organism = | | | | | | | | | | |
| | | Trypanosoma_ | | | | | | | | | | |
| | | cruzi_cruzi_ | | | | | | | | | | |
| | | strain_Dm28c | | | | | | | | | | |
| | | \| gene_ | | | | | | | | | | |
| | | product = | | | | | | | | | | |
| | | hypothetical | | | | | | | | | | |
| | | protein \| | | | | | | | | | | |
| | | transcript_ | | | | | | | | | | |
| | | product = | | | | | | | | | | |
| | | hypothetical | | | | | | | | | | |
| | | protein \| | | | | | | | | | | |
| | | location = | | | | | | | | | | |
| | | MBSY | | | | | | | | | | |
| | | 01000059: | | | | | | | | | | |
| | | 5809-7914(-) | | | | | | | | | | |
| | | \| protein_ | | | | | | | | | | |
| | | length = 701 | | | | | | | | | | |
| | | \| sequence_ | | | | | | | | | | |
| | | SO = | | | | | | | | | | |
| | | contig \| | | | | | | | | | | |
| | | SO = | | | | | | | | | | |
| | | protein_ | | | | | | | | | | |
| | | coding \| | | | | | | | | | | |
| | | is_pseudo = | | | | | | | | | | |
| | | false | | | | | | | | | | |
| | Sequest | Unambiguous | FLACV | C4(Carba | 1 | 2 | BCY84 | BCY84 | 0 | 3 | 0.4667 | 0 |
| | HT (A2) | | NDGG | midomet | | | _01456- | _01456- | | | | |
| | | | HcSLFT | hyl); | | | t36_1- | t36_1- | | | | |
| | | | QHEFP | C11(Car | | | p1 | p1; | | | | |
| | | | SEDEF | bamidom | | | | BCY84 | | | | |
| | | | STDFF | ethyl) | | | | _18460- | | | | |
| | | | NEHGK | | | | | t36_1- | | | | |
| | | | (SEQ | | | | | p1 | | | | |
| | | | ID NO. | | | | | | | | | |
| | | | 275) | | | | | | | | | |
| 8 | XP_8109 | hypothetical | | 0.053 | 1.0758 | 1 | 1 | 1 | 1 | 1952 | 210.9 | 6.77 |
| | 20.1 | protein | | | | | | | | | | |
| | | [Trypanosoma | | | | | | | | | | |
| | | cruzi strain | | | | | | | | | | |
| | | CL Brener] | | | | | | | | | | |
| | Sequest | Unambiguous | cTVDIS | C1(Carba | 1 | 1 | XP_81 | XP_810 | 0 | 3 | 0.2466 | 0 |
| | HT (A2) | | TAGST | midomet | | | 0920.1 | 920.1 | | | | |
| | | | ETNIG | hyl); | | | | | | | | |
| | | | DIScR | C20(Car | | | | | | | | |
| | | | (SEQ | bamidom | | | | | | | | |
| | | | ID NO. | ethyl) | | | | | | | | |
| | | | 276) | | | | | | | | | |
| 9 | XP_8119 | hypothetical | | 0.053 | 0.9756 | 1 | 1 | 1 | 1 | 1025 | 115.6 | 6.01 |
| | 85.1 | protein | | | | | | | | | | |
| | | [Trypanosoma | | | | | | | | | | |
| | | cruzi strain | | | | | | | | | | |
| | | CL Brener] | | | | | | | | | | |
| | Sequest | Selected | AVQA | C6(Carba | 1 | 1 | XP_81 | XP_811 | 1 | 2 | 0 | 0 |
| | HT (A2) | | McRDE | midomet | | | 1985.1 | 985.1 | | | | |
| | | | R | hyl) | | | | | | | | |
| | | | (SEQ | | | | | | | | | |
| | | | ID NO. | | | | | | | | | |
| | | | 277) | | | | | | | | | |
| 10 | XP_8153 | hypothetical | | 0.101 | 1.1578 | 1 | 1 | 1 | 1 | 950 | 108.1 | 8.79 |
| | 88.1 | protein | | | | | | | | | | |
| | | [Trypanosoma | | | | | | | | | | |
| | | cruzi strain | | | | | | | | | | |
| | | CL Brener] | | | | | | | | | | |
| | Sequest | Unambiguous | LIDFV | | 1 | 2 | XP_81 | XP_811 | 0 | 2 | 0.5443 | 0 |
| | HT (A2) | | ENNA | | | | 5388.1 | 863.1; | | | | |
| | | | MK | | | | | XP_815 | | | | |
| | | | (SEQ | | | | | 388.1 | | | | |
| | | | ID NO. | | | | | | | | | |
| | | | 278) | | | | | | | | | |
| 11 | XP_8102 | mucin- | | 0.068 | 2.0642 | 1 | 1 | 1 | 1 | 436 | 45.3 | 4.78 |
| | 36.1 | associated | | | | | | | | | | |
| | | surface | | | | | | | | | | |
| | | protein | | | | | | | | | | |
| | | (MASP) | | | | | | | | | | |
| | | [Trypanosoma | | | | | | | | | | |
| | | cruzi strain | | | | | | | | | | |
| | | CL Brener] | | | | | | | | | | |
| | Sequest | Selected | GAGAL | | 1 | 1 | XP_81 | XP_810 | 1 | 2 | 0.0297 | 0 |
| | HT (A2) | | EKVK | | | | 0236.1 | 236.1 | | | | |
| | | | (SEQ | | | | | | | | | |
| | | | ID NO. | | | | | | | | | |
| | | | 279) | | | | | | | | | |
| 12 | BCY84_0 | transcript = | | 0.066 | 1.1123 | 1 | 1 | 1 | 1 | 899 | 94.5 | 5.66 |
| | 8138- | BCY84_08138- | | | | | | | | | | |
| | t36_1-p1 | t36_1 \| | | | | | | | | | | |
| | | gene = | | | | | | | | | | |
| | | BCY84_ | | | | | | | | | | |
| | | 08138 \| | | | | | | | | | | |
| | | organism = | | | | | | | | | | |
| | | Trypanosoma_ | | | | | | | | | | |
| | | cruzi_cruzi_ | | | | | | | | | | |
| | | strain_Dm28c | | | | | | | | | | |
| | | \| gene_ | | | | | | | | | | |
| | | product = | | | | | | | | | | |
| | | trans- | | | | | | | | | | |
| | | sialidase | | | | | | | | | | |
| | | \| trans- | | | | | | | | | | |
| | | cript_ | | | | | | | | | | |
| | | product = | | | | | | | | | | |
| | | trans- | | | | | | | | | | |
| | | sialidase \| | | | | | | | | | | |
| | | location = | | | | | | | | | | |
| | | MBSY | | | | | | | | | | |
| | | 01000386: | | | | | | | | | | |
| | | 3903- | | | | | | | | | | |
| | | 6602(-) \| | | | | | | | | | | |
| | | protein_ | | | | | | | | | | |
| | | length = | | | | | | | | | | |
| | | 899 \| | | | | | | | | | | |
| | | sequence_ | | | | | | | | | | |
| | | SO = | | | | | | | | | | |
| | | contig \| | | | | | | | | | | |
| | | SO = | | | | | | | | | | |
| | | protein_ | | | | | | | | | | |
| | | coding \| | | | | | | | | | | |
| | | is_pseudo = | | | | | | | | | | |
| | | false | | | | | | | | | | |
| | Sequest | Unambiguous | NNDG | | 1 | 1 | BCY84 | BCY84 | 0 | 2 | 0.4958 | 0 |
| | HT (A2) | | ATGAT | | | | _08138- | _08138- | | | | |
| | | | R | | | | t36_1- | t36_1- | | | | |
| | | | (SEQ | | | | p1 | p1 | | | | |
| | | | ID NO. | | | | | | | | | |
| | | | 280) | | | | | | | | | |
| 13 | XP_8033 | hypothetical | | 0.056 | 3.1976 | 1 | 1 | 1 | 1 | 344 | 37.1 | 7.69 |
| | 09.1 | protein | | | | | | | | | | |
| | | [Trypanosoma | | | | | | | | | | |
| | | cruzi strain | | | | | | | | | | |
| | | CL Brener] | | | | | | | | | | |
| | Sequest | Unambiguous | KEAEA | | 1 | 1 | XP_80 | XP_803 | 1 | 2 | 0.3707 | 0 |
| | HT (A2) | | ENALN | | | | 3309.1 | 309.1 | | | | |
| | | | R | | | | | | | | | |
| | | | (SEQ | | | | | | | | | |
| | | | ID NO. | | | | | | | | | |
| | | | 281) | | | | | | | | | |
| 14 | TcCLB.5 | transcript = | | 0.047 | 2.3172 | 1 | 1 | 1 | 1 | 561 | 62.3 | 9.25 |
| | 07083.90: | TcCLB. | | | | | | | | | | |
| | mRNA- | 507083.90: | | | | | | | | | | |
| | p1 | mRNA \| | | | | | | | | | | |
| | | gene = | | | | | | | | | | |
| | | TcCLB. | | | | | | | | | | |
| | | 507083.90 | | | | | | | | | | |
| | | \| organism = | | | | | | | | | | |
| | | Trypanosoma_ | | | | | | | | | | |
| | | cruzi_CL_ | | | | | | | | | | |
| | | Brener_ | | | | | | | | | | |
| | | Esmeraldo- | | | | | | | | | | |
| | | like | | | | | | | | | | |
| | | \| gene_ | | | | | | | | | | |
| | | product = | | | | | | | | | | |
| | | hypothetical | | | | | | | | | | |
| | | protein, | | | | | | | | | | |
| | | conserved \| | | | | | | | | | | |
| | | transcript_ | | | | | | | | | | |
| | | product = | | | | | | | | | | |
| | | hypothetical | | | | | | | | | | |
| | | protein, | | | | | | | | | | |
| | | conserved \| | | | | | | | | | | |
| | | location = | | | | | | | | | | |
| | | TcChr25-S: | | | | | | | | | | |
| | | 339208- | | | | | | | | | | |
| | | 340893(+) \| | | | | | | | | | | |
| | | protein_ | | | | | | | | | | |
| | | length = | | | | | | | | | | |
| | | 561 \| | | | | | | | | | | |
| | | sequence_ | | | | | | | | | | |
| | | SO = | | | | | | | | | | |
| | | chromosome \| | | | | | | | | | | |
| | | SO = | | | | | | | | | | |
| | | protein_ | | | | | | | | | | |
| | | coding \| | | | | | | | | | | |
| | | is_pseudo = | | | | | | | | | | |
| | | false | | | | | | | | | | |
| | Sequest | Unambiguous | RPRLV | | 1 | 1 | TcCLB | TcCLB. | 1 | 4 | 0.2533 | 0 |
| | HT (A2) | | YSEDS | | | | .50708 | 507083. | | | | |
| | | | EVR | | | | 3.90:m | 90:mRN | | | | |
| | | | (SEQ | | | | RNA- | A-p1 | | | | |
| | | | ID NO. | | | | p1 | | | | | |
| | | | 282) | | | | | | | | | |
| 15 | BCY84_1 | transcript = | | 0.046 | 3.2573 | 1 | 1 | 1 | 1 | 307 | 34.4 | 4.7 |
| | 3262- | BCY84_13262- | | | | | | | | | | |
| | t36_1-p1 | t36_1 \| | | | | | | | | | | |
| | | gene = | | | | | | | | | | |
| | | BCY84_ | | | | | | | | | | |
| | | 13262 \| | | | | | | | | | | |
| | | organism = | | | | | | | | | | |
| | | Trypanosoma_ | | | | | | | | | | |
| | | cruzi_cruzi_ | | | | | | | | | | |
| | | strain_ | | | | | | | | | | |
| | | Dm28c \| | | | | | | | | | | |
| | | gene_ | | | | | | | | | | |
| | | product = | | | | | | | | | | |
| | | hypothetical | | | | | | | | | | |
| | | protein \| | | | | | | | | | | |
| | | transcript_ | | | | | | | | | | |
| | | product = | | | | | | | | | | |
| | | hypothetical | | | | | | | | | | |
| | | protein \| | | | | | | | | | | |
| | | location = | | | | | | | | | | |
| | | MBSY | | | | | | | | | | |
| | | 01000938: | | | | | | | | | | |
| | | 10794- | | | | | | | | | | |
| | | 11717(-) \| | | | | | | | | | | |
| | | protein_ | | | | | | | | | | |
| | | length = | | | | | | | | | | |
| | | 307 \| | | | | | | | | | | |
| | | sequence_ | | | | | | | | | | |
| | | SO = | | | | | | | | | | |
| | | contig \| | | | | | | | | | | |
| | | SO = | | | | | | | | | | |
| | | protein_ | | | | | | | | | | |
| | | coding \| | | | | | | | | | | |
| | | is_pseudo = | | | | | | | | | | |
| | | false | | | | | | | | | | |
| | Sequest | Unambiguous | IDLLQ | | 1 | 2 | BCY84 | XP_806 | 0 | 2 | 0.6825 | 0 |
| | HT (A2) | | AELHK | | | | _13262- | 363.1; | | | | |
| | | | (SEQ | | | | t36_1- | BCY84 | | | | |
| | | | ID NO. | | | | p1 | _13262- | | | | |
| | | | 283) | | | | | t36_1- | | | | |
| | | | | | | | | p1 | | | | |
| 16 | BCY84_0 | transcript = | | 0 | 1.9847 | 1 | 1 | 1 | 1 | 655 | 71 | 6.13 |
| | 1068- | BCY84_01068- | | | | | | | | | | |
| | t36_1-p1 | t36_1 \| | | | | | | | | | | |
| | | gene = | | | | | | | | | | |
| | | BCY84_ | | | | | | | | | | |
| | | 01068 \| | | | | | | | | | | |
| | | organism = | | | | | | | | | | |
| | | Trypanosoma_ | | | | | | | | | | |
| | | cruzi_cruzi_ | | | | | | | | | | |
| | | strain_ | | | | | | | | | | |
| | | Dm28c \| | | | | | | | | | | |
| | | gene_ | | | | | | | | | | |
| | | product = | | | | | | | | | | |
| | | heat shock | | | | | | | | | | |
| | | 70 kDa | | | | | | | | | | |
| | | protein, | | | | | | | | | | |
| | | mitochon- | | | | | | | | | | |
| | | drial | | | | | | | | | | |
| | | precursor \| | | | | | | | | | | |
| | | transcript_ | | | | | | | | | | |
| | | product = | | | | | | | | | | |
| | | heat shock | | | | | | | | | | |
| | | 70 kDa | | | | | | | | | | |
| | | protein, | | | | | | | | | | |
| | | mitochon- | | | | | | | | | | |
| | | drial | | | | | | | | | | |
| | | precursor \| | | | | | | | | | | |
| | | location = | | | | | | | | | | |
| | | MBSY | | | | | | | | | | |
| | | 01000053: | | | | | | | | | | |
| | | 615988- | | | | | | | | | | |
| | | 617955(-) \| | | | | | | | | | | |
| | | protein_ | | | | | | | | | | |
| | | length = | | | | | | | | | | |
| | | 655 \| | | | | | | | | | | |
| | | sequence_ | | | | | | | | | | |
| | | SO = | | | | | | | | | | |
| | | contig \| | | | | | | | | | | |
| | | SO = | | | | | | | | | | |
| | | protein_ | | | | | | | | | | |
| | | coding \| | | | | | | | | | | |
| | | is_pseudo = | | | | | | | | | | |
| | | false | | | | | | | | | | |
| | Sequest | Unambiguous | DSESH | | 1 | 6 | BCY84 | TcCLB. | 2 | 3 | 0.2514 | 0 |
| | HT (A2) | | AESDR | | | | _01068- | 511745. | | | | |
| | | | LKR | | | | t36_1- | 10:mRN | | | | |
| | | | (SEQ | | | | p1 | A-p1; | | | | |
| | | | ID NO. | | | | | XP_819 | | | | |
| | | | 284) | | | | | 091.1; | | | | |
| | | | | | | | | BCY84 | | | | |
| | | | | | | | | _01068- | | | | |
| | | | | | | | | t36_1- | | | | |
| | | | | | | | | p1; | | | | |
| | | | | | | | | XP_806 | | | | |
| | | | | | | | | 221.1; | | | | |
| | | | | | | | | XP_804 | | | | |
| | | | | | | | | 120.1; | | | | |
| | | | | | | | | BCY84 | | | | |
| | | | | | | | | _01069- | | | | |
| | | | | | | | | t36_1- | | | | |
| | | | | | | | | p1 | | | | |
| 17 | XP_8099 | mucin- | | 0 | 2.5316 | 1 | 1 | 1 | 1 | 395 | 39.8 | 5.05 |
| | 01.1 | associated | | | | | | | | | | |
| | | surface | | | | | | | | | | |
| | | protein | | | | | | | | | | |
| | | (MASP) | | | | | | | | | | |
| | | [Trypanosoma | | | | | | | | | | |
| | | cruzi strain | | | | | | | | | | |
| | | CL Brener] | | | | | | | | | | |
| | Sequest | Unambiguous | mNPEK | M1(Oxid | 1 | 1 | XP_80 | XP_809 | 2 | 3 | 0.2787 | 0 |
| | HT (A2) | | EKNGR | ation) | | | 9901.1 | 901.1 | | | | |
| | | | (SEQ | | | | | | | | | |
| | | | ID NO. | | | | | | | | | |
| | | | 285) | | | | | | | | | |
| 18 | XP_8208 | hypothetical | | 0 | 5.3333 | 1 | 1 | 1 | 1 | 225 | 25.3 | 7.05 |
| | 30.1 | protein | | | | | | | | | | |
| | | [Trypanosoma | | | | | | | | | | |
| | | cruzi strain | | | | | | | | | | |
| | | CL Brener] | | | | | | | | | | |
| | Sequest | Selected | QPHER | M6(Oxid | 1 | 3 | XP_82 | TcCLB. | 1 | 2 | 0.0263 | 0 |
| | HT (A2) | | mYcGT | ation); | | | 0830.1 | 510373. | | | | |
| | | | IK | C8(Carba | | | | 80:mRN | | | | |
| | | | (SEQ | midomet | | | | A-p1; | | | | |
| | | | ID NO. | hyl) | | | | XP_809 | | | | |
| | | | 286) | | | | | 165.1; | | | | |
| | | | | | | | | XP_820 | | | | |
| | | | | | | | | 830.1 | | | | |

The abbreviated words have same meaning as defined in Table 9.
All annotated peptides sequences were validated through Blast for Trypanosoma cruzi.

### Example 4: Screening Test for Lyme Disease in Children

Patients enrolled in this study fall into five categories: 1) patients with acute stage Borrelia infection defined by a two-tier serology criteria; 2) Persistent symptoms after treatment following EM rash (PTLDS) symptomatic patients with a clinical diagnosis of PTLDS; 3) patients treated in community centers and private practices with clinical suspicion of tick-borne illnesses but in the absence of clinical information regarding previous symptoms and treatments; 4) diseased controls, which include patients harboring non-tick-borne infections, who are hospitalized in Peru, a geographic region where ticks are very rare, and U.S. patients with a diagnosis of traumatic brain injury and acute respiratory distress syndrome; and 5) healthy controls.

Specificity of the mass spectrometry analytic method is ensured by a three-tier authentication algorithm which requires stringent filters for peptide identification, 100% amino acid sequence identity with tick-borne pathogen proteins, evolutionary taxonomic verification for related pathogens, and lack of overlap with human or non-tick-borne pathogenic organisms. Identified peptides are verified by concomitant urine western blot immunoassays, orthogonal mass spectrometry based parallel reaction monitoring (PRM), and an animal model of persistent Babesiosis. The parameters for the authentication algorithm are established on a set of acute Lyme patients and negative controls; the method is then applied to a non-overlapping set of non acute patients including PTLDS (category 2) and other patients suspected of tick-borne illnesses (category 3), and negative controls. The correlation between the number of pathogen-specific urinary peptides and the presence or absence of symptoms as assessed by health care professionals is investigated.

The control group consisted of 100 patients (M/F=1, median age=41, IQR=25.75), including healthy controls and disease control patients diagnosed with acute respiratory distress syndrome, tuberculosis, and traumatic brain injury. Peptides derived from Borrelia species known to be pathogenic in humans were found in 10/10 Lyme borreliosis patients. No peptides derived from other tick borne organism investigated (Babesia, Anaplasma, Rickettsia, Ehrlichia, Bartonella, Francisella, Powassan virus, encephalitis virus, and Colorado tick fever virus) were identified. The list of peptides identified in each patient is reported in Table 13. Identified peptides belonged to different proteins including membrane associated proteins (e.g. OspC), motility proteins (e.g. flagellar motor switch protein FliN), transport proteins (e.g., ABC transporter substrate binding protein, periplasmic oligopeptide binding protein, mechanosensitive ion channel family protein), chemotaxis proteins (e.g. MotA), protein translation and modification proteins (e.g., peptide chain release factor N(5), glutamine methyl transferase), metabolic enzymes (e.g. Chain A, Glyceraldehyde-3-phosphate dehydrogenase, nicotinate phosphoribosyltransferase), RNA and protein metabolism (e.g. translation elongation factor YigZ), and antigens known to elicit immune response (e.g., immunogenic protein P37). A patient was considered positive for a given pathogen if at least two unique peptides deriving unambiguously (100% sequence identity with the pathogen and less than 90% sequence identity with any other organism) from such pathogen were identified. Zero false positive peptides were identified in 100 healthy and diseased controls.

TABLE 11
Demographic information of patients enrolled in this study. Demographic
and clinical characteristics of sample cohort. Acute LB =
acute Lyme Borreliosis, PTLDS = post treatment Lyme disease
syndrome, NA-TBI = suspected non-acute tick borne infection.

| | | | | Controls | |
|---|---|---|---|---|---|
| | Patients | | | Diseased | Healthy |
| Characteristics | Acute LB | PTLDS | NA-TBI | Controls | Controls |
| N | 10 | 36 | 112 | 215 | 35 |
| Age (Median) | 52.5 | 50 | 46 | 35.5 | 31 |
| | (IQR = 14) | (IQR = 25) | (IQR = 32) | (IQR = 31) | (IQR = 60) |
| Sex | | | | | |
| Male/Female | 4/6 | 14/22 | 54/58 | 112/57 | 18/17 |
| NA | — | — | — | 46 | — |
| Tick discovered | 6Y/1N | 11Y/10N | 11Y/15N | — | — |
| Bull's eye rash | 10Y/0N | 26Y/5N | 24N | — | — |
| Joint pain | 7Y/0N | 22Y/4N | 15Y/N | — | — |
| Fatigue | 7Y/0N | 18Y/6N | 20Y/4N | — | — |
| Fever | 7Y/0N | 11Y/13 | 7Y/17N | | |
| Facial Palsy | 1Y/3N | 3Y/20N | 3Y/21N | — | — |
| Neurological | 4Y/0N | 16Y/7N | 9Y/14N | — | — |
| Serology (Pos) | 10Y/0N | 19Y/4N | 12Y/14N | | |

TABLE 12
Borrelia peptides identified in true positive
cases with EM rash and CDC positive serology.
Borrelia urinary peptides are identified before
EM rash development, and revert to negative
after therapy completion and symptoms resolu-
tion. Acute Lyme Borreliosis symptoms = joint
pain, fatigue, facial palsy, neurological
symptoms.

| | | | | Under | | |
|---|---|---|---|---|---|---|
| | | | | treat- | | |
| | | | | ment | Number | |
| | | | | (at | of | |
| | | Acute | | time | identi- | |
| | | LB | | of | fied | |
| Sample | EM | symp- | Sero- | collec- | Borrelia | Peptide |
| ID | Rash | toms | logy | tion) | peptides | sequence |
| 108838 | Y | Y | Pos | No | 3 | IDTEEAAVK |
| | | | | | | (SEQ ID. |
| | | | | | | No. 322); |
| | | | | | | NAGNFEIIR |
| | | | | | | SEQ ID. |
| | | | | | | No. 323; |
| | | | | | | VTLSDISTK |
| | | | | | | (SEQ ID. |
| | | | | | | No. 324) |
| 104821 | Y | Y | Pos | No | 2 | AILKTNGDK |
| | | | | | | TLGAAELEK |
| | | | | | | (SEQ ID. |
| | | | | | | No. 325); |
| | | | | | | NNFCLSKSD |
| | | | | | | LEEIR |
| | | | | | | (SEQ ID. |
| | | | | | | No. 326) |
| 790907 | Y | Y | Pos | No | 2 | SNQDNQTLL |
| | | | | | | LSLHQAIAK |
| | | | | | | (SEQ ID. |
| | | | | | | No. 327); |
| | | | | | | LKNSHAELG |
| | | | | | | VAGNGATTD |
| | | | | | | ENAQK |
| | | | | | | (SEQ ID. |
| | | | | | | No. 328) |
| 889597 | Y | Y | Pos | Yes | 2 | GGYLDHVNH |
| | | | | (2 | | AGAKKVILT |
| | | | | days | | VPAK |
| | | | | doxyci- | | (SEQ ID. |
| | | | | cline) | | No. 329) |
| | | | | | | LATVNEAEG |
| | | | | | | K |
| | | | | | | (SEQ ID. |
| | | | | | | No. 330) |
| 213567 | Y | Y | Pos | No | 2 | LATVNEAEG |
| | | | | | | K |
| | | | | | | (SEQ ID. |
| | | | | | | No. 331); |
| | | | | | | NDVSEEKPE |
| | | | | | | IK |
| | | | | | | (SEQ ID. |
| | | | | | | No. 332) |
| 453742 | Y* | Y* | Pos | No | 3 | VVILNEATG |
| | | | | | | K |
| | | | | | | (SEQ ID. |
| | | | | | | No. 333); |
| | | | | | | LATVNEAEG |
| | | | | | | K |
| | | | | | | (SEQ ID. |
| | | | | | | No. 334); |
| | | | | | | FVYIGNVDN |
| | | | | | | MGYTINFK |
| | | | | | | (SEQ ID. |
| | | | | | | No. 335); |
| 463256 | Y | N | Pos | No | 2 | NLSLFTDFY |
| | | | | | | EISMMNAYF |
| | | | | | | IK |
| | | | | | | (SEQ ID. |
| | | | | | | No. 336); |
| | | | | | | QKATGAINA |
| | | | | | | VSGEQIL |
| | | | | | | (SEQ ID. |
| | | | | | | No. 337) |
| 459235 | Y | Y | Pos | Yes | 2 | SAKEVINNT |
| | | | | (1 | | STMEK |
| | | | | day | | (SEQ ID. |
| | | | | doxyci- | | No. 338); |
| | | | | cline) | | SSSVDGFVS |
| | | | | | | FKEYKER |
| | | | | | | (SEQ ID. |
| | | | | | | No. 339) |
| 729340 | Y | Y | Pos | No | 1 | FEDAIVLRD |
| | | | | | | K |
| | | | | | | (SEQ ID. |
| | | | | | | No. 340) |
| 310741 | Y | Y | Pos | No | 2 | VNESDLGIK |
| | | | | | | ALDEK |
| | | | | | | (SEQ ID. |
| | | | | | | No. 341); |
| | | | | | | FNVEACFPT |
| | | | | | | LIVEK |

| | | | | |
|---|---|---|---|---|
| *developed | Y | Y | Pos | (SEQ ID. |
| after | | | | No. 342) |
| collection | | | | Yes (1 day |
| | | | | doxycicline) |

*POS signifies positive

Tick-borne pathogen peptides are present in the urine of 40% of nonacute patients with clinical suspicion of tick-borne illnesses

In the validation phase of the study, urine samples from 148 non acute patients (n=36 PTLDS; n=112 clinically suspected of tick-borne illnesses) and 150 new healthy and diseased controls were analyzed. Patients (M/F=0.45, median age=48, IQR=28.1) reported with symptoms including EM rash, fatigue, fever, joint pain/arthritis, brain fog, memory loss and other neurological symptoms. The control set (M/F=1.75, median age=35, IQR=22) included healthy controls and disease control patients with clinical history of Chagas disease, tuberculosis and traumatic brain injury.

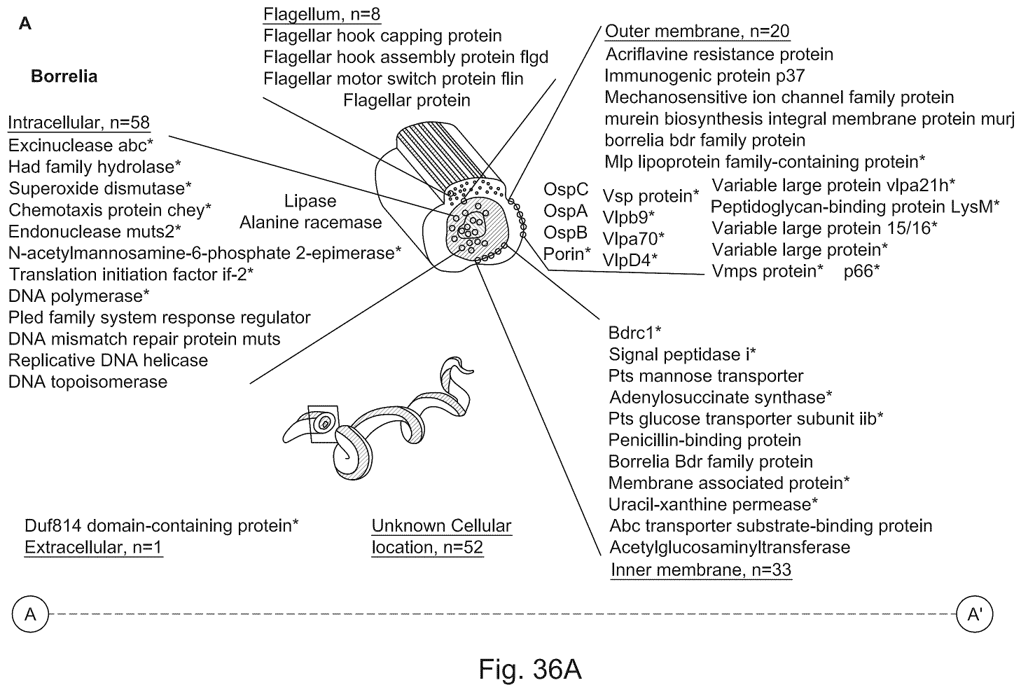

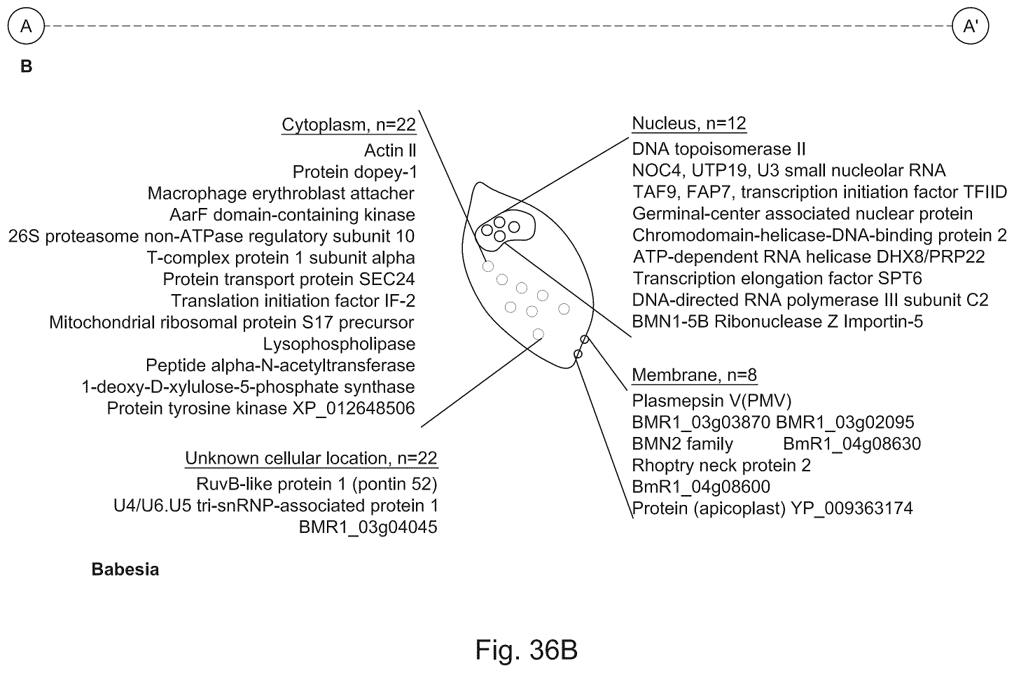

In an embodiment, 279 unique peptides specifically attributed to microorganisms belonging to the genus Borrelia, Babesia, Anaplasma, Ehrlichia, Bartonella, Rickettsia, and known to be pathogenic in humans were identified in n=108/148 patient samples. No peptides from TBEV and Powassan virus were identified in patients or controls. Peptides matching Borrelia sp. (n=160, FIG. 33A), Babesia sp. (n=62, FIG. 33B), Rickettsia sp. (n=8), Francisella sp. (n=6), Anaplasma sp. (n=8), Bartonella sp. (n=15), Ehrlichia (n=12) were identified. In 89/148 patients at least 1 peptide from Borrelia was identified. Within this subset, 29% (26/89) carried peptides from Babesia proteins, 8% (7/89) for Rickettsia, 7% (6/89) for Anaplasma, 10% (9/89) for Bartonella, 6% (5/89) for Ehrlichia. The case group included 8 pediatric patients. Peptides matching Borrelia were detected in 4/8 pediatric patients, while peptides from Babesia were found in 3/8 patients and one peptide from Rickettsia and 2 from Ehrlichia were found in 1/8 patients respectively. In the validation set, a single peptide from tick-borne organisms was found in 21/150 negative controls (15%). No negative control presented more than 1 peptide. As defined in the training phase of the study, a patient was considered positive for a given organism if at least two unique peptides deriving unambiguously from such organism were identified. According to this criterion, 59/148 non acute patients (40%) were positive for at least one tick-borne pathogen and all the controls were negative. n=48 were positive for Borrelia, n=17 positive for Babesia, n=4 were positive for Bartonella, n=2 were positive for Ehrlichia, n=8 were positive for Borrelia and Babesia, n=1 was positive for Borrelia and Bartonella, n=1 was positive for Babesia and Bartonella, n=1 was positive for Babesia, Bartonella and Anaplasma. Therefore, 48/148 patients (32%, FIGS. 36A and 36B) were positive for only one pathogen and 10/148 samples (7%) were positive for 2 pathogens, and 1/148 (0.7%) was positive for 3 pathogens suggesting the co-existence of multiple infections (FIG. 34A). Seven unique peptides belonging to the genus Francisella and common to the species tularensis, novicida, and hispaniensis, persica were also identified. Urinary peptides from species known to be non-pathogenic suggest a commensal host-microbe interaction.

Urinary Pathogen Peptides Revert to Undetectable Levels after Symptom Resolution.

Longitudinal study of three patients provided anectdotal evidence that urinary peptides revert to undetectable levels after symptom resolution. Pre and post treatment urine collection was obtained from two acute LB patients. In one case (patient No. of 108838), three Borrelia peptides were identified at the time of positive serology and EM rash. Complete clearing of Borrelia peptides was observed after symptoms resolution with two 14-day courses of doxycycline. In the second patient (No. of 453742), urine was collected at different time points: 1) after tick bite and before EM rash, 2) after development of the characteristic EM rash and before antibiotic treatment, 3) after 2 days of doxycycline treatment while the patient was still symptomatic. Three Borrelia peptides were detected in the urine before development of the EM rash. Borrelia peptides were then confirmed in the urine of the untreated, symptomatic patient. Borrelia peptides were also detected after two days of doxycycline treatment while the patient was still symptomatic (Table 13). A decline in peptide numbers following treatment was found for patient No. of 957477, positive for Erhlichia chaffeensis, whose urine was collected the first day of treatment as well as after two and four weeks (Table 13).

Table 13. Longitudinal study of two Lyme borreliosis patients, and one non-acute tick-borne disease patient. In patient 108838 (acute LB), Borrelia-specific peptides are identified in presence of acute symptoms (EM rash) and no peptide is detected after symptom resolution (4 weeks of doxycycline). In patient 453742 (acute LB), Borrelia-specific peptides were identified after tick bite but before development of an EM rash. Peptides were detectable in the pre-treatment stage and in presence of EM rash. Peptide count decreased during early treatment (2 days of doxycycline) when the patient was still symptomatic. In patient 957477 (non-acute tick-borne disease) 2 Ehrlichia peptides were identified in the presence of symptoms before starting treatment, 1 peptide after 14 days of doxycyxline and no peptides after 4 weeks of doxycycline.

TABLE 13
Detection of Borrelia-specific peptides

| | | | | Before | | |
|---|---|---|---|---|---|---|
| | | | Before | treatment - | | Resolution |
| | | | EM (after | presence | During | of |
| Sample ID | Clinical Status | Pathogen | tick bite) | of EM | treatment | symptoms |
| 108838 | Acute Lyme | Borrelia | N/A | 3 | N/A | 0 |
| | borreliosis | | | | | |
| 453742 | Acute Lyme | Borrelia | 3 | 2 | 2 | N/A |
| | borreliosis | | | | | |
| 957477 | PTLDS | Ehrlichia | N/A | 2* | 1 | 0 |

*EM rash was not present

Borrelia-Specific Urinary Peptides are Associated with Chemotaxis, Transmembrane Transport, Immune Evasion and Metabolism.

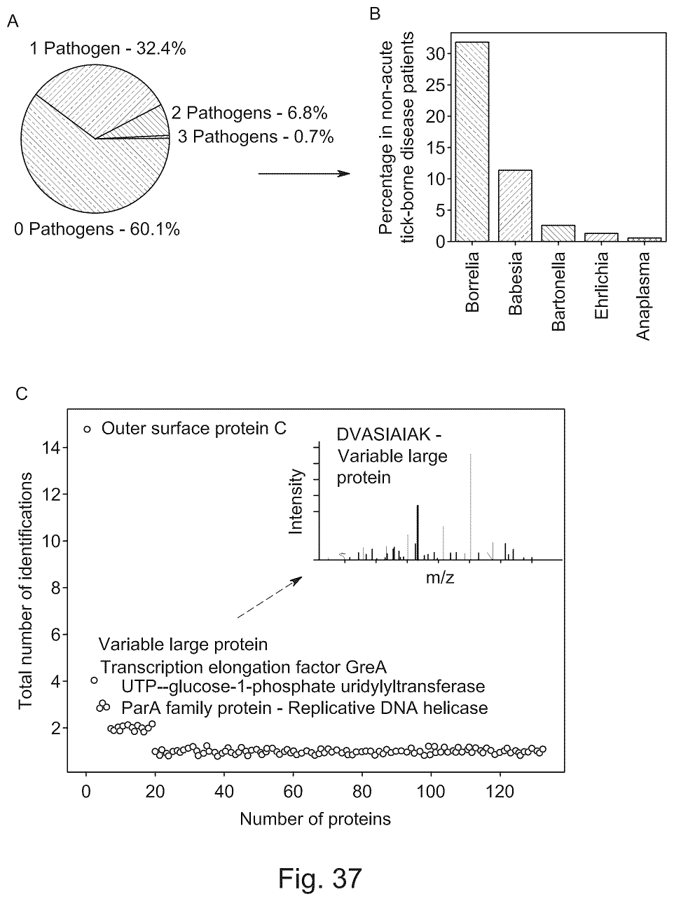

Peptides (N=160) from Borrelia species were the most abundant among the tick-borne infection pathogens investigated. Gene Ontology (GO) analysis of biological functions indicated that a large number of proteins were associated with chemotaxis, biosynthesis, transmembrane transport, immune evasion and DNA metabolism. Chemotaxis and motility are required for Borrelia to establish infection in the mammalian host. In this study, we identified peptides specific for chemotaxis and motility associated proteins including flagellin, CheA, and MotA. Transmembrane transport plays a role in drug resistance, in parasite-host interaction, in cell signaling and virulence. Urinary peptides associated with transmembrane transport proteins included ABC transporter permease, acriflavine resistance protein, and mechanosensitive ion channel. In response to mammalian host immunity, Borrelia modulates its transcriptional activity to facilitate dissemination and immune evasion. In this study, we identified DNA mismatch repair protein, DNA polymerases, and DNA ligases, which are proteins involved in DNA metabolism. Cell envelope proteins are involved in a number of processes required for Borrelia to establish infection in the mammalian host, including cell adhesion, cell invasion and immune escape. Examples of proteins in this category include outer surface protein A (OspA), outer surface protein B, and outer surface protein C (OspC). Among the proteins identified in the urine of non-acute patients there were 6 known seroreactive proteins: OspA, OspB, OspC, Flagellin, Porin, P37 and OppaIV. OspC and Flagellin are also included in the two-tiered Lyme borreliosis serology according to CDC criteria29. 55 identified proteins are known to be localized in the membrane region (of which n=10 are known to be localized in the outer membrane and n=4 in the inner membrane), 54 in the cytoplasm and 12 in the flagellum (FIG. 36A). The most represented Borrelia proteins in the peptide repertoire were OspC (17 peptide hits), variable large protein (4 peptide hits), and transcription elongation factor (4 peptide hits) (FIG. 37C). More than 65% of the Borrelia derived urinary peptides identified in this study (109/160) were detected only once (FIG. 37C).

Borrelia Peptides in the Cerebrospinal Fluid of a Clinically Suspected Neuroborreliosis Patient are Also Detectable in the Urine (Anectdotal).

The experimental protocol described in FIG. 2 and the algorithm of were applied to matched cerebrospinal fluid (CSF) and urine samples from a clinically suspected Neuroborreliosis patient. Ten months prior to sample collection, the patient experienced worsening of neurological symptoms, including fainting, ataxia, and tremors in the face, neck and hands. Two peptides were detected in the CSF: OspC peptide LKEKHQDLGVANGDTTDNNAK (SEQ ID NO. 343) and Acriflavine resistance protein peptide VTSNLDVEK (SEQ ID NO. 344). The same OspC peptide was detected in the urine.

The Number of Urinary Peptides Correlates with Presence or Absence of Symptoms in Non Acute Tick Borne Disease Patients.

Symptoms reported by non acute patients (PTLDS and patients with clinical suspicion of tick-borne illnesses) included previous EM rash, joint pain, fatigue, fever, facial palsy, and other neurological symptoms. A score of a 0 and 1 was attributed in absence or presence of any symptom designated in Table 11. Using an ordinal regression model, we found that for those subjects where clinical data were available (N=46), urinary peptide number was positively correlated with presence or absence of symptoms (p-value <0.001).

Alignment Analysis Informs Verification of Protein Database Annotation and Unambiguous Species Attribution of Urinary Peptides.

Alignment analysis within evolutionarily related organisms in the clade was conducted to achieve two goals: 1) verification of the protein database annotation, and 2) attribution of the peptide to an organism at the species level. In order to achieve the former, full length sequence of the protein associated with each urinary peptide was retrieved from the highest-ranking species in FASTA format and compared to homologous proteins (data from Basic Local Alignment Search Tool (BLAST)). In the case of Borrelia, annotated species of Borrelia were used, including: Borrelia burgdorferi, Borrelia garinii, Borrelia afzelii, Borrelia bavariensis, Borrelia mayonii, Borrelia miyamotoi, Borrelia hermsii, Borrelia turicatae, Borrelia chilensis, Borrelia duttonii, Borrelia. If the protein demonstrated greater than 60% homology, over the full query length, with other species in the query, then the database annotation was validated. In this study, protein database annotation was validated in all the instances and no rejection was necessary. In order to attribute peptides to an organism at the species level, the peptide sequence was studied in the context of homologous proteins in the clade. A peptide was unambiguously attributed to a species if the peptide sequence had 100% match with the given species and less than 90% sequence identity to any other species investigated. Species variation can be an important cause of diagnostic inaccuracy due to lack of reactivity of detection reagents.

Orthogonal Technologies Western Blot Analysis and Parallel Reaction Monitoring Confirm Urinary Peptide Identification.

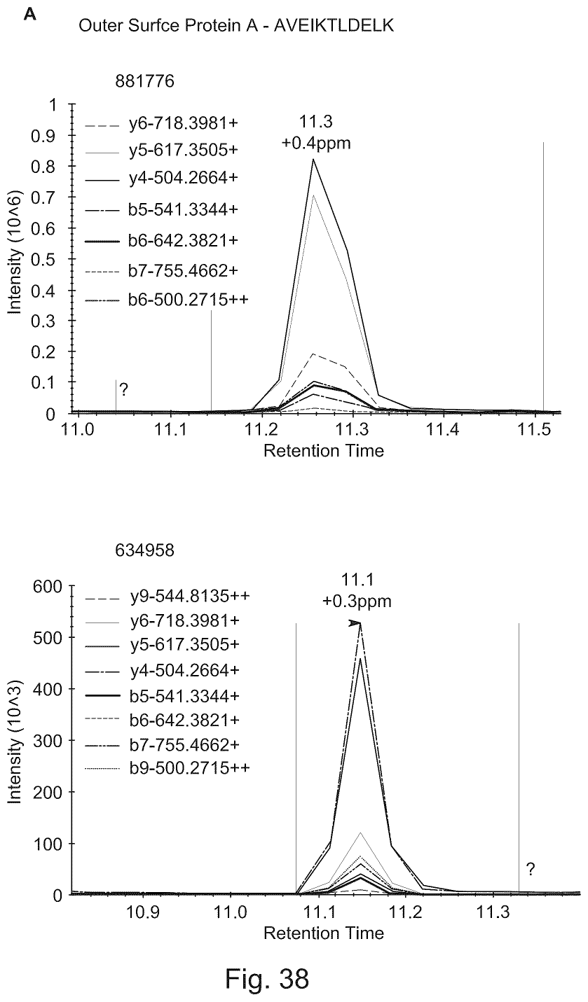

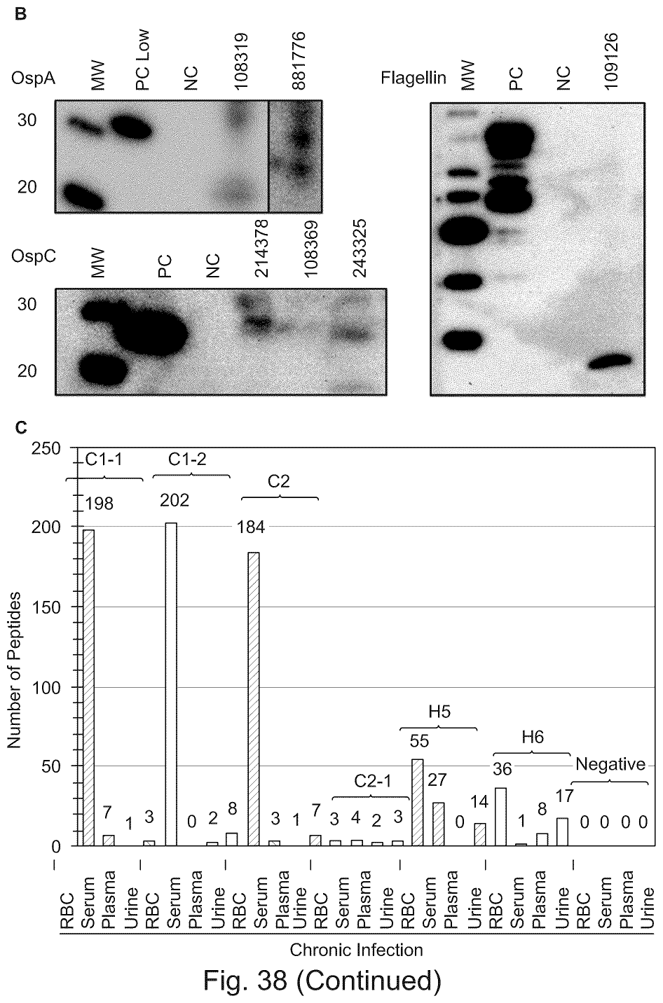

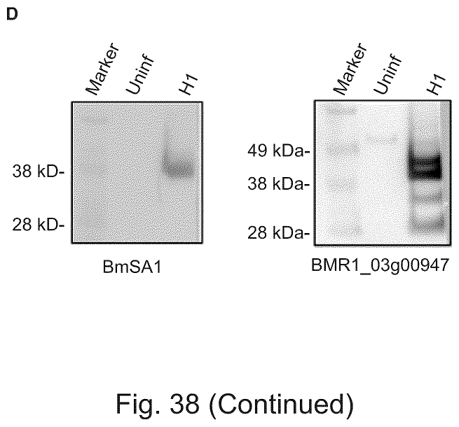

Pathogen-specific, urinary peptides were confirmed using affinity particle enrichment and two orthogonal approaches—Parallel Reaction Monitoring (PRM) and western blotting. For the former, N=3 peptides from OspA, OspC, and flagellar proteins were chosen based on the discovery full scan MS/MS results on patient samples and on additional LC-MS/MS analysis of healthy volunteer urine spiked with recombinant proteins. Peptide AVEIKTLDELK (SEQ ID NO. 345) deriving from OspA was confirmed by PRM analysis in four patient samples that yielded this peptide in the discovery MS/MS analysis (FIG. 38A). In these samples, peptide LKNSHAELGVAGNGATTDENAQK (SEQ ID. No. 328) from OspC and NDVSEEKPEIK from flagellar motor switch protein were not detected. To further verify MS/MS results, additional sample aliquots from six previously tested patients were processed with affinity particles and tested by western blot analysis using previously validated antibodies for OspA, OspC and Flagellin. Western blot results were concordant with MS/MS findings for OspC (2/3 patients), OspA (2/2 patients), and Flagellin (1/1 patient) (FIG. 38B).

Babesia microti Derived Peptides are Detected in the Urine and Blood of an Animal Model of Persistent Infection and Correlate with Parasitemia.

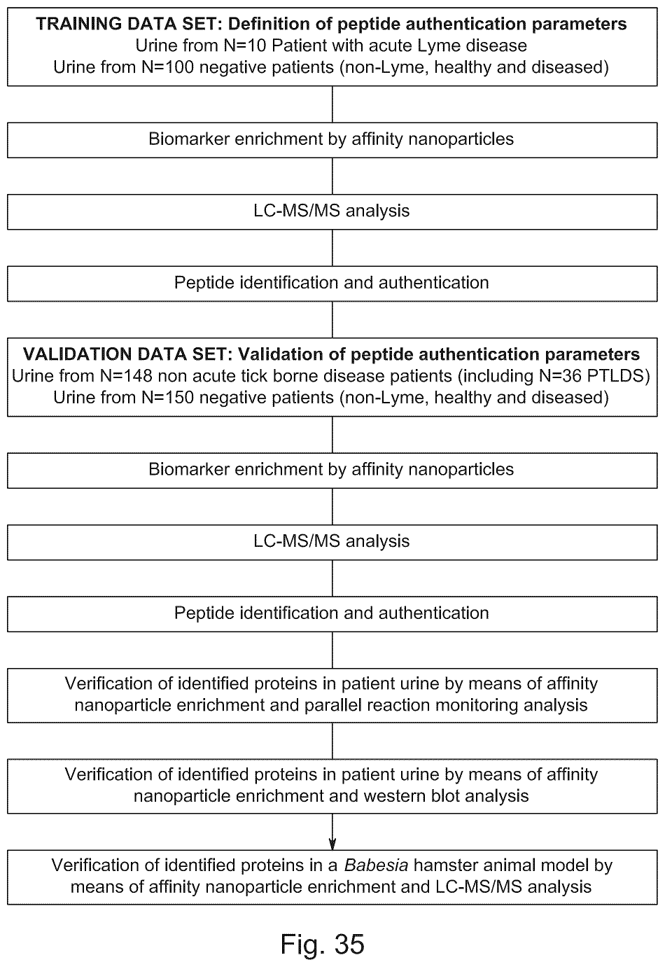

In order to substantiate the hypothesis that peptides derived from a tick-borne pathogen in later phases of infection can be detected in peripheral body fluids such as urine, we analyzed bodily fluids derived from golden hamsters (Mesocricetus auratus) infected with Babesia microti. Six golden hamsters with parasitemia ranging from 0% to 42% and one uninfected control were studied at different times after infection, ranging from 3 to 6 months to mimic chronic infection. PCR to Babesia ITS regions30 was used to confirm infection loads. 870 unique Babesia peptides belonging to 319 proteins were identified in red blood cells (RBC), plasma and urine. The number of Babesia proteins in the RBC compartment correlated with levels of parasitemia by linear regression analysis (p<0.0001). Even though the site of Babesia infection is the blood, Babesia derived peptides were detected in the urine of hamsters at late stage of infection (FIG. 35 C), where parasitemia was low or undetectable (H5 and H6 yielded 14 and 17 urinary peptides and parasitemia of 0% and 1%, respectively, FIG. 38C, 38D). Among the hundreds of peptides identified, members of the BMN2 family were detected in RBCs (BMR1_03g00020, BMR1_02g04265; BmR1_04g09980) and in urine of chronic hamsters (BMR1_02g04265). BMN2 family members are considered to be relevant in the immune evasion as suggested by their high mutation frequency and low immunogenicity, and were previously reported to be highly expressed antigens in Babesia microti. 3/62 (5%) Babesia peptides found in urine of non acute patients were also identified in body fluids of infected hamsters. These include importin-5 (BMR1_02g00750), guanine nucleotide-binding protein subunit beta-2-like 1 protein (BmR1_04g08285), hypothetical protein (BMR1_03g04255).

In one embodiment, the 3-tier authentication algorithm, which requires 100% amino acid sequence identity with tick-borne pathogen proteins, evolutionary taxonomic verification for related pathogens, and lack of overlap with human or any other organism, dramatically reduces the number of false positives that would have been otherwise called using direct MS sequencing by conventional MS software. Biologic and technical validation of the algorithm employs CDC criteria, serology positive acute Lyme patients, concomitant urine western blot immunoassays, orthogonal targeted identification using PRM, and an animal model of persistent Babesiosis. PRM is a targeted proteomic approach able to simultaneously monitor all fragment ions derived from selected peptides with high resolution and accuracy. Orthogonality between discovery phase and PRM can be obtained through a different combination of fragmentation strategies and mass analyzers. In discovery, precursors were fragmented with collision induced dissociation (CID), and product ions measured in the ion trap analyzer; in PRM, fragmentation was obtained with high energy collision induced dissociation (HCD) and product ions were measured in the Orbitrap™ mass spectrometer. Stringent mass tolerance filters (≤1 ppm) were applied to the product ions in the spectra, allowing for a highly confident peptide identification.

In one embodiment, the method yielded zero false positives in 250 diseased and healthy controls and identified up to five specific urinary Borrelia peptides in 10 acute LB patients, including proteins that are included in the panel for the standard Lyme serological test (Table 13). we were able to anectdotically observe a decrease in the number of peptides during antibiotic treatment and absence of tick-borne pathogen peptides after successful treatment and symptom remission.

In one embodiment, Addressing the question of persistent infection, 279 different urinary peptides, derived from the surface or subcellular compartments of pathogenic strains of tick-borne pathogens, were identified in non acute patients (PTLDS and patients with clinical suspicion of tick-borne illnesses). In 40% (n=59/148) of them we identified two or more peptides unique for at least one tick-borne pathogen and the number of urinary pathogen derived peptides correlated with the presence or absence of symptoms (p<0.001) reported by the treating physician when available. 32% (n=48/155) of patients presented peptides derived from one pathogen, while 7% presented (n=10/148) peptides from two pathogens, and less than 1% (n=1/148) presented 3 pathogens.

Borrelia was the most frequently represented organism. A large number of identified proteins are located on the membrane surface and several are known to be antigenic. It is important to note that Borrelia undergoes several changes during host infections which require the production of new membrane proteins that could be used for immune evasion or adaptation to the new environment. Multiple proteins identified herein are currently recognized as antigens in the standard serological test: OspC, Flagellin. Among the Borrelia genus, the highest number of peptides were derived from species related to Lyme borreliosis.

In this study, at least two different peptides associated with Borrelia were found in n=48/148 non acute patients suspected of tick-borne illnesses. Represented Borrelia species included Lyme-associated as well as TBRF-associated species. In many subjects both were found. 66 unique peptides specific for TBRF Borrelia species were found including 24 peptides from Borrelia miyamotoi which is being diagnosed in the United States in an increasing number of patients. Recent evidence shows that TBRF Borrelia species can also be carried by Ixodes ticks, the same vector that transmits Lyme borreliosis. TBRF is an often-neglected disease and may go underdiagnosed in many patients. In fact, TBRF patients can yield a positive serology for Lyme borreliosis because of proteins with overlapping antigenic similarities with Lyme Borrelia species, thus its true prevalence can be underestimated.

Method to Design Experiment 4

Study design. A method consisting of sample pre-analytical concentration, mass spectrometry analysis and a novel peptide authentication algorithm was applied to 408 urine patient specimens (Table 12) in order to discern the presence of peptides belonging to the proteome of selected tick-borne pathogens. Urine samples were subjected to pre-analytical concentration and mass spectrometry analysis. Urine specimens were divided in a training (N=110 patients, 10 cases and 100 controls) and a non-overlapping validation (N=298 patients, 148 cases and 150 controls) set that were used to establish the parameters of the peptide authentication algorithm to ensure that identified peptide sequences were uniquely attributable to tick-borne pathogens. The algorithm included four steps: 1) determination of physical and statistical parameters for mass spectrum matching, 2) BLAST searches of peptides longer 7 amino acids to ensure that the selected peptide sequence has percentage identity lower than 100% with proteins of non-tick pathogen organisms, and 3) validation of protein database annotation via alignment with homologous proteins of evolutionary related organisms in the clade. At the conclusion of the analysis, we performed manual quality check of spectra and we did not find any discrepancy or incorrect attributions. Peptides identified with the method were verified using Western Blot analysis¹⁰⁴, parallel reaction monitoring²² and an animal model of persistent Babesia microti infection¹⁰⁵. The correlation of urinary peptides derived from tick-borne pathogen with patient symptoms was investigated.

Patient Study Cohorts.

Urine samples were collected from consented (IRB Pro00008518, Chesapeake IRB) number patients who were suspected of having tick-borne diseases in different geographic regions at high risk for tick-borne diseases in the US and Europe (clinics: Hope McIntyre, MD, Maryland; Deborah Hoadley MD LLC, Massachusetts; Innatoss Laboratories B.V., Netherlands). Acceptance criteria for 1) acute LB patients (N=10) included the characteristic erythema migrans (EM) rash and positive two-tier LB serology according to CDC criteria. Non acute patients suspected of tick-borne illnesses include: 2) PTLDS patients (N=36); acceptance criteria included previous LB diagnosis and persistence of symptoms following antibiotic therapy for LB according to the Infectious Disease Society of America guidelines; 3) other non acute patients suspected of tick-borne disease (n=112): acceptance criteria for other non acute symptomatic patients included clinical suspicion of tick-borne illnesses by treating physisician based on symptoms including joint pain, fever, neurologic impairment, neuropathy, fatigue, and depression. This study met the requirements for IRB approval (Pro00008518, Chesapeake IRB). An informed consent form was signed by all patients enrolled in this study and by their treating physicians. If the patient was a minor, written consent and assent from the subject, was obtained from a parent or legal guardian. All methods were performed in accordance with relevant guidelines and regulations. Clinical and demographic data included age, sex, previous tick-borne disease diagnosis, self-reported symptoms, and physician assessed symptoms. Urine specimens were collected from 215 diseased controls and 35 healthy volunteers (Table S3) from the US and Peru. Diseased negative controls included hospitalized patient affected by HIV infection, tuberculosis, Chagas disease, and acute respiratory distress syndrome (ARDS) following traumatic brain injury. Diagnosis of pulmonary tuberculosis was verified by sputum smear microscopy and culture methods. HIV infection was confirmed by HIV nucleic acid amplification test and CD4 count. Chagas disease status was determined by microscopy examination of blood smears and quantitative PCR analysis of blood. ARDS patients were diagnosed using the Berlin Definition criteria that include bilateral lung infiltrates detected with chest X-rays, pulmonary capillary pressure ≤18 mmHg, and oxygenation levels PaO2/FiO2≤200 mmHg.

Collection of Bio-Fluids from Patients Under Evaluation for Acute Lyme Borreliosis and Non Acute Patients Suspected of Tick-Borne Illnesses

Matched coded clinical records and LB serology results were also provided under patient consent. Urine samples from US-based collection sites were refrigerated immediately after collection and sent to George Mason University in refrigerated containers within 24 hours from collection; samples were then frozen at −80° C. upon arrival. Urine samples from European collection sites were immediately frozen upon collection, transferred to George Mason University in dry ice and stored at −80° C. Cerebrospinal fluid (CSF) was collected by lumbar puncture. The CSF sample was immediately placed in dry ice, shipped from Massachusetts to Mason in dry ice, and kept at −80° C. until analysis.

Affinity Particle Processing of Biofluids from Patient Subjects

500 μl of cerebrospinal fluid from patients suspected of tick borne illnesses were centrifuged at 3,750×g for 15 minutes, the pellet was discarded, and supernatant was recovered and diluted with 500 μl Tris-HCl 50 mM, pH 7.2. Urine samples (at least 42 ml) were thawed in warm water (approx. 37° C.) on an orbital shaker. Urinalysis was performed using a Multistix 10 SG reagent strip. Urine was transferred into 50 ml tubes and centrifuged at 3,700×g for 15 minutes. Urine was decanted into a new tube and the pellet was discarded. pH was adjusted to 5.5 incrementally adding 1 M hydrochloric acid or 1 M sodium hydroxide. 40 ml of urine sample was transferred into a 50 ml polycarbonate tube. Urine and CSF samples were incubated with 200 μl affinity particles (10 mg/ml) for 30 minutes at RT. CSF samples were centrifuged at 16,100×g for 20 minutes while urine samples at 19,000×g (Beckman Avanti JXN-26 Centrifuge) for 45 minutes. Supernatants from CSF and urine samples were discarded. Particle pellet was washed twice by vigorously resuspending it in 1 ml 18 MQ-cm water followed by centrifugation at 16,100×g for 20 minutes. Supernatant was discarded and particle pellet was resuspended in 20 μl of elution buffer solution (4% Sodium Dodecyl Sulfate (SDS) in 50 mM ammonium bicarbonate), and incubated for 20 minutes at RT. Samples were centrifuged at 16,100×g for 20 minutes. Eluates were saved and transferred into new tubes and processed for mass spectrometry as described further.

Mass Spectrometry Analysis

Eluates were reduced using 200 mM dithiothreitol at room temperature for 15 minutes and alkylated using 50 mM iodacetamide at room temperature in the dark for 20 minutes. The enzymatic digestion ran overnight with 2 μl of (0.5 μg/μl) of sequencing grade trypsin (Promega, V5113) in 50 mM ammonium bicarbonate pH 8 at 37° C. Digestion was then stopped by adding 2 μl of 100% trifluoracetic acid (TFA). Digested samples were then desalted with C-18 spin columns (Pierce, No. of89870). Final eluates were dried with a nitrogen evaporator (Microvap 118, Organomation Associates, Inc). Samples were reconstituted in 10 μl of 0.1% Formic Acid. LC-MS/MS was performed on an Orbitrap Fusion™ Tribrid™ Mass Spectrometer (Thermo Scientific) coupled with a nanospray EASY-nLC 1200 UHPLC. Reversed-phase chromatography separation of the peptide mixture was performed using PepMap RSLC 75 μm i.d.×15 cm long with 2 μm, C18 resin LC column (ThermoFisher). 0.1% formic acid as mobile phase A, and 0.1% formic acid, 80% acetonitrile mobile phase B were used. Samples were peptides were eluted using a linear gradient of 5% mobile phase B to 50% mobile phase B in 90 min at 300 nL/min, then to 100% mobile phase B for an additional 2 min. The Thermo Orbitrap Fusion™ Tribrid™ Mass Spectrometer (Thermo Scientific) was operated in a data-dependent mode in which each full MS scan was followed by TopN MS/MS scans of the most abundant molecular ions with charge states form 2+ to 4+ were dynamically selected for collision induced dissociation (CID) using a normalized collision energy of 35%. Tandem mass spectra were searched against microorganism databases with Proteome Discoverer 2.1 software using tryptic cleavage constraints. Databases for the following microorganisms were downloaded from NCBI, UniProt, and PiroplasmaDB: Borrelia burgdorferi, Borrelia mayonii, Borrelia afzelii, Borrelia garinii, Borrelia spielmani, Borrelia bavariensis, Borrelia hermsii, B. turicatae, B. parkeri, B. miyamotoi, Babesia microti, Francisella tularensis, Ehrlichia chaffeensis, Rickettsia rickettsiae, Rickettsia parkeri, Rickettsia species 364D, Rickettsia akari, Anaplasma phagocytophilum, Bartonella henselae, Powassan virus, Tick-borne encephalitis virus, Colorado tick fever virus. In the training phase of the method databases were modified in order to exclude peptide sequences whose spectrum overlaps with sample contaminants.

Three-Tier Peptide Identification and Authentication Algorithm

We developed an algorithm to perform peptide authentication, which incorporates stringent filtering criteria in order to minimize the false positive rate. The algorithm includes the following steps:
A) Statistical and physical parameters for spectrum matching. 1) Peptide false discovery rate (FDR) based on forward-reverse decoy <1%, 2) Xcorr >2.0, >3.0 and >4.0 for 2+, 3+, 4+ precursor ions, 3) q-value <0.05, 4) precursor ion mass tolerance <2 ppm and fragment ion mass tolerance <0.5 Da, 5) If precursor is triply-charged: 5.1) Presence of a basic residue (K, R, H) within the sequence (excluding N-terminus and C-terminus residues), and 5.2) Presence of corresponding doubly charged precursor ion in the full mass chromatogram (MS1).
B) Unambiguous peptide attribution to one microorganism. In order to exclude peptides that share amino acid sequence with other organisms, each peptide attributed to a tick-borne pathogen was subjected to BLAST searches against the NCBI Reference Sequence database (RefSeq)¹⁰⁹, a comprehensive dataset containing the available protein sequence information for any given species. A peptide showing 100% identity to any additional species included in the RefSeq database beyond the intended tick-borne pathogen was discarded. Peptide sequences shorter than 7 amino acids were discarded in order to minimize random error of attribution when searching for short-peptide sequences¹¹⁰. Date and time of BLAST search and database download were recorded.
C) Validation of protein database annotation. The full-length protein, to which every peptide was attributed, was aligned with homologous proteins of evolutionary related organisms in the clade. If the full-length protein had greater than 60% identity with proteins in the query, the database annotation was considered valid.

Attribution of urinary peptides to an organism at the species level was conducted as follows. Full length homologous proteins in related microorganism were aligned using the JalView software. For Borrelia, the following species were taken into consideration: Borrelia burgdorferi, Borrelia garinii, Borrelia afzelii, Borrelia bavariensis, Borrelia mayonii, Borrelia miyamotoi, Borrelia hermsii, Borrelia turicatae, Borrelia recurrentis, Borrelia chilensis, Borrelia crocidurae, Borrelia duttonii, Borrelia bissettii. A peptide showing 100% identity to a single species and <90% to other species was attributed to the microorganism at the species level. A peptide showing 100% identity to one or more species and >90% identity to different species was not attributed to the microorganism at the species level, and all the species with 100% identity were reported.

Targeted Peptide Identification with Parallel Reaction Monitoring

LC-MS/MS was performed on an Orbitrap Fusion™ Tribrid™ Mass Spectrometer (Thermo Scientific) coupled with a nanospray EASY-nLC 1200 UHPLC. Reversed-phase chromatography was performed using PepMap RSLC 75 μm i.d.×15 cm long with 2 μm, C18 resin LC column (ThermoFisher). Peptides were eluted using a linear gradient of 5% mobile phase B to 50% mobile phase B in 15 min at 300 nL/min, then to 100% mobile phase B for an additional 2 min. The Orbitrap Fusion was operated in data independent acquisition parallel reaction monitoring mode. A targeted list of precursor ions of the peptides of interest AVEIKTLDELK (SEQ ID NO. 345) (m/z=420.24; z=3), LKNSHAELGVAGNGATTDENAQK SEQ ID. No. 328) (m/z=775.72; z=2), NDVSEEKPEIK (m/z=644.32) were isolated and fragmented by Higher-energy C-trap dissociation (HCD) with 35% normalized collision and detected at a mass resolution of 60,000. The data were then analyzed using Skyline v3.6 (University of Washington, MacCoss Lab) to determine the presence or absence of peptides of interest.

Propagation of Babesia microti in Hamsters

Babesia microti GI (BEI Resources NR-44070; ATCC® PRA-398™) was originally isolated from blood obtained from a human case of babesiosis in Nantucket, Massachusetts, USA, in 1983^{111,112} The isolate was maintained by in vivo propagation in Golden Syrian hamsters (Harlan Laboratories, stock: HsdHan:AURA) according to published protocols^{113,114} and procedures approved by the ATCC® IACUC. Ten hamsters were inoculated with ˜10⁸ parasitized erythrocytes in 0.5 ml of blood. Blood samples were collected by the peri-orbital route following inhalational anesthesia with isoflurane and parasitemia was determined by microscopic examination of Giemsa-stained blood films at different times of infection. A minimum of 500 erythrocytes were counted to calculate the percent parasitemia of each sample. This included all parasitized cells regardless of intraerythrocytic stage or number of parasites per cell. After 30 days of infection, four hamsters (acute group) were anesthetized by ketamine injection (50 mg/kg) and 0.5 ml of blood with and without heparin was collected from each animal. Urine samples (˜0.1 ml) were collected directly from the bladders with a syringe during abdominal surgery and animals were subsequently euthanized using carbon dioxide inhalation. The six remaining hamsters (chronic group) were monitored for 6 months and blood and urine samples were collected as described above.

### Statistical Analysis

Ordinal regression analysis was performed to evaluate correlation between the number of urinary pathogen derived peptides and presence or absence of clinical symptoms in non acute symptomatic patients suspected of tick-borne diseases. Linear regression analysis was performed to evaluate the correlation of the number of Babesia derived peptides with parasitemia in the hamster animal model experiment. T-test was used to test the significance of regression. Statistical analyses were performed using SPSS v.19.0(IBM Corp.). Descriptive statistical analysis of data derived from LD and non acute patients, controls, and hamsters was performed using Python 3 Pandas library and MicrosoftExcel. Visualization were obtained using Python 3 Matplotlib 3.1.1, Seaborn 9.0 libraries and Excel.

### INCORPORATION BY REFERENCE

Throughout this application, various references including publications, patents, and pre-grant patent application publications are referred to. Disclosures of these publications in their entireties are hereby incorporated by reference into this application to more fully describe the state of the art to which this invention pertains. It is specifically not admitted that any such reference constitutes prior art against the present application or against any claims thereof. All publications, patents, and pre-grant patent application publications cited in this specification are herein incorporated by reference, and for any and all purposes, as if each individual publication or patent application were specifically and individually indicated to be incorporated by reference. In the case of inconsistencies, the present disclosure will prevail.

### REFERENCES

1. Schmidt, R., et al., Single-molecule detection on a protein-array assay platform for the exposure of a tuberculosis antigen. J Proteome Res, 2011. 10(3): p. 1316-22.
2. Paris, L., et al., Urine lipoarabinomannan glycan in HIV-negative patients with pulmonary tuberculosis correlates with disease severity. Sci Transl Med, 2017. 9(420).
3. Marais, B. J., Childhood tuberculosis: epidemiology and natural history of disease. Indian J Pediatr, 2011. 78(3): p. 321-7.
4. Vass all, A., et al., Rapid diagnosis of tuberculosis with the Xpert MTB/RIF assay in high burden countries: a cost-effectiveness analysis. PLoS Med, 2011. 8(11): p. e1001120.
5. Cruz, A. T., P. A. Revell, and J. R. Starke, Gastric Aspirate Yield For Children With Suspected Pulmonary Tuberculosis. J Pediatric Infect Dis Soc, 2013. 2(2): p. 171-4.
6. Peter, J., et al., Urine for the diagnosis of tuberculosis: current approaches, clinical applicability, and new developments. Curr Opin Pulm Med, 2010. 16(3): p. 262-70.
7. Peter, J. G., G. Theron, and K. Dheda, Urine antigen test for diagnosis of HIV-associated tuberculosis. Lancet Infect Dis, 2012. 12(11): p. 825; author reply 826-7.
8. Shah, S. Biomagnetics Announces the First Urine-Based Test for Tuberculosis. MedGadget 2011; Available from: http://www.medgadget.com/2011/05/biomagnetics_announces_the_first_urinebased_test_for_tub erculosis.html.
9. Gaur, R. L., et al., LprG-mediated surface expression of lipoarabinomannan is essential for virulence of Mycobacterium tuberculosis. PLoS Pathog, 2014. 10(9): p. e1004376.
10. Kim, J. S., et al., Crystal structure and functional implications of LprF from Mycobacterium tuberculosis and M. bovis. Acta Crystallogr D Biol Crystallogr, 2014. 70(Pt 10): p. 2619-30.
11. Heydari, A. A., M. R. Movahhede Danesh, and K. Ghazvini, Urine PCR evaluation to diagnose pulmonary tuberculosis. Jundishapur J Microbiol, 2014. 7(3): p. e9311.
12. Castro-Sesquen, Y. E., et al., Use of a novel chagas urine nanoparticle test (chunap) for diagnosis of congenital chagas disease. PLoS Negl Trop Dis, 2014. 8(10): p. e3211.
13. Castro-Sesquen, Y. E., et al., Use of a Chagas Urine Nanoparticle Test (Chunap) to Correlate with Parasitemia Levels in T. cruzi/HIV Co-infected Patients. PLoS Negl Trop Dis, 2016. 10(2): p. e0004407.
14. Douglas, T. A., et al., The use of hydrogel microparticles to sequester and concentrate bacterial antigens in a urine test for Lyme disease. Biomaterials, 2011. 32(4): p. 1157-66.
15. Longo, C., et al., Core-Shell Hydrogel Particles Harvest, Concentrate and Preserve Labile Low Abundance Biomarkers. PLoS ONE, 2009. 4(3).
16. Luchini, A., V. Espina, and L. A. Liotta, Protein painting reveals solvent-excluded drug targets hidden within native protein-protein interfaces. Nat Commun, 2014. 5: p. 4413.
17. Magni, R., et al., Application of Nanocage technology for high sensitivity measurement of urinary outer surface protein A carboxyl-terminus domain in early stage Lyme borreliosis. J Transl Med, 2015. 13: p. 346.
18. Shafagati, N., et al., The use of Nanocage particles for biodefense and emerging infectious disease diagnostics. Pathog Dis, 2014. 71(2): p. 164-76.
19. Paris, L., et al., Urine lipoarabinomannan (LAM) glycan in HIV negative patients with pulmonary tuberculosis correlates with disease severity. Science Translational Medicine. In Press.
20. Aebischer, T., et al., A critical role for lipophosphoglycan in proinflammatory responses of dendritic cells to Leishmania mexicana. European Journal of Immunology, 2005. 35(2): p. 476-486.
21. Fredolini, C., et al., Concentration and Preservation of Very Low Abundance Biomarkers in Urine, such as Human Growth Hormone (hGH), by Cibacron Blue F3G-A Loaded Hydrogel Particles. Nano Research, 2008. 1(6): p. 502-518.
22. Fredolini, C., et al., Nanoparticle technology: amplifying the effective sensitivity of biomarker detection to create a urine test for hGH. Drug Test Anal, 2009. 1(9-10): p. 447-54.
23. Tamburro, D., et al., Multifunctional Core-Shell Nanoparticles: Discovery of Previously Invisible Biomarkers. J Am Chem Soc, 2011. 133: p. 19178-19188.
24. Luchini, A., et al., Smart hydrogel nanoparticles for serum cancer biomarkers harvesting. Proceedings of the American Association for Cancer Research Annual Meeting, 2007. 48: p. 587-588.
25. Nozawa, N., et al., Real-time PCR assay using specimens on filter disks as a template for detection of cytomegalovirus in urine. J Clin Microbiol, 2007. 45(4): p. 1305-7.
26. McDonough, J. A., et al., The twin-arginine translocation pathway of Mycobacterium smegmatis is functional and required for the export of mycobacterial beta-lactamases. J Bacteriol, 2005. 187(22): p. 7667-79.
27. Moore, D. A., et al., Microscopic observation drug susceptibility assay, a rapid, reliable diagnostic test for multidrug-resistant tuberculosis suitable for use in resource-poor settings. J Clin Microbiol, 2004. 42(10): p. 4432-7.
28. Shea, Y. R., et al., High sensitivity and specificity of acid-fast microscopy for diagnosis of pulmonary tuberculosis in an African population with a high prevalence of human immunodeficiency virus. J Clin Microbiol, 2009. 47(5): p. 1553-5.
29. Tamburro, D., et al., Multifunctional Core-Shell Nanoparticles: Discovery of Previously Invisible Biomarkers. Journal of the American Chemical Society, 2011. 133(47): p. 19178-19188.
30. Gunther, S., et al., IL-1 Family Cytokines Use Distinct Molecular Mechanisms to Signal through Their Shared Co-receptor. Immunity, 2017. 47(3): p. 510-523.e4.
31. Steinberg, H. E., et al., Toward detection of toxoplasmosis from urine in mice using hydro-gel nanoparticles concentration and parallel reaction monitoring mass spectrometry. Nanomedicine, 2017. 14(2): p. 461-469.
32. Shell, S. S., et al., Leaderless Transcripts and Small Proteins Are Common Features of the Mycobacterial Translational Landscape. PLoS Genet, 2015. 11(11): p. e1005641.
33. Higdon, R. and E. Kolker, A predictive model for identifying proteins by a single peptide match. Bioinformatics, 2007. 23(3): p. 277-80.
34. Silvanovich, A., et al., The value of short amino acid sequence matches for prediction of protein allergenicity. Toxicol Sci, 2006. 90(1): p. 252-8.
35. Nicodeme, P., Fast approximate motif statistics. J Comput Biol, 2001. 8(3): p. 235-48.
36. CDRH, F., USDHHS, Statistical Guidance on Reporting Results from Studies Evaluating Diagnostic Tests. 2007.
37. Acute Respiratory Distress Syndrome: The Berlin Definition. JAMA 307, 2526-2533 (2012).
38. Aebischer, T., et al., A critical role for lipophosphoglycan in proinflammatory responses of dendritic cells to Leishmania mexicana. European Journal of Immunology, 2005. 35(2): p. 476-486.
39. Aucott, J. N., Crowder, L. A. & Kortte, K. B. Development of a foundation for a case definition of post-treatment Lyme disease syndrome. Int. J. Infect. Dis. IJID Off. Publ. Int. Soc. Infect. Dis. 17, e443-449 (2013).
40. Aucott, J. N., Rebman, A. W., Crowder, L. A. & Kortte, K. B. Post-treatment Lyme disease syndrome symptomatology and the impact on life functioning: is there something here? Qual. Life Res. 22, 75-84 (2013).
41. Barbour, A. G. et al. A Genome-Wide Proteome Array Reveals a Limited Set of Immunogens in Natural Infections of Humans and White-Footed Mice with Borrelia burgdorferi. Infect. Immun. 76, 3374-3389 (2008).
42. Barbour, A. G., Adeolu, M. & Gupta, R. S. Division of the genus Borrelia into two genera (corresponding to Lyme disease and relapsing fever groups) reflects their genetic and phenotypic distinctiveness and will lead to a better understanding of these two groups of microbes (Margos et al. (2016) There is inadequate evidence to support the division of the genus Borrelia. Int. J. Syst. Evol. Microbiol. doi: 10.1099/ijsem.0.001717). Int. J. Syst. Evol. Microbiol. 67, 2058-2067 (2017).
43. Bergmann, A. R., Schmidt, B. L., Derler, A.-M. & Aberer, E. Importance of Sample Preparation for Molecular Diagnosis of Lyme Borreliosis from Urine. J. Clin. Microbiol. 40, 4581-4584 (2002).
44. Billeter, S. A., Levy, M. G., Chomel, B. B. & Breitschwerdt, E. B. Vector transmission of Bartonella species with emphasis on the potential for tick transmission. Med. Vet. Entomol. 22, 1-15 (2008).
45. Blanda, V. et al. A retrospective study of the characterization of Rickettsia species in ticks collected from humans. Ticks Tick-Borne Dis. 8, 610-614 (2017).
46. Bogovic, P. & Strle, F. Tick-borne encephalitis: A review of epidemiology, clinical characteristics, and management. World J. Clin. Cases WJCC 3, 430-441 (2015).
47. Breitschwerdt, E. B. Bartonellosis: One Health Perspectives for an Emerging Infectious Disease. ILAR J. 55, 46-58 (2014).
48. Brettschneider, S., Bruckbauer, H., Klugbauer, N. & Hofmann, H. Diagnostic Value of PCR for Detection of Borrelia burgdorferi in Skin Biopsy and Urine Samples from Patients with Skin Borreliosis. J. Clin. Microbiol. 36, 2658-2665 (1998).
49. Brown, L. D. & Macaluso, K. R. Rickettsia felis, an Emerging Flea-Borne Rickettsiosis. Curr.

Trop. Med. Rep. 3, 27-39 (2016).
50. CDRH, F., USDHHS, Statistical Guidance on Reporting Results from Studies Evaluating Diagnostic Tests. 2007.
51. Cerar, T. et al. Validation of cultivation and PCR methods for diagnosis of Lyme neuroborreliosis. J. Clin. Microbiol. 46, 3375-3379 (2008).
52. Clinicians I Tick-borne Relapsing Fever (TBRF) I CDC. (2018). Available at: https://www.cdc.gov/relapsing-fever/clinicians/index.html. (Accessed: 13 Apr. 2019)
53. Cluss, R. G., Silverman, D. A. & Stafford, T. R. Extracellular secretion of the Borrelia burgdorferi Oms28 porin and Bgp, a glycosaminoglycan binding protein. Infect. Immun. 72, 6279-6286 (2004).
54. Cornillot, E. et al. A targeted immunomic approach identifies diagnostic antigens in the human pathogen Babesia microti. Transfusion (Paris) 56, 2085-2099 (2016).
55. Crossland, N. A., Alvarez, X. & Embers, M. E. Late Disseminated Lyme Disease: Associated Pathology and Spirochete Persistence Posttreatment in Rhesus Macaques. Am. J. Pathol. 188, 672-682 (2018).
56. Cruz, A. T., P. A. Revell, and J. R. Starke, Gastric Aspirate Yield For Children With Suspected Pulmonary Tuberculosis. J Pediatric Infect Dis Soc, 2013. 2(2): p. 171-4.
57. Cullen, J. M. & Levine, J. F. Pathology of experimental Babesia microti infection in the Syrian hamster. Lab. Anim. Sci. 37, 640-643 (1987).
58. de Sousa, R. et al. Rickettsia slovaca Infection in Humans, Portugal. Emerg. Infect. Dis. 19, 1627-1629 (2013).
59. Diuk-Wasser, M. A., Vannier, E. & Krause, P. J. Coinfection by the tick-borne pathogens Babesia microti and Borrelia burgdorferi: ecological, epidemiological and clinical consequences. Trends Parasitol. 32, 30-42 (2016).
60. Donta, S. T. Issues in the Diagnosis and Treatment of Lyme Disease. Open Neurol. J. 6, 140-145 (2012).
61. Dworkin, M. S., Schwan, T. G., Anderson, D. E. & Borchardt, S. M. Tick-Borne Relapsing Fever. Infect. Dis. Clin. North Am. 22, 449-viii (2008).
62. Eldin, C., Jaulhac, B., Mediannikov, O., Arzouni, J.-P. & Raoult, D. Values of diagnostic tests for the various species of spirochetes. Med. Mal. Infect. (2019). doi:10.1016/j.medmal.2019.01.009
63. Elelu, N. Tick-borne relapsing fever as a potential veterinary medical problem. Vet. Med. Sci. 4, 271-279 (2018).
64. Elton, C. M., Rodriguez, M., Ben Mamoun, C., Lobo, C. A. & Wright, G. J. A library of recombinant Babesia microti cell surface and secreted proteins for diagnostics discovery and reverse vaccinology. Int. J. Parasitol. 49, 115-125 (2019).
65. Fatmi, S. S., Zehra, R. & Carpenter, D. O. Powassan Virus-A New Reemerging Tick-Borne Disease. Front. Public Health 5, (2017).
66. Fredolini, C., et al., Concentration and Preservation of Very Low Abundance Biomarkers in Urine, such as Human Growth Hormone (hGH), by Cibacron Blue F3G-A Loaded Hydrogel Particles. Nano Research, 2008. 1(6): p. 502-518.
67. Fredolini, C., et al., Nanoparticle technology: amplifying the effective sensitivity of biomarker detection to create a urine test for hGH. Drug Test Anal, 2009. 1(9-10): p. 447-54.
68. Gasmi, S. et al. Detection of municipalities at-risk of Lyme disease using passive surveillance of Ixodes scapularis as an early signal: A province-specific indicator in Canada. PloS One 14, e0212637 (2019).
69. Gaur, R. L., et al., LprG-mediated surface expression of lipoarabinomannan is essential for virulence of Mycobacterium tuberculosis. PLoS Pathog, 2014. 10(9): p. e1004376.
70. Girard, Y. A., Fedorova, N. & Lane, R. S. Genetic Diversity of Borrelia burgdorferi and Detection of B. bissettii-Like DNA in Serum of North-Coastal California Residents o. J. Clin. Microbiol. 49, 945-954 (2011).
71. Gray, J., Stedingk, L. V. von, Gurtelschmid, M. & Granström, M. Transmission Studies of Babesia microti in Ixodes ricinus Ticks and Gerbils. J. Clin. Microbiol. 40, 1259-1263 (2002).
72. Guedj, E., Eldin, C., Raoult, D., Azorin, J. M. & Belzeaux, R. Functional neuroimaging in patients presenting with somatoform disorders: A model for investigating persisting symptoms after tick bites and post-treatment Lyme disease syndrome? Med. Mal. Infect. (2019). doi:10.1016/j.medmal.2019.01.002
73. Gunther, S., et al., IL-1 Family Cytokines Use Distinct Molecular Mechanisms to Signal through Their Shared Co-receptor. Immunity, 2017. 47(3): p. 510-523.e4.
74. Halperin, J. J. Chronic Lyme disease: misconceptions and challenges for patient management. Infect. Drug Resist. 8, 119-128 (2015).
75. Haraldsson, B., Nystram, J. & Deen, W. M. Properties of the glomerular barrier and mechanisms of proteinuria. Physiol. Rev. 88, 451-487 (2008).
76. Heydari, A. A., M. R. Movahhede Danesh, and K. Ghazvini, Urine PCR evaluation to diagnose pulmonary tuberculosis. Jundishapur J Microbiol, 2014. 7(3): p. e9311.
77. Higdon, R. and E. Kolker, A predictive model for identifying proteins by a single peptide match. Bioinformatics, 2007. 23(3): p. 277-80.
78. Hirosue, S. & Dubrot, J. Modes of Antigen Presentation by Lymph Node Stromal Cells and Their Immunological Implications. Front. Immunol. 6, (2015).
79. Hodzic, E., Feng, S. & Barthold, S. W. Assessment of Transcriptional Activity of Borrelia burgdorferi and Host Cytokine Genes During Early and Late Infection in a Mouse Model. Vector-Borne Zoonotic Dis. 13, 694-711 (2013).
80. Hossain, M. et al. Enhanced sensitivity for selected reaction monitoring mass spectrometry-based targeted proteomics using a dual stage electrodynamic ion funnel interface. Mol. Cell. Proteomics MCP 10, M000062-MCP201 (2011).
81. Huber, B. et al. Description of Francisella hispaniensis sp. nov., isolated from human blood, reclassification of Francisella novicida (Larson et al. 1955) Olsufiev et al. 1959 as Francisella tularensis subsp. novicida comb. nov. and emended description of the genus Francisella. Int. J. Syst. Evol. Microbiol. 60, 1887-1896 (2010).
82. Ivanova, L. B. et al. Borrelia chilensis, a new member of the Borrelia burgdorferi sensu lato complex that extends the range of this genospecies in the Southern Hemisphere. Environ. Microbiol. 16, 1069-1080 (2014).
83. Jado, I. et al. Rickettsia monacensis and Human Disease, Spain. Emerg. Infect. Dis. 13, 1405-1407 (2007).
84. Jan, A. T. Outer Membrane Vesicles (OMVs) of Gram-negative Bacteria: A Perspective Update. Front. Microbiol. 8, (2017).
85. Jannetto, P. J. & Fitzgerald, R. L. Effective Use of Mass Spectrometry in the Clinical Laboratory. Clin. Chem. 62, 92-98 (2016).
86. Johnson, L., Wilcox, S., Mankoff, J. & Stricker, R. B. Severity of chronic Lyme disease compared to other chronic conditions: a quality of life survey. PeerJ 2, e322 (2014).
87. Jutras B L, Lochhead R B, Kloos Z A, Biboy J, Strle K, Booth C J, Govers S K, Gray J, Schumann P, Vollmer W, Bockenstedt L K, Steere A C, Jacobs-Wagner C. Borrelia burgdorferi peptidoglycan is a persistent antigen in patients with Lyme arthritis. Proc Natl Acad Sci USA. 2019 Jul. 2; 116(27):13498-13507. doi: 10.1073/pnas.1904170116. Epub 2019 Jun. 17.
88. Kedl, R. M. et al. Migratory dendritic cells acquire and present lymphatic endothelial cell-archived antigens during lymph node contraction. Nat. Commun. 8, 2034 (2017).
89. Kentsis, A. et al. Urine proteomics for profiling of human disease using high accuracy mass spectrometry. PROTEOMICS—Clin. Appl. 3, 1052-1061 (2009).
90. Kim, B. et al. Affinity enrichment for mass spectrometry: improving the yield of low abundance biomarkers. Expert Rev. Proteomics 15, 353-366 (2018).
91. Kim, J. S., et al., Crystal structure and functional implications of LprF from Mycobacterium tuberculosis and M. bovis. Acta Crystallogr D Biol Crystallogr, 2014. 70(Pt 10): p. 2619-30.
92. Knapp, K. L. & Rice, N. A. Human Coinfection with Borrelia burgdorferi and Babesia microti in the United States. J. Parasitol. Res. 2015, (2015).
93. Kodym, P. et al. Serological Diagnostics of Lyme Borreliosis: Comparison of Universal and Borrelia Species-Specific Tests Based on Whole-Cell and Recombinant Antigens. J. Clin. Microbiol. 56, e00601-18 (2018).
94. Kornacki, J. A. & Oliver, D. B. Lyme disease-causing Borrelia species encode multiple lipoproteins homologous to peptide-binding proteins of ABC-type transporters. Infect. Immun. 66, 4115-4122 (1998).
95. Kovalcuka, L. et al. Immunogenetic Manifestations of Lyme Borreliosis/Imuniengtiskas Izpausmes Laimboreliozes Gadijuma. Proc. Latv. Acad. Sci. Sect. B Nat. Exact Appl. Sci. 70, 215-219 (2016).
96. Krause, P. J. et al. Concurrent Lyme disease and babesiosis. Evidence for increased severity and duration of illness. JAMA 275, 1657-1660 (1996).
97. Krause, P. J. et al. Disease-specific diagnosis of coinfecting tickborne zoonoses: babesiosis, human granulocytic ehrlichiosis, and Lyme disease. Clin. Infect. Dis. Off. Publ. Infect. Dis. Soc.

Am. 34, 1184-1191 (2002).
98. Krause, P. J., Fish, D., Narasimhan, S. & Barbour, A. G. Borrelia miyamotoi Infection in Nature and in Humans. Clin. Microbiol. Infect. Off. Publ. Eur. Soc. Clin. Microbiol. Infect. Dis. 21, 631-639 (2015).
99. Krause, P. J., Fish, D., Narasimhan, S. & Barbour, A. G. Borrelia miyamotoi Infection in Nature and in Humans. Clin. Microbiol. Infect. Off. Publ. Eur. Soc. Clin. Microbiol. Infect. Dis. 21, 631-639 (2015).
100. Kruger, H., Heim, E., Schuknecht, B. & Scholz, S. Acute and chronic neuroborreliosis with and without CNS involvement: a clinical, MRI, and HLA study of 27 cases. J. Neurol. 238, 271-280 (1991).
101. Kugeler, K. J. et al. Discrimination between Francisella tularensis and Francisella-Like Endosymbionts when Screening Ticks by PCR. Appl. Environ. Microbiol. 71, 7594-7597 (2005).
102. Lacout, A., El Hajjam, M., Marcy, P.-Y. & Perronne, C. The Persistent Lyme Disease: ‘True Chronic Lyme Disease’ rather than ‘Post-treatment Lyme Disease Syndrome’. J. Glob. Infect. Dis. 10, 170-171 (2018).
103. Lantos, P. M. Chronic Lyme disease. Infect. Dis. Clin. North Am. 29, 325-340 (2015).
104. Liang, F. T. et al. Borrelia burgdorferi changes its surface antigenic expression in response to host immune responses. Infect. Immun. 72, 5759-5767 (2004).
105. Lindenbergh, M. F. S. & Stoorvogel, W. Antigen Presentation by Extracellular Vesicles from Professional Antigen-Presenting Cells. Annu. Rev. Immunol. 36, 435-459 (2018).
106. Longo, C. et al. Core-Shell Hydrogel Particles Harvest, Concentrate and Preserve Labile Low Abundance Biomarkers. PLoS ONE 4, (2009).
107. Longo, C., et al., Core-Shell Hydrogel Particles Harvest, Concentrate and Preserve Labile Low Abundance Biomarkers. PLoS ONE, 2009. 4(3).
108. Luchini, A. et al. Smart Hydrogel Particles: Biomarker Harvesting: One-step affinity purification, size exclusion, and protection against degradation. Nano Lett. 8, 350-361 (2008).
109. Magni, R., Luchini, A., Liotta, L. & Molestina, R. E. Analysis of the Babesia microti proteome in infected red blood cells by a combination of nanotechnology and mass spectrometry. Int. J. Parasitol. 49, 139-144 (2019).
110. Marais, B. J., Childhood tuberculosis: epidemiology and natural history of disease. Indian J Pediatr, 2011. 78(3): p. 321-7.
111. Mbow, M. L. et al. Major histocompatibility complex class II-independent generation of neutralizing antibodies against T-cell-dependent Borrelia burgdorferi antigens presented by dendritic cells: regulation by NK and gammadelta T cells. Infect. Immun. 69, 2407-2415 (2001).
112. McDonough, J. A., et al., The twin-arginine translocation pathway of Mycobacterium smegmatis is functional and required for the export of mycobacterial beta-lactamases. J Bacteriol, 2005. 187(22): p. 7667-79.
113. McFee, R. B. Tick borne illness—Rocky mountain spotted fever. Dis. Mon. 64, 185-194 (2018).
114. Mead, P. S. Epidemiology of Lyme disease. Infect. Dis. Clin. North Am. 29, 187-210 (2015).
115. Melenotte, C., Drancourt, M., Gorvel, J. P., Mege, J. L. & Raoult, D. Post-bacterial infection chronic fatigue syndrome is not a latent infection. Med. Mal. Infect. (2019). doi:10.1016/j.medmal.2019.01.006
116. Moody K D, Barthold S W, Terwilliger G A, Beck D S, Hansen G M, Jacoby R O. Experimental chronic Lyme borreliosis in Lewis rats. Am J Trop Med Hyg. 1990 February; 42(2):165-74.
117. Moody, D. B. & Cotton, R. N. Four pathways of CD1 antigen presentation to T cells. Curr. Opin. Immunol. 46, 127-133 (2017).
118. Moore, D. A., et al., Microscopic observation drug susceptibility assay, a rapid, reliable diagnostic test for multidrug-resistant tuberculosis suitable for use in resource-poor settings. J Clin Microbiol, 2004. 42(10): p. 4432-7.
119. Moutailler, S. et al. Co-infection of Ticks: The Rule Rather Than the Exception. PLoS Negl. Trop. Dis. 10, e0004539 (2016).
120. Multiple Acquisitions of Pathogen-Derived Francisella Endosymbionts in Soft Ticks. Available at: https://www.ncbi.nlm.nih.gov/pmc/articles/PMC5804916/. (Accessed: 1 Mar. 2019)
121. Nanagara, R., Duray, P. H. & Schumacher, H. R. Ultrastructural demonstration of spirochetal antigens in synovial fluid and synovial membrane in chronic Lyme disease: possible factors contributing to persistence of organisms. Hum. Pathol. 27, 1025-1034 (1996).
122. Nicodeme, P., Fast approximate motif statistics. J Comput Biol, 2001. 8(3): p. 235-48.
123. Noda, H., Munderloh, U. G. & Kurtti, T. J. Endosymbionts of Ticks and Their Relationship to Wolbachia spp. and Tick-Borne Pathogens of Humans and Animals. APPL Env. MICROBIOL 63, 7 (1997).
124. Notice to Readers Recommendations for Test Performance and Interpretation from the Second National Conference on Serologic Diagnosis of Lyme Disease. Available at: https://www.cdc.gov/mmwr/preview/mmwrhtml/00038469.htm. (Accessed: 19 Apr. 2019)
125. Nowalk, A. J., Gilmore, R. D. & Carroll, J. A. Serologic proteome analysis of Borrelia burgdorferi membrane-associated proteins. Infect. Immun. 74, 3864-3873 (2006).
126. Nozawa, N., et al., Real-time PCR assay using specimens on filter disks as a template for detection of cytomegalovirus in urine. J Clin Microbiol, 2007. 45(4): p. 1305-7.
127. Oz, H. S. & Hughes, W. T. Acute fulminating babesiosis in hamsters infected with Babesia microti. Int. J. Parasitol. 26, 667-670 (1996).
128. Padgett, K. A. et al. The Eco-epidemiology of Pacific Coast Tick Fever in California. PLoS Negl. Trop. Dis. 10, e0005020 (2016).
129. Paris, L. et al. Urine lipoarabinomannan glycan in HIV-negative patients with pulmonary tuberculosis correlates with disease severity. Sci. Transl. Med. 9, (2017).
130. Paris, L., et al., Urine lipoarabinomannan glycan in HIV-negative patients with pulmonary tuberculosis correlates with disease severity. Sci Transl Med, 2017. 9(420).
131. Peter, J., et al., Urine for the diagnosis of tuberculosis: current approaches, clinical applicability, and new developments. Curr Opin Pulm Med, 2010. 16(3): p. 262-70.
132. Peter, J. G., G. Theron, and K. Dheda, Urine antigen test for diagnosis of HIV-associated tuberculosis. Lancet Infect Dis, 2012. 12(11): p. 825; author reply 826-7.
133. Petnicki-Ocwieja, T. & Kern, A. Mechanisms of Borrelia burgdorferi internalization and intracellular innate immune signaling. Front. Cell. Infect. Microbiol. 4, (2014).
134. Piesman, J., Karakashian, S. J., Lewengrub, S., Rudzinska, M. A. & Spielmank, A. Development of Babesia microti sporozoites in adult Ixodes dammini. Int. J. Parasitol. 16, 381-385 (1986).
135. Poljak, A. et al. Identification and characterization of Borrelia antigens as potential vaccine candidates against Lyme borreliosis. Vaccine 30, 4398-4406 (2012).
136. Pretorius, A.-M. & Birtles, R. J. Rickettsia aeschlimannii: A New Pathogenic Spotted Fever Group Rickettsia, South Africa. Emerg. Infect. Dis. 8, 874 (2002).
137. Pritt, B. S. et al. Borrelia mayonii sp. nov., a member of the Borrelia burgdorferi sensu lato complex, detected in patients and ticks in the upper midwestern United States. Int. J. Syst. Evol. Microbiol. 66, 4878-4880 (2016).
138. Pruitt, K. D., Tatusova, T. & Maglott, D. R. NCBI Reference Sequence (RefSeq): a curated non-redundant sequence database of genomes, transcripts and proteins. Nucleic Acids Res. 33, D501-D504 (2005).
139. Pulkkinen, L. I. A., Butcher, S. J. & Anastasina, M. Tick-Borne Encephalitis Virus: A Structural View. Viruses 10, (2018).
140. Pulzova, L. & Bhide, M. Outer surface proteins of Borrelia: peerless immune evasion tools. Curr. Protein Pept. Sci. 15, 75-88 (2014).
141. Rappold, B. A. Mass Spectrometry Selectivity, Specifically. Clin. Chem. 62, 154-156 (2016).
142. Rebman, A. W. et al. The Clinical, Symptom, and Quality-of-Life Characterization of a Well-Defined Group of Patients with Posttreatment Lyme Disease Syndrome. Front. Med. 4, 224 (2017).
143. Reche, P. A. & Reinherz, E. L. Definition of MHC supertypes through clustering of MHC peptide-binding repertoires. Methods Mol. Biol. Clifton N J 409, 163-173 (2007).
144. Reche, P. A. & Reinherz, E. L. Prediction of peptide-MHC binding using profiles. Methods Mol. Biol. Clifton N J 409, 185-200 (2007).
145. Rudenko, N. et al. Delineation of a new species of the Borrelia burgdorferi Sensu Lato Complex, Borrelia americana sp. nov. J. Clin. Microbiol. 47, 3875-3880 (2009).
146. Sahni, S. K. & Rydkina, E. Host-cell interactions with pathogenic Rickettsia species. Future Microbiol. 4, 323-339 (2009).
147. Schmidt, R., et al., Single-molecule detection on a protein-array assay platform for the exposure of a tuberculosis antigen. J Proteome Res, 2011. 10(3): p. 1316-22.
148. Schroeder, C. L. C. et al. Transcriptional profiling of Rickettsia prowazekii coding and non-coding transcripts during in vitro host-pathogen and vector-pathogen interactions. Ticks Tick-Borne Dis. 8, 827-836 (2017).
149. Schutzer, S. E. et al. Direct Diagnostic Tests for Lyme Disease. Clin. Infect. Dis. Off. Publ. Infect. Dis. Soc. Am. (2018). doi:10.1093/cid/ciy614
150. Schutzer, S. E. et al. Whole-Genome Sequences of Borrelia bissettii, Borrelia valaisiana, and Borrelia spielmanii. J. Bacteriol. 194, 545-546 (2012).
151. Science, I. of M. (US) C. on L. D. and O. T.-B. D. T. S. of the. Diagnostics and Diagnosis. (National Academies Press (US), 2011).
152. Scoles, G. A. Phylogenetic Analysis of the Francisella-like Endosymbionts of Dermacentor Ticks. J. Med. Entomol. 41, 277-286 (2004).
153. Scott, J. D., Foley, J. E., Anderson, J. F., Clark, K. L. & Durden, L. A. Detection of Lyme Disease Bacterium, Borrelia burgdorferi sensu lato, in Blacklegged Ticks Collected in the Grand River Valley, Ontario, Canada. Int. J. Med. Sci. 14, 150-158 (2017).
154. Sentausa, E. et al. Genome Sequence of Rickettsia tamurae, a Recently Detected Human Pathogen in Japan. Genome Announc. 2, (2014).
155. Seward, R. J., Drouin, E. E., Steere, A. C. & Costello, C. E. Peptides Presented by HLA-DR Molecules in Synovia of Patients with Rheumatoid Arthritis or Antibiotic-Refractory Lyme Arthritis. Mol. Cell. Proteomics 10, M110.002477 (2011).
156. Shafagati, N., et al., The use of Nanocage particles for biodefense and emerging infectious disease diagnostics. Pathog Dis, 2014. 71(2): p. 164-76.
157. Shah, S. Biomagnetics Announces the First Urine-Based Test for Tuberculosis. MedGadget 2011; Available from: http://www.medgadget.com/2011/05/biomagnetics_announces_the_first_urinebased_test_for_tub erculosis.html.
158. Shapiro, D. S. & Schwartz, D. R. Exposure of Laboratory Workers to Francisella tularensis despite a Bioterrorism Procedure. J. Clin. Microbiol. 40, 2278-2281 (2002).
159. Sharma, N. et al. Detection of Francisella tularensis-Specific Antibodies in Patients with Tularemia by a Novel Competitive Enzyme-Linked Immunosorbent Assay. Clin. Vaccine Immunol. CVI 20, 9-16 (2013).
160. Shea, Y. R., et al., High sensitivity and specificity of acid-fast microscopy for diagnosis of pulmonary tuberculosis in an African population with a high prevalence of human immunodeficiency virus. J Clin Microbiol, 2009. 47(5): p. 1553-5.
161. Shell, S. S., et al., Leaderless Transcripts and Small Proteins Are Common Features of the Mycobacterial Translational Landscape. PLoS Genet, 2015. 11(11): p. e1005641.
162. Silva, J. C. et al. Genome-wide diversity and gene expression profiling of Babesia microti isolates identify polymorphic genes that mediate host-pathogen interactions. Sci. Rep. 6, 35284 (2016).
163. Silvanovich, A. et al. The value of short amino acid sequence matches for prediction of protein allergenicity. Toxicol. Sci. Off. J. Soc. Toxicol. 90, 252-258 (2006).
164. Silvanovich, A., et al., The value of short amino acid sequence matches for prediction of protein allergenicity. Toxicol Sci, 2006. 90(1): p. 252-8.
165. Silveira, I., Pacheco, R. C., Szab6, M. P. J., Ramos, H. G. C. & Labruna, M. B. Rickettsia parkeri in Brazil. Emerg. Infect. Dis. 13, 1111-1113 (2007).
166. Singh, S. K. & Girschick, H. J. Lyme borreliosis: from infection to autoimmunity. Clin. Microbiol. Infect. 10, 598-614 (2004).
167. Stanek, G. & Reiter, M. The expanding Lyme Borrelia complex-clinical significance of genomic species? Clin. Microbiol. Infect. 17, 487-493 (2011).
168. Steinberg, H. E. et al. Toward detection of toxoplasmosis from urine in mice using hydro-gel nanoparticles concentration and parallel reaction monitoring mass spectrometry. Nanomedicine Nanotechnol. Biol. Med. 14, 461-469 (2018).
169. Steinberg, H. E., et al., Toward detection of toxoplasmosis from urine in mice using hydro-gel nanoparticles concentration and parallel reaction monitoring mass spectrometry. Nanomedicine, 2017. 14(2): p. 461-469.
170. Sun, L. V., Scoles, G. A., Fish, D. & O'Neill, S. L. Francisella-like Endosymbionts of Ticks. J. Invertebr. Pathol. 76, 301-303 (2000).
171. Swanson, S. J., Neitzel, D., Reed, K. D. & Belongia, E. A. Coinfections acquired from ixodes ticks. Clin. Microbiol. Rev. 19, 708-727 (2006).
172. Sze, C. W., Zhang, K., Kariu, T., Pal, U. & Li, C. Borrelia burgdorferi Needs Chemotaxis To Establish Infection in Mammals and To Accomplish Its Enzootic Cycle. Infect. Immun. 80, 2485-2492 (2012).
173. Tan, K. E. et al. Prospective Evaluation of a Matrix-Assisted Laser Desorption Ionization-Time of Flight Mass Spectrometry System in a Hospital Clinical Microbiology Laboratory for Identification of Bacteria and Yeasts: a Bench-by-Bench Study for Assessing the Impact on Time to Identification and Cost-Effectiveness. J. Clin. Microbiol. 50, 3301-3308 (2012).
174. Telford, S. R. & Wormser, G. P. Bartonella spp. Transmission by Ticks Not Established. Emerg. Infect. Dis. 16, 379-384 (2010).
175. Thomas, S. N. & Zhang, H. Targeted proteomic assays for the verification of global proteomics insights. Expert Rev. Proteomics 13, 897-899 (2016).
176. Tickborne Diseases of the United States I Tick-borne Diseases I Ticks I CDC. (2018). Available at: https://www.cdc.gov/ticks/tickbomediseases/index.html. (Accessed: 1 Dec. 2018)
177. Tracy, K. E. & Baumgarth, N. Borrelia burgdorferi Manipulates Innate and Adaptive Immunity to Establish Persistence in Rodent Reservoir Hosts. Front. Immunol. 8, (2017).
178. Vassall, A., et al., Rapid diagnosis of tuberculosis with the Xpert MTB/RIF assay in high burden countries: a cost-effectiveness analysis. PLoS Med, 2011. 8(11): p. e1001120.
179. Vitale, G., Mansueto, S., Rolain, J.-M. & Raoult, D. Rickettsia massiliae Human Isolation. Emerg. Infect. Dis. 12, 174-175 (2006).
180. Wagemakers, A., Staarink, P. J., Sprong, H. & Hovius, J. W. R. Borrelia miyamotoi: a widespread tick-borne relapsing fever spirochete. Trends Parasitol. 31, 260-269 (2015).
181. Wang, J. et al. Lipid binding orientation within CDId affects recognition of Borrelia burgorferi antigens by NKT cells. Proc. Natl. Acad. Sci. 107, 1535-1540 (2010).
182. Wieser, A., Schneider, L., Jung, J. & Schubert, S. MALDI-TOF MS in microbiological diagnostics-identification of microorganisms and beyond (mini review). Appl. Microbiol. Biotechnol. 93, 965-974 (2012).
183. Wilson, N. S., El-Sukkari, D. & Villadangos, J. A. Dendritic cells constitutively present self antigens in their immature state in vivo and regulate antigen presentation by controlling the rates of MHC class II synthesis and endocytosis. Blood 103, 2187-2195 (2004).
184. Wormser, G. P., Shapiro, E. D. & Fish, D. Borrelia miyamotoi: An Emerging Tick-Borne Pathogen. Am. J. Med. 132, 136-137 (2019).
185. Yakimchuk, K. et al. Borrelia burgdorferi infection regulates CD1 expression in human cells and tissues via IL1-β. Eur. J. Immunol. 41, 694-705 (2011).

1. A nanoparticle comprising a core and a shell, wherein the core comprises a molecular bait, and wherein the nanoparticle is configured to capture a biomolecule, a nucleic acid, an exosome, and/or a virus.

2. (canceled)

3. The nanoparticle of claim 1, wherein the capture of the biomolecule, the nucleic acid, an exosome, and/or the virus is from a fluid sample comprising urine, blood, and/or saliva.

4. The nanoparticle of claim 3, wherein the capture is achieved by sequestering target analytes from a whole volume of the urine, the blood, and/or the saliva and bringing the target analytes into a small volume within the nanoparticle.

5. The nanoparticle of claim 1, wherein the core of the nanoparticle has surface area at least 1000 times greater than surface area of the shell of the nanoparticle.

6. The nanoparticle of claim 1, wherein the nanoparticles are an open mesh, non-aggregating, colloidal and >95% open void.

7. The nanoparticle of claim 1, wherein the nanoparticles are immobilized on a collapsible non hygroscopic net such that the target analyte is preserved in a dry state.

8. The nanoparticle of claim 7, wherein a collection device comprising the collapsible non hygroscopic net is configured to collect a fluid sample such that the fluid sample is in dry state.

9. (canceled)

10. The nanoparticle of claim 12, wherein an enzyme is immobilized with the nanoparticles, wherein the enzyme is configured to produce an enzymatically amplified color reaction inside the nanoparticles containing the target analyte.

11. (canceled)

12. The nanoparticle of claim 19, wherein a target analyte captured by the nanoparticle is configured to be displayed on a solid phase antibody for production of the enzymatically amplified color reaction inside the nanoparticles.

13. The nanoparticle of claim 12, where the nanoparticles have sensitivity of about 95% and specificity about 80% of the target analytes from a whole volume of the urine, the blood, and/or the saliva.

14-17. (canceled)

18. A method comprises:
a) fabricating nanoparticles comprising a core and a shell, wherein the core comprises a molecular bait, and wherein the nanoparticle is configured to capture the biomolecule, the nucleic acid, the exosome, and/or the virus;
b) immobilizing the nanoparticle on a collapsible non hygroscopic net;
c) collecting a fluid sample within a collecting device comprising the collapsible non hygroscopic net of step (b);
d) sequestering target analytes from a whole volume of the fluid sample and bringing the target analytes into a small volume within the nanoparticles;
e) analyzing a target analytes present in the fluid sample.

19. The method of claim 18, wherein the nanoparticles comprises an immobilized enzyme, wherein the immobilized enzyme is configured to produce an enzymatically amplified color reaction inside the nanoparticles containing the target analyte.

20. (canceled)

21. The method of claim 18, wherein the target analyte captured by the nanoparticle is configured to be displayed on a solid phase antibody for production of the enzymatically amplified color reaction inside the nanoparticles.

22. The method of claim 18, wherein the nanoparticle is functionalized with a molecular bait, wherein the molecular bait is configured to captures the target analytes in a solution displacing contaminant.

23. The method of claim 18, wherein the fluid sample is urine, blood, and/or saliva of a subject.

24. The method of claim 18, wherein the nanoparticles are an open mesh, non-aggregating, colloidal and >95% open void and wherein the nanoparticles have sensitivity of about 95% and specificity about 80% of the target analytes from the whole volume of the urine, the blood, and/or the saliva.

25-26. (canceled)

27. The biomarker of claim 52, wherein identification of a Chagas disease comprising a peptide comprising an amino acid sequence selected from the group consisting of SEQ ID No. 1 to SEQ ID No. 229 and/or SEQ ID No. 269 to SEQ ID No. 286.

28. The biomarker of claim 52, wherein identification of Mycobacterium species comprising a peptide comprising an amino acid sequence selected from the group consisting of SEQ ID No. 230 to SEQ ID No. 268 and/or SEQ ID No. 287 to SEQ ID No. 312.

29. (canceled)

30. The biomarker of claim 52, wherein identification of lyme disease comprising a peptide comprising an amino acid sequence selected from the group consisting of SEQ ID No. 322 to SEQ ID No. 345.

31-51. (canceled)

52. A biomarker, comprising an amino acid sequence selected from the group consisting of SEQ ID No. 1 to SEQ ID No. 345, for selection of a disease selected from the group consisting of a Chagas disease, a Mycobacterium disease and a Lyme disease.


SEQUENCE LISTING (345 sequences)

SEQ ID NO: 1 - protein, 9 aa, Trypanosoma cruzi
AAFAAEAAR

SEQ ID NO: 2 - protein, 16 aa, Trypanosoma cruzi
ACDVSEAKVEDSWSRR

SEQ ID NO: 3 - protein, 25 aa, Trypanosoma cruzi
ADVAYTTIMENTSSYFTMNYFVVLM

SEQ ID NO: 4 - protein, 9 aa, Trypanosoma cruzi
AEEAAAMLR

SEQ ID NO: 5 - protein, 9 aa, Trypanosoma cruzi
AEQITALLR

SEQ ID NO: 6 - protein, 9 aa, Trypanosoma cruzi
AESSVSVSR

SEQ ID NO: 7 - protein, 9 aa, Trypanosoma cruzi
AGAFVGSEK

SEQ ID NO: 8 - protein, 8 aa, Trypanosoma cruzi
AGDDEIQR

SEQ ID NO: 9 - protein, 17 aa, Trypanosoma cruzi
ALMDTNNFPAHVGAGER

SEQ ID NO: 10 - protein, 15 aa, Trypanosoma cruzi
AMMRPDLQYTEVLLR

SEQ ID NO: 11 - protein, 25 aa, Trypanosoma cruzi
APEQTGPFAADVLCIILLYIVVDMR

SEQ ID NO: 12 - protein, 11 aa, Trypanosoma cruzi
AQDISDLDLGR

SEQ ID NO: 13 - protein, 9 aa, Trypanosoma cruzi
AQPLETELR

SEQ ID NO: 14 - protein, 16 aa, Trypanosoma cruzi
AVEMVGADGTDTMFVD

SEQ ID NO: 15 - protein, 11 aa, Trypanosoma cruzi
AVSTVTTSPAG

SEQ ID NO: 16 - protein, 8 aa, Trypanosoma cruzi
AVVDGLLR

SEQ ID NO: 17 - protein, 8 aa, Trypanosoma cruzi
AVYVNSAR

SEQ ID NO: 18 - protein, 20 aa, Trypanosoma cruzi
CFHDADVVIGPHGSNLANAM

SEQ ID NO: 19 - protein, 10 aa, Trypanosoma cruzi
CGGCLCCSGR

SEQ ID NO: 20 - protein, 17 aa, Trypanosoma cruzi
CLFNSTVLLFAVVMMCC

SEQ ID NO: 21 - protein, 21 aa, Trypanosoma cruzi
CLTLNSSNPTAIMAAASVMQN

SEQ ID NO: 22 - protein, 15 aa, Trypanosoma cruzi
CPVEEGGANFSVGQR

SEQ ID NO: 23 - protein, 18 aa, Trypanosoma cruzi
CSMIIYSADNGSTWSLSK

SEQ ID NO: 24 - protein, 8 aa, Trypanosoma cruzi
CYYLALLR

SEQ ID NO: 25 - protein, 12 aa, Trypanosoma cruzi
DAGTIAGLNVIR

SEQ ID NO: 26 - protein, 10 aa, Trypanosoma cruzi
DATSSANSLR

SEQ ID NO: 27 - protein, 15 aa, Trypanosoma cruzi
DAVDTFGNFAEDVER

SEQ ID NO: 28 - protein, 11 aa, Trypanosoma cruzi
DGGTTTPDDDR

SEQ ID NO: 29 - protein, 16 aa, Trypanosoma cruzi
DHATWVADPAAPPSSR

SEQ ID NO: 30 - protein, 19 aa, Trypanosoma cruzi
DIAPYVAGGGAEARAEVAR

SEQ ID NO: 31 - protein, 14 aa, Trypanosoma cruzi
DMIMYPGPSAAAHR

SEQ ID NO: 32 - protein, 18 aa, Trypanosoma cruzi
DNLWAPVVLAQVCANYGV

SEQ ID NO: 33 - protein, 35 aa, Trypanosoma cruzi
DSPDAPTPAPSEPAPPPAPGPVHPPAPGDDDLDDL

SEQ ID NO: 34 - protein, 30 aa, Trypanosoma cruzi
EAEGMGYYHVSGGLPQSCGTEDHRPALHLR

SEQ ID NO: 35 - protein, 30 aa, Trypanosoma cruzi; identical to SEQ ID NO: 34

SEQ ID NO: 36 - protein, 17 aa, Trypanosoma cruzi
EAFSLFDKDGDGTITTK

SEQ ID NO: 37 - protein, 9 aa, Trypanosoma cruzi
EDAANNYAR

SEQ ID NO: 38 - protein, 14 aa, Trypanosoma cruzi
EDKESEGEVLVLTR

SEQ ID NO: 39 - protein, 17 aa, Trypanosoma cruzi
EEPNSDTEGEEAGVQEE

SEQ ID NO: 40 - protein, 30 aa, Trypanosoma cruzi
EEVEELVGFAPGGVCPFGVNENVRIYLDIS

SEQ ID NO: 41 - protein, 11 aa, Trypanosoma cruzi
EGILLEGSTLK

SEQ ID NO: 42 - protein, 10 aa, Trypanosoma cruzi
EGVVLGADTR

SEQ ID NO: 43 - protein, 15 aa, Trypanosoma cruzi
EHLFSMDVGDSLLFR

SEQ ID NO: 44 - protein, 11 aa, Trypanosoma cruzi
EINGTTQYVQR

SEQ ID NO: 45 - protein, 7 aa, Trypanosoma cruzi
ELDNQVK

SEQ ID NO: 46 - protein, 20 aa, Trypanosoma cruzi
EMCTLQLQLQQTPEKETFEK

SEQ ID NO: 47 - protein, 18 aa, Trypanosoma cruzi
EMLYGVALLSAALQQAEK

SEQ ID NO: 48 - protein, 17 aa, Trypanosoma cruzi
EQWALHGMMLNLSVPSR

SEQ ID NO: 49 - protein, 8 aa, Trypanosoma cruzi
ESGVDMLK

SEQ ID NO: 50 - protein, 12 aa, Trypanosoma cruzi
EVDEQMLNVQNK

SEQ ID NO: 51 - protein, 21 aa, Trypanosoma cruzi
FFAMAFGLFVLFVWMCCHCCR

SEQ ID NO: 52 - protein, 22 aa, Trypanosoma cruzi
FFHVNNTNLFVLMDGVSMFTEG

SEQ ID NO: 53 - protein, 16 aa, Trypanosoma cruzi
FGAVMLLATLQAYTIR

SEQ ID NO: 54 - protein, 13 aa, Trypanosoma cruzi
FGGMDCPDSVLAR

SEQ ID NO: 55 - protein, 17 aa, Trypanosoma cruzi
FIMFFCGCAVLLGLNCR

SEQ ID NO: 56 - protein, 12 aa, Trypanosoma cruzi
FITATVGDGDDK

SEQ ID NO: 57 - protein, 7 aa, Trypanosoma cruzi
FLDFVAR

SEQ ID NO: 58 - protein, 22 aa, Trypanosoma cruzi
FLFCLQETVGSSSMDYCYEKQQ

SEQ ID NO: 59 - protein, 13 aa, Trypanosoma cruzi
FMSTHEEQLEHSQ

SEQ ID NO: 60 - protein, 12 aa, Trypanosoma cruzi
FSDVVAGYIDSK

SEQ ID NO: 61 - protein, 20 aa, Trypanosoma cruzi
FTGDGETLLEAPCVAETTLR

SEQ ID NO: 62 - protein, 9 aa, Trypanosoma cruzi
FVDPSGLTR

SEQ ID NO: 63 - protein, 16 aa, Trypanosoma cruzi
FVGGAEAAVSSGVVMR

SEQ ID NO: 64 - protein, 15 aa, Trypanosoma cruzi
GASAIPCPADMQAGK

SEQ ID NO: 65 - protein, 15 aa, Trypanosoma cruzi
GDDEDDDDDDDEEEA

SEQ ID NO: 66 - protein, 20 aa, Trypanosoma cruzi
GDGTWYDCNEGETAAPSGSR

SEQ ID NO: 67 - protein, 28 aa, Trypanosoma cruzi
GEAVMDLLLAGCVPSGANPDSGVITFMK

SEQ ID NO: 68 - protein, 12 aa, Trypanosoma cruzi
GEDGSNNAVLEK

SEQ ID NO: 69 - protein, 9 aa, Trypanosoma cruzi
GEIIESNIR

SEQ ID NO: 70 - protein, 9 aa, Trypanosoma cruzi
GIGITLDEK

SEQ ID NO: 71 - protein, 12 aa, Trypanosoma cruzi
GILLFGPPGTGK

SEQ ID NO: 72 - protein, 8 aa, Trypanosoma cruzi
GLHLMAGR

SEQ ID NO: 73 - protein, 12 aa, Trypanosoma cruzi
GLLLFGPPGTGK

SEQ ID NO: 74 - protein, 20 aa, Trypanosoma cruzi
GSGAVMGDGTIVFPMQAKNK

SEQ ID NO: 75 - protein, 21 aa, Trypanosoma cruzi
GSMGSVGFAATLLHSMELFER

SEQ ID NO: 76 - protein, 22 aa, Trypanosoma cruzi
GSSGTATWGTTIVAAAEEFRAK

SEQ ID NO: 77 - protein, 10 aa, Trypanosoma cruzi
GVGTGVLMSR

SEQ ID NO: 78 - protein, 30 aa, Trypanosoma cruzi
GYAYRDTEMENVVMDICYNMGMGAQFGGKY

SEQ ID NO: 79 - protein, 23 aa, Trypanosoma cruzi
HAFGVPSYQYSIYNIADPLCSLF

SEQ ID NO: 80 - protein, 12 aa, Trypanosoma cruzi
HEVHSAVYDLTR

SEQ ID NO: 81 - protein, 24 aa, Trypanosoma cruzi
HHSDVVAFYDAFIHPEEELEVETR

SEQ ID NO: 82 - protein, 28 aa, Trypanosoma cruzi
HLVLSTLSASELQHAHDGDPMEGFFCSE

SEQ ID NO: 83 - protein, 21 aa, Trypanosoma cruzi
HSHSAIIVEGQHDFAVWDSSY

SEQ ID NO: 84 - protein, 30 aa, Trypanosoma cruzi
HTYDGDGDDDENFLQVTMATTTMGNYEVMK

SEQ ID NO: 85 - protein, 23 aa, Trypanosoma cruzi
HVMFFIAAVFFIWILVVDLIAVR

SEQ ID NO: 86 - protein, 21 aa, Trypanosoma cruzi
IEDNTMSSVNGVILFGDTTVR

SEQ ID NO: 87 - protein, 9 aa, Trypanosoma cruzi
IEQWLECQR

SEQ ID NO: 88 - protein, 17 aa, Trypanosoma cruzi
IFEDSEGITNAEETEVR

SEQ ID NO: 89 - protein, 16 aa, Trypanosoma cruzi
IFETILEMVYGLVLDS

SEQ ID NO: 90 - protein, 8 aa, Trypanosoma cruzi
IGEVIGPK

SEQ ID NO: 91 - protein, 11 aa, Trypanosoma cruzi
IGGIGTVPVGR

SEQ ID NO: 92 - protein, 8 aa, Trypanosoma cruzi
IGVSELQR

SEQ ID NO: 93 - protein, 8 aa, Trypanosoma cruzi
IIDAPGHR

SEQ ID NO: 94 - protein, 17 aa, Trypanosoma cruzi
IISNASCTTNCLAPLAK

SEQ ID NO: 95 - protein, 17 aa, Trypanosoma cruzi
ILSFIEAILVQGQTDPP

SEQ ID NO: 96 - protein, 8 aa, Trypanosoma cruzi
INIADFIR

SEQ ID NO: 97 - protein, 11 aa, Trypanosoma cruzi
IVVLGSGGVGK

SEQ ID NO: 98 - protein, 12 aa, Trypanosoma cruzi
KMLLEHFLLLAE

SEQ ID NO: 99 - protein, 20 aa, Trypanosoma cruzi
KPGSMVFTPPPTAVPLDDYR

SEQ ID NO: 100 - protein, 14 aa, Trypanosoma cruzi
KPQTNWMEVPFEHR

SEQ ID NO: 101 - protein, 10 aa, Trypanosoma cruzi
LALSEVELWR

SEQ ID NO: 102 - protein, 14 aa, Trypanosoma cruzi
LCCCPSVFATNVEK

SEQ ID NO: 103 - protein, 8 aa, Trypanosoma cruzi
LCDFGLAR

SEQ ID NO: 104 - protein, 26 aa, Trypanosoma cruzi
LDGAAQQLGGDAGAAAAAVGCSHFLL

SEQ ID NO: 105 - protein, 8 aa, Trypanosoma cruzi
LDLAELLR

SEQ ID NO: 106 - protein, 8 aa, Trypanosoma cruzi
LEEEDFLK

SEQ ID NO: 107 - protein, 11 aa, Trypanosoma cruzi
LFGVTTLDLVR

SEQ ID NO: 108 - protein, 21 aa, Trypanosoma cruzi
LGEIVTTIPTIGFNVETVEYK

SEQ ID NO: 109 - protein, 11 aa, Trypanosoma cruzi
LGGLSGLVPGR

SEQ ID NO: 110 - protein, 13 aa, Trypanosoma cruzi
LGGTCVNVGCVPK

SEQ ID NO: 111 - protein, 18 aa, Trypanosoma cruzi
LGTEAPAVAIGEDSVQWK

SEQ ID NO: 112 - protein, 8 aa, Trypanosoma cruzi
LGTVPLSR

SEQ ID NO: 113 - protein, 8 aa, Trypanosoma cruzi
LIAASQAR

SEQ ID NO: 114 - protein, 9 aa, Trypanosoma cruzi
LLEAGLAGK

SEQ ID NO: 115 - protein, 8 aa, Trypanosoma cruzi
LLILAEGR

SEQ ID NO: 116 - protein, 11 aa, Trypanosoma cruzi
LLSFYSIIASR

SEQ ID NO: 117 - protein, 18 aa, Trypanosoma cruzi
LMEEEYEGDTRPPDMVSM

SEQ ID NO: 118 - protein, 11 aa, Trypanosoma cruzi
LNGCAESAEAR

SEQ ID NO: 119 - protein, 8 aa, Trypanosoma cruzi
LPAIQGPR

SEQ ID NO: 120 - protein, 9 aa, Trypanosoma cruzi
LQGLTEELR

SEQ ID NO: 121 - protein, 11 aa, Trypanosoma cruzi
LQIWDTAGQER

SEQ ID NO: 122 - protein, 14 aa, Trypanosoma cruzi
LSVLIAGATGSIGR

SEQ ID NO: 123 - protein, 20 aa, Trypanosoma cruzi
LSVNYLQDEDAILLDFEVVT

SEQ ID NO: 124 - protein, 18 aa, Trypanosoma cruzi
LVTAEGHEPTCDETPMLR

SEQ ID NO: 125 - protein, 24 aa, Trypanosoma cruzi
MCQCGAVWVSCHVMVADSVPVCMR

SEQ ID NO: 126 - protein, 15 aa, Trypanosoma cruzi
MDWYTGLGITPENLR

SEQ ID NO: 127 - protein, 19 aa, Trypanosoma cruzi
MEGNHANESNNNNNNGEAG

SEQ ID NO: 128 - protein, 17 aa, Trypanosoma cruzi
MEVVTSDGMMPSLIGQM

SEQ ID NO: 129 - protein, 20 aa, Trypanosoma cruzi
MGTITQGMFAGSFIGVVYTA

SEQ ID NO: 130 - protein, 16 aa, Trypanosoma cruzi
MLEILLYLPSSSGSAY

SEQ ID NO: 131 - protein, 25 aa, Trypanosoma cruzi
MLTSYSDDEALLDAIMAGASGFVLK

SEQ ID NO: 132 - protein, 16 aa, Trypanosoma cruzi
MLVTEATPPLNLDQLR

SEQ ID NO: 133 - protein, 24 aa, Trypanosoma cruzi
MQEAKPVTSGSGSGGGGANEAEAK

SEQ ID NO: 134 - protein, 18 aa, Trypanosoma cruzi
MSHDSALTQTLDETGVER

SEQ ID NO: 135 - protein, 9 aa, Trypanosoma cruzi
NDQWARPPR

SEQ ID NO: 136 - protein, 18 aa, Trypanosoma cruzi
NGNTSGGSDMIAFPDSTC

SEQ ID NO: 137 - protein, 13 aa, Trypanosoma cruzi
NGTEGALADIANR

SEQ ID NO: 138 - protein, 14 aa, Trypanosoma cruzi
NLDIERPTYTNLNR

SEQ ID NO: 139 - protein, 13 aa, Trypanosoma cruzi
NMAELGRMVAMVH

SEQ ID NO: 140 - protein, 15 aa, Trypanosoma cruzi
NQSSGAPTFSFYLCK

SEQ ID NO: 141 - protein, 14 aa, Trypanosoma cruzi
NVSSSNHGAVMEAR

SEQ ID NO: 142 - protein, 27 aa, Trypanosoma cruzi
NVTFSGGGMILTVLIGAMTGDVANATF

SEQ ID NO: 143 - protein, 9 aa, Trypanosoma cruzi
NVVQEIEGR

SEQ ID NO: 144 - protein, 18 aa, Trypanosoma cruzi
PIIAQVLEMEYANNGDLR

SEQ ID NO: 145 - protein, 28 aa, Trypanosoma cruzi
PTINGTGENLDGDEEEGDVSQDESQEDR

SEQ ID NO: 146 - protein, 22 aa, Trypanosoma cruzi
QALCVTEAESFSLIINMMFQDR

SEQ ID NO: 147 - protein, 11 aa, Trypanosoma cruzi
QAPLHVSGADV

SEQ ID NO: 148 - protein, 17 aa, Trypanosoma cruzi
QDDLENVPGPPAAPDLR

SEQ ID NO: 149 - protein, 11 aa, Trypanosoma cruzi
QEETTEQEPEK

SEQ ID NO: 150 - protein, 8 aa, Trypanosoma cruzi
QEFVDLTK

SEQ ID NO: 151 - protein, 10 aa, Trypanosoma cruzi
QEQMECIPTR

SEQ ID NO: 152 - protein, 13 aa, Trypanosoma cruzi
QGDITMPSSLLEK

SEQ ID NO: 153 - protein, 20 aa, Trypanosoma cruzi
QGETTGSEPPTLINAFYPGD

SEQ ID NO: 154 - protein, 9 aa, Trypanosoma cruzi
QGILQTYVK

SEQ ID NO: 155 - protein, 9 aa, Trypanosoma cruzi
QHYQQQPPR

SEQ ID NO: 156 - protein, 14 aa, Trypanosoma cruzi
QITQVYGFYDECLR

SEQ ID NO: 157 - protein, 11 aa, Trypanosoma cruzi
QITVNDLPVGR

SEQ ID NO: 158 - protein, 15 aa, Trypanosoma cruzi
QLAITEFCDVMYGQR

SEQ ID NO: 159 - protein, 8 aa, Trypanosoma cruzi
QLEAGGHR

SEQ ID NO: 160 - protein, 8 aa, Trypanosoma cruzi
QLLEQDVR

SEQ ID NO: 161 - protein, 7 aa, Trypanosoma cruzi
QMHLLAR

SEQ ID NO: 162 - protein, 17 aa, Trypanosoma cruzi
QNVLLFEDTLWTEMVHK

SEQ ID NO: 163 - protein, 12 aa, Trypanosoma cruzi
QQAGAQSPSCPR

SEQ ID NO: 164 - protein, 16 aa, Trypanosoma cruzi
QSEEAETTTNAEQAVR

SEQ ID NO: 165 - protein, 13 aa, Trypanosoma cruzi
QSNELLSIIEDAK

SEQ ID NO: 166 - protein, 8 aa, Trypanosoma cruzi
QTELDLAR

SEQ ID NO: 167 - protein, 28 aa, Trypanosoma cruzi
QVNYGMAATTAAGLPVNNANGSTGLEYK

SEQ ID NO: 168 - protein, 31 aa, Trypanosoma cruzi
QWMDDAVEGSELHFVLDDADMLEDDAVHVNH

SEQ ID NO: 169 - protein, 10 aa, Trypanosoma cruzi
SALGALGVPK

SEQ ID NO: 170 - protein, 8 aa, Trypanosoma cruzi
SAPLLILR

SEQ ID NO: 171 - protein, 8 aa, Trypanosoma cruzi
SAVVAVLR

SEQ ID NO: 172 - protein, 12 aa, Trypanosoma cruzi
SIATPGCDSPLR

SEQ ID NO: 173 - protein, 18 aa, Trypanosoma cruzi
SMDDIVIQGMNLLSAACR

SEQ ID NO: 174 - protein, 22 aa, Trypanosoma cruzi
SQELQQDCIDCAAHALHELGLR

SEQ ID NO: 175 - protein, 21 aa, Trypanosoma cruzi
SSVLMEDGTLVFPLMATNAAK

SEQ ID NO: 176 - protein, 22 aa, Trypanosoma cruzi
STSSVSASLSMETVTAPADGEH

SEQ ID NO: 177 - protein, 25 aa, Trypanosoma cruzi
SYSILDPHVSNEPESAESSFSNGSL

SEQ ID NO: 178 - protein, 17 aa, Trypanosoma cruzi
TDALEFLVDLAAVIGYR

SEQ ID NO: 179 - protein, 10 aa, Trypanosoma cruzi
TGATIGFACK

SEQ ID NO: 180 - protein, 11 aa, Trypanosoma cruzi
THAVLLLIGSR

SEQ ID NO: 181 - protein, 22 aa, Trypanosoma cruzi
THHCYEGVTFSGVGAVLTFFLN

SEQ ID NO: 182 - protein, 34 aa, Trypanosoma cruzi
THQLILQMNTNEWYVYFDGMQVYDGYYMGSLFDE

SEQ ID NO: 183 - protein, 7 aa, Trypanosoma cruzi
TLEEALR

SEQ ID NO: 184 - protein, 22 aa, Trypanosoma cruzi
TTHQLAIVLQNGNQGSAYVDGR

SEQ ID NO: 185 - protein, 13 aa, Trypanosoma cruzi
TTPSYVAFTDTER

SEQ ID NO: 186 - protein, 13 aa, Trypanosoma cruzi
TVEAEAAHGTVTR

SEQ ID NO: 187 - protein, 19 aa, Trypanosoma cruzi
TVYSVVVWYAEDHHWQELR

SEQ ID NO: 188 - protein, 9 aa, Trypanosoma cruzi
VAESVPLPK

SEQ ID NO: 189 - protein, 11 aa, Trypanosoma cruzi
VAGGVSAPTAK

SEQ ID NO: 190 - protein, 10 aa, Trypanosoma cruzi
VATGTILSHR

SEQ ID NO: 191 - protein, 14 aa, Trypanosoma cruzi
VDAANLIVLVVAVA

SEQ ID NO: 192 - protein, 10 aa, Trypanosoma cruzi
VDAETVEQLR

SEQ ID NO: 193 - protein, 13 aa, Trypanosoma cruzi
VDGQSIVASSGDR

SEQ ID NO: 194 - protein, 16 aa, Trypanosoma cruzi
VEECVPGGFSPRPSHS

SEQ ID NO: 195 - protein, 8 aa, Trypanosoma cruzi
VEIAEILR

SEQ ID NO: 196 - protein, 11 aa, Trypanosoma cruzi
VEIIANDQGNR

SEQ ID NO: 197 - protein, 12 aa, Trypanosoma cruzi
VEQLVSAVEGQK

SEQ ID NO: 198 - protein, 11 aa, Trypanosoma cruzi
VGSSAMVTMVR

SEQ ID NO: 199 - protein, 10 aa, Trypanosoma cruzi
VHNLSLNTPK

SEQ ID NO: 200 - protein, 11 aa, Trypanosoma cruzi
VIVEGGGIIVK

SEQ ID NO: 201 - protein, 11 aa, Trypanosoma cruzi
VLLLGDSGVGK

SEQ ID NO: 202 - protein, 16 aa, Trypanosoma cruzi
VLYVDSGPYEEVDTAR

SEQ ID NO: 203 - protein, 23 aa, Trypanosoma cruzi
VMIFCGTGGGAQAAVEEPSSDPK

SEQ ID NO: 204 - protein, 15 aa, Trypanosoma cruzi
VMYLMEAETGLLYLF

SEQ ID NO: 205 - protein, 7 aa, Trypanosoma cruzi
VNDLFER

SEQ ID NO: 206 - protein, 20 aa, Trypanosoma cruzi
VQWACAPHVMRLAILADVWH

SEQ ID NO: 207 - protein, 10 aa, Trypanosoma cruzi
VSGMAEDVIR

SEQ ID NO: 208 - protein, 8 aa, Trypanosoma cruzi
VTDFLLSR

SEQ ID NO: 209 - protein, 14 aa, Trypanosoma cruzi
VTVLSSQAEDPQVR

SEQ ID NO: 210 - protein, 13 aa, Trypanosoma cruzi
VVDAADADLVGSL

SEQ ID NO: 211 - protein, 16 aa, Trypanosoma cruzi
VVHGSVIYMLVGMYNR

SEQ ID NO: 212 - protein, 15 aa, Trypanosoma cruzi
VVIAYEPVWAIGTGK

SEQ ID NO: 213 - protein, 11 aa, Trypanosoma cruzi
VVLLGDSGVGK

SEQ ID NO: 214 - protein, 18 aa, Trypanosoma cruzi
VVPAPDFAEAALAAVTPR

SEQ ID NO: 215 - protein, 8 aa, Trypanosoma cruzi
WGVAQQTL

SEQ ID NO: 216 - protein, 16 aa, Trypanosoma cruzi
WMQEVTVYATAMQAVR

SEQ ID NO: 217 - protein, 11 aa, Trypanosoma cruzi
WSSSLEGNLWK

SEQ ID NO: 218 - protein, 18 aa, Trypanosoma cruzi
WTGENLVPPAPCANLSGK

SEQ ID NO: 219 - protein, 12 aa, Trypanosoma cruzi
YDANYVLTTEDF

SEQ ID NO: 220 - protein, 16 aa, Trypanosoma cruzi
YEPAGDQPAAIDDLER

SEQ ID NO: 221 - protein, 21 aa, Trypanosoma cruzi
YEYTSGDLGCCITQIHMFLEK

SEQ ID NO: 222 - protein, 13 aa, Trypanosoma cruzi
YHFYNRSYLTGEK

SEQ ID NO: 223 - protein, 16 aa, Trypanosoma cruzi
YIGGAAGANSGGDANK

SEQ ID NO: 224 - protein, 7 aa, Trypanosoma cruzi
YLAYLFQ

SEQ ID NO: 225 - protein, 9 aa, Trypanosoma cruzi
YLFGDFVAR

SEQ ID NO: 226 - protein, 9 aa, Trypanosoma cruzi
YLGLLENIR

SEQ ID NO: 227 - protein, 7 aa, Trypanosoma cruzi
YLQLVFR

SEQ ID NO: 228 - protein, 7 aa, Trypanosoma cruzi
YLYEALR

SEQ ID NO: 229 - protein, 21 aa, Trypanosoma cruzi
YQGVTEEIATIKNTMSGVAAR

SEQ ID NO: 230 - protein, 10 aa, Mycobacterium tuberculosis
TPLTATGLSK

SEQ ID NO: 231 - protein, 13 aa, Mycobacterium tuberculosis
AGQRWISIVEPLR

SEQ ID NO: 232 - protein, 14 aa, Mycobacterium tuberculosis
VTRETLALLIDLAR

SEQ ID NO: 233 - protein, 20 aa, Mycobacterium tuberculosis
SLWLAVVAGMIVAMLVYVLA

SEQ ID NO: 234 - protein, 21 aa, Mycobacterium tuberculosis
LQTPMQVYSADGELIAQFGEK

SEQ ID NO: 235 - protein, 10 aa, Mycobacterium tuberculosis
MLECYLQLSR

SEQ ID NO: 236 - protein, 18 aa, Mycobacterium tuberculosis
AAEQYWQAEQATNGYMLK

SEQ ID NO: 237 - protein, 21 aa, Mycobacterium tuberculosis
EIRPNLDELETGALSPYPIMR

SEQ ID NO: 238 - protein, 24 aa, Mycobacterium tuberculosis
AIFVLGDEGMEGGGGTLTSAAVTK

SEQ ID NO: 239 - protein, 8 aa, Mycobacterium tuberculosis
WFAELTTK

SEQ ID NO: 240 - protein, 9 aa, Mycobacterium tuberculosis
DKAALSSLR

SEQ ID NO: 241 - protein, 10 aa, Mycobacterium tuberculosis
QDEIIEATGR

SEQ ID NO: 242 - protein, 9 aa, Mycobacterium tuberculosis
MQAAVAQGR

SEQ ID NO: 243 - protein, 9 aa, Mycobacterium tuberculosis
NDLGVFDFR

SEQ ID NO: 244 - protein, 30 aa, Mycobacterium tuberculosis
AIAENPDDIAEYCTSGLTLTGFLGLSDTPR

SEQ ID NO: 245 - protein, 10 aa, Mycobacterium tuberculosis
VAVIELDADR

SEQ ID NO: 246 - protein, 10 aa, Mycobacterium tuberculosis
VLAAASSPEK

SEQ ID NO: 247 - protein, 19 aa, Mycobacterium tuberculosis
EFLLDYLGIQHEPGVVMSR

SEQ ID NO: 248 - protein, 19 aa, Mycobacterium tuberculosis
VVFINPADIEALGLTPGGR

SEQ ID NO: 249 - protein, 15 aa, Mycobacterium tuberculosis
FSVAELVRPWLDTWR

SEQ ID NO: 250 - protein, 27 aa, Mycobacterium tuberculosis
IAIVDSSFNHTFCAIIYCFHSFNYCFK

SEQ ID NO: 251 - protein, 21 aa, Mycobacterium tuberculosis
VTAICGFADLEALVEAGRTFR

SEQ ID NO: 252 - protein, 24 aa, Mycobacterium tuberculosis
GQHFCIGYHLARLEISVLLQEWFR

SEQ ID NO: 253 - protein, 8 aa, Mycobacterium tuberculosis
LTGADFTR

SEQ ID NO: 254 - protein, 7 aa, Mycobacterium tuberculosis
QALLTGR

SEQ ID NO: 255 - protein, 11 aa, Mycobacterium tuberculosis
MADITGNKQPR

SEQ ID NO: 256 - protein, 23 aa, Mycobacterium tuberculosis
ASVSPLSTAPFASIGSDDAVPSR

SEQ ID NO: 257 - protein, 17 aa, Mycobacterium tuberculosis
MQPGTTTAIVGPSGCGK

SEQ ID NO: 258 - protein, 27 aa, Mycobacterium tuberculosis
AFMTPSIAAALGRWFWWPLNTFNIVQR

SEQ ID NO: 259 - protein, 20 aa, Mycobacterium tuberculosis
LLQMVIMPLVFASILSAVAR

SEQ ID NO: 260 - protein, 19 aa, Mycobacterium tuberculosis
MDPDALVAQLVDEAQNVLR

SEQ ID NO: 261 - protein, 8 aa, Mycobacterium tuberculosis
VSVVSLPR

SEQ ID NO: 262 - protein, 20 aa, Mycobacterium tuberculosis
DFHAGFMSSGDVGYLDENGR

SEQ ID NO: 263 - protein, 22 aa, Mycobacterium tuberculosis
LATVIAAMCAGADCIDDVDVLR

SEQ ID NO: 264 - protein, 9 aa, Mycobacterium tuberculosis
DHPAPLIVR

SEQ ID NO: 265 - protein, 9 aa, Mycobacterium tuberculosis
YLIGCDGGR

SEQ ID NO: 266 - protein, 33 aa, Mycobacterium tuberculosis
TGLSPSDMDVACMYDCFTYTVMATMEGFGFCEK

SEQ ID NO: 267 - protein, 12 aa, Mycobacterium tuberculosis
SVTAAEGDALAR

SEQ ID NO: 268 - protein, 19 aa, Mycobacterium tuberculosis
GARVPALDLEQMQVIYQMR

SEQ ID NO: 269 - protein, 20 aa, Trypanosoma cruzi
YVSMGTNPHPSQSMSPVSMR

SEQ ID NO: 270 - protein, 16 aa, Trypanosoma cruzi
RFVHRDIKPSNILISR

SEQ ID NO: 271 - protein, 30 aa, Trypanosoma cruzi
YAQFMESFSNFDFSIMWYCNAGCLSTDVPR

SEQ ID NO: 272 - protein, 9 aa, Trypanosoma cruzi
HFTGSTAPR

SEQ ID NO: 273 - protein, 10 aa, Trypanosoma cruzi
LHLTGRLIAK

SEQ ID NO: 274 - protein, 12 aa, Trypanosoma cruzi
TAGDAAAAGGPR

SEQ ID NO: 275 - protein, 35 aa, Trypanosoma cruzi
FLACVNDGGHCSLFTQHEFPSEDEFSTDFFNEHGK

SEQ ID NO: 276 - protein, 21 aa, Trypanosoma cruzi
CTVDISTAGSTETNIGDISCR

SEQ ID NO: 277 - protein, 10 aa, Trypanosoma cruzi
AVQAMCRDER

SEQ ID NO: 278 - protein, 11 aa, Trypanosoma cruzi
LIDFVENNAMK

SEQ ID NO: 279 - protein, 9 aa, Trypanosoma cruzi
GAGALEKVK

SEQ ID NO: 280 - protein, 10 aa, Trypanosoma cruzi
NNDGATGATR

SEQ ID NO: 281 - protein, 11 aa, Trypanosoma cruzi
KEAEAENALNR

SEQ ID NO: 282 - protein, 13 aa, Trypanosoma cruzi
RPRLVYSEDSEVR

SEQ ID NO: 283 - protein, 10 aa, Trypanosoma cruzi
IDLLQAELHK

SEQ ID NO: 284 - protein, 13 aa, Trypanosoma cruzi
DSESHAESDRLKR

SEQ ID NO: 285 - protein, 10 aa, Trypanosoma cruzi
MNPEKEKNGR

SEQ ID NO: 286 - protein, 12 aa, Trypanosoma cruzi
QPHERMYCGTIK

SEQ ID NO: 287 - protein, 16 aa, Mycobacterium tuberculosis
EELMLSVWGTGQGVSR

SEQ ID NO: 288 - protein, 12 aa, Mycobacterium tuberculosis
TSNWEYFIGGTK

SEQ ID NO: 289 - protein, 10 aa, Mycobacterium tuberculosis
ELITLEADIR

SEQ ID NO: 290 - protein, 20 aa, Mycobacterium tuberculosis
TVQDGRDKAVDDLATHVMAR

SEQ ID NO: 291 - protein, 12 aa, Mycobacterium tuberculosis
WLQWQLDEQLCR

SEQ ID NO: 292 - protein, 25 aa, Mycobacterium tuberculosis
MLPVNDVNNAGLMAQIPAVLAGPNR

SEQ ID NO: 293 - protein, 15 aa, Mycobacterium tuberculosis
MFGTIQADGLTTLDK

SEQ ID NO: 294 - protein, 9 aa, Mycobacterium tuberculosis
VEEFLDLPR

SEQ ID NO: 295 - protein, 21 aa, Mycobacterium tuberculosis
MSTGIAAMVVQFGLAMMPLGR

SEQ ID NO: 296 - protein, 9 aa, Mycobacterium tuberculosis
IATVVVGVR

SEQ ID NO: 297 - protein, 9 aa, Mycobacterium tuberculosis
NQTLLLQGK

SEQ ID NO: 298 - protein, 9 aa, Mycobacterium tuberculosis
YIDGLTIDR

SEQ ID NO: 299 - protein, 11 aa, Mycobacterium tuberculosis
AWDGGDGALVR

SEQ ID NO: 300 - protein, 17 aa, Mycobacterium tuberculosis
MNPPMTFSVPNTTATNR

SEQ ID NO: 301 - protein, 13 aa, Mycobacterium tuberculosis
VDPQVVVGLLVDR

SEQ ID NO: 302 - protein, 16 aa, Mycobacterium tuberculosis
NSWSMLLLMTSISIEK

SEQ ID NO: 303 - protein, 17 aa, Mycobacterium tuberculosis
SWVIESMPSVAIDGIPK

SEQ ID NO: 304 - protein, 15 aa, Mycobacterium tuberculosis
LETDRAHHNQLVGDR

SEQ ID NO: 305 - protein, 11 aa, Mycobacterium tuberculosis
DELANAQAALR

SEQ ID NO: 306 - protein, 21 aa, Mycobacterium tuberculosis
HPGLKVIVGHMGELLPYCFSR

SEQ ID NO: 307 - protein, 17 aa, Mycobacterium tuberculosis
TLTEGGAGHYVSLLTNR

SEQ ID NO: 308 - protein, 11 aa, Mycobacterium tuberculosis
AEATAADALAR

SEQ ID NO: 309 - protein, 11 aa, Mycobacterium tuberculosis
LDSPDTSNAPR

SEQ ID NO: 310 - protein, 8 aa, Mycobacterium tuberculosis
IGEVIGPK

SEQ ID NO: 311 - protein, 9 aa, Mycobacterium tuberculosis
LGDDDVTPK

SEQ ID NO: 312 - protein, 8 aa, Mycobacterium tuberculosis
ATTTDMLR

SEQ ID NO: 313 - protein, 17 aa, Mycobacterium tuberculosis
IFTEPAGDAAQGTEQRK

SEQ ID NO: 314 - protein, 7 aa, Mycobacterium tuberculosis
VTALETR

SEQ ID NO: 315 - protein, 20 aa, Mycobacterium tuberculosis
QASAWTPMLSASVHLTDDLR

SEQ ID NO: 316 - protein, 7 aa, Mycobacterium tuberculosis
SVALNLR

SEQ ID NO: 317 - protein, 18 aa, Mycobacterium tuberculosis
SLWNSLFNEGEAEGVIMK

SEQ ID NO: 318 - protein, 10 aa, Mycobacterium tuberculosis
EVADAVLWPR

SEQ ID NO: 319 - protein, 7 aa, Mycobacterium tuberculosis
VLNEQLR

SEQ ID NO: 320 - protein, 7 aa, Mycobacterium tuberculosis
LSVAVIR

SEQ ID NO: 321 - protein, 7 aa, Mycobacterium tuberculosis
DPGLVER

SEQ ID NO: 322 - protein, 9 aa, Babesia microti
IDTEEAAVK

SEQ ID NO: 323 - protein, 9 aa, Babesia microti
NAGNFEIIR

SEQ ID NO: 324 - protein, 9 aa, Babesia microti
VTLSDISTK

SEQ ID NO: 325 - protein, 18 aa, Babesia microti
AILKTNGDKTLGAAELEK

SEQ ID NO: 326 - protein, 14 aa, Babesia microti
NNFCLSKSDLEEIR

SEQ ID NO: 327 - protein, 18 aa, Babesia microti
SNQDNQTLLLSLHQAIAK

SEQ ID NO: 328 - protein, 23 aa, Babesia microti
LKNSHAELGVAGNGATTDENAQK

SEQ ID NO: 329 - protein, 22 aa, Babesia microti
GGYLDHVNHAGAKKVILTVPAK

SEQ ID NO: 330 - protein, 10 aa, Babesia microti
LATVNEAEGK

SEQ ID NO: 331 - protein, 10 aa, Babesia microti
LATVNEAEGK

SEQ ID NO: 332 - protein, 11 aa, Babesia microti
NDVSEEKPEIK

SEQ ID NO: 333 - protein, 10 aa, Babesia microti
VVILNEATGK

SEQ ID NO: 334 - protein, 10 aa, Babesia microti
LATVNEAEGK

SEQ ID NO: 335 - protein, 17 aa, Babesia microti
FVYIGNVDNMGYTINFK

SEQ ID NO: 336 - protein, 20 aa, Babesia microti
NLSLFTDFYEISMMNAYFIK

SEQ ID NO: 337 - protein, 16 aa, Babesia microti
QKATGAINAVSGEQIL

SEQ ID NO: 338 - protein, 14 aa, Babesia microti
SAKEVINNTSTMEK

SEQ ID NO: 339 - protein, 16 aa, Babesia microti
SSSVDGFVSFKEYKER

SEQ ID NO: 340 - protein, 10 aa, Babesia microti
FEDAIVLRDK

SEQ ID NO: 341 - protein, 14 aa, Babesia microti
VNESDLGIKALDEK

SEQ ID NO: 342 - protein, 14 aa, Babesia microti
FNVEACFPTLIVEK

SEQ ID NO: 343 - protein, 21 aa, Babesia microti
LKEKHQDLGVANGDTTDNNAK

SEQ ID NO: 344 - protein, 9 aa, Babesia microti
VTSNLDVEK

SEQ ID NO: 345 - protein, 9 aa, Babesia microti
VTSNLDVEK